(19) United States
(12) Patent Application Publication
(10) Pub. No.: US 2025/0145639 A1
(43) Pub. Date: May 8, 2025

(54) MUTANT KRAS MODULATORS AND USES THEREOF

(71) Applicant: NEWAVE PHARMACEUTICAL INC., Pleasanton, CA (US)

(72) Inventor: Yi Chen, Pleasanton, CA (US)

(21) Appl. No.: 18/572,261

(22) PCT Filed: Jun. 22, 2022
(86) PCT No.: PCT/US2022/034523

§ 371 Date: Dec. 20, 2023

Related U.S. Application Data
(60) Provisional application No. 63/300,181, filed on Jan. 17, 2022.
(60) Provisional application No. 63/294,566, filed on Dec. 29, 2021.
(60) Provisional application No. 63/277,939, filed on Nov. 10, 2021.
(60) Provisional application No. 63/214,046, filed on Jun. 23, 2021.

(51) Int. Cl. C07D 519/00; A61K 31/519; C07D 487/04

(52) U.S. Cl. CPC C07D 519/00; A61K 31/519; C07D 487/04

(57) ABSTRACT

The disclosure includes compounds of Formula (1)-(4) wherein each of Warhead, R₁, R₂, R₃, L₁, L₂, L₃, L₄, L₅, L₆, R, Q₁, Q₂, Q₃, W₁, W₂, m, n, and i, are defined herein. Also disclosed is a method for treating a neoplastic disease, autoimmune disease, and inflammatory disorder with these compounds.

### RELATED APPLICATIONS

This International Patent Application claims the benefit of the filing date and priority to U.S. Provisional Application Nos. 63/214,046, filed on Jun. 23, 2021; 63/277,939, filed on Nov. 10, 2021; 63/294,566, filed on Dec. 29, 2021; and 63/300,181, filed on Jan. 17, 2022. The entire contents of each of the foregoing applications are expressly incorporated herein by reference.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma. KRAS G12D mutation is present in 25.0% of all pancreatic ductal adenocarcinoma patients, 13.3% of all colorectal carcinoma patients, 10.1% of all rectal carcinoma patients, 4.1% of all non-small cell lung carcinoma patients and 1.7% of all small cell lung carcinoma patients (e.g., see The AACR Project GENIE Consortium, (2017) Cancer Discovery; 7(8):818-831. Dataset Version 4).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8):1797-1801).

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop modulators of KRas, particularly the activating KRas mutants, such as KRas G12C, G12D, GD12R, and G12V.

### SUMMARY OF THE INVENTION

In a first aspect, this invention relates to a compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof:

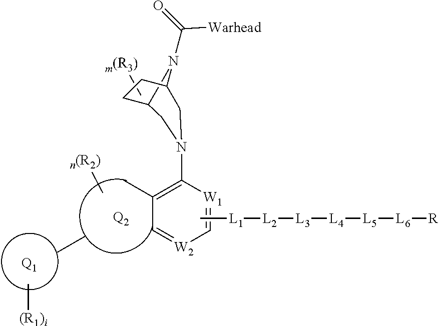

wherein
Warhead is chemical group that can form a covalent bond with a Cysteine of the target protein;
each of Q₁, and Q₂, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
R is R₀ or a small molecule (e.g., with a molecular weight of no more than 1000 Da, 800 Da, 500 Da, 300 Da, or 200 Da) E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}, with the priviso that at least one R₃ is not H;
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

In preferred embodiments, the compound is represented by Formula (1-A):

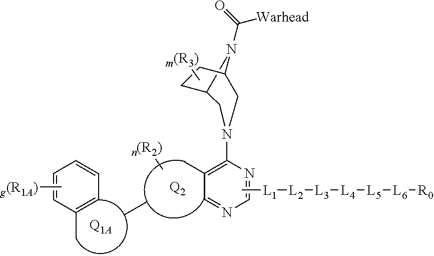

wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁;
Warhead is

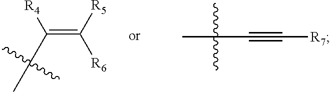

and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.
   The remaining groups are as defined in Formula (1).

In preferred embodiments, the compound is represented by Formula (1-B):

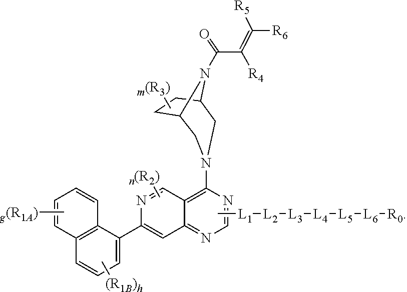

The remaining groups are as defined in Formula (1-A).

In preferred embodiments, the compound is represented by Formula (1-C):

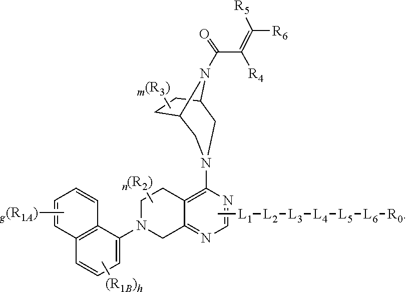

The remaining groups are as defined in Formula (1-A).

In preferred embodiments, the compound is represented by Formula (1-D):

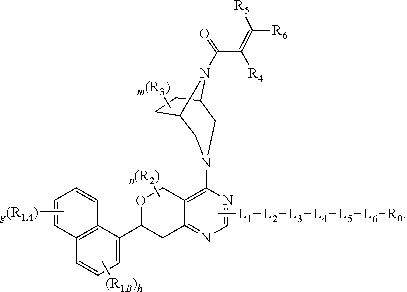

The remaining groups are as defined in Formula (1-A).

In preferred embodiments, the compound is represented by Formula (1-E):

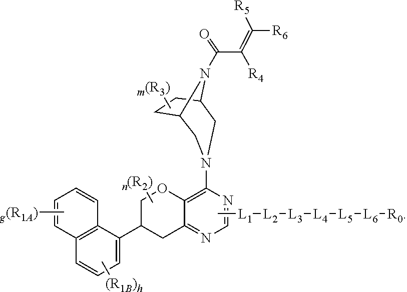

The remaining groups are as defined in Formula (1-A).

In preferred embodiments, the compound is represented by Formula (1-F):

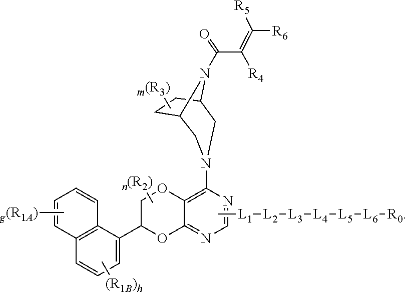

The remaining groups are as defined in Formula (1-A).

In preferred embodiments, the compound is represented by Formula (1-G):

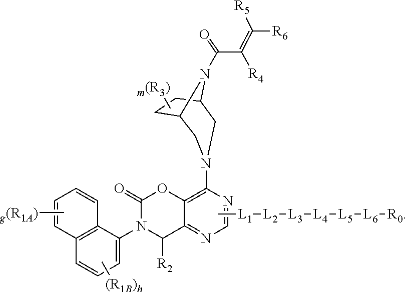

The remaining groups are as defined in Formula (1-A).

In preferred embodiments, the compound is represented by Formula (1-H):

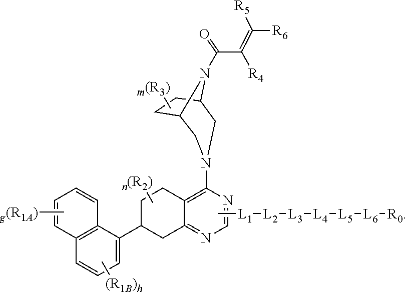

The remaining groups are as defined in Formula (1-A).

In preferred embodiments, the compound is represented by Formula (1-I):

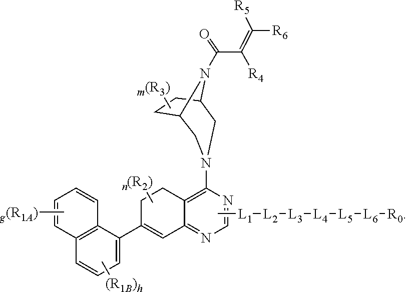

The remaining groups are as defined in Formula (1-A).

In preferred embodiments, the compound is represented by Formula (1-J):

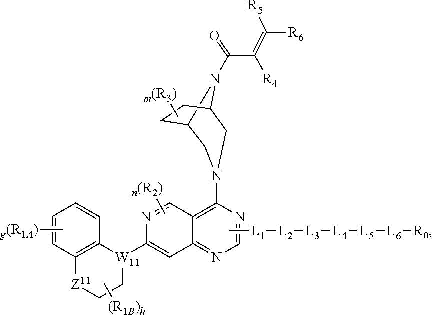

wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.
   The remaining groups are as defined in Formula (1-A).

In preferred embodiments, the compound is represented by Formula (1-K):

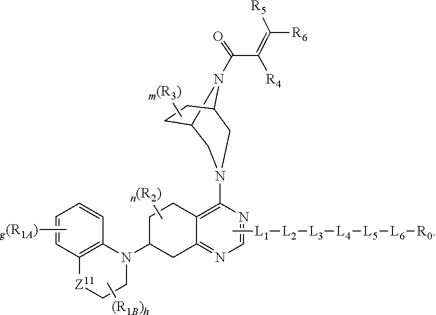

The remaining groups are as defined in Formula (1-A), and Z¹¹ is as defined in Formula (1-J).

In preferred embodiments, the compound is represented by Formula (1-L):

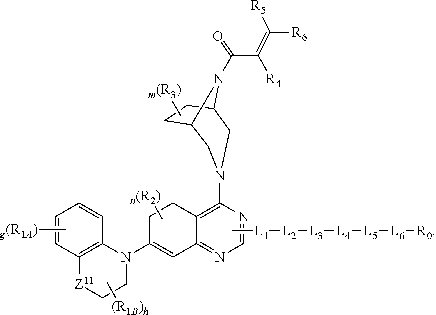

The remaining groups are as defined in Formula (1-A), and Z¹¹ is as defined in Formula (1-J).

In preferred embodiments, the compound is represented by Formula (1-M):

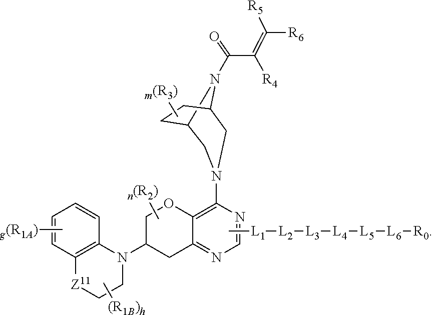

The remaining groups are as defined in Formula (1-A), and Z¹¹ is as defined in Formula (1-J).

In preferred embodiments, the compound is represented by Formula (1-N):

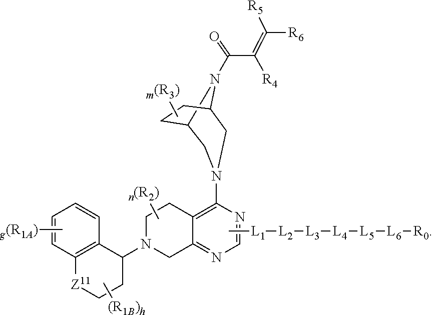

The remaining groups are as defined in Formula (1-A), and Z¹¹ is as defined in Formula (1-J).

In a second aspect, this invention relates to a compound of Formula (2), or an N-oxide thereof, or a pharmaceutically acceptable salt, solvate, polymorph, tautomer, stereoisomer, an isotopic form, or a prodrug of said compound of Formula (2) or N-oxide thereof:

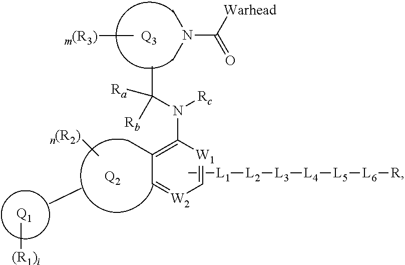

wherein
Warhead is chemical group that can form a covalent bond with a Cysteine of the target protein;
each of Q₁, and Q₂, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
Q₃ is heterocycloalkyl, heterocycloalkenyl, spiro-heterocyclic, fused-heterocyclic, or bridged-heterocyclic, each of the aforementioned is independently optionally substituted with one or more R_{d};
R is R₀ or a small molecule (as defined above) E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c}, and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

In preferred embodiments, the compound is represented by Formula (2-A):

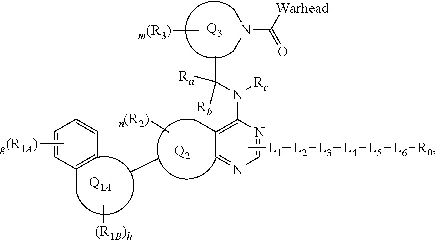

wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is

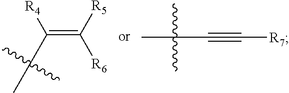

and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.
   The remaining groups are as defined in Formula (2).

In preferred embodiments, the compound is represented by Formula (2-B):

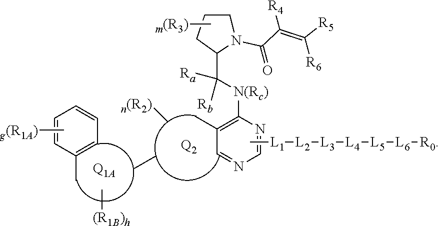

The remaining groups are as defined in Formula (2-A).

In preferred embodiments, the compound is represented by Formula (2-C):

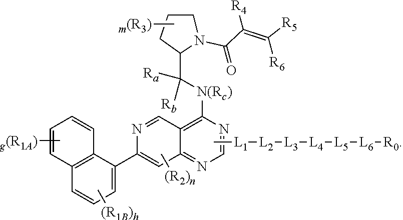

The remaining groups are as defined in Formulae (2-A) and (2-B).

In preferred embodiments, the compound is represented by Formula (2-D):

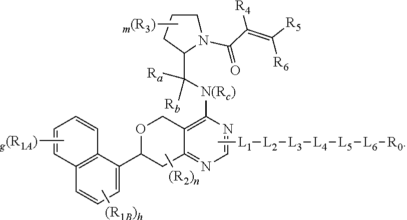

The remaining groups are as defined in Formulae (2-A) and (2-B).

In preferred embodiments, the compound is represented by Formula (2-E):

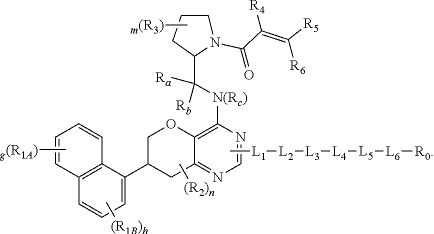

The remaining groups are as defined in Formulae (2-A) and (2-B).

In preferred embodiments, the compound is represented by Formula (2-F):

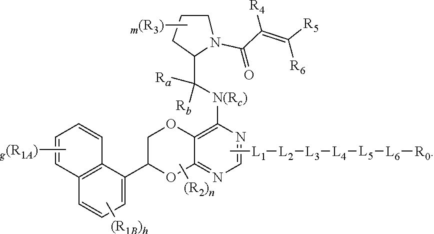

The remaining groups are as defined in Formulae (2-A) and (2-B).

In preferred embodiments, the compound is represented by Formula (2-G):

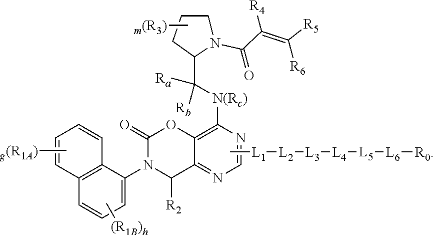

The remaining groups are as defined in Formulae (2-A) and (2-B).

In preferred embodiments, the compound is represented by Formula (2-H):

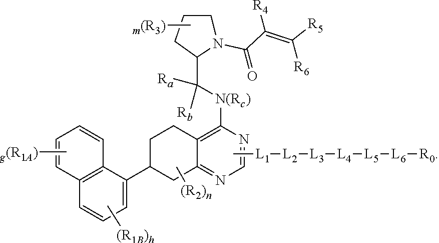

The remaining groups are as defined in Formulae (2-A) and (2-B).

In preferred embodiments, the compound is represented by Formula (2-I):

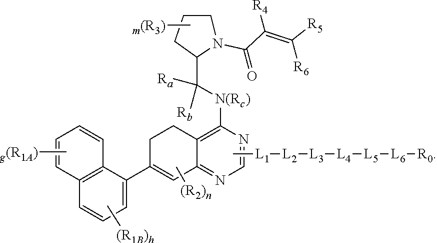

The remaining groups are as defined in Formulae (2-A) and (2-B).

In preferred embodiments, the compound is represented by Formula (2-J):

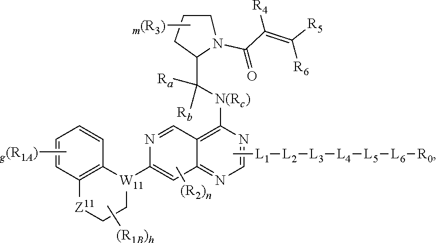

wherein
W₁₁ is N, or C(Rₐ); and
Z¹¹ is absent, a bond, (CRₐR_{b})ₚ, N(R_{c}), O, S, C(O), S(O₂), —O(CRₐR_{b})ₚ—, —N(R_{c})(CRₐR_{b})_{P}, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.
   The remaining groups are as defined in Formulae (2-A) and (2-B).

In preferred embodiments, the compound is represented by Formula (2-K):

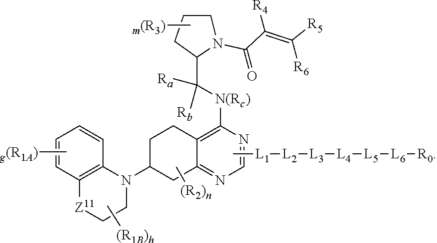

The remaining groups are as defined in Formulae (2-A) and (2-B), and Z¹¹ is as defined in Formula (2-J).

In preferred embodiments, the compound is represented by Formula (2-L):

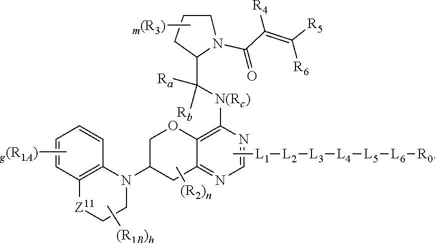

The remaining groups are as defined in Formulae (2-A) and (2-B), and Z¹¹ is as defined in Formula (2-J).

In preferred embodiments, the compound is represented by Formula (2-M):

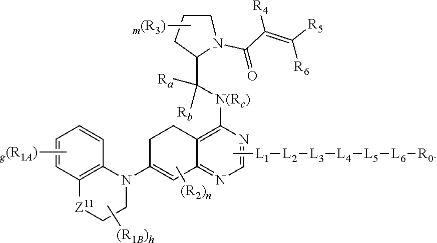

The remaining groups are as defined in Formulae (2-A) and (2-B), and Z¹¹ is as defined in Formula (2-J).

In preferred embodiments, the compound is represented by Formula (2-N):

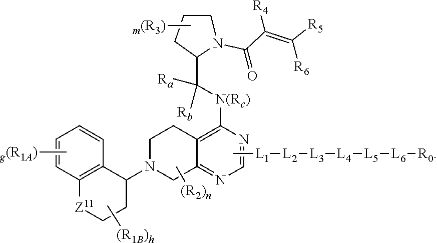

The remaining groups are as defined in Formulae (2-A) and (2-B), and Z¹¹ is as defined in Formula (2-J).

In preferred embodiments, the compound is represented by Formula (2-O):

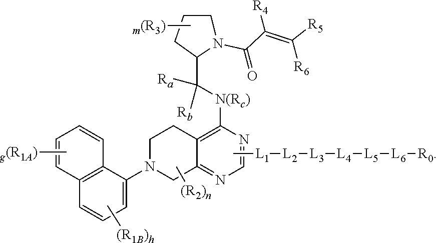

The remaining groups are as defined in Formulae (2-A) and (2-B).

In a third aspect, this invention relates to a compound of Formula (3), or an N-oxide thereof, or a pharmaceutically acceptable salt, solvate, polymorph, tautomer, stereoisomer, an isotopic form, or a prodrug of said compound of Formula (3) or N-oxide thereof:

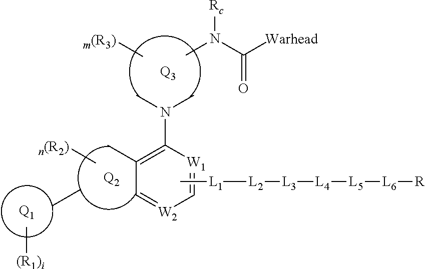

wherein
Warhead is chemical group that can form a covalent bond with a Cysteine of the target protein;
each of Q₁ and Q₂, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
Q₃ is heterocycloalkyl, heterocycloalkenyl, spiro-heterocyclic, fused-heterocyclic, bridged-heterocyclic, or heteroaryl; R is R₀ or a small molecule (as defined above) E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c)}C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

In preferred embodiments, the compound is represented by Formula (3-A):

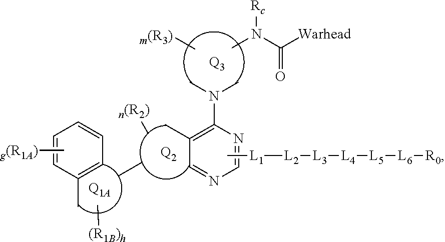

wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is

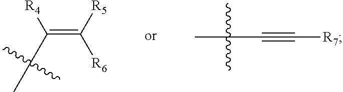

and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.
   The remaining groups are as defined in Formula (3).

In preferred embodiments, the compound is represented by Formula (3-B):

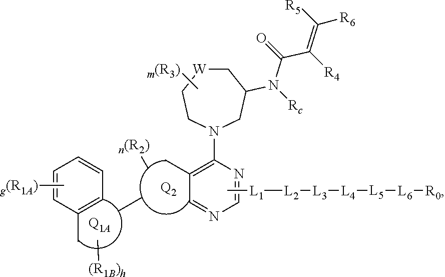

wherein
W is C(RₐR_{b}), N(R_{c}), O, S, or S(O₂).
   The remaining groups are as defined in Formula (3-A).

In preferred embodiments, the compound is represented by Formula (3-C):

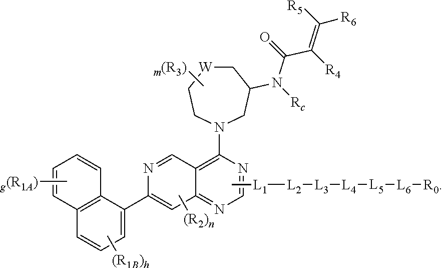

The remaining groups are as defined in Formulae (3-A) and (3-B).

In preferred embodiments, the compound is represented by Formula (3-D):

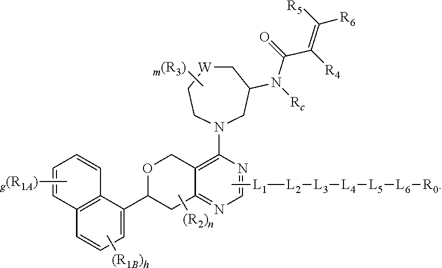

The remaining groups are as defined in Formulae (3-A) and (3-B).

In preferred embodiments, the compound is represented by Formula (3-E):

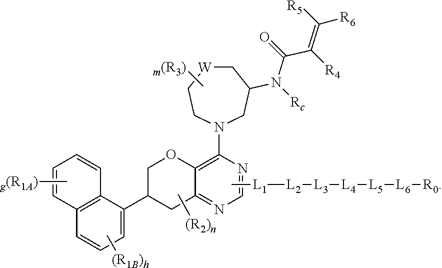

The remaining groups are as defined in Formulae (3-A) and (3-B).

In preferred embodiments, the compound is represented by Formula (3-F):

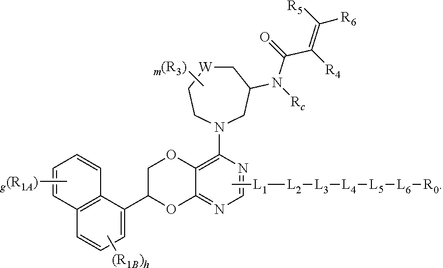

The remaining groups are as defined in Formulae (3-A) and (3-B).

In preferred embodiments, the compound is represented by Formula (3-G):

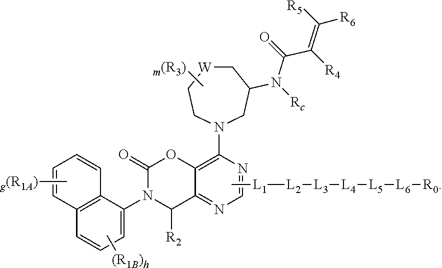

The remaining groups are as defined in Formulae (3-A) and (3-B).

In preferred embodiments, the compound is represented by Formula (3-H):

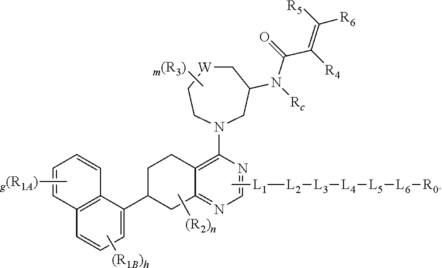

The remaining groups are as defined in Formulae (3-A) and (3-B).

In preferred embodiments, the compound is represented by Formula (3-I):

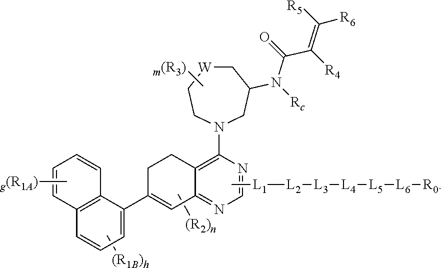

The remaining groups are as defined in Formulae (3-A) and (3-B).

In preferred embodiments, the compound is represented by Formula (3-J):

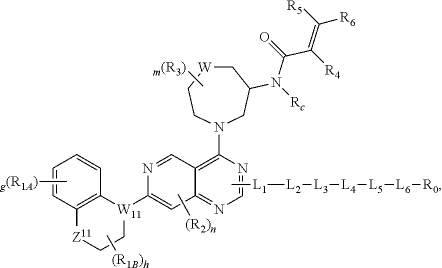

wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.
   The remaining groups are as defined in Formulae (3-A) and (3-B).

In preferred embodiments, the compound is represented by Formula (3-K):

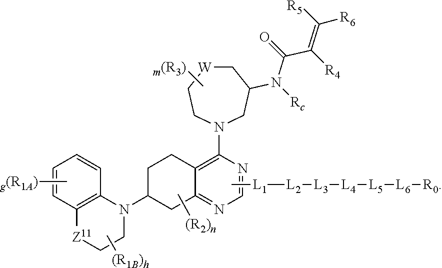

The remaining groups are as defined in Formulae (3-A) and (3-B), and Z¹¹ is as defined in Formula (3-J).

In preferred embodiments, the compound is represented by Formula (3-L):

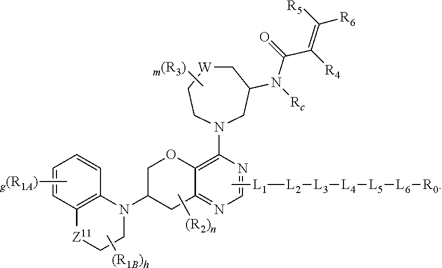

The remaining groups are as defined in Formulae (3-A) and (3-B), and Z¹¹ is as defined in Formula (3-J).

In preferred embodiments, the compound is represented by Formula (3-M):

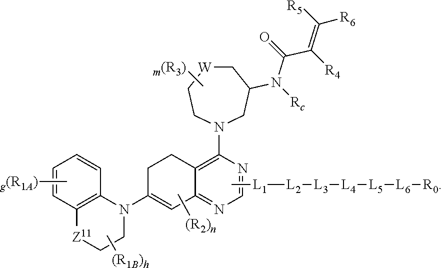

The remaining groups are as defined in Formulae (3-A) and (3-B), and Z¹¹ is as defined in Formula (3-J).

In preferred embodiments, the compound is represented by Formula (3-N):

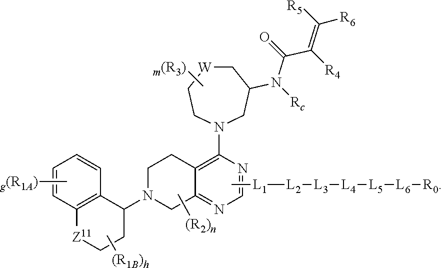

The remaining groups are as defined in Formulae (3-A) and (3-B), and Z¹¹ is as defined in Formula (3-J).

In preferred embodiments, the compound is represented by Formula (3-O):

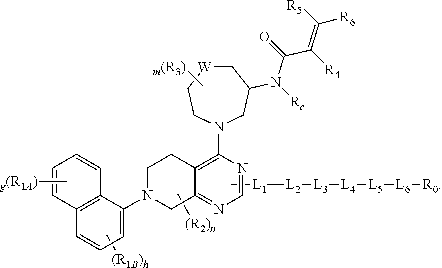

The remaining groups are as defined in Formulae (3-A) and (3-B).

In a fourth aspect, this invention relates to a compound of Formula (4), or an N-oxide thereof, or a pharmaceutically acceptable salt, solvate, polymorph, tautomer, stereoisomer, an isotopic form, or a prodrug of said compound of Formula (4) or N-oxide thereof:

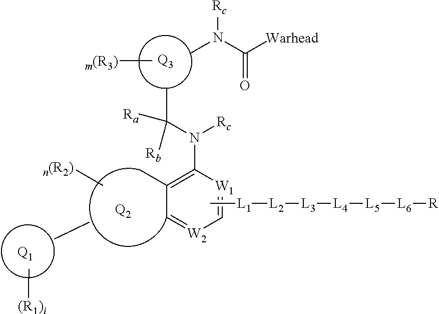

wherein
Warhead is chemical group that can form a covalent bond with a Cysteine of the target protein;
each of Q₁, Q₂ and Q₃, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
R is R₀ or a small molecule (as defined above) E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c)}C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

In preferred embodiments, the compound is represented by Formula (4-A):

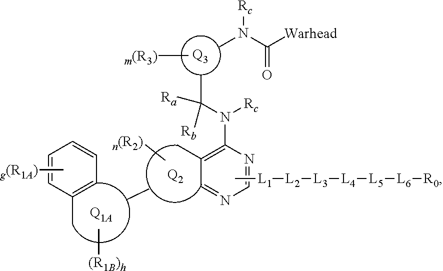

wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is

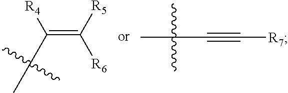

and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.
   The remaining groups are as defined in Formula (4).

In preferred embodiments, the compound is represented by Formula (4-B):

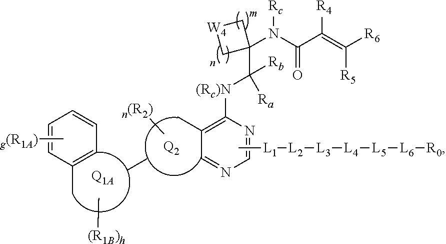

wherein
W₄ is C(RₐR_{b}) or N(Rₐ);
each of m, and n, independently, is 0, 1, 2, or 3.
   The remaining groups are as defined in Formula (4-A).

In preferred embodiments, the compound is represented by Formula (4-C):

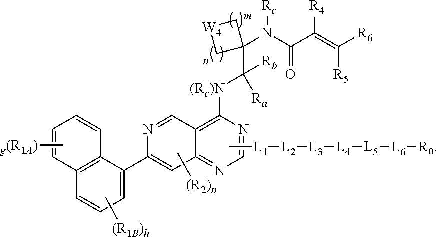

The remaining groups are as defined in Formulae (4-A) and (4-B).

In preferred embodiments, the compound is represented by Formula (4-D):

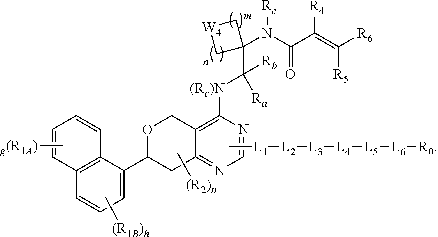

The remaining groups are as defined in Formulae (4-A) and (4-B).

In preferred embodiments, the compound is represented by Formula (4-E):

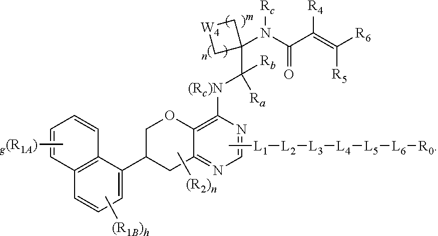

The remaining groups are as defined in Formulae (4-A) and (4-B).

In preferred embodiments, the compound is represented by Formula (4-F):

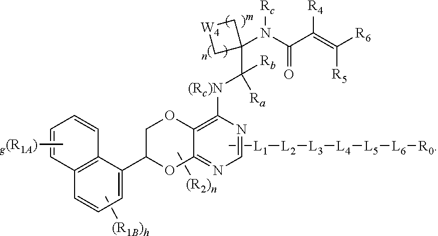

The remaining groups are as defined in Formulae (4-A) and (4-B).

In preferred embodiments, the compound is represented by Formula (4-G):

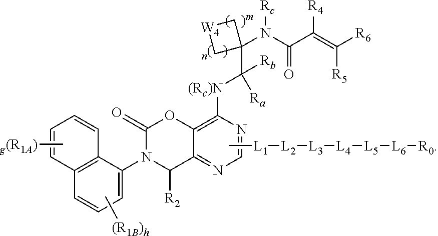

The remaining groups are as defined in Formulae (4-A) and (4-B).

In preferred embodiments, the compound is represented by Formula (4-H):

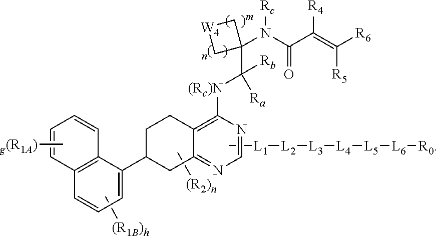

The remaining groups are as defined in Formulae (4-A) and (4-B).

In preferred embodiments, the compound is represented by Formula (4-I):

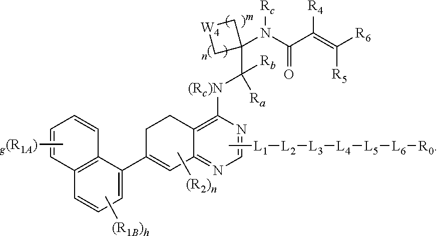

The remaining groups are as defined in Formulae (4-A) and (4-B).

In preferred embodiments, the compound is represented by Formula (4-J):

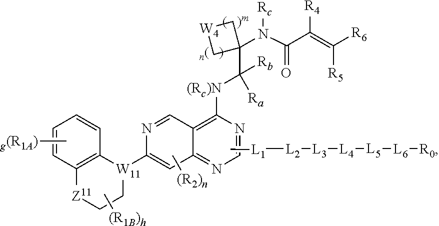

wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.
   The remaining groups are as defined in Formulae (4-A) and (4-B).

In preferred embodiments, the compound is represented by Formula (4-K):

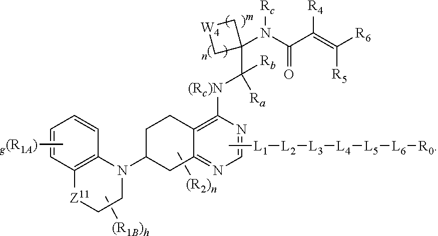

The remaining groups are as defined in Formulae (4-A) and (4-B), and Z¹¹ is as defined in Formula (4-J).

In preferred embodiments, the compound is represented by Formula (4-L):

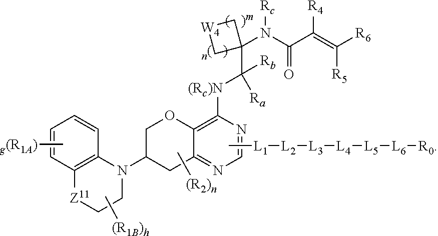

The remaining groups are as defined in Formulae (4-A) and (4-B), and Z¹¹ is as defined in Formula (4-J).

In preferred embodiments, the compound is represented by Formula (4-M):

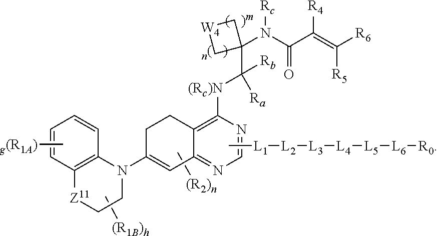

The remaining groups are as defined in Formulae (4-A) and (4-B), and Z¹¹ is as defined in Formula (4-J).

In preferred embodiments, the compound is represented by Formula (4-N):

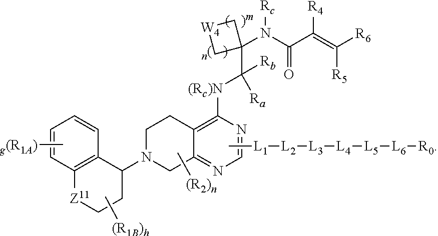

The remaining groups are as defined in Formulae (4-A) and (4-B), and Z¹¹ is as defined in Formula (4-J).

In preferred embodiments, the compound is represented by Formula (4-O):

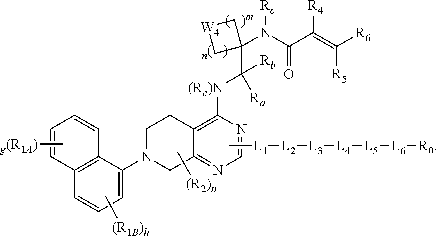

The remaining groups are as defined in Formulae (4-A) and (4-B).

In a fifth aspect, this invention relates to a compound of Formula (5), or an N-oxide thereof, or a pharmaceutically acceptable salt, solvate, polymorph, tautomer, stereoisomer, an isotopic form, or a prodrug of said compound of Formula (5) or N-oxide thereof:

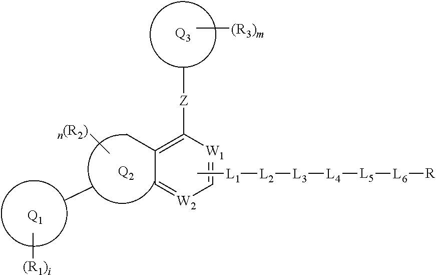

wherein
R is a small molecule (as defined above) E3 ubiquitin ligase binding moiety that binds an E3 ubiquitin ligase;
each of Q₁, Q₂ and Q₃, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

In preferred embodiments, the compound is represented by Formula (5-1):

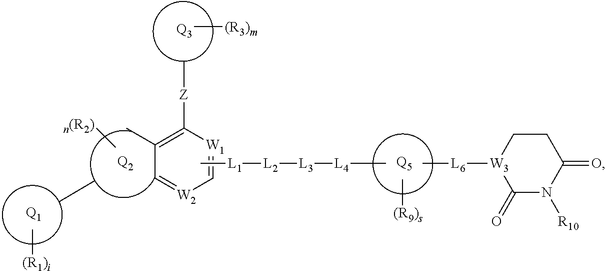

wherein
R₁₀ is H, D, -alkyl-O—P(O)(Rₐ)(R_{b}), or -alkyl-OC(O)—Rₐ;
W₃ is N or CH;
L₆ is absent, NH, CONH, or O;
Q₅ is absent, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
R₉ is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.
R₉ and L₄ groups, taken together with the atom to which they are attached, may optionally form a cycloalkyl or heterocycloalkyl optionally substituted with one or more R_{d}; and
s is 0, 1, 2, 3, or 4.
   The remaining groups are as defined in Formula (5).

In preferred embodiments, the compound is represented by Formula (5-2):

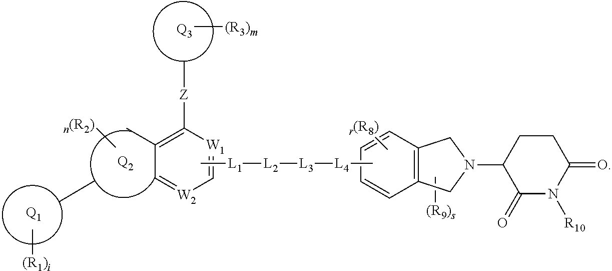

wherein
Rₑ is absent, H, D, alkyl, alkenyl, alkynyl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
R₈ and L₄ groups, taken together with the atom to which they are attached, may optionally form a cycloalkyl or heterocycloalkyl optionally substituted with one or more R_{d}; and
r is 0, 1, 2, 3, or 4.
   The remaining groups are as defined in Formula (5-1).

In preferred embodiment, the compound is represented by Formula (5-A)

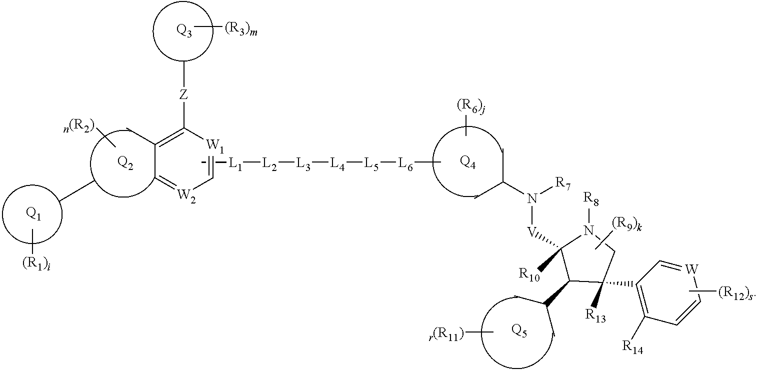

wherein
V is C(O), S(O2), P(O)(Rₐ);
W is CH or N;
each of Q₄, and Q₅, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
each of R₆, R₇, R₈, R₉, R₁₀, R₁₁, R₁₂, R₁₃, and R₁₄, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, heterocycloalkyl, spiroheterocycloalkyl, heterocycloalkenyl, aryl, or heteroaryl, in which said alkyl, spiroalkyl, alkenyl, alkynyl, cycloalkyl, cycloalkenyl, heterocycloalkyl, heterocycloalkenyl, aryl, heteroaryl is optionally substituted with one or more R_{d};
two of R₆ groups, taken together with the atom to which they are attached, may optionally form a cycloalkyl or heterocycloalkyl optionally substituted with one or more R_{d};
two of R₉ groups, taken together with the atom to which they are attached, may optionally form a cycloalkyl or heterocycloalkyl optionally substituted with one or more R_{d};
two of R₁₁ groups, taken together with the atom to which they are attached, may optionally form a cycloalkyl or heterocycloalkyl optionally substituted with one or more R_{d};
two of R₁₂ groups, taken together with the atom to which they are attached, may optionally form a cycloalkyl or heterocycloalkyl optionally substituted with one or more R_{d};
R₁₃ and R₁₄ groups, taken together with the atom to which they are attached, may optionally form a cycloalkyl or heterocycloalkyl optionally substituted with one or more R_{d}; and
each of j, k, r, and s, independently, is 0, 1, 2, 3, 4, 5, or 6.
   The remaining groups are as defined in Formula (5).

In preferred embodiment, the compound is represented by Formula (5-B)

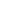

The remaining groups are as defined in Formula (5-A).

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
(1R,5S,6S)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octane-6-carbonitrile,
(1R,5S,6R)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octane-6-carbonitrile,
(1S,5R,6S)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octane-6-carbonitrile,
(1S,5R,6R)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octane-6-carbonitrile,
1-((1R,5R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6,6-difluoro-3,8-diazabicyclo[3.2.1]octan-8-yl)prop-2-en-1-one,
1-((1S,5S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6,6-difluoro-3,8-diazabicyclo[3.2.1]octan-8-yl)prop-2-en-1-one,
1-((1R,5S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6,6-dimethyl-3,8-diazabicyclo[3.2.1]octan-8-yl)prop-2-en-1-one,
1-((1S,5R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6,6-dimethyl-3,8-diazabicyclo[3.2.1]octan-8-yl)prop-2-en-1-one,
1-((1R,5R,6S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-6-methyl-3,8-diazabicyclo[3.2.1]octan-8-yl)prop-2-en-1-one,
1-((1R,5R,6R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-6-methyl-3,8-diazabicyclo[3.2.1]octan-8-yl)prop-2-en-1-one,
1-((1S,5S,6S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-6-methyl-3,8-diazabicyclo[3.2.1]octan-8-yl)prop-2-en-1-one,
1-((1S,5S,6R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-6-methyl-3,8-diazabicyclo[3.2.1]octan-8-yl)prop-2-en-1-one,
(1R,5S,6S)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-6-carbonitrile,
(1R,5S,6R)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-6-carbonitrile,
(1S,5R,6R)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-6-carbonitrile,
(1S,5R,6S)-8-acryloyl-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-6-carbonitrile,
1-((1R,5R,6S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-3,8-diazabicyclo[3.2.1]octan-8-yl)prop-2-en-1-one,
1-((1R,5R,6R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-3,8-diazabicyclo[3.2.1]octan-8-yl)prop-2-en-1-one,
1-((1S,5S,6S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-3,8-diazabicyclo[3.2.1]octan-8-yl)prop-2-en-1-one,
1-((1S,5S,6R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-hydroxy-3,8-diazabicyclo[3.2.1]octan-8-yl)prop-2-en-1-one,
1-((1R,5S,6S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octan-8-yl)prop-2-en-1-one,
1-((1R,5S,6R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octan-8-yl)prop-2-en-1-one,
1-((1S,5R,6R)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octan-8-yl)prop-2-en-1-one,
1-((1S,5R,6S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-6-methyl-3,8-diazabicyclo[3.2.1]octan-8-yl)prop-2-en-1-one,
(1R,5S)-8-acryloyl-3-(7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octane-6-carbonitrile,
(1R,5S)-3-(7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)-8-(2-fluoroacryloyl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1-methylpyrrolidin-2-yl)methoxy)pyrido[4,3-d]pyrimidin-4-yl)-3,8-diazabicyclo[3.2.1]octane-6-carbonitrile,
(1R,5S)-3-(7-(8-ethynyl-7-fluoro-3-hydroxynaphthalen-1-yl)-8-fluoro-2-(((2S,4R)-4-fluoro-1-methylpyrrolidin-2-yl)methoxy)pyrido[4,3-d]pyrimidin-4-yl)-8-(2-fluoroacryloyl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-6-carbonitrile,
(1R,5S)-3-(7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)-8-(2-fluoroacryloyl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1-(4-hydroxy-4-methylcyclohexyl)-2-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1-(4-fluorophenyl)-2-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyridin-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyridin-3-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyridin-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyrimidin-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyrimidin-5-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyrimidin-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(6-(trifluoromethyl)pyridin-3-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-((tetrahydro-2H-pyran-4-yl)methyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1-(4-fluorobenzyl)-2-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyridin-2-ylmethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyridin-3-ylmethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyridin-4-ylmethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyrimidin-4-ylmethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyrimidin-2-ylmethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(pyrimidin-2-ylmethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(1-(tetrahydro-2H-pyran-4-yl)piperidin-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-((1R,4R)-4-((S)-3-methylmorpholino)cyclohexyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-((1S,4S)-4-((S)-3-methylmorpholino)cyclohexyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(2-((R)-3-methylmorpholino)pyridin-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(7-oxaspiro[3.5]nonan-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2R,4R)-4-fluoro-2-isopropyl-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-1-(tetrahydro-2H-pyran-4-yl)-2-(trifluoromethyl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-((S)-1-hydroxyethyl)-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-8-fluoro-2-(((2S,4R)-4-fluoro-2-(2-hydroxypropan-2-yl)-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(2-(((2R,4R)-2-((S)-1-cyanoethyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octane-6-carbonitrile,
(1R,5S)-8-acryloyl-3-(2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octane-6-carbonitrile,
4-(4-((1R,5R)-8-acryloyl-6-hydroxy-6-methyl-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl isobutyrate,
4-(4-((1R,5R)-8-acryloyl-6-hydroxy-6-methyl-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl isopropyl carbonate,
4-(4-((1R,5R)-8-acryloyl-6-hydroxy-6-methyl-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl dimethylcarbamate,
4-(4-((1R,5R)-8-acryloyl-6-hydroxy-6-methyl-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl diisopropyl phosphate,
isopropyl (((4-(4-((1R,5R)-8-acryloyl-6-hydroxy-6-methyl-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl)oxy)(isopropoxy)phosphoryl)-L-alaninate,
isopropyl (((4-(4-((1R,5R)-8-acryloyl-6-hydroxy-6-methyl-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl)oxy)(phenoxy)phosphoryl)-L-alaninate,
(1R,5S)-8-acryloyl-3-(7-(3-cyano-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)-6-methyl-3,8-diazabicyclo[3.2.1]octane-6-carbonitrile,
(1R,5S)-8-acryloyl-3-(7-(8-ethynyl-7-fluoro-3-methoxynaphthalen-1-yl)-8-fluoro-2-(((2S,4R)-4-fluoro-1,2-dimethylpyrrolidin-2-yl)methoxy)pyrido[4,3-d]pyrimidin-4-yl)-6-methyl-3,8-diazabicyclo[3.2.1]octane-6-carbonitrile,
(1R,5S)-8-acryloyl-3-(7-(3-(dimethylphosphoryl)-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)-6-methyl-3,8-diazabicyclo[3.2.1]octane-6-carbonitrile,
(1R,5S)-3-(7-(3-acetyl-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)-8-acryloyl-6-methyl-3,8-diazabicyclo[3.2.1]octane-6-carbonitrile,
(1R,5S)-8-acryloyl-3-(7-(8-ethynyl-7-fluoro-3-((R)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)-6-methyl-3,8-diazabicyclo[3.2.1]octane-6-carbonitrile,
(1R,5S)-8-acryloyl-3-(7-(8-ethynyl-7-fluoro-3-((S)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)-6-methyl-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-methyl-3,8-diazabicyclo[3.2.1]octane-6-carbonitrile,
(1S,5R)-8-acryloyl-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-methyl-3,8-diazabicyclo[3.2.1]octane-6-carbonitrile,
1-((1R,5R)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6,6-difluoro-3,8-diazabicyclo[3.2.1]octan-8-yl)prop-2-en-1-one,
1-((1S,5S)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6,6-difluoro-3,8-diazabicyclo[3.2.1]octan-8-yl)prop-2-en-1-one,
1-((1R,5S)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6,6-dimethyl-3,8-diazabicyclo[3.2.1]octan-8-yl)prop-2-en-1-one,
1-((1S,5R)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6,6-dimethyl-3,8-diazabicyclo[3.2.1]octan-8-yl)prop-2-en-1-one,
1-((1R,5R)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-hydroxy-6-methyl-3,8-diazabicyclo[3.2.1]octan-8-yl)prop-2-en-1-one,
1-((1S,5S)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-hydroxy-6-methyl-3,8-diazabicyclo[3.2.1]octan-8-yl)prop-2-en-1-one,
(1R,5S)-8-acryloyl-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-3,8-diazabicyclo[3.2.1]octane-6-carbonitrile,
(1S,5R)-8-acryloyl-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-3,8-diazabicyclo[3.2.1]octane-6-carbonitrile,
1-((1R,5R)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
1-((1S,5S)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
1-((1R,5S)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-methyl-3,8-diazabicyclo[3.2.1]octan-8-yl)prop-2-en-1-one,
1-((1S,5R)-3-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-6-methyl-3,8-diazabicyclo[3.2.1]octan-8-yl)prop-2-en-1-one,
(1R,5S)-8-acryloyl-3-(7-(8-ethynyl-7-fluoro-3-hydroxynaphthalen-1-yl)-2-(((S)-1-methylpyrrolidin-2-yl)methoxy)-5,6,7,8-tetrahydropyrido[3,4-d]pyrimidin-4-yl)-3,8-diazabicyclo[3.2.1]octane-6-carbonitrile,
1-((1R,5S)-3-(7-(8-ethynyl-7-fluoro-3-hydroxynaphthalen-1-yl)-2-(((2S,4R)-4-fluoro-1-methylpyrrolidin-2-yl)methoxy)-5,6,7,8-tetrahydropyrido[3,4-d]pyrimidin-4-yl)-6-methyl-3,8-diazabicyclo[3.2.1]octan-8-yl)prop-2-en-1-one,
1-((1R,5R)-3-(7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
(1R,5S)-8-acryloyl-3-(7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
1-((1S,5S)-3-(7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
1-((1S,5R)-3-(7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)-6-methyl-3,8-diazabicyclo[3.2.1]octan-8-yl)prop-2-en-1-one,
(1R,5S)-8-acryloyl-3-(7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
1-((1R,5R)-3-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
1-((1R,5S)-3-(7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6,7,8-tetrahydropyrido[3,4-d]pyrimidin-4-yl)-6-methyl-3,8-diazabicyclo[3.2.1]octan-8-yl)prop-2-en-1-one,
(1R,5S)-8-acryloyl-3-(7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
1-((1R,5R)-3-(7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
1-((1R,5S)-3-(2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6,7,8-tetrahydropyrido[3,4-d]pyrimidin-4-yl)-6-methyl-3,8-diazabicyclo[3.2.1]octan-8-yl)prop-2-en-1-one,
1-((1S,5S)-3-(7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-5,6,7,8-tetrahydropyrido[3,4-d]pyrimidin-4-yl)-6-hydroxy-3,8-diazabicyclo[3.2.1]octan-8-yl)prop-2-en-1-one,
(1S,5R)-8-acryloyl-3-(7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6,7,8-tetrahydropyrido[3,4-d]pyrimidin-4-yl)-3,8-diazabicyclo[3.2.1]octane-6-carbonitrile,
2-((2R,4R)-2-(((4-((1S,5R)-8-acryloyl-6-methyl-3,8-diazabicyclo[3.2.1]octan-3-yl)-7-(8-ethynyl-7-fluoro-3-hydroxynaphthalen-1-yl)-5,6,7,8-tetrahydropyrido[3,4-d]pyrimidin-2-yl)oxy)methyl)-4-fluoro-1-(tetrahydro-2H-pyran-4-yl)pyrrolidin-2-yl)-2-methylpropanenitrile,
4-(4-((1R,5R)-8-acryloyl-6-hydroxy-6-methyl-3,8-diazabicyclo[3.2.1]octan-3-yl)-2-(((2S,4R)-4-fluoro-1,2-dimethyl pyrrolidin-2-yl)methoxy)-5,8-dihydropyrido[3,4-d]pyrimidin-7(6H)-yl)-5-ethynyl-6-fluoronaphthalen-2-yl isobutyrate,
4-(4-((1R,5S)-8-acryloyl-6,6-dimethyl-3,8-diazabicyclo[3.2.1]octan-3-yl)-2-(((2S,4R)-4-fluoro-1,2-dimethyl pyrrolidin-2-yl)methoxy)-5,8-dihydropyrido[3,4-d]pyrimidin-7(6H)-yl)-5-ethynyl-6-fluoronaphthalen-2-yl isopropyl carbonate,
isopropyl (((4-(4-((1R,5S)-8-acryloyl-6-cyano-6-methyl-3,8-diazabicyclo[3.2.1]octan-3-yl)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl)oxy)(isopropoxy)phosphoryl)-L-alaninate,
1-((1R,5R)-3-((S)-7-(8-ethynyl-7-fluoro-3-hydroxynaphthalen-1-yl)-2-((tetrahydro-1H-pyrrolizin-7a(5H)-yl)methoxy)-7,8-dihydro-5H-pyrano[4,3-d]pyrimidin-4-yl)-6,6-difluoro-3,8-diazabicyclo[3.2.1]octan-8-yl)prop-2-en-1-one,
1-((1R,5R)-3-((R)-7-(8-ethynyl-7-fluoro-3-hydroxynaphthalen-1-yl)-2-((tetrahydro-1H-pyrrolizin-7a(5H)-yl)methoxy)-7,8-dihydro-5H-pyrano[4,3-d]pyrimidin-4-yl)-6,6-difluoro-3,8-diazabicyclo[3.2.1]octan-8-yl)prop-2-en-1-one,
1-((1S,5S)-3-((S)-7-(8-ethynyl-7-fluoro-3-hydroxynaphthalen-1-yl)-2-((tetrahydro-1H-pyrrolizin-7a(5H)-yl)methoxy)-7,8-dihydro-5H-pyrano[4,3-d]pyrimidin-4-yl)-6,6-difluoro-3,8-diazabicyclo[3.2.1]octan-8-yl)prop-2-en-1-one,
1-((1S,5S)-3-((R)-7-(8-ethynyl-7-fluoro-3-hydroxynaphthalen-1-yl)-2-((tetrahydro-1H-pyrrolizin-7a(5H)-yl)methoxy)-7,8-dihydro-5H-pyrano[4,3-d]pyrimidin-4-yl)-6,6-difluoro-3,8-diazabicyclo[3.2.1]octan-8-yl)prop-2-en-1-one,
1-((1R,5S)-3-((R)-7-(8-ethynyl-7-fluoro-3-hydroxynaphthalen-1-yl)-2-((tetrahydro-1H-pyrrolizin-7a(5H)-yl)methoxy)-7,8-dihydro-6H-pyrano[3,2-d]pyrimidin-4-yl)-6,6-dimethyl-3,8-diazabicyclo[3.2.1]octan-8-yl)prop-2-en-1-one,
1-((1R,5S)-3-((S)-7-(8-ethynyl-7-fluoro-3-hydroxynaphthalen-1-yl)-2-((tetrahydro-1H-pyrrolizin-7a(5H)-yl)methoxy)-7,8-dihydro-6H-pyrano[3,2-d]pyrimidin-4-yl)-6,6-dimethyl-3,8-diazabicyclo[3.2.1]octan-8-yl)prop-2-en-1-one,
1-((1S,5R)-3-((R)-7-(8-ethynyl-7-fluoro-3-hydroxynaphthalen-1-yl)-2-((tetrahydro-1H-pyrrolizin-7a(5H)-yl)methoxy)-7,8-dihydro-6H-pyrano[3,2-d]pyrimidin-4-yl)-6,6-dimethyl-3,8-diazabicyclo[3.2.1]octan-8-yl)prop-2-en-1-one,
1-((1S,5R)-3-((S)-7-(8-ethynyl-7-fluoro-3-hydroxynaphthalen-1-yl)-2-((tetrahydro-1H-pyrrolizin-7a(5H)-yl)methoxy)-7,8-dihydro-6H-pyrano[3,2-d]pyrimidin-4-yl)-6,6-dimethyl-3,8-diazabicyclo[3.2.1]octan-8-yl)prop-2-en-1-one,
1-((1R,5R,6S)-3-(7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)-6-hydroxy-6-methyl-3,8-diazabicyclo[3.2.1]octan-8-yl)prop-2-en-1-one,
1-((1R,5R,6R)-3-(7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)-6-hydroxy-6-methyl-3,8-diazabicyclo[3.2.1]octan-8-yl)prop-2-en-1-one,
1-((1S,5S,6S)-3-(7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)-6-hydroxy-6-methyl-3,8-diazabicyclo[3.2.1]octan-8-yl)prop-2-en-1-one,
1-((1S,5S,6R)-3-(7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)-6-hydroxy-6-methyl-3,8-diazabicyclo[3.2.1]octan-8-yl)prop-2-en-1-one,
(1R,5S,6S)-8-acryloyl-3-(3-(8-ethynyl-7-fluoro-3-hydroxynaphthalen-1-yl)-2-oxo-6-((tetrahydro-1H-pyrrolizin-7a(5H)-yl)methoxy)-3,4-dihydro-2H-pyrimido[4,5-e][1,3]oxazin-8-yl)-3,8-diazabicyclo[3.2.1]octane-6-carbonitrile,
(1R,5S,6R)-8-acryloyl-3-(3-(8-ethynyl-7-fluoro-3-hydroxynaphthalen-1-yl)-2-oxo-6-((tetrahydro-1H-pyrrolizin-7a(5H)-yl)methoxy)-3,4-dihydro-2H-pyrimido[4,5-e][1,3]oxazin-8-yl)-3,8-diazabicyclo[3.2.1]octane-6-carbonitrile,
(1S,5R,6R)-8-acryloyl-3-(3-(8-ethynyl-7-fluoro-3-hydroxynaphthalen-1-yl)-2-oxo-6-((tetrahydro-1H-pyrrolizin-7a(5H)-yl)methoxy)-3,4-dihydro-2H-pyrimido[4,5-e][1,3]oxazin-8-yl)-3,8-diazabicyclo[3.2.1]octane-6-carbonitrile,
(1S,5R,6S)-8-acryloyl-3-(3-(8-ethynyl-7-fluoro-3-hydroxynaphthalen-1-yl)-2-oxo-6-((tetrahydro-1H-pyrrolizin-7a(5H)-yl)methoxy)-3,4-dihydro-2H-pyrimido[4,5-e][1,3]oxazin-8-yl)-3,8-diazabicyclo[3.2.1]octane-6-carbonitrile,
1-((1R,5R,6S)-3-((R)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R,6S)-3-((S)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R,6R)-3-((R)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R,6R)-3-((S)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1S,5S,6S)-3-((R)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1S,5S,6S)-3-((S)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1S,5S,6R)-3-((R)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1S,5S,6R)-3-((S)-7-(8-ethynyl-7-fluoro-3-hydroxynaphthalen-1-yl)-2-((tetrahydro-1H-pyrrolizin-7a(5H)-yl)methoxy)-5,6,7,8-tetrahydroquinazolin-4-yl)-6-hydroxy-3,8-diazabicyclo[3.2.1]octan-8-yl)prop-2-en-1-one,
1-((1R,5S,6S)-3-(7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)-6-methyl-3,8-diazabicyclo[3.2.1]octan-8-yl)prop-2-en-1-one,
1-((1R,5S,6R)-3-(7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)-6-methyl-3,8-diazabicyclo[3.2.1]octan-8-yl)prop-2-en-1-one,
1-((1S,5R,6R)-3-(7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)-6-methyl-3,8-diazabicyclo[3.2.1]octan-8-yl)prop-2-en-1-one,
1-((1S,5R,6S)-3-(7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)-6-methyl-3,8-diazabicyclo[3.2.1]octan-8-yl)prop-2-en-1-one,
(1R,5S)-8-acryloyl-3-(7-(8-ethynyl-7-fluoro-3,4-dihydroquinolin-1(2H)-yl)-8-fluoro-2-(((S)-1-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-(5-ethynyl-6-fluoro-2,3-dihydro-4H-benzo[b][1,4]oxazin-4-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((S)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((R)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((R)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((S)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((R)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((S)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1S,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1R,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1S,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1R,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1R,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1R,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1S,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)-3,8-diazabicyclo[3.2.1]octane-6-carbonitrile,
(1R,5S)-8-acryloyl-3-(7-((1S,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)-3,8-diazabicyclo[3.2.1]octane-6-carbonitrile,
1-((1R,5R)-3-((R)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((S)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((R)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((S)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((R)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((R)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((S)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((S)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((R)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((R)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((S)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5R)-3-((S)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)-6-hydroxy-3,8-diazabicyclo[3.2.1]octan-8-yl)prop-2-en-1-one,
1-((1R,5S)-3-(7-(8-ethynyl-7-fluoro-3,4-dihydroquinolin-1(2H)-yl)-2-(((2S,4R)-4-fluoro-1-(4-fluorophenyl)-2-methylpyrrolidin-2-yl)methoxy)-5,6-dihydroquinazolin-4-yl)-6-methyl-3,8-diazabicyclo[3.2.1]octan-8-yl)prop-2-en-1-one,
1-((1R,5S)-3-(7-(5-ethynyl-6-fluoro-2,3-dihydro-4H-benzo[b][1,4]oxazin-4-yl)-2-(((2S,4R)-4-fluoro-1-(4-fluorophenyl)-2-methylpyrrolidin-2-yl)methoxy)-5,6-dihydroquinazolin-4-yl)-6-methyl-3,8-diazabicyclo[3.2.1]octan-8-yl)prop-2-en-1-one,
1-((1R,5S)-3-(7-((R)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)-6-methyl-3,8-diazabicyclo[3.2.1]octan-8-yl)prop-2-en-1-one,
1-((1R,5S)-3-(7-((S)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)-6-methyl-3,8-diazabicyclo[3.2.1]octan-8-yl)prop-2-en-1-one,
1-((1R,5S)-3-(7-((R)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)-6-methyl-3,8-diazabicyclo[3.2.1]octan-8-yl)prop-2-en-1-one,
1-((1R,5S)-3-(7-((S)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)-6-methyl-3,8-diazabicyclo[3.2.1]octan-8-yl)prop-2-en-1-one,
(1R,5S)-8-acryloyl-3-((S)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((R)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((S)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((R)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((R)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((R)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((S)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((S)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((R)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((R)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((S)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-((S)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)-3,8-diazabicyclo[3.2.1]octane-6-carbonitrile,
(1R,5S)-8-acryloyl-3-(7-((R)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((S)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((S)-5-ethynyl-6-fluorochroman-4-yl)-2-(((2S,4R)-4-fluoro-2-methyl-1-(pyridin-2-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((R)-5-ethynyl-6-fluorochroman-4-yl)-2-(((2S,4R)-4-fluoro-2-methyl-1-(pyridin-2-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1S,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1S,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1R,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1R,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1S,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1S,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1R,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(1R,5S)-8-acryloyl-3-(7-((1R,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)-3,8-diazabicyclo[3.2.1]octane-6-carbonitrile,
(S)-1-(2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)pyrrolidin-1-yl)prop-2-en-1-one,
(S)-1-(2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)pyrrolidin-1-yl)-2-fluoroprop-2-en-1-one,
1-((2S)-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidin-1-yl)prop-2-en-1-one,
1-((2R)-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-hydroxypyrrolidin-1-yl)prop-2-en-1-one,
(2S)-1-acryloyl-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)pyrrolidine-3-carbonitrile,
(R)-1-(2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-difluoropyrrolidin-1-yl)prop-2-en-1-one,
(S)-1-(2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-dimethylpyrrolidin-1-yl)prop-2-en-1-one,
(2S)-1-acryloyl-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
1-((2R)-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-hydroxy-3-methylpyrrolidin-1-yl)prop-2-en-1-one,
1-((2S)-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-4-methylpyrrolidin-1-yl)prop-2-en-1-one,
1-((2S)-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-4-hydroxypyrrolidin-1-yl)prop-2-en-1-one,
(5S)-1-acryloyl-5-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)pyrrolidine-3-carbonitrile,
(S)-1-(2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-4,4-difluoropyrrolidin-1-yl)prop-2-en-1-one,
(S)-1-(2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-4,4-dimethylpyrrolidin-1-yl)prop-2-en-1-one,
1-((2S)-2-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-4-hydroxy-4-methylpyrrolidin-1-yl)prop-2-en-1-one,
(5S)-1-acryloyl-5-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
1-((S)-2-(((7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)(methyl)amino)methyl)pyrrolidin-1-yl)prop-2-en-1-one,
(2S)-1-acryloyl-2-(((7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)(methyl)amino)methyl)-3-methylpyrrolidine-3-carbonitrile,
1-((S)-2-(((7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)(methyl)amino)methyl)-3,3-dimethylpyrrolidin-1-yl)prop-2-en-1-one,
1-((2R)-2-(((7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)(methyl)amino)methyl)-3-hydroxy-3-methylpyrrolidin-1-yl)prop-2-en-1-one,
1-((R)-2-(((7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)(methyl)amino)methyl)-3,3-difluoropyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1-(4-hydroxy-4-methylcyclohexyl)-2-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1-(4-fluorophenyl)-2-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyridin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyridin-3-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyridin-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyrimidin-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyrimidin-5-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyrimidin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(6-(trifluoromethyl)pyridin-3-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-((tetrahydro-2H-pyran-4-yl)methyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1-(4-fluorobenzyl)-2-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyridin-2-ylmethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyridin-3-ylmethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyridin-4-ylmethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyrimidin-4-ylmethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyrimidin-2-ylmethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(pyrimidin-5-ylmethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(1-(tetrahydro-2H-pyran-4-yl)piperidin-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-((1R,4R)-4-((S)-3-methylmorpholino)cyclohexyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-((1S,4S)-4-((S)-3-methylmorpholino)cyclohexyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(2-((R)-3-methylmorpholino)pyridin-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(7-oxaspiro[3.5]nonan-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2R,4R)-4-fluoro-2-isopropyl-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-1-(tetrahydro-2H-pyran-4-yl)-2-(trifluoromethyl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-((S)-1-hydroxyethyl)-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-8-fluoro-2-(((2S,4R)-4-fluoro-2-(2-hydroxypropan-2-yl)-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
(S)-2-((2R,4R)-2-(((4-((((S)-1-acryloylpyrrolidin-2-yl)methyl)(methyl)amino)-7-(8-ethynyl-7-fluoro-3-hydroxynaphthalen-1-yl)-8-fluoropyrido[4,3-d]pyrimidin-2-yl)oxy)methyl)-4-fluoro-1-(tetrahydro-2H-pyran-4-yl)pyrrolidin-2-yl)propanenitrile,
2-((2R,4R)-2-(((4-((((S)-1-acryloylpyrrolidin-2-yl)methyl)(methyl)amino)-7-(8-ethynyl-7-fluoro-3-hydroxynaphthalen-1-yl)-8-fluoropyrido[4,3-d]pyrimidin-2-yl)oxy)methyl)-4-fluoro-1-(tetrahydro-2H-pyran-4-yl)pyrrolidin-2-yl)-2-methylpropanenitrile,
4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl isobutyrate,
4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl isopropyl carbonate,
4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl dimethylcarbamate,
4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl diisopropyl phosphate,
A name could not be generated for this structure,
isopropyl (((4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl)oxy)(isopropoxy)phosphoryl)-L-alaninate,
isopropyl (((4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl)oxy)(phenoxy)phosphoryl)-L-alaninate,
4-(4-((((S)-1-acryloylpyrrolidin-2-yl)methyl)(methyl)amino)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoro-2-naphthonitrile,
1-((S)-2-(((7-(8-ethynyl-7-fluoro-3-methoxynaphthalen-1-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(3-(dimethylphosphoryl)-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(3-acetyl-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-((R)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoro-3-((S)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
(S)-1-(2-(((7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((S)-1-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
2-((2R,4R)-2-(((4-((((S)-1-acryloylpyrrolidin-2-yl)methyl)(methyl)amino)-7-(8-ethynyl-7-fluoro-3-hydroxynaphthalen-1-yl)-5,6,7,8-tetrahydropyrido[3,4-d]pyrimidin-2-yl)oxy)methyl)-4-fluoro-1-(tetrahydro-2H-pyran-4-yl)pyrrolidin-2-yl)-2-methylpropanenitrile,
4-(4-((((S)-1-acryloylpyrrolidin-2-yl)methyl)(methyl)amino)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl isobutyrate,
4-(4-((((S)-1-acryloylpyrrolidin-2-yl)methyl)(methyl)amino)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl isopropyl carbonate,
isopropyl (((4-(4-((((S)-1-acryloylpyrrolidin-2-yl)methyl)(methyl)amino)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl)oxy)(isopropoxy)phosphoryl)-L-alaninate,
1-((S)-2-((((R)-7-(8-ethynyl-7-fluoro-3-hydroxynaphthalen-1-yl)-2-(((2S,4R)-4-fluoro-1,2-dimethylpyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-1,2-dimethylpyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-7,8-dihydro-6H-pyrano[3,2-d]pyrimidin-4-yl)(methyl)amino)methyl)pyrrolidin-1-yl)prop-2-en-1-one,
1-((S)-2-((((R)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
4-((R)-4-((((S)-1-acryloylpyrrolidin-2-yl)methyl)(methyl)amino)-2-(((2S,4R)-4-fluoro-1,2-dimethyl pyrrolidin-2-yl)methoxy)-7,8-dihydro-6H-pyrano[3,2-d]pyrimidin-7-yl)-5-ethynyl-6-fluoronaphthalen-2-yl isobutyrate,
4-((S)-4-((((S)-1-acryloylpyrrolidin-2-yl)methyl)(methyl)amino)-2-(((2S,4R)-4-fluoro-1,2-dimethyl pyrrolidin-2-yl)methoxy)-7,8-dihydro-6H-pyrano[3,2-d]pyrimidin-7-yl)-5-ethynyl-6-fluoronaphthalen-2-yl isobutyrate,
isopropyl (((4-((S)-4-((((S)-1-acryloylpyrrolidin-2-yl)methyl)(methyl)amino)-2-(((2S,4R)-4-fluoro-1,2-dimethylpyrrolidin-2-yl)methoxy)-7,8-dihydro-6H-pyrano[3,2-d]pyrimidin-7-yl)-5-ethynyl-6-fluoronaphthalen-2-yl)oxy)(isopropoxy)phosphoryl)-L-alaninate,
isopropyl (((4-((R)-4-((((S)-1-acryloylpyrrolidin-2-yl)methyl)(methyl)amino)-2-(((2S,4R)-4-fluoro-1,2-dimethylpyrrolidin-2-yl)methoxy)-7,8-dihydro-6H-pyrano[3,2-d]pyrimidin-7-yl)-5-ethynyl-6-fluoronaphthalen-2-yl)oxy)(isopropoxy)phosphoryl)-L-alaninate,
1-((2S)-2-(((7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((S)-1-methylpyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-1-methylpyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-6,7-dihydro-[1,4]dioxino[2,3-d]pyrimidin-4-yl)(methyl)amino)methyl)pyrrolidin-1-yl)prop-2-en-1-one,
2-((2R,4R)-2-(((4-((((S)-1-acryloylpyrrolidin-2-yl)methyl)(methyl)amino)-7-(8-ethynyl-7-fluoro-3-hydroxynaphthalen-1-yl)-6,7-dihydro-[1,4]dioxino[2,3-d]pyrimidin-2-yl)oxy)methyl)-4-fluoro-1-(tetrahydro-2H-pyran-4-yl)pyrrolidin-2-yl)-2-methylpropanenitrile,
4-(4-((((S)-1-acryloylpyrrolidin-2-yl)methyl)(methyl)amino)-2-(((2S,4R)-4-fluoro-1,2-dimethylpyrrolidin-2-yl)methoxy)-6,7-dihydro-[1,4]dioxino[2,3-d]pyrimidin-7-yl)-5-ethynyl-6-fluoronaphthalen-2-yl isobutyrate,
4-(4-((((S)-1-acryloylpyrrolidin-2-yl)methyl)(methyl)amino)-2-(((2S,4R)-4-fluoro-1,2-dimethylpyrrolidin-2-yl)methoxy)-6,7-dihydro-[1,4]dioxino[2,3-d]pyrimidin-7-yl)-5-ethynyl-6-fluoronaphthalen-2-yl isopropyl carbonate,
isopropyl (((4-(4-((((S)-1-acryloylpyrrolidin-2-yl)methyl)(methyl)amino)-2-(((2S,4R)-4-fluoro-1,2-dimethylpyrrolidin-2-yl)methoxy)-6,7-dihydro-[1,4]dioxino[2,3-d]pyrimidin-7-yl)-5-ethynyl-6-fluoronaphthalen-2-yl)oxy)(isopropoxy)phosphoryl)-L-alaninate,
1-((2S)-2-(((7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)(methyl)amino)methyl)pyrrolidin-1-yl)prop-2-en-1-one,
8-((((S)-1-acryloylpyrrolidin-2-yl)methyl)(methyl)amino)-6-(((S)-1,2-dimethylpyrrolidin-2-yl)methoxy)-3-(8-ethynyl-7-fluoro-3-hydroxynaphthalen-1-yl)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-1,2-dimethylpyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-1-(3-hydroxypropyl)-2-methylpyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(tetrahydro-2H-pyran-4-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-1-(4-hydroxy-4-methylcyclohexyl)-2-methylpyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-1-(4-fluorophenyl)-2-methylpyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(pyridin-4-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(6-(trifluoromethyl)pyridin-3-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(6-((R)-3-methylmorpholino)pyridin-3-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-((tetrahydro-2H-pyran-4-yl)methyl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(pyridin-3-ylmethyl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(1-(tetrahydro-2H-pyran-4-yl)piperidin-4-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-methyl-1-(6-((R)-3-methylmorpholino)pyridin-3-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2R,4R)-4-fluoro-2-isopropyl-1-(tetrahydro-2H-pyran-4-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-((S)-1-hydroxyethyl)-1-(tetrahydro-2H-pyran-4-yl)pyrrolidin-2-yl)methoxy)-3,4-dihydro-2H-pyrimido[4,5-e][1,3]oxazin-2-one,
8-((((S)-1-acryloylpyrrolidin-2-yl)methyl)(methyl)amino)-3-(8-ethynyl-7-fluoro-3-hydroxynaphthalen-1-yl)-6-(((2S,4R)-4-fluoro-2-(2-hydroxypropan-2-yl)-1-(tetrahydro-2H-pyran-4-yl)pyrrolidin-2-yl)methoxy)-3,4-dihydro-2H-pyrimido[4,5-e][1,3]oxazin-2-one,
2-((2R,4R)-2-(((8-((((S)-1-acryloylpyrrolidin-2-yl)methyl)(methyl)amino)-3-(8-ethynyl-7-fluoro-3-hydroxynaphthalen-1-yl)-2-oxo-3,4-dihydro-2H-pyrimido[4,5-e][1,3]oxazin-6-yl)oxy)methyl)-4-fluoro-1-(tetrahydro-2H-pyran-4-yl)pyrrolidin-2-yl)-2-methylpropanenitrile,
4-(8-((((S)-1-acryloylpyrrolidin-2-yl)methyl)(methyl)amino)-6-(((2S,4R)-4-fluoro-1,2-dimethylpyrrolidin-2-yl)methoxy)-2-oxo-2H-pyrimido[4,5-e][1,3]oxazin-3(4H)-yl)-5-ethynyl-6-fluoronaphthalen-2-yl isobutyrate,
4-(8-((((S)-1-acryloylpyrrolidin-2-yl)methyl)(methyl)amino)-6-(((2S,4R)-4-fluoro-1,2-dimethylpyrrolidin-2-yl)methoxy)-2-oxo-2H-pyrimido[4,5-e][1,3]oxazin-3(4H)-yl)-5-ethynyl-6-fluoronaphthalen-2-yl isopropyl carbonate,
isopropyl (((4-(8-((((S)-1-acryloylpyrrolidin-2-yl)methyl)(methyl)amino)-6-(((2S,4R)-4-fluoro-1,2-dimethylpyrrolidin-2-yl)methoxy)-2-oxo-2H-pyrimido[4,5-e][1,3]oxazin-3(4H)-yl)-5-ethynyl-6-fluoronaphthalen-2-yl)oxy)(isopropoxy)phosphoryl)-L-alaninate,
1-((S)-2-((((R)-7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-5,6,7,8-tetrahydroquinazolin-4-yl)(methyl)amino)methyl)pyrrolidin-1-yl)prop-2-en-1-one,
1-((S)-2-((((R)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-5,6,7,8-tetrahydroquinazolin-4-yl)(methyl)amino)methyl)pyrrolidin-1-yl)prop-2-en-1-one,
4-((R)-4-((((S)-1-acryloylpyrrolidin-2-yl)methyl)(methyl)amino)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl isobutyrate,
4-((S)-4-((((S)-1-acryloylpyrrolidin-2-yl)methyl)(methyl)amino)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl isobutyrate,
isopropyl (((4-((S)-4-((((S)-1-acryloylpyrrolidin-2-yl)methyl)(methyl)amino)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl)oxy)(isopropoxy)phosphoryl)-L-alaninate,
isopropyl (((4-((R)-4-((((S)-1-acryloylpyrrolidin-2-yl)methyl)(methyl)amino)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl)oxy)(isopropoxy)phosphoryl)-L-alaninate,
(S)-1-(2-(((7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((S)-1-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6-dihydroquinazolin-4-yl)(methyl)amino)methyl)pyrrolidin-1-yl)prop-2-en-1-one,
1-((S)-2-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6-dihydroquinazolin-4-yl)(methyl)amino)methyl)pyrrolidin-1-yl)prop-2-en-1-one,
2-((2R,4R)-2-(((4-((((S)-1-acryloylpyrrolidin-2-yl)methyl)(methyl)amino)-7-(8-ethynyl-7-fluoro-3-hydroxynaphthalen-1-yl)-5,6-dihydroquinazolin-2-yl)oxy)methyl)-4-fluoro-1-(tetrahydro-2H-pyran-4-yl)pyrrolidin-2-yl)-2-methylpropanenitrile,
4-(4-((((S)-1-acryloylpyrrolidin-2-yl)methyl)(methyl)amino)-2-(((2S,4R)-4-fluoro-1,2-dimethylpyrrolidin-2-yl)methoxy)-5,6-dihydroquinazolin-7-yl)-5-ethynyl-6-fluoronaphthalen-2-yl isobutyrate,
4-(4-((((S)-1-acryloylpyrrolidin-2-yl)methyl)(methyl)amino)-2-(((2S,4R)-4-fluoro-1,2-dimethylpyrrolidin-2-yl)methoxy)-5,6-dihydroquinazolin-7-yl)-5-ethynyl-6-fluoronaphthalen-2-yl isopropyl carbonate,
isopropyl (((4-(4-((((S)-1-acryloylpyrrolidin-2-yl)methyl)(methyl)amino)-2-(((2S,4R)-4-fluoro-1,2-dimethylpyrrolidin-2-yl)methoxy)-5,6-dihydroquinazolin-7-yl)-5-ethynyl-6-fluoronaphthalen-2-yl)oxy)(isopropoxy)phosphoryl)-L-alaninate,
1-((S)-2-(((7-(8-ethynyl-7-fluoro-3,4-dihydroquinolin-1(2H)-yl)-8-fluoro-2-(((S)-1-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(5-ethynyl-6-fluoro-2,3-dihydro-4H-benzo[b][1, 4]oxazin-4-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((S)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((R)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((R)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((S)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((R)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((S)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1S,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1R,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1S,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1R,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1R,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1R,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1S,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-(((7-((1S,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)(methyl)amino)methyl)pyrrolidin-1-yl)prop-2-en-1-one,
1-((S)-2-((((R)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((R)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-((((S)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)(methyl)amino)methyl)pyrrolidin-1-yl)prop-2-en-1-one,
1-((S)-2-(((7-(8-ethynyl-7-fluoro-3,4-dihydroquinolin-1(2H)-yl)-2-(((2S,4R)-4-fluoro-1-(4-fluorophenyl)-2-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-(5-ethynyl-6-fluoro-2,3-dihydro-4H-benzo[b][1, 4]oxazin-4-yl)-2-(((2S,4R)-4-fluoro-1-(4-fluorophenyl)-2-methylpyrrolidin-2-yl)methoxy)-5,6-dihydroquinazolin-4-yl)(methyl)amino)methyl)pyrrolidin-1-yl)prop-2-en-1-one,
1-((S)-2-(((7-((R)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-((S)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-((R)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)(methyl)amino)methyl)pyrrolidin-1-yl)prop-2-en-1-one,
1-((S)-2-(((7-((S)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)(methyl)amino)methyl)pyrrolidin-1-yl)prop-2-en-1-one,
1-((S)-2-((((S)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((R)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-((((S)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)(methyl)amino)methyl)pyrrolidin-1-yl)prop-2-en-1-one,
1-((S)-2-(((7-((R)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((S)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((S)-5-ethynyl-6-fluorochroman-4-yl)-2-(((2S,4R)-4-fluoro-2-methyl-1-(pyridin-2-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((R)-5-ethynyl-6-fluorochroman-4-yl)-2-(((2S,4R)-4-fluoro-2-methyl-1-(pyridin-2-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1S,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1S,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1R,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1R,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1S,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1S,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1R,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
1-((S)-2-(((7-((1R,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)(methyl)amino)methyl)pyrrolidin-1-yl)prop-2-en-1-one,
(S)—N-(1-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
(R)—N-(1-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
(S)—N-(4-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)acrylamide,
(R)—N-(4-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)acrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methyl-1,4-diazepan-6-yl)acrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methyl-1,4-diazepan-6-yl)acrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-(tetrahydro-2H-pyran-4-yl)-1,4-diazepan-6-yl)acrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-(tetrahydro-2H-pyran-4-yl)-1,4-diazepan-6-yl)acrylamide,
(S)—N-(1-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)-2-fluoroacrylamide,
(R)—N-(1-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)-2-fluoroacrylamide,
(S)—N-(4-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)-2-fluoroacrylamide,
(R)—N-(4-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)-2-fluoroacrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methyl-1,4-diazepan-6-yl)-2-fluoroacrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methyl-1,4-diazepan-6-yl)-2-fluoroacrylamide,
N-((3S,4R)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3S,4S)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3R,4R)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3R,4S)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3S,5R)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3S,5S)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3R,5R)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3R,5S)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-((3S,4R)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)acrylamide,
N-((3S,4S)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)acrylamide,
N-((3R,4R)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)acrylamide,
N-((3R,4S)-4-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)acrylamide,
N-((3S,5S)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-methylazepan-3-yl)acrylamide,
N-((3S,5R)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-methylazepan-3-yl)acrylamide,
N-((3R,5S)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-methylazepan-3-yl)acrylamide,
N-((3R,5R)-5-cyano-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-methylazepan-3-yl)acrylamide,
N-((3R,4R)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)acrylamide,
N-((3R,4S)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)acrylamide,
N-((3S,4R)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)acrylamide,
N-((3S,4S)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)acrylamide,
N-((3S,5S)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-hydroxy-5-methylazepan-3-yl)acrylamide,
N-((3S,5R)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-hydroxy-5-methylazepan-3-yl)acrylamide,
N-((3R,5S)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-hydroxy-5-methylazepan-3-yl)acrylamide,
N-((3R,5R)-1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5-hydroxy-5-methylazepan-3-yl)acrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4,4-dimethylazepan-3-yl)acrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4,4-dimethylazepan-3-yl)acrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5,5-dimethylazepan-3-yl)acrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5,5-dimethylazepan-3-yl)acrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4,4-difluoroazepan-3-yl)acrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-4,4-difluoroazepan-3-yl)acrylamide,
(S)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5,5-difluoroazepan-3-yl)acrylamide,
(R)—N-(1-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-5,5-difluoroazepan-3-yl)acrylamide,
N-(1-(7-(8-ethynyl-7-fluoro-3-hydroxynaphthalen-1-yl)-8-fluoro-2-(((2S,4R)-4-fluoro-1-methylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-1-(4-hydroxy-4-methylcyclohexyl)-2-methylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-1-(4-fluorophenyl)-2-methylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyridin-2-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyridin-3-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyridin-4-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyrimidin-4-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyrimidin-5-yl)pyrrolidin-2-yl)methoxy)pyrido[4,3-d]pyrimidin-4-yl)azepan-3-yl)acrylamide,
N-(1-(8-fluoro-2-(((2S,4R)-4-fluoro-2-methyl-1-(pyrimidin-2-yl)pyrrolidin-2-yl)methoxy)-7-(7-fluoro-3-hydroxy-8-methylnaphthalen-1-yl)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(6-(trifluoromethyl)pyridin-3-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-((tetrahydro-2H-pyran-4-yl)methyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-1-(4-fluorobenzyl)-2-methylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyridin-2-ylmethyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyridin-3-ylmethyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyridin-4-ylmethyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyrimidin-4-ylmethyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyrimidin-2-ylmethyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(pyrimidin-5-ylmethyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(1-(tetrahydro-2H-pyran-4-yl)piperidin-4-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-((1R,4R)-4-((S)-3-methylmorpholino)cyclohexyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-((1S,4S)-4-((S)-3-methylmorpholino)cyclohexyl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(2-((R)-3-methylmorpholino)pyridin-4-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(7-oxaspiro[3.5]nonan-2-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2R,4R)-4-fluoro-2-isopropyl-1-(tetrahydro-2H-pyran-4-yl)pyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-1-(tetrahydro-2H-pyran-4-yl)-2-(trifluoromethyl)pyrrolidin-2-yl)methoxy)pyrido[4,3-d]pyrimidin-4-yl)azepan-3-yl)acrylamide,
N-(1-(2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)azepan-3-yl)acrylamide,
N-(1-(7-(8-ethynyl-7-fluoro-3-hydroxynaphthalen-1-yl)-8-fluoro-2-(((2S,4R)-4-fluoro-2-((S)-1-hydroxyethyl)-1-(tetrahydro-2H-pyran-4-yl)pyrrolidin-2-yl)methoxy)pyrido[4,3-d]pyrimidin-4-yl)azepan-3-yl)acrylamide,
N-(1-(7(8-ethynyl-7-fluoro-3-hydroxynaphthalen-1-yl)-8-fluoro-2-(((2S,4R)-4-fluoro-2-(2-hydroxypropan-2-yl)-1-(tetrahydro-2H-pyran-4-yl)pyrrolidin-2-yl)methoxy)pyrido[4,3-d]pyrimidin-4-yl)azepan-3-yl)acrylamide,
N-(1-(2-(((2R,4R)-2-((S)-1-cyanoethyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)azepan-3-yl)acrylamide,
N-(1-(2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)azepan-3-yl)acrylamide,
4-(4-(3-acrylamidoazepan-1-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl isobutyrate,
4-(4-(3-acrylamidoazepan-1-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl isopropyl carbonate,
4-(4-(3-acrylamidoazepan-1-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl dimethylcarbamate,
4-(4-(3-acrylamidoazepan-1-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl diisopropyl phosphate,
A name could not be generated for this structure,
isopropyl (((4-(4-(3-acrylamidoazepan-1-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl)oxy)(isopropoxy)phosphoryl)-L-alaninate,
isopropyl (((4-(4-(3-acrylamidoazepan-1-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-yl)oxy)(phenoxy)phosphoryl)-L-alaninate,
N-(1-(7-(3-cyano-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(8-ethynyl-7-fluoro-3-methoxynaphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(3-(dimethylphosphoryl)-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(3-acetyl-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(8-ethynyl-7-fluoro-3-((R)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(8-ethynyl-7-fluoro-3-((S)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((S)-1-methylpyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-methylpyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(4-fl(uorophenyl)-2-methylpyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)azepan-3-yl)acrylamide,
N-(1-(6-(((8-ethynyl-7-fluoro-3-hydroxynaphthalen-1-yl)(methyl)amino)methyl)-2-(((2S,4R)-4-fluoro-2-methyl-1-(1-(tetrahydro-2H-pyran-4-yl)piperidin-4-yl)pyrrolidin-2-yl)methoxy)pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6,7,8-tetrahydropyrido[3,4-d]pyrimidin-4-yl)azepan-3-yl)acrylamide,
N-(1-(2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-5,6,7,8-tetrahydropyrido[3,4-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6,7,8-tetrahydropyrido[3,4-d]pyrimidin-4-yl)azepan-3-yl)acrylamide,
N-(1-(2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6,7,8-tetrahydropyrido[3,4-d]pyrimidin-4-yl)azepan-3-yl)acrylamide,
4-(4-(3-acrylamidoazepan-1-yl)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl isobutyrate,
4-(4-(3-acrylamidoazepan-1-yl)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl isopropyl carbonate,
isopropyl (((4-(4-(3-acrylamidoazepan-1-yl)-2-(((2S,4R)-4-fluoro-1,2-dimethylpyrrolidin-2-yl)methoxy)-5,8-dihydropyrido[3,4-d]pyrimidin-7(6H)-yl)-5-ethynyl-6-fluoronaphthalen-2-yl)oxy)(isopropoxy)phosphoryl)-L-alaninate,
N-(1-((R)-7-(8-ethynyl-7-fluoro-3-hydroxynaphthalen-1-yl)-2-(((2S,4R)-4-fluoro-1,2-dimethylpyrrolidin-2-yl)methoxy)-7,8-dihydro-6H-pyrano[3,2-d]pyrimidin-4-yl)azepan-3-yl)acrylamide,
N-(1-((S)-7-(8-ethynyl-7-fluoro-3-hydroxynaphthalen-1-yl)-2-(((2S,4R)-4-fluoro-1,2-dimethylpyrrolidin-2-yl)methoxy)-7,8-dihydro-6H-pyrano[3,2-d]pyrimidin-4-yl)azepan-3-yl)acrylamide,
N-(1-((R)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-7,8-dihydro-6H-pyrano[3,2-d]pyrimidin-4-yl)azepan-3-yl)acrylamide,
N-(1-((S)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-7,8-dihydro-6H-pyrano[3,2-d]pyrimidin-4-yl)azepan-3-yl)acrylamide,
N-(1-((R)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)azepan-3-yl)acrylamide,
N-(1-((S)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)azepan-3-yl)acrylamide,
N-(1-((S)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)azepan-3-yl)acrylamide,
N-(1-((R)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)azepan-3-yl)acrylamide,
N-(1-((S)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-7,8-dihydro-6H-pyrano[3,2-d]pyrimidin-4-yl)azepan-3-yl)acrylamide,
N-(1-((R)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-7,8-dihydro-6H-pyrano[3,2-d]pyrimidin-4-yl)azepan-3-yl)acrylamide,
N-(1-((S)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-7,8-dihydro-6H-pyrano[3,2-d]pyrimidin-4-yl)azepan-3-yl)acrylamide,
N-(1-((R)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-7,8-dihydro-6H-pyrano[3,2-d]pyrimidin-4-yl)azepan-3-yl)acrylamide,
N-(1-((S)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-7,8-dihydro-6H-pyrano[3,2-d]pyrimidin-4-yl)azepan-3-yl)acrylamide,
N-(1-((R)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-7,8-dihydro-6H-pyrano[3,2-d]pyrimidin-4-yl)azepan-3-yl)acrylamide,
4-((7R)-4-(3-acrylamidoazepan-1-yl)-2-(((2S,4R)-4-fluoro-1,2-dimethylpyrrolidin-2-yl)methoxy)-7,8-dihydro-6H-pyrano[3,2-d]pyrimidin-7-yl)-5-ethynyl-6-fluoronaphthalen-2-yl isobutyrate,
4-((7S)-4-(3-acrylamidoazepan-1-yl)-2-(((2S,4R)-4-fluoro-1,2-dimethylpyrrolidin-2-yl)methoxy)-7,8-dihydro-6H-pyrano[3,2-d]pyrimidin-7-yl)-5-ethynyl-6-fluoronaphthalen-2-yl isobutyrate,
isopropyl (((4-((7S)-4-(3-acrylamidoazepan-1-yl)-2-(((2S,4R)-4-fluoro-1,2-dimethylpyrrolidin-2-yl)methoxy)-7,8-dihydro-6H-pyrano[3,2-d]pyrimidin-7-yl)-5-ethynyl-6-fluoronaphthalen-2-yl)oxy)(isopropoxy)phosphoryl)-L-alaninate,
isopropyl (((4-((7R)-4-(3-acrylamidoazepan-1-yl)-2-(((2S,4R)-4-fluoro-1,2-dimethylpyrrolidin-2-yl)methoxy)-7,8-dihydro-6H-pyrano[3,2-d]pyrimidin-7-yl)-5-ethynyl-6-fluoronaphthalen-2-yl)oxy)(isopropoxy)phosphoryl)-L-alaninate,
N-(1-(7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((S)-1-methylpyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-methylpyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-6,7-dihydro-[1,4]dioxino[2,3-d]pyrimidin-4-yl)azepan-3-yl)acrylamide,
N-(1-(2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-6,7-dihydro-[1,4]dioxino[2,3-d]pyrimidin-4-yl)azepan-3-yl)acrylamide,
N-(1-(7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-6,7-dihydro-[1,4]dioxino[2,3-d]pyrimidin-4-yl)azepan-3-yl)acrylamide,
N-(1-(2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-6,7-dihydro-[1,4]dioxino[2,3-d]pyrimidin-4-yl)azepan-3-yl)acrylamide,
4-(4-(3-acrylamidoazepan-1-yl)-2-(((2S,4R)-4-fluoro-1,2-dimethylpyrrolidin-2-yl)methoxy)-6,7-dihydro-[1,4]dioxino[2,3-d]pyrimidin-7-yl)-5-ethynyl-6-fluoronaphthalen-2-yl isobutyrate,
4-(4-(3-acrylamidoazepan-1-yl)-2-(((2S,4R)-4-fluoro-1,2-dimethylpyrrolidin-2-yl)methoxy)-6,7-dihydro-[1,4]dioxino[2,3-d]pyrimidin-7-yl)-5-ethynyl-6-fluoronaphthalen-2-yl isopropyl carbonate,
isopropyl (((4-(4-(3-acrylamidoazepan-1-yl)-2-(((2S,4R)-4-fluoro-1,2-dimethylpyrrolidin-2-yl)methoxy)-6,7-dihydro-[1,4]dioxino[2,3-d]pyrimidin-7-yl)-5-ethynyl-6-fluoronaphthalen-2-yl)oxy)(isopropoxy)phosphoryl)-L-alaninate,
N-(1-(7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)azepan-3-yl)acrylamide,
N-(1-(6-(((S)-1,2-dimethylpyrrolidin-2-yl)methoxy)-3-(8-ethynyl-7-fluoro-3-hydroxynaphthalen-1-yl)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-1,2-dimethylpyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-1-(3-hydroxypropyl)-2-methylpyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(tetrahydro-2H-pyran-4-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-1-(4-hydroxy-4-methylcyclohexyl)-2-methylpyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-1-(4-fluorophenyl)-2-methylpyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(pyridin-4-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(6-(trifluoromethyl)pyridin-3-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(6-((R)-3-methylmorpholino)pyridin-3-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-((tetrahydro-2H-pyran-4-yl)methyl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(pyridin-3-ylmethyl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(1-(tetrahydro-2H-pyran-4-yl)piperidin-4-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-methyl-1-(6-((R)-3-methylmorpholino)pyridin-3-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2R,4R)-4-fluoro-2-isopropyl-1-(tetrahydro-2H-pyran-4-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-((S)-1-hydroxyethyl)-1-(tetrahydro-2H-pyran-4-yl)pyrrolidin-2-yl)methoxy)-2-oxo-3,4-dihydro-2H-pyrimido[4,5-e][1,3]oxazin-8-yl)azepan-3-yl)acrylamide,
N-(1-(3-(8-ethynyl-7-fluoro-3-hydroxynaphthalen-1-yl)-6-(((2S,4R)-4-fluoro-2-(2-hydroxypropan-2-yl)-1-(tetrahydro-2H-pyran-4-yl)pyrrolidin-2-yl)methoxy)-2-oxo-3,4-dihydro-2H-pyrimido[4,5-e][1,3]oxazin-8-yl)azepan-3-yl)acrylamide,
N-(1-(6-(((2R,4R)-2-(2-cyanopropan-2-yl)-4-fluoro-1-(tetrahydro-2H-pyran-4-yl)pyrrolidin-2-yl)methoxy)-3-(8-ethynyl-7-fluoro-3-hydroxynaphthalen-1-yl)-2-oxo-3,4-dihydro-2H-pyrimido[4,5-e][1,3]oxazin-8-yl)azepan-3-yl)acrylamide,
4-(8-(3-acrylamidoazepan-1-yl)-6-(((2S,4R)-4-fluoro-1,2-dimethylpyrrolidin-2-yl)methoxy)-2-oxo-2H-pyrimido[4,5-e][1,3]oxazin-3(4H)-yl)-5-ethynyl-6-fluoronaphthalen-2-yl isobutyrate,
4-(8-(3-acrylamidoazepan-1-yl)-6-(((2S,4R)-4-fluoro-1,2-dimethylpyrrolidin-2-yl)methoxy)-2-oxo-2H-pyrimido[4,5-e][1,3]oxazin-3(4H)-yl)-5-ethynyl-6-fluoronaphthalen-2-yl isopropyl carbonate,
isopropyl (((4-(8-(3-acrylamidoazepan-1-yl)-6-(((2S,4R)-4-fluoro-1,2-dimethylpyrrolidin-2-yl)methoxy)-2-oxo-2H-pyrimido[4,5-e][1,3]oxazin-3(4H)-yl)-5-ethynyl-6-fluoronaphthalen-2-yl)oxy)(isopropoxy)phosphoryl)-L-alaninate,
N-(1-((R)-7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydroquinazolin-4-yl)azepan-3-yl)acrylamide,
N-(1-((S)-7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydroquinazolin-4-yl)azepan-3-yl)acrylamide,
N-(1-((R)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydroquinazolin-4-yl)azepan-3-yl)acrylamide,
N-(1-((S)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydroquinazolin-4-yl)azepan-3-yl)acrylamide,
N-(1-((R)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydroquinazolin-4-yl)azepan-3-yl)acrylamide,
N-(1-((S)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydroquinazolin-4-yl)azepan-3-yl)acrylamide,
N-(1-((S)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydroquinazolin-4-yl)azepan-3-yl)acrylamide,
N-(1-((R)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydroquinazolin-4-yl)azepan-3-yl)acrylamide,
N-(1-((S)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-5,6,7,8-tetrahydroquinazolin-4-yl)azepan-3-yl)acrylamide,
N-(1-((R)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-5,6,7,8-tetrahydroquinazolin-4-yl)azepan-3-yl)acrylamide,
N-(1-((S)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-5,6,7,8-tetrahydroquinazolin-4-yl)azepan-3-yl)acrylamide,
N-(1-((R)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-5,6,7,8-tetrahydroquinazolin-4-yl)azepan-3-yl)acrylamide,
N-(1-((S)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-5,6,7,8-tetrahydroquinazolin-4-yl)azepan-3-yl)acrylamide,
N-(1-((R)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-5,6,7,8-tetrahydroquinazolin-4-yl)azepan-3-yl)acrylamide,
4-((7R)-4-(3-acrylamidoazepan-1-yl)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl isobutyrate,
4-((7S)-4-(3-acrylamidoazepan-1-yl)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl isobutyrate,
isopropyl (((4-((7S)-4-(3-acrylamidoazepan-1-yl)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl)oxy)(isopropoxy)phosphoryl)-L-alaninate,
isopropyl (((4-((7R)-4-(3-acrylamidoazepan-1-yl)-2-(((2S,4R)-4-fluoro-1,2-dimethylpyrrolidin-2-yl)methoxy)-5,6,7,8-tetrahydroquinazolin-7-yl)-5-ethynyl-6-fluoronaphthalen-2-yl)oxy)(isopropoxy)phosphoryl)-L-alaninate,
N-(1-(7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((S)-1-methylpyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-1-methylpyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6-dihydroquinazolin-4-yl)azepan-3-yl)acrylamide,
N-(1-(2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6-dihydroquinazolin-4-yl)azepan-3-yl)acrylamide,
N-(1-(7(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-5,6-dihydroquinazolin-4-yl)azepan-3-yl)acrylamide,
N-(1-(7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6-dihydroquinazolin-4-yl)azepan-3-yl)acrylamide,
N-(1-(2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6-dihydroquinazolin-4-yl)azepan-3-yl)acrylamide,
4-(4-(3-acrylamidoazepan-1-yl)-2-(((2S,4R)-4-fluoro-1,2-dimethylpyrrolidin-2-yl)methoxy)-5,6-dihydroquinazolin-7-yl)-5-ethynyl-6-fluoronaphthalen-2-yl isobutyrate,
4-(4-(3-acrylamidoazepan-1-yl)-2-(((2S,4R)-4-fluoro-1,2-dimethylpyrrolidin-2-yl)methoxy)-5,6-dihydroquinazolin-7-yl)-5-ethynyl-6-fluoronaphthalen-2-yl isopropyl carbonate,
isopropyl (((4-(4-(3-acrylamidoazepan-1-yl)-2-(((2S,4R)-4-fluoro-1,2-dimethylpyrrolidin-2-yl)methoxy)-5,6-dihydroquinazolin-7-yl)-5-ethynyl-6-fluoronaphthalen-2-yl)oxy)(isopropoxy)phosphoryl)-L-alaninate,
N-(1-(7-(8-ethynyl-7-fluoro-3,4-dihydroquinolin-1(2H)-yl)-8-fluoro-2-(((S)-1-methylpyrrolidin-2-yl)methoxy)pyrido[4,3-d]pyrimidin-4-yl)azepan-3-yl)acrylamide,
N-(1-(7-(5-ethynyl-6-fluoro-2,3-dihydro-4H-benzo[b][1,4]oxazin-4-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)azepan-3-yl)acrylamide,
N-(1-(7-((S)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)azepan-3-yl)acrylamide,
N-(1-(7-((R)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)azepan-3-yl)acrylamide,
N-(1-(7-((R)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-((S)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)azepan-3-yl)acrylamide,
N-(1-(7-((R)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)azepan-3-yl)acrylamide,
N-(1-(7-((S)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)azepan-3-yl)acrylamide,
N-(1-(7-((1S,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)azepan-3-yl)acrylamide,
N-(1-(7-((1R,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)azepan-3-yl)acrylamide,
N-(1-(7-((1S,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)azepan-3-yl)acrylamide,
N-(1-(7-((1R,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)azepan-3-yl)acrylamide,
N-(1-(7-((1R,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)azepan-3-yl)acrylamide,
N-(1-(7-((1R,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)azepan-3-yl)acrylamide,
N-(1-(7-((1S,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)azepan-3-yl)acrylamide,
N-(1-(7-((1S,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)azepan-3-yl)acrylamide,
N-(1-((R)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)azepan-3-yl)acrylamide,
N-(1-((S)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)azepan-3-yl)acrylamide,
N-(1-((R)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)azepan-3-yl)acrylamide,
N-(1-((S)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)azepan-3-yl)acrylamide,
N-(1-((R)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)azepan-3-yl)acrylamide,
N-(1-((R)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)azepan-3-yl)acrylamide,
N-(1-((S)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)azepan-3-yl)acrylamide,
N-(1-((S)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)azepan-3-yl)acrylamide,
N-(1-((R)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)azepan-3-yl)acrylamide,
N-(1-((R)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)azepan-3-yl)acrylamide,
N-(1-((S)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)azepan-3-yl)acrylamide,
N-(1-((S)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)azepan-3-yl)acrylamide,
N-(1-(7-(8-ethynyl-7-fluoro-3,4-dihydroquinolin-1(2H)-yl)-2-(((2S,4R)-4-fluoro-1-(4-fluorophenyl)-2-methylpyrrolidin-2-yl)methoxy)-5,6-dihydroquinazolin-4-yl)azepan-3-yl)acrylamide,
N-(1-(7-(5-ethynyl-6-fluoro-2,3-dihydro-4H-benzo[b][1,4]oxazin-4-yl)-2-(((2S,4R)-4-fluoro-1-(4-fluorophenyl)-2-methylpyrrolidin-2-yl)methoxy)-5,6-dihydroquinazolin-4-yl)azepan-3-yl)acrylamide,
N-(1-(7-((R)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)azepan-3-yl)acrylamide,
N-(1-(7-((S)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)azepan-3-yl)acrylamide,
N-(1-(7-((R)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)azepan-3-yl)acrylamide,
N-(1-(7-((S)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)azepan-3-yl)acrylamide,
N-(1-((S)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)azepan-3-yl)acrylamide,
N-(1-((R)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)azepan-3-yl)acrylamide,
N-(1-((S)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)azepan-3-yl)acrylamide,
N-(1-((R)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)azepan-3-yl)acrylamide,
N-(1-((R)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)azepan-3-yl)acrylamide,
N-(1-((R)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)azepan-3-yl)acrylamide,
N-(1-((S)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)azepan-3-yl)acrylamide,
N-(1-((S)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)azepan-3-yl)acrylamide,
N-(1-((R)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)azepan-3-yl)acrylamide,
N-(1-((R)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)azepan-3-yl)acrylamide,
N-(1-((S)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)azepan-3-yl)acrylamide,
N-(1-((S)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)azepan-3-yl)acrylamide,
N-(1-(7-((R)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)azepan-3-yl)acrylamide,
N-(1-(7-((S)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)azepan-3-yl)acrylamide,
N-(1-(7-((S)-5-ethynyl-6-fluorochroman-4-yl)-2-(((2S,4R)-4-fluoro-2-methyl-1-(pyridin-2-yl methyl) pyrrolidin-2-yl)methoxy)-5,6,7,8-tetrahydropyrido[3,4-d]pyrimidin-4-yl)azepan-3-yl)acrylamide,
N-(1-(7-((R)-5-ethynyl-6-fluorochroman-4-yl)-2-(((2S,4R)-4-fluoro-2-methyl-1-(pyridin-2-ylmethyl)pyrrolidin-2-yl)methoxy)-5,6,7,8-tetrahydropyrido[3,4-d]pyrimidin-4-yl)azepan-3-yl)acrylamide,
N-(1-(7-((1S,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)azepan-3-yl)acrylamide,
N-(1-(7-((1S,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)azepan-3-yl)acrylamide,
N-(1-(7-((1R,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)azepan-3-yl)acrylamide,
N-(1-(7-((1R,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)azepan-3-yl)acrylamide,
N-(1-(7-((1S,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)azepan-3-yl)acrylamide,
N-(1-(7-((1S,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)azepan-3-yl)acrylamide,
N-(1-(7-((1R,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)azepan-3-yl)acrylamide,
N-(1-(7-((1R,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)azepan-3-yl)acrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)-N-methylacrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)cyclopentyl)-N-methylacrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylcyclopentyl)-N-methylacrylamide,
N-(11-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-hydroxycyclopentyl)-N-methylacrylamide,
N-(3-cyano-1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)cyclopentyl)-N-methylacrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-difluorocyclopentyl)-N-acrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-dimethylcyclopentyl)-N-methylacrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-hydroxy-3-methylcyclopentyl)-N-methylacrylamide,
N-(3-cyano-1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylcyclopentyl)-N-methylacrylamide,
N-(1-(((7-(8-ethynyl-7-fluoro-3-hydroxynaphthalen-1-yl)-8-fluoro-2-((tetrahydr-1H-pyrrolizin-7a(5H)-yl)methoxy)pyrido[4,3-d]pyrimidin-4-yl)amino)methyl)cyclobutyl)-2-fluoro-N-methylacrylamide,
N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)cyclopentyl)-2-fluoro-N-methylacrylamide,
N-(1-(((7-(8-ethynyl-7-fluoro-3-hydroxynaphthalen-1-yl)-8-fluoro-2-(((2S,4R)-4-fluoro-1-methylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-1-(4-hydroxy-4-methylcyclohexyl)-2-methylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-1-(4-fluorophenyl)-2-methylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyridin-2-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyridin-3-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyridin-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyrimidin-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyrimidin-5-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyrimidin-2-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(6-(trifluoromethyl)pyridin-3-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-((tetrahydro-2H-pyran-4-yl)methyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-1-(4-fluorobenzyl)-2-methylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyridin-2-ylmethyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyridin-3-ylmethyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyridin-4-ylmethyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyrimidin-4-ylmethyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyrimidin-2-ylmethyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(pyrimidin-5-ylmethyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(1-(tetrahydro-2H-pyran-4-yl)piperidin-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-((1R,4R)-4-((S)-3-methylmorpholino)cyclohexyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-((1S,4S)-4-((S)-3-methylmorpholino)cyclohexyl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(6-((R)-3-methylmorpholino)pyridin-3-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(2-((R)-3-methylmorpholino)pyridin-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(7-oxaspiro[3.5]nonan-2-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2R,4R)-4-fluoro-2-isopropyl-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-1-(tetrahydro-2H-pyran-4-yl)-2-(trifluoromethyl)pyrrolidin-2-yl)methoxy)pyrido[4,3-d]pyrimidin-4-yl)amino)methyl)cyclopentyl)-N-methylacrylamide,
N-(1-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-((S)-1-hydroxyethyl)-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-8-fluoro-2-(((2S,4R)-4-fluoro-2-(2-hydroxypropan-2-yl)-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((2-(((2R,4R)-2-((S)-1-cyanoethyl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)amino)methyl)cyclopentyl)-N-methylacrylamide,
N-(1-(((2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-8-fluoropyrido[4,3-d]pyrimidin-4-yl)amino)methyl)cyclopentyl)-N-methylacrylamide,
5-ethynyl-6-fluoro-4-(8-fluoro-2-(((2S,4R)-4-fluoro-1,2-dimethylpyrrolidin-2-yl)methoxy)-4-(((1-(N-methylacrylamido)cyclopentyl)methyl)amino)pyrido[4,3-d]pyrimidin-7-yl)naphthalen-2-yl isobutyrate,
5-ethynyl-6-fluoro-4-(8-fluoro-2-(((2S,4R)-4-fluoro-1,2-dimethylpyrrolidin-2-yl)methoxy)-4-(((1-(N-methylacrylamido)cyclopentyl)methyl)amino)pyrido[4,3-d]pyrimidin-7-yl)naphthalen-2-yl isopropyl carbonate,
5-ethynyl-6-fluoro-4-(8-fluoro-2-(((2S,4R)-4-fluoro-1,2-dimethylpyrrolidin-2-yl)methoxy)-4-(((1-(N-methylacrylamido)cyclopentyl)methyl)amino)pyrido[4,3-d]pyrimidin-7-yl)naphthalen-2-yl dimethylcarbamate,
5-ethynyl-6-fluoro-4-(8-fluoro-2-(((2S,4R)-4-fluoro-1,2-dimethylpyrrolidin-2-yl)methoxy)-4-(((1-(N-methylacrylamido)cyclopentyl)methyl)amino)pyrido[4,3-d]pyrimidin-7-yl)naphthalen-2-yl diisopropyl phosphate,
A name could not be generated for this structure,
isopropyl (((5-ethynyl-6-fluoro-4-(8-fluoro-2-(((2S,4R)-4-fluoro-1,2-dimethylpyrrolidin-2-yl)methoxy)-4-(((1-(N-methylacrylamido)cyclopentyl)methyl)amino)pyrido[4,3-d]pyrimidin-7-yl)naphthalen-2-yl)oxy)(isopropoxy)phosphoryl)-L-alaninate,
isopropyl (((5-ethynyl-6-fluoro-4-(8-fluoro-2-(((2S,4R)-4-fluoro-1,2-dimethylpyrrolidin-2-yl)methoxy)-4-(((1-(N-methylacrylamido)cyclopentyl)methyl)amino)pyrido[4,3-d]pyrimidin-7-yl)naphthalen-2-yl)oxy)(phenoxy)phosphoryl)-L-alaninate,
N-(1-(((7-(3-cyano-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-methoxynaphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(3-(dimethylphosphoryl)-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(3-acetyl-8-ethynyl-7-fluoronaphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-((R)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-((S)-1-hydroxyethyl)naphthalen-1-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)amino)methyl)cyclopentyl)-N-methylacrylamide,
(S)—N-(1-(((7-(8-ethynyl-7-fluoro-3-hydroxynaphthalen-1-yl)-2-((1-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-methylpyrrolidin-2-yl) methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6,7,8-tetrahydropyrido[3,4-d]pyrimidin-4-yl)amino)methyl)cyclopentyl)-N-methylacrylamide,
5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-5,8-dihydropyrido[3,4-d]pyrimidin-7(6H)-yl)naphthalen-2-yl isobutyrate,
5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-5,8-dihydropyrido[3,4-d]pyrimidin-7(6H)-yl)naphthalen-2-yl isopropyl carbonate,
isopropyl (((5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-5,8-dihydropyrido[3,4-d]pyrimidin-7(6H)-yl)naphthalen-2-yl)oxy)(isopropoxy)phosphoryl)-L-alaninate,
N-(1-((((R)-7-(8-ethynyl-7-fluoro-3-hydroxynaphthalen-1-yl)-2-(((2S,4R)-4-fluoro-1,2-dimethylpyrrolidin-2-yl)methoxy)-7,8-dihydro-6H-pyrano[3,2-d]pyrimidin-4-yl)amino)methyl)cyclopentyl)-N-methyl acrylamide,
N-(1-((((S)-7-(8-ethynyl-7-fluoro-3-hydroxynaphthalen-1-yl)-2-(((2S,4R)-4-fluoro-1,2-dimethylpyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-7,8-dihydro-6H-pyrano[3,2-d]pyrimidin-4-yl)amino)methyl)cyclopentyl)-N-methylacrylamide,
N-(1-((((R)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-7,8-dihydro-6H-pyrano[3,2-d]pyrimidin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-7,8-dihydro-6H-pyrano[3,2-d]pyrimidin-4-yl)amino)methyl)cyclopentyl)-N-methylacrylamide,
5-ethynyl-6-fluoro-4-((R)-2-(((2S,4R)-4-fluoro-1,2-dimethylpyrrolidin-2-yl)methoxy)-4-(((1-(N-methylacrylamido)cyclopentyl)methyl)amino)-7,8-dihydro-6H-pyrano[3,2-d]pyrimidin-7-yl)naphthalen-2-yl isobutyrate,
5-ethynyl-6-fluoro-4-((S)-2-(((2S,4R)-4-fluoro-1,2-dimethylpyrrolidin-2-yl)methoxy)-4-(((1-(N-methylacrylamido)cyclopentyl)methyl)amino)-7,8-dihydro-6H-pyrano[3,2-d]pyrimidin-7-yl)naphthalen-2-yl isobutyrate,
isopropyl (((5-ethynyl-6-fluoro-4-((S)-2-(((2S,4R)-4-fluoro-1,2-dimethylpyrrolidin-2-yl)methoxy)-4-(((1-(N-methylacrylamido)cyclopentyl)methyl)amino)-7,8-dihydro-6H-pyrano[3,2-d]pyrimidin-7-yl)naphthalen-2-yl)oxy)(isopropoxy)phosphoryl)-L-alaninate,
isopropyl (((5-ethynyl-6-fluoro-4-((R)-2-(((2S,4R)-4-fluoro-1,2-dimethylpyrrolidin-2-yl)methoxy)-4-(((1-(N-methylacrylamido)cyclopentyl)methyl)amino)-7,8-dihydro-6H-pyrano[3,2-d]pyrimidin-7-yl)naphthalen-2-yl)oxy)(isopropoxy)phosphoryl)-L-alaninate,
N-(1-(((7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((S)-1-methylpyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-methylpyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-6,7-dihydro-[1,4]dioxino[2,3-d]pyrimidin-4-yl)amino)methyl)cyclopentyl)-N-methylacrylamide,
N-(1-(((2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-6,7-dihydro-[1,4]dioxino[2,3-d]pyrimidin-4-yl)amino)methyl)cyclopentyl)-N-methylacrylamide,
5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-6,7-dihydro-[1,4]dioxino[2,3-d]pyrimidin-7-yl)naphthalen-2-yl isobutyrate,
5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-6,7-dihydro-[1,4]dioxino[2,3-d]pyrimidin-7-yl)naphthalen-2-yl isopropyl carbonate,
isopropyl (((5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-6,7-dihydro-[1,4]dioxino[2,3-d]pyrimidin-7-yl)naphthalen-2-yl)oxy)(isopropoxy)phosphoryl)-L-alaninate,
N-(1-(((7-(8-ethynyl-7-fluoro-3-hydroxynaphthalen-1-yl)-2-((tetrahydro-1H-pyrrolizin-7a(5H)-yl)methoxy)-6,7-dihydro-[1,4]dioxino[2,3-d]pyrimidin-4-yl)amino)methyl)cyclopentyl)-N-methylacrylamide,
(S)—N-(1-(((6-((1,2-dimethylpyrrolidin-2-yl)methoxy)-3-(8-ethynyl-7-fluoro-3-hydroxynaphthalen-1-yl)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-1,2-dimethylpyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-1-(3-hydroxypropyl)-2-methylpyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(tetrahydro-2H-pyran-4-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-1-(4-hydroxy-4-methylcyclohexyl)-2-methylpyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-1-(4-fluorophenyl)-2-methylpyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(pyridin-4-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(6-(trifluoromethyl)pyridin-3-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(6-((R)-3-methylmorpholino)pyridin-3-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-((tetrahydro-2H-pyran-4-yl)methyl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(pyridin-3-ylmethyl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(1-(tetrahydro-2H-pyran-4-yl)piperidin-4-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-methyl-1-(6-((R)-3-methylmorpholino)pyridin-3-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2R,4R)-4-fluoro-2-isopropyl-1-(tetrahydro-2H-pyran-4-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-((S)-1-hydroxyethyl)-1-(tetrahydro-2H-pyran-4-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((3-(8-ethynyl-7-fluoro-3-hydroxynaphthalen-1-yl)-6-(((2S,4R)-4-fluoro-2-(2-hydroxypropan-2-yl)-1-(tetrahydro-2H-pyran-4-yl)pyrrolidin-2-yl)methoxy)-2-oxo-3,4-dihydro-2H-pyrimido[4,5-e][1,3]oxazin-8-yl)amino)methyl)cyclopentyl)-N-methylacrylamide,
N-(1-(((6-(((2R,4R)-2-(2-cyanopropan-2-yl)-4-fluoro-1-(tetrahydro-2H-pyran-4-yl)pyrrolidin-2-yl)methoxy)-3-(8-ethynyl-7-fluoro-3-hydroxynaphthalen-1-yl)-2-oxo-3,4-dihydro-2H-pyrimido[4,5-e][1,3]oxazin-8-yl)amino)methyl)cyclopentyl)-N-methylacrylamide,
5-ethynyl-6-fluoro-4-(6-(((2S,4R)-4-fluoro-1,2-dimethylpyrrolidin-2-yl)methoxy)-8-(((1-(N-methylacrylamido)cyclopentyl)methyl)amino)-2-oxo-2H-pyrimido[4,5-e][1,3]oxazin-3(4H)-yl)naphthalen-2-yl isobutyrate,
5-ethynyl-6-fluoro-4-(6-(((2S,4R)-4-fluoro-1,2-dimethylpyrrolidin-2-yl)methoxy)-8-(((1-(N-methylacrylamido)cyclopentyl)methyl)amino)-2-oxo-2H-pyrimido[4,5-e][1,3]oxazin-3(4H)-yl)naphthalen-2-yl isopropyl carbonate,
isopropyl (((5-ethynyl-6-fluoro-4-(6-(((2S,4R)-4-fluoro-1,2-dimethylpyrrolidin-2-yl)methoxy)-8-(((1-(N-methylacrylamido)cyclopentyl)methyl)amino)-2-oxo-2H-pyrimido[4,5-e][1,3]oxazin-3(4H)-yl)naphthalen-2-yl)oxy)(isopropoxy)phosphoryl)-L-alaninate,
N-(1-((((R)-7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydroquinazolin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-1,2-dimethylpyrrolidin-2-yl)methoxy)-5,6,7,8-tetrahydroquinazolin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydroquinazolin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6,7,8-tetrahydroquinazolin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydroquinazolin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6,7,8-tetrahydroquinazolin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydroquinazolin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6,7,8-tetrahydroquinazolin-4-yl)amino)methyl)cyclopentyl)-N-methylacrylamide,
N-(1-((((S)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-5,6,7,8-tetrahydroquinazolin-4-yl)amino)methyl)cyclopentyl)-N-methylacrylamide,
N-(1-((((R)-7-(8-ethynyl-7-fluoro-3-hydroxynaphthalen-1-yl)-2-(((2R,4R)-4-fluoro-1-((4-hydroxy-4-methylcyclohexyl)methyl)-2-isopropylpyrrolidin-2-yl)methoxy)-5,6,7,8-tetrahydroquinazolin-4-yl)amino)methyl)cyclopentyl)-N-methylacrylamide,
N-(1-((((S)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-5,6,7,8-tetrahydroquinazolin-4-yl)amino)methyl)cyclopentyl)-N-methylacrylamide,
N-(1-((((R)-2-(((2R,4R)-2-(tert-butyl)-4-fluoro-1-(4-((S)-3-methylmorpholino)cyclohexyl)pyrrolidin-2-yl)methoxy)-7-(8-ethynyl-7-fluoro-3-hydroxynaphthalen-1-yl)-5,6,7,8-tetrahydroquinazolin-4-yl)amino)methyl)cyclopentyl)-N-methylacrylamide,
N-(1-((((S)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-5,6,7,8-tetrahydroquinazolin-4-yl)amino)methyl)cyclopentyl)-N-methylacrylamide,
N-(1-((((R)-7-(8-ethynyl-7-fluoro-3-hydroxynaphthalen-1-yl)-2-(((2S,4R)-4-fluoro-2-(2-hydroxypropan-2-yl)-1-(4-((S)-3-methylmorpholino)cyclohexyl)pyrrolidin-2-yl)methoxy)-5,6,7,8-tetrahydroquinazolin-4-yl)amino)methyl)cyclopentyl)-N-methylacrylamide,
5-ethynyl-6-fluoro-4-((R)-2-(((2S,4R)-4-fluoro-1,2-dimethylpyrrolidin-2-yl)methoxy)-4-(((1-(N-methylacrylamido)cyclopentyl)methyl)amino)-5,6,7,8-tetrahydroquinazolin-7-yl)naphthalen-2-yl isobutyrate,
5-ethynyl-6-fluoro-4-((S)-2-(((2S,4R)-4-fluoro-1,2-dimethylpyrrolidin-2-yl)methoxy)-4-(((1-(N-methylacrylamido)cyclopentyl)methyl)amino)-5,6,7,8-tetrahydroquinazolin-7-yl)naphthalen-2-yl isobutyrate,
isopropyl (((5-ethynyl-6-fluoro-4-((S)-2-(((2S,4R)-4-fluoro-1,2-dimethylpyrrolidin-2-yl)methoxy)-4-(((1-(N-methylacrylamido)cyclopentyl)methyl)amino)-5,6,7,8-tetrahydroquinazolin-7-yl)naphthalen-2-yl)oxy)(isopropoxy)phosphoryl)-L-alaninate,
isopropyl (((5-ethynyl-6-fluoro-4-((R)-2-(((2S,4R)-4-fluoro-1,2-dimethylpyrrolidin-2-yl)methoxy)-4-(((1-(N-methylacrylamido)cyclopentyl)methyl)amino)-5,6,7,8-tetrahydroquinazolin-7-yl)naphthalen-2-yl)oxy)(isopropoxy)phosphoryl)-L-alaninate,
N-(1-(((7-(8-ethynyl-7-fluoro-3-hydroxynaphthalen-1-yl)-2-((tetrahydro-1H-pyrrolizin-7a(5H)-yl)methoxy)-5,6-dihydroquinazolin-4-yl)amino)methyl)cyclopentyl)-N-methylacrylamide,
(S)—N-(1-(((7-(7-fluoro-3-hydroxy-8-methylnaphthalen-1-yl)-2-((1-methylpyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-methylpyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(2-hydroxyethyl)-2-methylpyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(tetrahydro-2H-pyran-4-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-hydroxy-4-methylcyclohexyl)-2-methylpyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-fluorophenyl)-2-methylpyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(pyridin-4-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-(trifluoromethyl)pyridin-3-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-((tetrahydro-2H-pyran-4-yl)methyl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(4-fluorobenzyl)-2-methylpyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(pyridin-3-ylmethyl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(1-(tetrahydro-2H-pyran-4-yl)piperidin-4-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(6-((R)-3-methylmorpholino)pyridin-3-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-methyl-1-(7-oxaspiro[3.5]nonan-2-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2R,4R)-4-fluoro-2-isopropyl-1-(tetrahydro-2H-pyran-4-yl)pyrrolidin-2-yl)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-1-(tetrahydro-2H-pyran-4-yl)-2-(trifluoromethyl)pyrrolidin-2-yl)methoxy)-5,6-dihydroquinazolin-4-yl)amino)methyl)cyclopentyl)-N-methylacrylamide,
N-(1-(((2-(((2R,4R)-2-(tert-butyl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-((S)-1-hydroxyethyl)-1-(tetrahydro-2H-pyran-4-yl)pyrrolidin-yl)l)methoxy)-5,6-dihydroquinazolin-4-yl)amino)methyl)cyclopentyl)-N-methylacrylamide,
N-(1-(((7-(8-ethynyl-7-fluoro-3-hydroxynaphthalen-1-yl)-2-(((2S,4R)-4-fluoro-2-(2-hydroxypropan-2-yl)-1-(tetrahydro-2H-pyran-4-yl)pyrrolidin-2-yl)methoxy)-5,6-dihydroquinazolin-4-yl)amino)methyl)cyclopentyl)-N-methylacrylamide,
N-(1-(((2-(((2R,4R)-2-(2-cyanopropan-2-yl)-4-fluoro-1-(tetrahydro-2H-pyran-4-yl)pyrrolidin-2-yl)methoxy)-7-(8-ethynyl-7-fluoro-3-hydroxynaphthalen-1-yl)-5,6-dihydroquinazolin-4-yl)amino)methyl)cyclopentyl)-N-methylacrylamide,
5-ethynyl-6-fluoro-4-(2-(((2S,4-fluoro-1,2-dimethylpyrrolidin-2-yl)methoxy)-4-(((1-(N-methylacrylamido)cyclopentyl)methyl)amino)-5,6-dihydroquinazolin-7-yl)naphthalen-2-yl isobutyrate,
5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-5,6-dihydroquinazolin-7-yl)naphthalen-2-yl isopropyl carbonate,
isopropyl (((5-ethynyl-6-fluoro-4-(2-(((2S,4R)-4-fluoro-1,2-dimethylpyrrolidin-2-yl)methoxy)-4-(((1-(N-methylacrylamido)cyclopentyl)methyl)amino)-5,6-dihydroquinazolin-7-yl)naphthalen-2-yl)oxy)(isopropoxy)phosphoryl)-L-alaninate,
(S)—N-(1-(((7-(8-ethynyl-7-fluoro-3,4-dihydroquinolin-1(2H)-yl)-8-fluoro-2-((1-methylpyrrolidin-2-yl)methoxy)pyrido[4,3-d]pyrimidin-4-yl)amino)methyl)cyclopentyl)-N-methylacrylamide,
N-(1-(((7-(5-ethynyl-6-fluoro-2,3-dihydro-4H-benzo[b][1,4]oxazin-4-yl)-8-fluoro-2-(((2S,4R)-4-fluoro-1-(2-hydroxyethyl)-2-methylpyrrolidin-2-yl)methoxy)pyrido[4,3-d]pyrimidin-4-yl)amino)methyl)cyclopentyl)-N-methylacrylamide,
N-(1-(((7-((S)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)amino)methyl)cyclopentyl)-N-methylacrylamide,
N-(1-(((7-((R)-8-ethynyl-7-fluoro-3-hydroxy-3,4-dihydroquinolin-1(2H)-yl)-8-fluoro-2-(((2S,4R)-4-fluoro-1-methylpyrrolidin-2-yl)methoxy)pyrido[4,3-d]pyrimidin-4-yl)amino)methyl)cyclopentyl)-N-methylacrylamide,
N-(1-(((7-((R)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-((S)-8-ethynyl-7-fluoro-3-hydroxy-3-methyl-3,4-dihydroquinolin-1(2H)-yl)-8-fluoro-2-(((2S,4R)-4-fluoro-1,2-dimethylpyrrolidin-2-yl)methoxy)pyrido[4,3-d]pyrimidin-4-yl)amino)methyl)cyclopentyl)-N-methylacrylamide,
N-(1-(((7-((R)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)amino)methyl)cyclopentyl)-N-methylacrylamide,
N-(1-(((7-((S)-5-ethynyl-6-fluorochroman-4-yl)-8-fluoro-2-(((2S,4R)-4-fluoro-2-methyl-1-(7-(tetrahydro-2H-pyran-4-yl)-7-azaspiro[3.5]nonan-2-yl)pyrrolidin-2-yl)methoxy)pyrido[4,3-d]pyrimidin-4-yl)amino)methyl)cyclopentyl)-N-methylacrylamide,
N-(1-(((7-((1S,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-((1R,3R)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-((1S,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-((1R,3S)-8-ethynyl-7-fluoro-3-hydroxy-1,2,3,4-tetrahydronaphthalen-1-yl)-8-fluoro-2-(((2S,4R)-4-fluoro-2-methyl-1-(tetrahydro-2H-pyran-4-yl)pyrrolidin-2-yl)methoxy)pyrido[4,3-d]pyrimidin-4-yl)amino)methyl)cyclopentyl)-N-methylacrylamide,
N-(1-(((7-((1R,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)amino)methyl)cyclopentyl)-N-methylacrylamide,
N-(1-(((7-((1R,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)amino)methyl)cyclopentyl)-N-methylacrylamide,
N-(1-(((7-((1S,3R)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)amino)methyl)cyclopentyl)-N-methylacrylamide,
N-(1-(((7-((1S,3S)-8-ethynyl-7-fluoro-3-hydroxy-3-methyl-1,2,3,4-tetrahydronaphthalen-1-yl)-8-fluoro-2-(((2S,4R)-4-fluoro-2-methyl-1-(5-((S)-3-methylmorpholino)pyridin-2-yl)pyrrolidin-2-yl)methoxy)pyrido[4,3-d]pyrimidin-4-yl)amino)methyl)cyclopentyl)-N-methylacrylamide,
N-(1-((((R)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)amino)methyl)cyclopentyl)-N-methylacrylamide,
N-(1-((((S)-7-(8-ethynyl-7-fluoro-3,4-dihydroquinolin-1(2H)-yl)-2-(((2S,4R)-4-fluoro-1-(4-hydroxy-4-methylcyclohexyl)-2-methylpyrrolidin-2-yl)methoxy)-5,6,7,8-tetrahydroquinazolin-4-yl)amino)methyl)cyclopentyl)-N-methylacrylamide,
N-(1-((((R)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)amino)methyl)cyclopentyl)-N-methylacrylamide,
N-(1-((((S)-7-(5-ethynyl-6-fluoro-2,3-dihydro-4H-benzo[b][1,4]oxazin-4-yl)-2-(((2S,4R)-4-fluoro-2-methyl-1-(pyrimidin-4-yl)pyrrolidin-2-yl)methoxy)-5,6,7,8-tetrahydroquinazolin-4-yl)amino)methyl)cyclopentyl)-N-methylacrylamide,
N-(1-((((R)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)amino)methyl)cyclopentyl)-N-methylacrylamide,
N-(1-((((R)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)amino)methyl)cyclopentyl)-N-methylacrylamide,
N-(1-((((S)-7-((S)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)amino)methyl)cyclopentyl)-N-methylacrylamide,
N-(1-((((S)-7-((R)-8-ethynyl-7-fluoro-3-hydroxy-3,4-dihydroquinolin-1(2H)-yl)-2-(((2S,4R)-4-fluoro-2-methyl-1-(pyridin-4-yl)pyrrolidin-2-yl)methoxy)-5,6,7,8-tetrahydroquinazolin-4-yl)amino)methyl)cyclopentyl)-N-methylacrylamide,
N-(1-((((R)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)amino)methyl)cyclopentyl)-N-methylacrylamide,
N-(1-((((R)-7-((S)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)amino)methyl)cyclopentyl)-N-methylacrylamide,
N-(1-((((S)-7-((R)-8-ethynyl-7-fluoro-3-hydroxy-3-methyl-3,4-dihydroquinolin-1(2H)-yl)-2-(((2S,4R)-4-fluoro-2-methyl-1-(pyrimidin-5-yl)pyrrolidin-2-yl)methoxy)-5,6,7,8-tetrahydroquinazolin-4-yl)amino)methyl)cyclopentyl)-N-methylacrylamide,
N-(1-((((S)-2-(((2S,4R)-4-fluoro-2-methyl-1-(pyrimidin-5-yl)pyrrolidin-2-yl)methoxy)-7-((S)-7-fluoro-3-hydroxy-3,8-dimethyl-3,4-dihydroquinolin-1(2H)-yl)-5,6,7,8-tetrahydroquinazolin-4-yl)amino)methyl)cyclopentyl)-N-methylacrylamide,
N-(1-(((7-(8-ethynyl-7-fluoro-3,4-dihydroquinolin-1(2H)-yl)-2-(((2S,4R)-4-fluoro-1-(4-fluorophenyl)-2-methylpyrrolidin-2-yl)methoxy)-5,6-dihydroquinazolin-4-yl)amino)methyl)cyclopentyl)-N-methylacrylamide,
N-(1-(((7-(5-ethynyl-6-fluoro-2,3-dihydro-4H-benzo[b][1,4]oxazin-4-yl)-2-(((2S,4R)-4-fluoro-1-(4-fluorophenyl)-2-methylpyrrolidin-2-yl)methoxy)-5,6-dihydroquinazolin-4-yl)amino)methyl)cyclopentyl)-N-methylacrylamide,
N-(1-(((7-((R)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)amino)methyl)cyclopentyl)-N-methylacrylamide,
N-(1-(((7-((S)-8-ethynyl-7-fluoro-3-hydroxy-3,4-dihydroquinolin-1(2H)-yl)-2-(((2S,4R)-4-fluoro-2-methyl-1-(pyridin-2-yl)pyrrolidin-2-yl)methoxy)-5,6-dihydroquinazolin-4-yl)amino)methyl)cyclopentyl)-N-methylacrylamide,
N-(1-(((7-((R)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)amino)methyl)cyclopentyl)-N-methylacrylamide,
N-(1-(((7-((S)-8-ethynyl-7-fluoro-3-hydroxy-3-methyl-3,4-dihydroquinolin-1(2H)-yl)-2-(((2S,4R)-4-fluoro-2-methyl-1-(pyridin-3-yl)pyrrolidin-2-yl)methoxy)-5,6-dihydroquinazolin-4-yl)amino)methyl)cyclopentyl)-N-methylacrylamide,
N-(1-((((S)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(8-ethynyl-7-fluoro-3,4-dihydroquinolin-1(2H)-yl)-2-(((2S,4R)-4-fluoro-2-methyl-1-(pyrimidin-2-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-(5-ethynyl-6-fluoro-2,3-dihydro-4H-benzo[b][1,4]oxazin-4-yl)-2-(((2S,4R)-4-fluoro-2-methyl-1-(6-(trifluoromethyl)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-((S)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-((R)-8-ethynyl-7-fluoro-3-hydroxy-3,4-dihydroquinolin-1(2H)-yl)-2-(((2S,4R)-4-fluoro-2-methyl-1-(6-((R)-3-methylmorpholino)pyridin-3-yl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((R)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-((R)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)amino)methyl)cyclopentyl)-N-methylacrylamide,
N-(1-((((S)-7-((S)-8-ethynyl-7-fluoro-3-hydroxy-3-methyl-3,4-dihydroquinolin-1(2H)-yl)-2-(((2S,4R)-4-fluoro-2-methyl-1-((tetrahydro-2H-pyran-4-yl)methyl)pyrrolidin-2-yl)methoxy)-7,8-dihydro-6H-pyrano[3,2-d]pyrimidin-4-yl)amino)methyl)cyclopentyl)-N-methylacrylamide,
N-(1-(((7-((R)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((S)-8-ethynyl-7-fluoro-1,2,3,4-tetrahydronaphthalen-1-yl)-2-(((2S,4R)-4-fluoro-1-(4-fluorobenzyl)-2-methylpyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((S)-5-ethynyl-6-fluorochroman-4-yl)-2-(((2S,4R)-4-fluoro-2-methyl-1-(pyridin-2-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((R)-5-ethynyl-6-fluorochroman-4-yl)-2-(((2S,4R)-4-fluoro-2-methyl-1-(pyridin-2-ylmethyl) pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1S,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1S,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1R,3S)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1R,3R)-8-ethynyl-7-fluoro-3-hydroxy-1,2,3,4-tetrahydronaphthalen-1-yl)-2-(((2S,4R)-4-fluoro-2-methyl-1-(pyridin-3-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1S,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1S,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1R,3R)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)amino)methyl)cyclopentyl)-N-methylacrylamide,
N-(1-(((7-((1R,3S)-8-ethynyl-7-fluoro-3-hydroxy-3-methyl-1,2,3,4-tetrahydronaphthalen-1-yl)-2-(((2S,4R)-4-fluoro-2-methyl-1-(pyridin-4-ylmethyl)pyrrolidin-2-yl)methoxy)-5,6,7,8-tetrahydropyrido[3,4-d]pyrimidin-4-yl)amino)methyl)cyclopentyl)-N-methylacrylamide
5-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)-2-(2,6-dioxopiperidin-3-yl)isoindoline-1,3-dione,
5-(((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)methyl)-2-(2,6-dioxopiperidin-3-yl)isoindoline-1,3-dione,
5-(2-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)ethyl)-2-(2,6-dioxopiperidin-3-yl)isoindoline-1,3-dione,
5-(3-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)propyl)-2-(2,6-dioxopiperidin-3-yl)isoindoline-1,3-dione,
5-(4-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)butyl)-2-(2,6-dioxopiperidin-3-yl)isoindoline-1,3-dione,
5-(5-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)pentyl)-2-(2,6-dioxopiperidin-3-yl)isoindoline-1,3-dione,
5-(6-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)hexyl)-2-(2,6-dioxopiperidin-3-yl)isoindoline-1,3-dione,
(2′S,3S,4′S,5′R)—N-(4-((2-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)ethyl)carbamoyl)-2-methoxyphenyl)-6-chloro-4′-(3-chloro-2-fluorophenyl)-2′-neopentylspiro[indoline-3,3′-pyrrolidine]-5′-carboxamide,
(2′S,3S,4′S,5′R)—N-(4-((3-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)propyl)carbamoyl)-2-methoxyphenyl)-6-chloro-4′-(3-chloro-2-fluorophenyl)-2′-neopentylspiro[indoline-3,3′-pyrrolidine]-5′-carboxamide,
(2′S,3S,4′S,5′R)—N-(4-((4-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)butyl)carbamoyl)-2-methoxyphenyl)-6-chloro-4′-(3-chloro-2-fluorophenyl)-2′-neopentylspiro[indoline-3,3′-pyrrolidine]-5′-carboxamide,
(2′S,3S,4′S,5′R)—N-(4-((5-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)pentyl)carbamoyl)-2-methoxyphenyl)-6-chloro-4′-(3-chloro-2-fluorophenyl)-2′-neopentylspiro[indoline-3,3′-pyrrolidine]-5′-carboxamide,
(2′S,3S,4′S,5′R)—N-(4-((6-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)hexyl)carbamoyl)-2-methoxyphenyl)-6-chloro-4′-(3-chloro-2-fluorophenyl)-2′-neopentylspiro[indoline-3,3′-pyrrolidine]-5′-carboxamide,
5-(3-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)cyclobutyl)-2-(2,6-dioxopiperidin-3-yl)isoindoline-1,3-dione,
5-(4-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)piperidin-1-yl)-2-(2,6-dioxopiperidin-3-yl)isoindoline-1,3-dione,
5-(2-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)-7-azaspiro[3.5]nonan-7-yl)-2-(2,6-dioxopiperidin-3-yl)isoindoline-1,3-dione,
5-(4-(3-((S)-2-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)pyrrolidin-1-yl)cyclobutyl)piperazin-1-yl)-2-(2,6-dioxopiperidin-3-yl)isoindoline-1,3-dione,
4-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)-2-(2,6-dioxopiperidin-3-yl)isoindoline-1,3-dione,
4-((((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)methyl)-2-(2,6-dioxopiperidin-3-yl)isoindoline-1,3-dione,
4-(2-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)ethyl)-2-(2,6-dioxopiperidin-3-yl)isoindoline-1,3-dione,
4-(2-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)ethoxy)-2-(2,6-dioxopiperidin-3-yl)isoindoline-1,3-dione,
5-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)-2-(2,6-dioxopiperidin-3-yl)isoindoline-1,3-dione,
5-((((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)methyl)-2-(2,6-dioxopiperidin-3-yl)isoindoline-1,3-dione,
5-(2-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)ethyl)-2-(2,6-dioxopiperidin-3-yl)isoindoline-1,3-dione,
5-(2-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)ethoxy)-2-(2,6-dioxopiperidin-3-yl)isoindoline-1,3-dione,
(2′S,3S,4′S,5′R)—N-(4-((2-(((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methoxy)ethyl)carbamoyl)-2-methoxyphenyl)-6-chloro-4′-(3-chloro-2-fluorophenyl)-2′-neopentylspiro[indoline-3,3′-pyrrolidine]-5′-carboxamide,
((7aS)-7a-(((4-((1R,5S)-3,8-diazabicyclo[3.2.1]octan-3-yl)-7-(8-ethynyl-3-hydroxynaphthalen-1-yl)-8-fluoropyrido[4,3-d]pyrimidin-2-yl)oxy)methyl)hexahydro-1H-pyrrolizin-3-yl)methyl 4-(4-((2′S,3S,4′S,5′R)-6-chloro-4′-(3-chloro-2-fluorophenyl)-2′-neopentylspiro[indoline-3,3′-pyrrolidine]-5′-carboxamido)-3-methoxybenzoyl)piperazine-1-carboxylate,
5-ethynyl-6-fluoro-4-(8-fluoro-2-(((2R,7aS)-2-fluorotetrahydro-1H-pyrrolizin-7a(5H)-yl)methoxy)-4-(methyl(((S)-pyrrolidin-2-yl)methyl)amino)pyrido[4,3-d]pyrimidin-7-yl)naphthalen-2-ol,
5-ethynyl-6-fluoro-4-(8-fluoro-2-(((2R,7aS)-2-fluorotetrahydro-1H-pyrrolizin-7a(5H)-yl)methoxy)-4-(methyl(((R)-pyrrolidin-2-yl)methyl)amino)pyrido[4,3-d]pyrimidin-7-yl)naphthalen-2-ol,
5-ethynyl-6-fluoro-4-(8-fluoro-2-(((2R,7aS)-2-fluorotetrahydro-1H-pyrrolizin-7a(5H)-yl)methoxy)-4-(methyl(((R)-piperidin-2-yl)methyl)amino)pyrido[4,3-d]pyrimidin-7-yl)naphthalen-2-ol,
5-ethynyl-6-fluoro-4-(8-fluoro-2-(((2R,7aS)-2-fluorotetrahydro-1H-pyrrolizin-7a(5H)-yl)methoxy)-4-(methyl(((S)-piperidin-2-yl)methyl)amino)pyrido[4,3-d]pyrimidin-7-yl)naphthalen-2-ol,
7-(8-ethynyl-7-fluoronaphthalen-1-yl)-8-fluoro-2-(((2R,7aS)-2-fluorotetrahydro-1H-pyrrolizin-7a(5H)-yl)methoxy)-N-methyl-N—(((S)-pyrrolidin-2-yl)methyl)pyrido[4,3-d]pyrimidin-4-amine,
7-(8-ethynyl-7-fluoronaphthalen-1-yl)-8-fluoro-2-(((2R,7aS)-2-fluorotetrahydro-1H-pyrrolizin-7a(5H)-yl)methoxy)-N-methyl-N—(((R)-pyrrolidin-2-yl)methyl)pyrido[4,3-d]pyrimidin-4-amine,
7-(8-ethynyl-7-fluoronaphthalen-1-yl)-8-fluoro-2-(((2R,7aS)-2-fluorotetrahydro-1H-pyrrolizin-7a(5H)-yl)methoxy)-N-methyl-N—(((R)-piperidin-2-yl)methyl)pyrido[4,3-d]pyrimidin-4-amine,
7-(8-ethynyl-7-fluoronaphthalen-1-yl)-8-fluoro-2-(((2R,7aS)-2-fluorotetrahydro-1H-pyrrolizin-7a(5H)-yl)methoxy)-N-methyl-N—(((S)-piperidin-2-yl)methyl)pyrido[4,3-d]pyrimidin-4-amine,
7-(5-ethynyl-6-fluoroisoquinolin-4-yl)-8-fluoro-2-(((2R,7aS)-2-fluorotetrahydro-1H-pyrrolizin-7a(5H)-yl)methoxy)-N-methyl-N—(((S)-pyrrolidin-2-yl)methyl)pyrido[4,3-d]pyrimidin-4-amine,
7-(5-ethynyl-6-fluoroisoquinolin-4-yl)-8-fluoro-2-(((2R,7aS)-2-fluorotetrahydro-1H-pyrrolizin-7a(5H)-yl)methoxy)-N-methyl-N—(((R)-pyrrolidin-2-yl)methyl)pyrido[4,3-d]pyrimidin-4-amine,
7-(5-ethynyl-6-fluoroisoquinolin-4-yl)-8-fluoro-2-(((2R,7aS)-2-fluorotetrahydro-1H-pyrrolizin-7a(5H)-yl)methoxy)-N-methyl-N—(((R)-piperidin-2-yl)methyl)pyrido[4,3-d]pyrimidin-4-amine,
7-(5-ethynyl-6-fluoroisoquinolin-4-yl)-8-fluoro-2-(((2R,7aS)-2-fluorotetrahydro-1H-pyrrolizin-7a(5H)-yl)methoxy)-N-methyl-N—(((S)-piperidin-2-yl)methyl)pyrido[4,3-d]pyrimidin-4-amine,
5-ethynyl-6-fluoro-4-(8-fluoro-2-(((2R,7aS)-2-fluorotetrahydro-1H-pyrrolizin-7a(5H)-yl)methoxy)-4-(methyl(((S)-pyrrolidin-2-yl)methyl)amino)pyrido[4,3-d]pyrimidin-7-yl)-2-naphthonitrile,
5-ethynyl-6-fluoro-4-(8-fluoro-2-(((2R,7aS)-2-fluorotetrahydro-1H-pyrrolizin-7a(5H)-yl)methoxy)-4-(methyl(((R)-pyrrolidin-2-yl)methyl)amino)pyrido[4,3-d]pyrimidin-7-yl)-2-naphthonitrile,
5-ethynyl-6-fluoro-4-(8-fluoro-2-(((2R,7aS)-2-fluorotetrahydro-1H-pyrrolizin-7a(5H)-yl)methoxy)-4-(methyl(((R)-piperidin-2-yl)methyl)amino)pyrido[4,3-d]pyrimidin-7-yl)-2-naphthonitrile,
5-ethynyl-6-fluoro-4-(8-fluoro-2-(((2R,7aS)-2-fluorotetrahydro-1H-pyrrolizin-7a(5H)-yl)methoxy)-4-(methyl(((S)-piperidin-2-yl)methyl)amino)pyrido[4,3-d]pyrimidin-7-yl)-2-naphthonitrile,
7-(8-ethynyl-7-fluoro-3-(trifluoromethyl)naphthalen-1-yl)-8-fluoro-2-(((2R,7aS)-2-fluorotetrahydro-1H-pyrrolizin-7a(5H)-yl)methoxy)-N-methyl-N—(((S)-pyrrolidin-2-yl)methyl)pyrido[4,3-d]pyrimidin-4-amine,
7-(8-ethynyl-7-fluoro-3-(trifluoromethyl)naphthalen-1-yl)-8-fluoro-2-(((2R,7aS)-2-fluorotetrahydro-1H-pyrrolizin-7a(5H)-yl)methoxy)-N-methyl-N—(((R)-pyrrolidin-2-yl)methyl)pyrido[4,3-d]pyrimidin-4-amine,
7-(8-ethynyl-7-fluoro-3-(trifluoromethyl)naphthalen-1-yl)-8-fluoro-2-(((2R,7aS)-2-fluorotetrahydro-1H-pyrrolizin-7a(5H)-yl)methoxy)-N-methyl-N—(((R)-piperidin-2-yl)methyl)pyrido[4,3-d]pyrimidin-4-amine,
7-(8-ethynyl-7-fluoro-3-(trifluoromethyl)naphthalen-1-yl)-8-fluoro-2-(((2R,7aS)-2-fluorotetrahydro-1H-pyrrolizin-7a(5H)-yl)methoxy)-N-methyl-N—(((S)-piperidin-2-yl)methyl)pyrido[4,3-d]pyrimidin-4-amine,
2-(5-ethynyl-6-fluoro-4-(8-fluoro-2-(((2R,7aS)-2-fluorotetrahydro-1H-pyrrolizin-7a(5H)-yl)methoxy)-4-(methyl(((S)-pyrrolidin-2-yl)methyl)amino)pyrido[4,3-d]pyrimidin-7-yl)naphthalen-2-yl)propan-2-ol,
2-(5-ethynyl-6-fluoro-4-(8-fluoro-2-(((2R,7aS)-2-fluorotetrahydro-1H-pyrrolizin-7a(5H)-yl)methoxy)-4-(methyl(((R)-pyrrolidin-2-yl)methyl)amino)pyrido[4,3-d]pyrimidin-7-yl)naphthalen-2-yl)propan-2-ol,
2-(5-ethynyl-6-fluoro-4-(8-fluoro-2-(((2R,7aS)-2-fluorotetrahydro-1H-pyrrolizin-7a(5H)-yl)methoxy)-4-(methyl(((R)-piperidin-2-yl)methyl)amino)pyrido[4,3-d]pyrimidin-7-yl)naphthalen-2-yl)propan-2-ol,
2-(5-ethynyl-6-fluoro-4-(8-fluoro-2-(((2R,7aS)-2-fluorotetrahydro-1H-pyrrolizin-7a(5H)-yl)methoxy)-4-(methyl(((S)-piperidin-2-yl)methyl)amino)pyrido[4,3-d]pyrimidin-7-yl)naphthalen-2-yl)propan-2-ol,
2-(5-ethynyl-6-fluoro-4-(8-fluoro-2-(((2R,7aS)-2-fluorotetrahydro-1H-pyrrolizin-7a(5H)-yl)methoxy)-4-(methyl(((S)-pyrrolidin-2-yl)methyl)amino)pyrido[4,3-d]pyrimidin-7-yl)naphthalen-2-yl)-2-methylpropanenitrile,
2-(5-ethynyl-6-fluoro-4-(8-fluoro-2-(((2R,7aS)-2-fluorotetrahydro-1H-pyrrolizin-7a(5H)-yl)methoxy)-4-(methyl(((R)-pyrrolidin-2-yl)methyl)amino)pyrido[4,3-d]pyrimidin-7-yl)naphthalen-2-yl)-2-methylpropanenitrile,
2-(5-ethynyl-6-fluoro-4-(8-fluoro-2-(((2R,7aS)-2-fluorotetrahydro-1H-pyrrolizin-7a(5H)-yl)methoxy)-4-(methyl(((R)-piperidin-2-yl)methyl)amino)pyrido[4,3-d]pyrimidin-7-yl)naphthalen-2-yl)-2-methylpropanenitrile,
2-(5-ethynyl-6-fluoro-4-(8-fluoro-2-(((2R,7aS)-2-fluorotetrahydro-1H-pyrrolizin-7a(5H)-yl)methoxy)-4-(methyl(((S)-piperidin-2-yl)methyl)amino)pyrido[4,3-d]pyrimidin-7-yl)naphthalen-2-yl)-2-methylpropanenitrile,
8-ethynyl-7-fluoro-1-(8-fluoro-2-(((2R,7aS)-2-fluorotetrahydro-1H-pyrrolizin-7a(5H)-yl)methoxy)-4-(methyl(((S)-pyrrolidin-2-yl)methyl)amino)pyrido[4,3-d]pyrimidin-7-yl)isoquinolin-3(2H)-one,
8-ethynyl-7-fluoro-1-(8-fluoro-2-(((2R,7aS)-2-fluorotetrahydro-1H-pyrrolizin-7a(5H)-yl)methoxy)-4-(methyl(((R)-pyrrolidin-2-yl)methyl)amino)pyrido[4,3-d]pyrimidin-7-yl)isoquinolin-3(2H)-one,
8-ethynyl-7-fluoro-1-(8-fluoro-2-(((2R,7aS)-2-fluorotetrahydro-1H-pyrrolizin-7a(5H)-yl)methoxy)-4-(methyl(((R)-piperidin-2-yl)methyl)amino)pyrido[4,3-d]pyrimidin-7-yl)isoquinolin-3(2H)-one,
8-ethynyl-7-fluoro-1-(8-fluoro-2-(((2R,7aS)-2-fluorotetrahydro-1H-pyrrolizin-7a(5H)-yl)methoxy)-4-(methyl(((S)-piperidin-2-yl)methyl)amino)pyrido[4,3-d]pyrimidin-7-yl)isoquinolin-3(2H)-one,
5-ethynyl-6-fluoro-4-(8-fluoro-2-(((2R,7aS)-2-fluorotetrahydro-1H-pyrrolizin-7a(5H)-yl)methoxy)-4-(methyl(((S)-pyrrolidin-2-yl)methyl)amino)pyrido[4,3-d]pyrimidin-7-yl)quinolin-2(1H)-one,
5-ethynyl-6-fluoro-4-(8-fluoro-2-(((2R,7aS)-2-fluorotetrahydro-1H-pyrrolizin-7a(5H)-yl)methoxy)-4-(methyl(((R)-pyrrolidin-2-yl)methyl)amino)pyrido[4,3-d]pyrimidin-7-yl)quinolin-2(1H)-one,
5-ethynyl-6-fluoro-4-(8-fluoro-2-(((2R,7aS)-2-fluorotetrahydro-1H-pyrrolizin-7a(5H)-yl)methoxy)-4-(methyl(((R)-piperidin-2-yl)methyl)amino)pyrido[4,3-d]pyrimidin-7-yl)quinolin-2(1H)-one,
5-ethynyl-6-fluoro-4-(8-fluoro-2-(((2R,7aS)-2-fluorotetrahydro-1H-pyrrolizin-7a(5H)-yl)methoxy)-4-(methyl(((S)-piperidin-2-yl)methyl)amino)pyrido[4,3-d]pyrimidin-7-yl)quinolin-2(1H)-one,
5-ethynyl-6-fluoro-4-(8-fluoro-4-(methyl(((S)-pyrrolidin-2-yl)methyl)amino)-2-(8-methyl-3,8-diazabicyclo[3.2.1]octan-3-yl)pyrido[4,3-d]pyrimidin-7-yl)naphthalen-2-ol,
5-ethynyl-6-fluoro-4-(8-fluoro-4-(methyl(((R)-pyrrolidin-2-yl)methyl)amino)-2-(8-methyl-3,8-diazabicyclo[3.2.1]octan-3-yl)pyrido[4,3-d]pyrimidin-7-yl)naphthalen-2-ol,
5-ethynyl-6-fluoro-4-(8-fluoro-4-(methyl(((R)-piperidin-2-yl)methyl)amino)-2-(8-methyl-3,8-diazabicyclo[3.2.1]octan-3-yl)pyrido[4,3-d]pyrimidin-7-yl)naphthalen-2-ol,
5-ethynyl-6-fluoro-4-(8-fluoro-4-(methyl(((S)-piperidin-2-yl)methyl)amino)-2-(8-methyl-3,8-diazabicyclo[3.2.1]octan-3-yl)pyrido[4,3-d]pyrimidin-7-yl)naphthalen-2-ol,
7-(8-ethynyl-7-fluoronaphthalen-1-yl)-8-fluoro-N-methyl-2-(8-methyl-3,8-diazabicyclo[3.2.1]octan-3-yl)-N—(((S)-pyrrolidin-2-yl)methyl)pyrido[4,3-d]pyrimidin-4-amine,
7-(8-ethynyl-7-fluoronaphthalen-1-yl)-8-fluoro-N-methyl-2-(8-methyl-3,8-diazabicyclo[3.2.1]octan-3-yl)-N—(((R)-pyrrolidin-2-yl)methyl)pyrido[4,3-d]pyrimidin-4-amine,
7-(8-ethynyl-7-fluoronaphthalen-1-yl)-8-fluoro-N-methyl-2-(8-methyl-3,8-diazabicyclo[3.2.1]octan-3-yl)-N—(((R)-piperidin-2-yl)methyl)pyrido[4,3-d]pyrimidin-4-amine,
7-(8-ethynyl-7-fluoronaphthalen-1-yl)-8-fluoro-N-methyl-2-(8-methyl-3,8-diazabicyclo[3.2.1]octan-3-yl)-N—(((S)-piperidin-2-yl)methyl)pyrido[4,3-d]pyrimidin-4-amine,
7-(5-ethynyl-6-fluoroisoquinolin-4-yl)-8-fluoro-N-methyl-2-(8-methyl-3,8-diazabicyclo[3.2.1]octan-3-yl)-N—(((S)-pyrrolidin-2-yl)methyl)pyrido[4,3-d]pyrimidin-4-amine,
7-(5-ethynyl-6-fluoroisoquinolin-4-yl)-8-fluoro-N-methyl-2-(8-methyl-3,8-diazabicyclo[3.2.1]octan-3-yl)-N—(((R)-pyrrolidin-2-yl)methyl)pyrido[4,3-d]pyrimidin-4-amine,
7-(5-ethynyl-6-fluoroisoquinolin-4-yl)-8-fluoro-N-methyl-2-(8-methyl-3,8-diazabicyclo[3.2.1]octan-3-yl)-N—(((R)-piperidin-2-yl)methyl)pyrido[4,3-d]pyrimidin-4-amine,
7-(5-ethynyl-6-fluoroisoquinolin-4-yl)-8-fluoro-N-methyl-2-(8-methyl-3,8-diazabicyclo[3.2.1]octan-3-yl)-N—(((S)-piperidin-2-yl)methyl)pyrido[4,3-d]pyrimidin-4-amine,
5-ethynyl-6-fluoro-4-(8-fluoro-4-(methyl(((S)-pyrrolidin-2-yl)methyl)amino)-2-(8-methyl-3,8-diazabicyclo[3.2.1]octan-3-yl)pyrido[4,3-d]pyrimidin-7-yl)-2-naphthonitrile,
5-ethynyl-6-fluoro-4-(8-fluoro-4-(methyl(((R)-pyrrolidin-2-yl)methyl)amino)-2-(8-methyl-3,8-diazabicyclo[3.2.1]octan-3-yl)pyrido[4,3-d]pyrimidin-7-yl)-2-naphthonitrile,
5-ethynyl-6-fluoro-4-(8-fluoro-4-(methyl(((R)-piperidin-2-yl)methyl)amino)-2-(8-methyl-3,8-diazabicyclo[3.2.1]octan-3-yl)pyrido[4,3-d]pyrimidin-7-yl)-2-naphthonitrile,
5-ethynyl-6-fluoro-4-(8-fluoro-4-(methyl(((S)-piperidin-2-yl)methyl)amino)-2-(8-methyl-3,8-diazabicyclo[3.2.1]octan-3-yl)pyrido[4,3-d]pyrimidin-7-yl)-2-naphthonitrile,
7-(8-ethynyl-7-fluoro-3-(trifluoromethyl)naphthalen-1-yl)-8-fluoro-N-methyl-2-(8-methyl-3,8-diazabicyclo[3.2.1]octan-3-yl)-N—(((S)-pyrrolidin-2-yl)methyl)pyrido[4,3-d]pyrimidin-4-amine,
7-(8-ethynyl-7-fluoro-3-(trifluoromethyl)naphthalen-1-yl)-8-fluoro-N-methyl-2-(8-methyl-3,8-diazabicyclo[3.2.1]octan-3-yl)-N—(((R)-pyrrolidin-2-yl)methyl)pyrido[4,3-d]pyrimidin-4-amine,
7-(8-ethynyl-7-fluoro-3-(trifluoromethyl)naphthalen-1-yl)-8-fluoro-N-methyl-2-(8-methyl-3,8-diazabicyclo[3.2.1]octan-3-yl)-N—(((R)-piperidin-2-yl)methyl)pyrido[4,3-d]pyrimidin-4-amine,
7-(8-ethynyl-7-fluoro-3-(trifluoromethyl)naphthalen-1-yl)-8-fluoro-N-methyl-2-(8-methyl-3,8-diazabicyclo[3.2.1]octan-3-yl)-N—(((S)-piperidin-2-yl)methyl)pyrido[4,3-d]pyrimidin-4-amine,
2-(5-ethynyl-6-fluoro-4-(8-fluoro-4-(methyl(((S)-pyrrolidin-2-yl)methyl)amino)-2-(8-methyl-3,8-diazabicyclo[3.2.1]octan-3-yl)pyrido[4,3-d]pyrimidin-7-yl)naphthalen-2-yl)propan-2-ol,
2-(5-ethynyl-6-fluoro-4-(8-fluoro-4-(methyl(((R)-pyrrolidin-2-yl)methyl)amino)-2-(8-methyl-3,8-diazabicyclo[3.2.1]octan-3-yl)pyrido[4,3-d]pyrimidin-7-yl)naphthalen-2-yl)propan-2-ol,
2-(5-ethynyl-6-fluoro-4-(8-fluoro-4-(methyl(((R)-piperidin-2-yl)methyl)amino)-2-(8-methyl-3,8-diazabicyclo[3.2.1]octan-3-yl)pyrido[4,3-d]pyrimidin-7-yl)naphthalen-2-yl)propan-2-ol,
2-(5-ethynyl-6-fluoro-4-(8-fluoro-4-(methyl(((S)-piperidin-2-yl)methyl)amino)-2-(8-methyl-3,8-diazabicyclo[3.2.1]octan-3-yl)pyrido[4,3-d]pyrimidin-7-yl)naphthalen-2-yl)propan-2-ol,
2-(5-ethynyl-6-fluoro-4-(8-fluoro-4-(methyl(((S)-pyrrolidin-2-yl)methyl)amino)-2-(8-methyl-3,8-diazabicyclo[3.2.1]octan-3-yl)pyrido[4,3-d]pyrimidin-7-yl)naphthalen-2-yl)-2-methylpropanenitrile,
2-(5-ethynyl-6-fluoro-4-(8-fluoro-4-(methyl(((R)-pyrrolidin-2-yl)methyl)amino)-2-(8-methyl-3,8-diazabicyclo[3.2.1]octan-3-yl)pyrido[4,3-d]pyrimidin-7-yl)naphthalen-2-yl)-2-methylpropanenitrile,
2-(5-ethynyl-6-fluoro-4-(8-fluoro-4-(methyl(((R)-piperidin-2-yl)methyl)amino)-2-(8-methyl-3,8-diazabicyclo[3.2.1]octan-3-yl)pyrido[4,3-d]pyrimidin-7-yl)naphthalen-2-yl)-2-methylpropanenitrile,
2-(5-ethynyl-6-fluoro-4-(8-fluoro-4-(methyl(((S)-piperidin-2-yl)methyl)amino)-2-(8-methyl-3,8-diazabicyclo[3.2.1]octan-3-yl)pyrido[4,3-d]pyrimidin-7-yl)naphthalen-2-yl)-2-methylpropanenitrile,
8-ethynyl-7-fluoro-1-(8-fluoro-4-(methyl(((S)-pyrrolidin-2-yl)methyl)amino)-2-(8-methyl-3,8-diazabicyclo[3.2.1]octan-3-yl)pyrido[4,3-d]pyrimidin-7-yl)isoquinolin-3(2H)-one,
8-ethynyl-7-fluoro-1-(8-fluoro-4-(methyl(((R)-pyrrolidin-2-yl)methyl)amino)-2-(8-methyl-3,8-diazabicyclo[3.2.1]octan-3-yl)pyrido[4,3-d]pyrimidin-7-yl)isoquinolin-3(2H)-one,
8-ethynyl-7-fluoro-1-(8-fluoro-4-(methyl(((R)-piperidin-2-yl)methyl)amino)-2-(8-methyl-3,8-diazabicyclo[3.2.1]octan-3-yl)pyrido[4,3-d]pyrimidin-7-yl)isoquinolin-3(2H)-one,
8-ethynyl-7-fluoro-1-(8-fluoro-4-(methyl(((S)-piperidin-2-yl)methyl)amino)-2-(8-methyl-3,8-diazabicyclo[3.2.1]octan-3-yl)pyrido[4,3-d]pyrimidin-7-yl)isoquinolin-3(2H)-one,
5-ethynyl-6-fluoro-4-(8-fluoro-4-(methyl(((S)-pyrrolidin-2-yl)methyl)amino)-2-(8-methyl-3,8-diazabicyclo[3.2.1]octan-3-yl)pyrido[4,3-d]pyrimidin-7-yl)quinolin-2(1H)-one,
5-ethynyl-6-fluoro-4-(8-fluoro-4-(methyl(((R)-pyrrolidin-2-yl)methyl)amino)-2-(8-methyl-3,8-diazabicyclo[3.2.1]octan-3-yl)pyrido[4,3-d]pyrimidin-7-yl)quinolin-2(1H)-one,
5-ethynyl-6-fluoro-4-(8-fluoro-4-(methyl(((R)-piperidin-2-yl)methyl)amino)-2-(8-methyl-3,8-diazabicyclo[3.2.1]octan-3-yl)pyrido[4,3-d]pyrimidin-7-yl)quinolin-2(1H)-one,
5-ethynyl-6-fluoro-4-(8-fluoro-4-(methyl(((S)-piperidin-2-yl)methyl)amino)-2-(8-methyl-3,8-diazabicyclo[3.2.1]octan-3-yl)pyrido[4,3-d]pyrimidin-7-yl)quinolin-2(1H)-one,
(S)-5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)naphthalen-2-ol,
(R)-5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)naphthalen-2-ol,
(R)-5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)naphthalen-2-ol,
(S)-5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)naphthalen-2-ol,
(S)-7-(8-ethynyl-7-fluoronaphthalen-1-yl)-8-fluoro-N-methyl-2-(4-methylpiperazin-1-yl)-N-(pyrrolidin-2-ylmethyl)pyrido[4,3-d]pyrimidin-4-amine,
(R)-7-(8-ethynyl-7-fluoronaphthalen-1-yl)-8-fluoro-N-methyl-2-(4-methylpiperazin-1-yl)-N-(pyrrolidin-2-ylmethyl)pyrido[4,3-d]pyrimidin-4-amine,
(R)-7-(8-ethynyl-7-fluoronaphthalen-1-yl)-8-fluoro-N-methyl-2-(4-methylpiperazin-1-yl)-N-(piperidin-2-ylmethyl)pyrido[4,3-d]pyrimidin-4-amine,
(S)-7-(8-ethynyl-7-fluoronaphthalen-1-yl)-8-fluoro-N-methyl-2-(4-methylpiperazin-1-yl)-N-(piperidin-2-ylmethyl)pyrido[4,3-d]pyrimidin-4-amine,
(S)-7-(5-ethynyl-6-fluoroisoquinolin-4-yl)-8-fluoro-N-methyl-2-(4-methylpiperazin-1-yl)-N-(pyrrolidin-2-ylmethyl)pyrido[4,3-d]pyrimidin-4-amine,
(R)-7-(5-ethynyl-6-fluoroisoquinolin-4-yl)-8-fluoro-N-methyl-2-(4-methylpiperazin-1-yl)-N-(pyrrolidin-2-ylmethyl)pyrido[4,3-d]pyrimidin-4-amine,
(R)-7-(5-ethynyl-6-fluoroisoquinolin-4-yl)-8-fluoro-N-methyl-2-(4-methylpiperazin-1-yl)-N-(piperidin-2-ylmethyl)pyrido[4,3-d]pyrimidin-4-amine,
(S)-7-(5-ethynyl-6-fluoroisoquinolin-4-yl)-8-fluoro-N-methyl-2-(4-methylpiperazin-1-yl)-N-(piperidin-2-ylmethyl)pyrido[4,3-d]pyrimidin-4-amine,
(S)-5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)-2-naphthonitrile,
(R)-5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)-2-naphthonitrile,
(R)-5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)-2-naphthonitrile,
(S)-5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)-2-naphthonitrile,
(S)-7-(8-ethynyl-7-fluoro-3-(trifluoromethyl)naphthalen-1-yl)-8-fluoro-N-methyl-2-(4-methylpiperazin-1-yl)-N-(pyrrolidin-2-ylmethyl)pyrido[4,3-d]pyrimidin-4-amine,
(R)-7-(8-ethynyl-7-fluoro-3-(trifluoromethyl)naphthalen-1-yl)-8-fluoro-N-methyl-2-(4-methylpiperazin-1-yl)-N-(pyrrolidin-2-ylmethyl)pyrido[4,3-d]pyrimidin-4-amine,
(R)-7-(8-ethynyl-7-fluoro-3-(trifluoromethyl)naphthalen-1-yl)-8-fluoro-N-methyl-2-(4-methylpiperazin-1-yl)-N-(piperidin-2-ylmethyl)pyrido[4,3-d]pyrimidin-4-amine,
(S)-7-(8-ethynyl-7-fluoro-3-(trifluoromethyl)naphthalen-1-yl)-8-fluoro-N-methyl-2-(4-methylpiperazin-1-yl)-N-(piperidin-2-ylmethyl)pyrido[4,3-d]pyrimidin-4-amine,
(S)-2-(5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)naphthalen-2-yl)propan-2-ol,
(R)-2-(5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)naphthalen-2-yl)propan-2-ol,
(R)-2-(5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)naphthalen-2-yl)propan-2-ol,
(S)-2-(5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)naphthalen-2-yl)propan-2-ol,
(S)-2-(5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)naphthalen-2-yl)-2-methylpropanenitrile,
(R)-2-(5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)naphthalen-2-yl)-2-methylpropanenitrile,
(R)-2-(5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)naphthalen-2-yl)-2-methylpropanenitrile,
(S)-2-(5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)naphthalen-2-yl)-2-methylpropanenitrile,
(S)-8-ethynyl-7-fluoro-1-(8-fluoro-4-(methyl(pyrrolidin-2-ylmethyl)amino)-2-(4-methylpiperazin-1-yl)pyrido[4,3-d]pyrimidin-7-yl)isoquinolin-3(2H)-one,
(R)-8-ethynyl-7-fluoro-1-(8-fluoro-4-(methyl(pyrrolidin-2-ylmethyl)amino)-2-(4-methylpiperazin-1-yl)pyrido[4,3-d]pyrimidin-7-yl)isoquinolin-3(2H)-one,
(R)-8-ethynyl-7-fluoro-1-(8-fluoro-4-(methyl(piperidin-2-ylmethyl)amino)-2-(4-methylpiperazin-1-yl)pyrido[4,3-d]pyrimidin-7-yl)isoquinolin-3(2H)-one,
(S)-8-ethynyl-7-fluoro-1-(8-fluoro-4-(methyl(piperidin-2-ylmethyl)amino)-2-(4-methylpiperazin-1-yl)pyrido[4,3-d]pyrimidin-7-yl)isoquinolin-3(2H)-one,
(S)-5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)quinolin-2(1H)-one,
(R)-5-ethynyl-6-fluoro-4-(8-fluoro-4-(methyl(pyrrolidin-2-ylmethyl)amino)-2-(4-methylpiperazin-1-yl)pyrido[4,3-d]pyrimidin-7-yl)quinolin-2(1H)-one,
(R)-5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)quinolin-2(1H)-one,
(S)-5-ethynyl-6-fluoro-4-(8-fluoro-4-(methyl(piperidin-2-ylmethyl)amino)-2-(4-methylpiperazin-1-yl)pyrido[4,3-d]pyrimidin-7-yl)quinolin-2(1H)-one,
(S)-5-ethynyl-6-fluoro-4-(8-fluoro-4-(methyl(pyrrolidin-2-ylmethyl)amino)-2-morpholinopyrido[4,3-d]pyrimidin-7-yl)naphthalen-2-ol,
(R)-5-ethynyl-6-fluoro-4-(8-fluoro-4-(methyl(pyrrolidin-2-ylmethyl)amino)-2-morpholinopyrido[4,3-d]pyrimidin-7-yl)naphthalen-2-ol,
(R)-5-ethynyl-6-fluoro-4-(8-fluoro-4-(methyl(piperidin-2-ylmethyl)amino)-2-morpholinopyrido[4,3-d]pyrimidin-7-yl)naphthalen-2-ol,
(S)-5-ethynyl-6-fluoro-4-(8-fluoro-4-(methyl(piperidin-2-ylmethyl)amino)-2-morpholinopyrido[4,3-d]pyrimidin-7-yl)naphthalen-2-ol,
(S)-7-(8-ethynyl-7-fluoronaphthalen-1-yl)-8-fluoro-N-methyl-2-morpholino-N-(pyrrolidin-2-ylmethyl)pyrido[4,3-d]pyrimidin-4-amine,
(R)-7-(8-ethynyl-7-fluoronaphthalen-1-yl)-8-fluoro-N-methyl-2-morpholino-N-(pyrrolidin-2-ylmethyl)pyrido[4,3-d]pyrimidin-4-amine,
(R)-7-(8-ethynyl-7-fluoronaphthalen-1-yl)-8-fluoro-N-methyl-2-morpholino-N-(piperidin-2-ylmethyl)pyrido[4,3-d]pyrimidin-4-amine,
(S)-7-(8-ethynyl-7-fluoronaphthalen-1-yl)-8-fluoro-N-methyl-2-morpholino-N-(piperidin-2-ylmethyl)pyrido[4,3-d]pyrimidin-4-amine,
(S)-7-(5-ethynyl-6-fluoroisoquinolin-4-yl)-8-fluoro-N-methyl-2-morpholino-N-(pyrrolidin-2-ylmethyl)pyrido[4,3-d]pyrimidin-4-amine,
(R)-7-(5-ethynyl-6-fluoroisoquinolin-4-yl)-8-fluoro-N-methyl-2-morpholino-N-(pyrrolidin-2-ylmethyl)pyrido[4,3-d]pyrimidin-4-amine,
(R)-7-(5-ethynyl-6-fluoroisoquinolin-4-yl)-8-fluoro-N-methyl-2-morpholino-N-(piperidin-2-ylmethyl)pyrido[4,3-d]pyrimidin-4-amine,
(S)-7-(5-ethynyl-6-fluoroisoquinolin-4-yl)-8-fluoro-N-methyl-2-morpholino-N-(piperidin-2-ylmethyl)pyrido[4,3-d]pyrimidin-4-amine,
(S)-5-ethynyl-6-fluoro-4-(8-fluoro-4-(methyl(pyrrolidin-2-ylmethyl)amino)-2-morpholinopyrido[4,3-d]pyrimidin-7-yl)-2-naphthonitrile,
(R)-5-ethynyl-6-fluoro-4-(8-fluoro-4-(methyl(pyrrolidin-2-ylmethyl)amino)-2-morpholinopyrido[4,3-d]pyrimidin-7-yl)-2-naphthonitrile,
(R)-5-ethynyl-6-fluoro-4-(8-fluoro-4-(methyl(piperidin-2-ylmethyl)amino)-2-morpholinopyrido[4,3-d]pyrimidin-7-yl)-2-naphthonitrile,
(S)-5-ethynyl-6-fluoro-4-(8-fluoro-4-(methyl(piperidin-2-ylmethyl)amino)-2-morpholinopyrido[4,3-d]pyrimidin-7-yl)-2-naphthonitrile,
(S)-7-(8-ethynyl-7-fluoro-3-(trifluoromethyl)naphthalen-1-yl)-8-fluoro-N-methyl-2-morpholino-N-(pyrrolidin-2-ylmethyl)pyrido[4,3-d]pyrimidin-4-amine,
(R)-7-(8-ethynyl-7-fluoro-3-(trifluoromethyl)naphthalen-1-yl)-8-fluoro-N-methyl-2-morpholino-N-(pyrrolidin-2-ylmethyl)pyrido[4,3-d]pyrimidin-4-amine,
(R)-7-(8-ethynyl-7-fluoro-3-(trifluoromethyl)naphthalen-1-yl)-8-fluoro-N-methyl-2-morpholino-N-(piperidin-2-ylmethyl)pyrido[4,3-d]pyrimidin-4-amine,
(S)-7-(8-ethynyl-7-fluoro-3-(trifluoromethyl)naphthalen-1-yl)-8-fluoro-N-methyl-2-morpholino-N-(piperidin-2-ylmethyl)pyrido[4,3-d]pyrimidin-4-amine,
(S)-2-(5-ethynyl-6-fluoro-4-(8-fluoro-4-(methyl(pyrrolidin-2-ylmethyl)amino)-2-morpholinopyrido[4,3-d]pyrimidin-7-yl)naphthalen-2-yl)propan-2-ol,
(R)-2-(5-ethynyl-6-fluoro-4-(8-fluoro-4-(methyl(pyrrolidin-2-ylmethyl)amino)-2-morpholinopyrido[4,3-d]pyrimidin-7-yl)naphthalen-2-yl)propan-2-ol,
(R)-2-(5-ethynyl-6-fluoro-4-(8-fluoro-4-(methyl(piperidin-2-ylmethyl)amino)-2-morpholinopyrido[4,3-d]pyrimidin-7-yl)naphthalen-2-yl)propan-2-ol,
(S)-2-(5-ethynyl-6-fluoro-4-(8-fluoro-4-(methyl(piperidin-2-ylmethyl)amino)-2-morpholinopyrido[4,3-d]pyrimidin-7-yl)naphthalen-2-yl)propan-2-ol,
(S)-2-(5-ethynyl-6-fluoro-4-(8-fluoro-4-(methyl(pyrrolidin-2-ylmethyl)amino)-2-morpholinopyrido[4,3-d]pyrimidin-7-yl)naphthalen-2-yl)-2-methylpropanenitrile,
(R)-2-(5-ethynyl-6-fluoro-4-(8-fluoro-4-(methyl(pyrrolidin-2-ylmethyl)amino)-2-morpholinopyrido[4,3-d]pyrimidin-7-yl)naphthalen-2-yl)-2-methylpropanenitrile,
(R)-2-(5-ethynyl-6-fluoro-4-(8-fluoro-4-(methyl(piperidin-2-ylmethyl)amino)-2-morpholinopyrido[4,3-d]pyrimidin-7-yl)naphthalen-2-yl)-2-methylpropanenitrile,
(S)-2-(5-ethynyl-6-fluoro-4-(8-fluoro-4-(methyl(piperidin-2-ylmethyl)amino)-2-morpholinopyrido[4,3-d]pyrimidin-7-yl)naphthalen-2-yl)-2-methylpropanenitrile,
(S)-8-ethynyl-7-fluoro-1-(8-fluoro-4-(methyl(pyrrolidin-2-ylmethyl)amino)-2-morpholinopyrido[4,3-d]pyrimidin-7-yl)isoquinolin-3(2H)-one,
(R)-8-ethynyl-7-fluoro-1-(8-fluoro-4-(methyl(pyrrolidin-2-ylmethyl)amino)-2-morpholinopyrido[4,3-d]pyrimidin-7-yl)isoquinolin-3(2H)-one,
(R)-8-ethynyl-7-fluoro-1-(8-fluoro-4-(methyl(piperidin-2-ylmethyl)amino)-2-morpholinopyrido[4,3-d]pyrimidin-7-yl)isoquinolin-3(2H)-one,
(S)-8-ethynyl-7-fluoro-1-(8-fluoro-4-(methyl(piperidin-2-ylmethyl)amino)-2-morpholinopyrido[4,3-d]pyrimidin-7-yl)isoquinolin-3(2H)-one,
(S)-5-ethynyl-6-fluoro-4-(8-fluoro-4-(methyl(pyrrolidin-2-ylmethyl)amino)-2-morpholinopyrido[4,3-d]pyrimidin-7-yl)quinolin-2(1H)-one,
(R)-5-ethynyl-6-fluoro-4-(8-fluoro-4-(methyl(pyrrolidin-2-ylmethyl)amino)-2-morpholinopyrido[4,3-d]pyrimidin-7-yl)quinolin-2(1H)-one,
(R)-5-ethynyl-6-fluoro-4-(8-fluoro-4-(methyl(piperidin-2-ylmethyl)amino)-2-morpholinopyrido[4,3-d]pyrimidin-7-yl)quinolin-2(1H)-one,
(S)-5-ethynyl-6-fluoro-4-(8-fluoro-4-(methyl(piperidin-2-ylmethyl)amino)-2-morpholinopyrido[4,3-d]pyrimidin-7-yl)quinolin-2(1H)-one,
(S)-1-(7-(8-ethynyl-7-fluoro-3-hydroxynaphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoro-3-hydroxynaphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoro-3-hydroxynaphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(8-ethynyl-7-fluoro-3-hydroxynaphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(8-ethynyl-7-fluoronaphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoronaphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoronaphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(8-ethynyl-7-fluoronaphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(5-ethynyl-6-fluoroisoquinolin-4-yl)-8-fluoro-4-(methyl(pyrrolidin-2-ylmethyl)amino)pyrido[4,3-d]pyrimidin-2-yl)-4-methylpiperidin-4-ol,
(R)-1-(7-(5-ethynyl-6-fluoroisoquinolin-4-yl)-8-fluoro-4-(methyl(pyrrolidin-2-ylmethyl)amino)pyrido[4,3-d]pyrimidin-2-yl)-4-methylpiperidin-4-ol,
(R)-1-(7-(5-ethynyl-6-fluoroisoquinolin-4-yl)-8-fluoro-4-(methyl(piperidin-2-ylmethyl)amino)pyrido[4,3-d]pyrimidin-2-yl)-4-methylpiperidin-4-ol,
(S)-1-(7-(5-ethynyl-6-fluoroisoquinolin-4-yl)-8-fluoro-4-(methyl(piperidin-2-ylmethyl)amino)pyrido[4,3-d]pyrimidin-2-yl)-4-methylpiperidin-4-ol,
(S)-5-ethynyl-6-fluoro-4-(8-fluoro-2-(4-hydroxy-4-methylpiperidin-1-yl)-4-(methyl(pyrrolidin-2-ylmethyl)amino)pyrido[4,3-d]pyrimidin-7-yl)-2-naphthonitrile,
(R)-5-ethynyl-6-fluoro-4-(8-fluoro-2-(4-hydroxy-4-methylpiperidin-1-yl)-4-(methyl(pyrrolidin-2-ylmethyl)amino)pyrido[4,3-d]pyrimidin-7-yl)-2-naphthonitrile,
(R)-5-ethynyl-6-fluoro-4-(8-fluoro-2-(4-hydroxy-4-methylpiperidin-1-yl)-4-(methyl(piperidin-2-ylmethyl)amino)pyrido[4,3-d]pyrimidin-7-yl)-2-naphthonitrile,
(S)-5-ethynyl-6-fluoro-4-(8-fluoro-2-(4-hydroxy-4-methylpiperidin-1-yl)-4-(methyl(piperidin-2-ylmethyl)amino)pyrido[4,3-d]pyrimidin-7-yl)-2-naphthonitrile,
(S)-1-(7-(8-ethynyl-7-fluoro-3-(trifluoromethyl)naphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoro-3-(trifluoromethyl)naphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoro-3-(trifluoromethyl)naphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(8-ethynyl-7-fluoro-3-(trifluoromethyl)naphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(8-ethynyl-7-fluoro-3-(2-hydroxypropan-2-yl)naphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoro-3-(2-hydroxypropan-2-yl)naphthalen-1-yl)-8-fluoro-4-(methyl(pyrrolidin-2-ylmethyl)amino)pyrido[4,3-d]pyrimidin-2-yl)-4-methylpiperidin-4-ol,
(R)-1-(7-(8-ethynyl-7-fluoro-3-(2-hydroxypropan-2-yl)naphthalen-1-yl)-8-fluoro-4-(methyl(piperidin-2-ylmethyl)amino)pyrido[4,3-d]pyrimidin-2-yl)-4-methylpiperidin-4-ol,
(S)-1-(7-(8-ethynyl-7-fluoro-3-(2-hydroxypropan-2-yl)naphthalen-1-yl)-8-fluoro-4-(methyl(piperidin-2-ylmethyl)amino)pyrido[4,3-d]pyrimidin-2-yl)-4-methylpiperidin-4-ol,
(S)-2-(5-ethynyl-6-fluoro-4-(8-fluoro-2-(4-hydroxy-4-methylpiperidin-1-yl)-4-(methyl(pyrrolidin-2-ylmethyl)amino)pyrido[4,3-d]pyrimidin-7-yl)naphthalen-2-yl)-2-methylpropanenitrile,
(R)-2-(5-ethynyl-6-fluoro-4-(8-fluoro-2-(4-hydroxy-4-methylpiperidin-1-yl)-4-(methyl(pyrrolidin-2-ylmethyl)amino)pyrido[4,3-d]pyrimidin-7-yl)naphthalen-2-yl)-2-methylpropanenitrile,
(R)-2-(5-ethynyl-6-fluoro-4-(8-fluoro-2-(4-hydroxy-4-methylpiperidin-1-yl)-4-(methyl(piperidin-2-ylmethyl)amino)pyrido[4,3-d]pyrimidin-7-yl)naphthalen-2-yl)-2-methylpropanenitrile,
(S)-2-(5-ethynyl-6-fluoro-4-(8-fluoro-2-(4-hydroxy-4-methylpiperidin-1-yl)-4-(methyl(piperidin-2-ylmethyl)amino)pyrido[4,3-d]pyrimidin-7-yl)naphthalen-2-yl)-2-methylpropanenitrile,
(S)-8-ethynyl-7-fluoro-1-(8-fluoro-2-(4-hydroxy-4-methylpiperidin-1-yl)-4-(methyl(pyrrolidin-2-ylmethyl)amino)pyrido[4,3-d]pyrimidin-7-yl)isoquinolin-3(2H)-one,
(R)-8-ethynyl-7-fluoro-1-(8-fluoro-2-(4-hydroxy-4-methylpiperidin-1-yl)-4-(methyl(pyrrolidin-2-ylmethyl)amino)pyrido[4,3-d]pyrimidin-7-yl)isoquinolin-3(2H)-one,
(R)-8-ethynyl-7-fluoro-1-(8-fluoro-2-(4-hydroxy-4-methylpiperidin-1-yl)-4-(methyl(piperidin-2-ylmethyl)amino)pyrido[4,3-d]pyrimidin-7-yl)isoquinolin-3(2H)-one,
(S)-8-ethynyl-7-fluoro-1-(8-fluoro-2-(4-hydroxy-4-methylpiperidin-1-yl)-4-(methyl(piperidin-2-ylmethyl)amino)pyrido[4,3-d]pyrimidin-7-yl)isoquinolin-3(2H)-one,
(S)-5-ethynyl-6-fluoro-4-(8-fluoro-2-(4-hydroxy-4-methylpiperidin-1-yl)-4-(methyl(pyrrolidin-2-ylmethyl)amino)pyrido[4,3-d]pyrimidin-7-yl)quinolin-2(1H)-one,
(R)-5-ethynyl-6-fluoro-4-(8-fluoro-2-(4-hydroxy-4-methylpiperidin-1-yl)-4-(methyl(pyrrolidin-2-ylmethyl)amino)pyrido[4,3-d]pyrimidin-7-yl)quinolin-2(1H)-one,
(R)-5-ethynyl-6-fluoro-4-(8-fluoro-2-(4-hydroxy-4-methylpiperidin-1-yl)-4-(methyl(piperidin-2-ylmethyl)amino)pyrido[4,3-d]pyrimidin-7-yl)quinolin-2(1H)-one,
(S)-5-ethynyl-6-fluoro-4-(8-fluoro-2-(4-hydroxy-4-methylpiperidin-1-yl)-4-(methyl(piperidin-2-ylmethyl)amino)pyrido[4,3-d]pyrimidin-7-yl)quinolin-2(1H)-one,
4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol,
(1R,5S)-3-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane,
(1R,5S)-3-(7-(5-ethynyl-6-fluoroisoquinolin-4-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane,
4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoro-2-naphthonitrile,
(1R,5S)-3-(7-(8-ethynyl-7-fluoro-3-(trifluoromethyl)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[3.2.1]octane,
2-(4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-yl)propan-2-ol,
2-(4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-yl)-2-methylpropanenitrile,
4-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoroquinolin-2(1H)-one,
1-(4-((1R,5S)-8-oxa-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-8-ethynyl-7-fluoroisoquinolin-3(2H)-one,
4-(4-((1R,5S)-8-oxa-3-azabicyclo[3.2.1]octan-3-yl)-8-fluoro-2-(8-methyl-3,8-diazabicyclo[3.2.1]octan-3-yl)pyrido[4,3-d]pyrimidin-7-yl)-5-ethynyl-6-fluoronaphthalen-2-ol,
(1R,5S)-3-(7-(8-ethynyl-7-fluoronaphthalen-1-yl)-8-fluoro-2-(8-methyl-3,8-diazabicyclo[3.2.1]octan-3-yl)pyrido[4,3-d]pyrimidin-4-yl)-8-oxa-3-azabicyclo[3.2.1]octane,
(1R,5S)-3-(7-(5-ethynyl-6-fluoroisoquinolin-4-yl)-8-fluoro-2-(8-methyl-3,8-diazabicyclo[3.2.1]octan-3-yl)pyrido[4,3-d]pyrimidin-4-yl)-8-oxa-3-azabicyclo[3.2.1]octane,
4-(4-((1R,5S)-8-oxa-3-azabicyclo[3.2.1]octan-3-yl)-8-fluoro-2-(8-methyl-3,8-diazabicyclo[3.2.1]octan-3-yl)pyrido[4,3-d]pyrimidin-7-yl)-5-ethynyl-6-fluoro-2-naphthonitrile,
(1R,5S)-3-(7-(8-ethynyl-7-fluoro-3-(trifluoromethyl)naphthalen-1-yl)-8-fluoro-2-(8-methyl-3,8-diazabicyclo[3.2.1]octan-3-yl)pyrido[4,3-d]pyrimidin-4-yl)-8-oxa-3-azabicyclo[3.2.1]octane,
2-(4-(4-((1R,5S)-8-oxa-3-azabicyclo[3.2.1]octan-3-yl)-8-fluoro-2-(8-methyl-3,8-diazabicyclo[3.2.1]octan-3-yl)pyrido[4,3-d]pyrimidin-7-yl)-5-ethynyl-6-fluoronaphthalen-2-yl)propan-2-ol,
2-(4-(4-((1R,5S)-8-oxa-3-azabicyclo[3.2.1]octan-3-yl)-8-fluoro-2-(8-methyl-3,8-diazabicyclo[3.2.1]octan-3-yl)pyrido[4,3-d]pyrimidin-7-yl)-5-ethynyl-6-fluoronaphthalen-2-yl)-2-methylpropanenitrile,
4-(4-((1R,5S)-8-oxa-3-azabicyclo[3.2.1]octan-3-yl)-8-fluoro-2-(8-methyl-3,8-diazabicyclo[3.2.1]octan-3-yl)pyrido[4,3-d]pyrimidin-7-yl)-5-ethynyl-6-fluoroquinolin-2(1H)-one,
1-(4-((1R,5S)-8-oxa-3-azabicyclo[3.2.1]octan-3-yl)-8-fluoro-2-(8-methyl-3,8-diazabicyclo[3.2.1]octan-3-yl)pyrido[4,3-d]pyrimidin-7-yl)-8-ethynyl-7-fluoroisoquinolin-3(2H)-one,
4-(4-((1R,5S)-8-oxa-3-azabicyclo[3.2.1]octan-3-yl)-8-fluoro-2-(4-methylpiperazin-1-yl)pyrido[4,3-d]pyrimidin-7-yl)-5-ethynyl-6-fluoronaphthalen-2-ol,
(1R,5S)-3-(7-(8-ethynyl-7-fluoronaphthalen-1-yl)-8-fluoro-2-(4-methylpiperazin-1-yl)pyrido[4,3-d]pyrimidin-4-yl)-8-oxa-3-azabicyclo[3.2.1]octane,
(1R,5S)-3-(7-(5-ethynyl-6-fluoroisoquinolin-4-yl)-8-fluoro-2-(4-methylpiperazin-1-yl)pyrido[4,3-d]pyrimidin-4-yl)-8-oxa-3-azabicyclo[3.2.1]octane,
4-(4-((1R,5S)-8-oxa-3-azabicyclo[3.2.1]octan-3-yl)-8-fluoro-2-(4-methylpiperazin-1-yl)pyrido[4,3-d]pyrimidin-7-yl)-5-ethynyl-6-fluoro-2-naphthonitrile,
(1R,5S)-3-(7-(8-ethynyl-7-fluoro-3-(trifluoromethyl)naphthalen-1-yl)-8-fluoro-2-(4-methylpiperazin-1-yl)pyrido[4,3-d]pyrimidin-4-yl)-8-oxa-3-azabicyclo[3.2.1]octane,
2-(4-(4-((1R,5S)-8-oxa-3-azabicyclo[3.2.1]octan-3-yl)-8-fluoro-2-(4-methylpiperazin-1-yl)pyrido[4,3-d]pyrimidin-7-yl)-5-ethynyl-6-fluoronaphthalen-2-yl)propan-2-ol,
2-(4-(4-((1R,5S)-8-oxa-3-azabicyclo[3.2.1]octan-3-yl)-8-fluoro-2-(4-methylpiperazin-1-yl)pyrido[4,3-d]pyrimidin-7-yl)-5-ethynyl-6-fluoronaphthalen-2-yl)-2-methylpropanenitrile,
4-(4-((1R,5S)-8-oxa-3-azabicyclo[3.2.1]octan-3-yl)-8-fluoro-2-(4-methylpiperazin-1-yl)pyrido[4,3-d]pyrimidin-7-yl)-5-ethynyl-6-fluoroquinolin-2(1H)-one,
1-(4-((1R,5S)-8-oxa-3-azabicyclo[3.2.1]octan-3-yl)-8-fluoro-2-(4-methylpiperazin-1-yl)pyrido[4,3-d]pyrimidin-7-yl)-8-ethynyl-7-fluoroisoquinolin-3(2H)-one,
4-(4-((1R,5S)-8-oxa-3-azabicyclo[3.2.1]octan-3-yl)-8-fluoro-2-morpholinopyrido[4,3-d]pyrimidin-7-yl)-5-ethynyl-6-fluoronaphthalen-2-ol,
(1R,5S)-3-(7-(8-ethynyl-7-fluoronaphthalen-1-yl)-8-fluoro-2-morpholinopyrido[4,3-d]pyrimidin-4-yl)-8-oxa-3-azabicyclo[3.2.1]octane,
(1R,5S)-3-(7-(5-ethynyl-6-fluoroisoquinolin-4-yl)-8-fluoro-2-morpholinopyrido[4,3-d]pyrimidin-4-yl)-8-oxa-3-azabicyclo[3.2.1]octane,
4-(4-((1R,5S)-8-oxa-3-azabicyclo[3.2.1]octan-3-yl)-8-fluoro-2-morpholinopyrido[4,3-d]pyrimidin-7-yl)-5-ethynyl-6-fluoro-2-naphthonitrile,
(1R,5S)-3-(7-(8-ethynyl-7-fluoro-3-(trifluoromethyl)naphthalen-1-yl)-8-fluoro-2-morpholinopyrido[4,3-d]pyrimidin-4-yl)-8-oxa-3-azabicyclo[3.2.1]octane,
2-(4-(4-((1R,5S)-8-oxa-3-azabicyclo[3.2.1]octan-3-yl)-8-fluoro-2-morpholinopyrido[4,3-d]pyrimidin-7-yl)-5-ethynyl-6-fluoronaphthalen-2-yl)propan-2-ol,
2-(4-(4-((1R,5S)-8-oxa-3-azabicyclo[3.2.1]octan-3-yl)-8-fluoro-2-morpholinopyrido[4,3-d]pyrimidin-7-yl)-5-ethynyl-6-fluoronaphthalen-2-yl)-2-methylpropanenitrile,
4-(4-((1R,5S)-8-oxa-3-azabicyclo[3.2.1]octan-3-yl)-8-fluoro-2-morpholinopyrido[4,3-d]pyrimidin-7-yl)-5-ethynyl-6-fluoroquinolin-2(1H)-one,
1-(4-((1R,5S)-8-oxa-3-azabicyclo[3.2.1]octan-3-yl)-8-fluoro-2-morpholinopyrido[4,3-d]pyrimidin-7-yl)-8-ethynyl-7-fluoroisoquinolin-3(2H)-one,
1-(4-((1R,5S)-8-oxa-3-azabicyclo[3.2.1]octan-3-yl)-7-(8-ethynyl-7-fluoro-3-hydroxynaphthalen-1-yl)-8-fluoropyrido[4,3-d]pyrimidin-2-yl)-4-methylpiperidin-4-ol,
1-(4-((1R,5S)-8-oxa-3-azabicyclo[3.2.1]octan-3-yl)-7-(8-ethynyl-7-fluoronaphthalen-1-yl)-8-fluoropyrido[4,3-d]pyrimidin-2-yl)-4-methylpiperidin-4-ol,
1-(4-((1R,5S)-8-oxa-3-azabicyclo[3.2.1]octan-3-yl)-7-(5-ethynyl-6-fluoroisoquinolin-4-yl)-8-fluoropyrido[4,3-d]pyrimidin-2-yl)-4-methylpiperidin-4-ol,
4-(4-((1R,5S)-8-oxa-3-azabicyclo[3.2.1]octan-3-yl)-8-fluoro-2-(4-hydroxy-4-methylpiperidin-1-yl)pyrido[4,3-d]pyrimidin-7-yl)-5-ethynyl-6-fluoro-2-naphthonitrile,
1-(4-((1R,5S)-8-oxa-3-azabicyclo[3.2.1]octan-3-yl)-7-(8-ethynyl-7-fluoro-3-(trifluoromethyl)naphthalen-1-yl)-8-fluoropyrido[4,3-d]pyrimidin-2-yl)-4-methylpiperidin-4-ol,
1-(4-((1R,5S)-8-oxa-3-azabicyclo[3.2.1]octan-3-yl)-7-(8-ethynyl-7-fluoro-3-(2-hydroxypropan-2-yl)naphthalen-1-yl)-8-fluoropyrido[4,3-d]pyrimidin-2-yl)-4-methylpiperidin-4-ol,
2-(4-(4-((1R,5S)-8-oxa-3-azabicyclo[3.2.1]octan-3-yl)-8-fluoro-2-(4-hydroxy-4-methylpiperidin-1-yl)pyrido[4,3-d]pyrimidin-7-yl)-5-ethynyl-6-fluoronaphthalen-2-yl)-2-methylpropanenitrile,
4-(4-((1R,5S)-8-oxa-3-azabicyclo[3.2.1]octan-3-yl)-8-fluoro-2-(4-hydroxy-4-methylpiperidin-1-yl)pyrido[4,3-d]pyrimidin-7-yl)-5-ethynyl-6-fluoroquinolin-2(1H)-one,
1-(4-((1R,5S)-8-oxa-3-azabicyclo[3.2.1]octan-3-yl)-8-fluoro-2-(4-hydroxy-4-methylpiperidin-1-yl)pyrido[4,3-d]pyrimidin-7-yl)-8-ethynyl-7-fluoroisoquinolin-3(2H)-one,
(1R,5S)-3-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-thia-3-azabicyclo[3.2.1]octane 8,8-dioxide,
4-(4-((1R,5R)-6,6-difluoro-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol,
4-(4-((1R,5S)-6,6-dimethyl-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol,
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-1,6-naphthyridin-7-yl)-5-ethynyl-6-fluoronaphthalen-2-ol,
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-3-methyl-1,6-naphthyridin-7-yl)-5-ethynyl-6-fluoronaphthalen-2-ol,
4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-3-(trifluoromethyl)-1,6-naphthyridin-7-yl)-5-ethynyl-6-fluoronaphthalen-2-ol,
4-((1R,5S)-3,8-diazabicyclo[3.2.1]octan-3-yl)-7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)-1,6-naphthyridine-3-carbonitrile,
4-(4-((1R,5S)-3,8-diazabicyclo[3.2.1]octan-3-yl)-3-chloro-8-fluoro-2-(((2R,7aS)-2-fluorotetrahydro-1H-pyrrolizin-7a(5H)-yl)methoxy)-1,6-naphthyridin-7-yl)-5-ethynyl-6-fluoronaphthalen-2-ol,
4-(9-((1R,5S)-3,8-diazabicyclo[3.2.1]octan-3-yl)-5-fluoro-2-((2R,7aS)-2-fluorotetrahydro-1H-pyrrolizin-7a(5H)-yl)oxazolo[5,4-b][1,6]naphthyridin-6-yl)-5-ethynyl-6-fluoronaphthalen-2-ol,
4-(9-((1R,5S)-3,8-diazabicyclo[3.2.1]octan-3-yl)-5-fluoro-2-((2R,7aS)-2-fluorotetrahydro-1H-pyrrolizin-7a(5H)-yl)-3H-imidazo[4,5-b][1,6]naphthyridin-6-yl)-5-ethynyl-6-fluoronaphthalen-2-ol,
4-(10-((1R,5S)-3,8-diazabicyclo[3.2.1]octan-3-yl)-6-fluoro-3-((2R,7aS)-2-fluorotetrahydro-1H-pyrrolizin-7a(5H)-yl)pyrazino[2,3-b][1,6]naphthyridin-7-yl)-5-ethynyl-6-fluoronaphthalen-2-ol,
1-((1R,5S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one,
1-((1R,5S)-3-(7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octan-8-yl)prop-2-en-1-one,
1-((1R,5S)-3-(7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)-3,8-diazabicyclo[3.2.1]octan-8-yl)prop-2-en-1-one,
1-((1R,5S)-3-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one,
1-((1R,5S)-3-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one,
1-((1R,5S)-3-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one,
1-((1R,5S)-3-(7-(8-ethynyl-7-fluoronaphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octan-8-yl)prop-2-en-1-one,
1-((1R,5S)-3-(7-(8-ethynyl-7-fluoronaphthalen-1-yl)-8-fluoro-2-(((S)-1-methylpiperidin-2-yl)methoxy)pyrido[4,3-d]pyrimidin-4-yl)-3,8-diazabicyclo[3.2.1]octan-8-yl)prop-2-en-1-one,
4-(4-((1R,5S)-8-acryloyl-3,8-diazabicyclo[3.2.1]octan-3-yl)-8-fluoro-2-((tetrahydro-1H-pyrrolizin-7a(5H)-yl)methoxy)pyrido[4,3-d]pyrimidin-7-yl)-5-ethynyl-6-fluoro-2-naphthonitrile,
2-(4-(4-((1R,5S)-8-acryloyl-3,8-diazabicyclo[3.2.1]octan-3-yl)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-yl)-2-methylpropanenitrile,
1-((1R,5S)-3-(7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one,
4-((1R,5S)-8-acryloyl-3,8-diazabicyclo[3.2.1]octan-3-yl)-7-(8-ethynyl-7-fluoro-3-hydroxynaphthalen-1-yl)-8-fluoro-2-((tetrahydro-1H-pyrrolizin-7a(5H)-yl)methoxy)quinazoline-6-carbonitrile,
1-((1R,5S)-3-(6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-3,8-diazabicyclo[3.2.1]octan-8-yl)prop-2-en-1-one,
1-((1R,5S)-3-(7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)-3,8-diazabicyclo[3.2.1]octan-8-yl)prop-2-en-1-one,
2-(4-((1R,5S)-8-acryloyl-3,8-diazabicyclo[3.2.1]octan-3-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-6-yl)-2-methylpropanenitrile,
1-((1R,5S)-3-(6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-3,8-diazabicyclo[3.2.1]octan-8-yl)prop-2-en-1-one,
1-((1R,5S)-3-(7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one,
1-((S)-2-(((7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)(methyl)amino)methyl)pyrrolidin-1-yl)prop-2-en-1-one,
(S)-1-(2-(((2-(3-(dimethylamino)azetidin-1-yl)-7-(8-ethynyl-7-fluoro-3-hydroxynaphthalen-1-yl)-8-fluoropyrido[4,3-d]pyrimidin-4-yl)(methyl)amino)methyl)pyrrolidin-1-yl)prop-2-en-1-one,
(S)-4-(4-(((1-acryloylpyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoro-2-naphthonitrile,
(S)-2-(4-(4-(((1-acryloylpyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-yl)-2-methylpropanenitrile,
(S)-1-(2-(((7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)pyrrolidin-1-yl)prop-2-en-1-one,
(S)-1-(2-(((7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoronaphthalen-1-yl)-8-fluoro-2-(((S)-1-methylpyrrolidin-2-yl)methoxy)pyrido[4,3-d]pyrimidin-4-yl)(methyl)amino)methyl)pyrrolidin-1-yl)prop-2-en-1-one,
1-((S)-2-(((7-(8-ethynyl-7-fluoronaphthalen-1-yl)-8-fluoro-2-(((S)-1-methylpiperidin-2-yl)methoxy)pyrido[4,3-d]pyrimidin-4-yl)(methyl)amino)methyl)pyrrolidin-1-yl)prop-2-en-1-one,
4-((((S)-1-acryloylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazoline-6-carbonitrile,
1-((2S)-2-(((6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)pyrrolidin-1-yl)prop-2-en-1-one,
1-((2S)-2-(((7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)(methyl)amino)methyl)pyrrolidin-1-yl)prop-2-en-1-one,
2-(4-((((S)-1-acryloylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazolin-6-yl)-2-methylpropanenitrile,
1-((2S)-2-(((6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)pyrrolidin-1-yl)prop-2-en-1-one,
(S)-1-(2-(((7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)(methyl)amino)methyl)pyrrolidin-1-yl)prop-2-en-1-one,
(2S)-1-acryloyl-2-(((7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)(methyl)amino)methyl)-3-methylpyrrolidine-3-carbonitrile,
(2S)-1-acryloyl-2-(((2-(3-(dimethylamino)azetidin-1-yl)-7-(8-ethynyl-7-fluoro-3-hydroxynaphthalen-1-yl)-8-fluoropyrido[4,3-d]pyrimidin-4-yl)(methyl)amino)methyl)-3-methylpyrrolidine-3-carbonitrile,
(2S)-1-acryloyl-2-(((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
(2S)-1-acryloyl-2-(((7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
(2S)-1-acryloyl-2-(((7-(8-ethynyl-7-fluoronaphthalen-1-yl)-8-fluoro-2-(((S)-1-methylpyrrolidin-2-yl)methoxy)pyrido[4,3-d]pyrimidin-4-yl)(methyl)amino)methyl)-3-methylpyrrolidine-3-carbonitrile,
(2S)-1-acryloyl-2-(((7-(8-ethynyl-7-fluoronaphthalen-1-yl)-8-fluoro-2-(((S)-1-methylpiperidin-2-yl)methoxy)pyrido[4,3-d]pyrimidin-4-yl)(methyl)amino)methyl)-3-methylpyrrolidine-3-carbonitrile,
(2S)-1-acryloyl-2-(((7-(3-cyano-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
(2S)-1-acryloyl-2-(((7-(3-(2-cyanopropan-2-yl)-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
(2S)-1-acryloyl-2-(((7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
4-((((2S)-1-acryloyl-3-cyano-3-methylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazoline-6-carbonitrile,
(2S)-1-acryloyl-2-(((6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3-methylpyrrolidine-3-carbonitrile,
(2S)-1-acryloyl-2-(((7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)(methyl)amino)methyl)-3-methylpyrrolidine-3-carbonitrile,
(2S)-1-acryloyl-2-(((6-(2-cyanopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3-methylpyrrolidine-3-carbonitrile,
(2S)-1-acryloyl-2-(((6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3-methylpyrrolidine-3-carbonitrile,
(2S)-1-acryloyl-2-(((7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)(methyl)amino)methyl)-3-methylpyrrolidine-3-carbonitrile,
1-((S)-2-(((7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)(methyl)amino)methyl)-3,3-dimethylpyrrolidin-1-yl)prop-2-en-1-one,
(S)-1-(2-(((2-(3-(dimethylamino)azetidin-1-yl)-7-(8-ethynyl-7-fluoro-3-hydroxynaphthalen-1-yl)-8-fluoropyrido[4,3-d]pyrimidin-4-yl)(methyl)amino)methyl)-3,3-dimethylpyrrolidin-1-yl)prop-2-en-1-one,
1-((S)-2-(((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)-3,3-dimethylpyrrolidin-1-yl)prop-2-en-1-one,
(S)-1-(2-(((7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-dimethylpyrrolidin-1-yl)prop-2-en-1-one,
1-((S)-2-(((7-(8-ethynyl-7-fluoronaphthalen-1-yl)-8-fluoro-2-(((S)-1-methylpyrrolidin-2-yl)methoxy)pyrido[4,3-d]pyrimidin-4-yl)(methyl)amino)methyl)-3,3-dimethylpyrrolidin-1-yl)prop-2-en-1-one,
1-((S)-2-(((7-(8-ethynyl-7-fluoronaphthalen-1-yl)-8-fluoro-2-(((S)-1-methylpiperidin-2-yl)methoxy)pyrido[4,3-d]pyrimidin-4-yl)(methyl)amino)methyl)-3,3-dimethylpyrrolidin-1-yl)prop-2-en-1-one,
(S)-4-(4-(((1-acryloyl-3,3-dimethylpyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoro-2-naphthonitrile,
(S)-2-(4-(4-(((1-acryloyl-3,3-dimethylpyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-yl)-2-methylpropanenitrile,
(S)-1-(2-(((7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-dimethylpyrrolidin-1-yl)prop-2-en-1-one,
4-((((S)-1-acryloyl-3,3-dimethylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazoline-6-carbonitrile,
1-((2S)-2-(((6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3,3-dimethylpyrrolidin-1-yl)prop-2-en-1-one,
1-((2S)-2-(((7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)(methyl)amino)methyl)-3,3-dimethylpyrrolidin-1-yl)prop-2-en-1-one,
2-(4-((((S)-1-acryloyl-3,3-dimethylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazolin-6-yl)-2-methylpropanenitrile,
1-((2S)-2-(((6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3,3-dimethylpyrrolidin-1-yl)prop-2-en-1-one,
(S)-1-(2-(((7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)(methyl)amino)methyl)-3,3-dimethylpyrrolidin-1-yl)prop-2-en-1-one,
1-((2R)-2-(((7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)(methyl)amino)methyl)-3-hydroxy-3-methylpyrrolidin-1-yl)prop-2-en-1-one,
1-((2R)-2-(((2-(3-(dimethylamino)azetidin-1-yl)-7-(8-ethynyl-7-fluoro-3-hydroxynaphthalen-1-yl)-8-fluoropyrido[4,3-d]pyrimidin-4-yl)(methyl)amino)methyl)-3-hydroxy-3-methylpyrrolidin-1-yl)prop-2-en-1-one,
1-((2R)-2-(((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)-3-hydroxy-3-methylpyrrolidin-1-yl)prop-2-en-1-one,
1-((2R)-2-(((7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-hydroxy-3-methylpyrrolidin-1-yl)prop-2-en-1-one,
1-((2R)-2-(((7-(8-ethynyl-7-fluoronaphthalen-1-yl)-8-fluoro-2-(((S)-1-methylpyrrolidin-2-yl)methoxy)pyrido[4,3-d]pyrimidin-4-yl)(methyl)amino)methyl)-3-hydroxy-3-methylpyrrolidin-1-yl)prop-2-en-1-one,
1-((2R)-2-(((7-(8-ethynyl-7-fluoronaphthalen-1-yl)-8-fluoro-2-(((S)-1-methylpiperidin-2-yl)methoxy)pyrido[4,3-d]pyrimidin-4-yl)(methyl)amino)methyl)-3-hydroxy-3-methylpyrrolidin-1-yl)prop-2-en-1-one,
4-(4-((((2R)-1-acryloyl-3-hydroxy-3-methylpyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoro-2-naphthonitrile,
2-(4-(4-((((2R)-1-acryloyl-3-hydroxy-3-methylpyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-yl)-2-methylpropanenitrile,
1-((2R)-2-(((7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3-hydroxy-3-methylpyrrolidin-1-yl)prop-2-en-1-one,
4-((((2R)-1-acryloyl-3-hydroxy-3-methylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazoline-6-carbonitrile,
1-((2R)-2-(((6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3-hydroxy-3-methylpyrrolidin-1-yl)prop-2-en-1-one,
1-((2R)-2-(((7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)(methyl)amino)methyl)-3-hydroxy-3-methylpyrrolidin-1-yl)prop-2-en-1-one,
2-(4-((((2R)-1-acryloyl-3-hydroxy-3-methylpyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazolin-6-yl)-2-methylpropanenitrile,
1-((2R)-2-(((6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3-hydroxy-3-methylpyrrolidin-1-yl)prop-2-en-1-one,
1-((2R)-2-(((7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)(methyl)amino)methyl)-3-hydroxy-3-methylpyrrolidin-1-yl)prop-2-en-1-one,
1-((R)-2-(((7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)(methyl)amino)methyl)-3,3-difluoropyrrolidin-1-yl)prop-2-en-1-one,
(R)-1-(2-(((2-(3-(dimethylamino)azetidin-1-yl)-7-(8-ethynyl-7-fluoro-3-hydroxynaphthalen-1-yl)-8-fluoropyrido[4,3-d]pyrimidin-4-yl)(methyl)amino)methyl)-3,3-difluoropyrrolidin-1-yl)prop-2-en-1-one,
1-((R)-2-(((7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)-3,3-difluoropyrrolidin-1-yl)prop-2-en-1-one,
(R)-1-(2-(((7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-difluoropyrrolidin-1-yl)prop-2-en-1-one,
1-((R)-2-(((7-(8-ethynyl-7-fluoronaphthalen-1-yl)-8-fluoro-2-(((S)-1-methylpyrrolidin-2-yl)methoxy)pyrido[4,3-d]pyrimidin-4-yl)(methyl)amino)methyl)-3,3-difluoropyrrolidin-1-yl)prop-2-en-1-one,
1-((R)-2-(((7-(8-ethynyl-7-fluoronaphthalen-1-yl)-8-fluoro-2-(((S)-1-methylpiperidin-2-yl)methoxy)pyrido[4,3-d]pyrimidin-4-yl)(methyl)amino)methyl)-3,3-difluoropyrrolidin-1-yl)prop-2-en-1-one,
(R)-4-(4-(((1-acryloyl-3,3-difluoropyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoro-2-naphthonitrile,
(R)-2-(4-(4-(((1-acryloyl-3,3-difluoropyrrolidin-2-yl)methyl)(methyl)amino)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-yl)-2-methylpropanenitrile,
(R)-1-(2-(((7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)(methyl)amino)methyl)-3,3-difluoropyrrolidin-1-yl)prop-2-en-1-one,
4-((((R)-1-acryloyl-3,3-difluoropyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazoline-6-carbonitrile,
1-((2R)-2-(((6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3,3-difluoropyrrolidin-1-yl)prop-2-en-1-one,
1-((2R)-2-(((7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)(methyl)amino)methyl)-3,3-difluoropyrrolidin-1-yl)prop-2-en-1-one,
2-(4-((((R)-1-acryloyl-3,3-difluoropyrrolidin-2-yl)methyl)(methyl)amino)-7-(8-ethynyl-7-fluoro-3-hydroxynaphthalen-1-yl)-8-fluoro-2-((tetrahydro-1H-pyrrolizin-7a(5H)-yl)methoxy)quinazolin-6-yl)-2-methylpropanenitrile,
1-((2R)-2-(((6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)(methyl)amino)methyl)-3,3-difluoropyrrolidin-1-yl)prop-2-en-1-one,
(R)-1-(2-(((7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)(methyl)amino)methyl)-3,3-difluoropyrrolidin-1-yl)prop-2-en-1-one,
N-(1-(7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)azepan-3-yl)acrylamide,
N-(1-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)azepan-3-yl)acrylamide,
N-(1-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)azepan-3-yl)acrylamide,
N-(1-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-(1-(7-(8-ethynyl-7-fluoronaphthalen-1-yl)-8-fluoro-2-(((S)-1-methylpyrrolidin-2-yl)methoxy)pyrido[4,3-d]pyrimidin-4-yl)azepan-3-yl)acrylamide,
N-(1-(7-(8-ethynyl-7-fluoronaphthalen-1-yl)-8-fluoro-2-(((S)-1-methylpiperidin-2-yl)methoxy)pyrido[4,3-d]pyrimidin-4-yl)azepan-3-yl)acrylamide,
N-(1-(7-(3-cyano-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-(1-(7-(3-(2-cyanopropan-2-yl)-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-(1-(7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)azepan-3-yl)acrylamide,
N-(1-(6-cyano-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)azepan-3-yl)acrylamide,
N-(1-(6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)azepan-3-yl)acrylamide,
N-(1-(7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)azepan-3-yl)acrylamide,
N-(1-(6-(2-cyanopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)azepan-3-yl)acrylamide,
N-(1-(6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)azepan-3-yl)acrylamide,
N-(1-(7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)azepan-3-yl)acrylamide,
N-(4-cyano-1-(7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)-4-methylazepan-3-yl)-N-methylacrylamide,
N-(4-cyano-1-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-4-methylazepan-3-yl)-N-methylacrylamide,
N-(4-cyano-1-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylazepan-3-yl)-N-methylacrylamide,
N-(4-cyano-1-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)-N-methylacrylamide,
N-(4-cyano-1-(7-(8-ethynyl-7-fluoronaphthalen-1-yl)-8-fluoro-2-(((S)-1-methylpyrrolidin-2-yl)methoxy)pyrido[4,3-d]pyrimidin-4-yl)-4-methylazepan-3-yl)-N-methylacrylamide,
N-(4-cyano-1-(7-(8-ethynyl-7-fluoronaphthalen-1-yl)-8-fluoro-2-(((S)-1-methylpiperidin-2-yl)methoxy)pyrido[4,3-d]pyrimidin-4-yl)-4-methylazepan-3-yl)-N-methylacrylamide,
N-(4-cyano-1-(7-(3-cyano-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)-N-methylacrylamide,
N-(4-cyano-1-(7-(3-(2-cyanopropan-2-yl)-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)-N-methylacrylamide,
N-(4-cyano-1-(7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)-4-methylazepan-3-yl)-N-methylacrylamide,
N-(4-cyano-1-(6-cyano-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-methylazepan-3-yl)-N-methylacrylamide,
N-(1-(6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-cyano-4-methylazepan-3-yl)-N-methylacrylamide,
N-(4-cyano-1-(7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)-4-methylazepan-3-yl)-N-methylacrylamide,
N-(4-cyano-1-(6-(2-cyanopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-methylazepan-3-yl)-N-methylacrylamide,
N-(1-(6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-cyano-4-methylazepan-3-yl)-N-methylacrylamide,
N-(4-cyano-1-(7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)-4-methylazepan-3-yl)-N-methylacrylamide,
N-(1-(7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)-4-hydroxy-4-methylazepan-3-yl)-N-methylacrylamide,
N-(1-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-4-hydroxy-4-methylazepan-3-yl)-N-methylacrylamide,
N-(1-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-hydroxy-4-methylazepan-3-yl)-N-methylacrylamide,
N-(1-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)-N-methylacrylamide,
N-(1-(7-(8-ethynyl-7-fluoronaphthalen-1-yl)-8-fluoro-2-(((S)-1-methylpyrrolidin-2-yl)methoxy)pyrido[4,3-d]pyrimidin-4-yl)-4-hydroxy-4-methylazepan-3-yl)-N-methylacrylamide,
N-(1-(7-(8-ethynyl-7-fluoronaphthalen-1-yl)-8-fluoro-2-(((S)-1-methylpiperidin-2-yl)methoxy)pyrido[4,3-d]pyrimidin-4-yl)-4-hydroxy-4-methylazepan-3-yl)-N-methylacrylamide,
N-(1-(7-(3-cyano-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)-N-methylacrylamide,
N-(1-(7-(3-(2-cyanopropan-2-yl)-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)-N-methylacrylamide,
N-(1-(7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)-4-hydroxy-4-methylazepan-3-yl)-N-methylacrylamide,
N-(1-(6-cyano-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-hydroxy-4-methylazepan-3-yl)-N-methylacrylamide,
N-(1-(6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-hydroxy-4-methylazepan-3-yl)-N-methylacrylamide,
N-(1-(7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)-4-hydroxy-4-methylazepan-3-yl)-N-methylacrylamide,
N-(1-(6-(2-cyanopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-hydroxy-4-methylazepan-3-yl)-N-methylacrylamide,
N-(1-(6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4-hydroxy-4-methylazepan-3-yl)-N-methylacrylamide,
N-(1-(7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)-4-hydroxy-4-methylazepan-3-yl)-N-methylacrylamide,
N-(1-(7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)-4,4-dimethylazepan-3-yl)-N-methylacrylamide,
N-(1-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-4,4-dimethylazepan-3-yl)-N-methylacrylamide,
N-(1-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylazepan-3-yl)-N-methylacrylamide,
N-(1-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4,4-dimethylazepan-3-yl)-N-methylacrylamide,
N-(1-(7-(8-ethynyl-7-fluoronaphthalen-1-yl)-8-fluoro-2-(((S)-1-methylpyrrolidin-2-yl)methoxy)pyrido[4,3-d]pyrimidin-4-yl)-4,4-dimethylazepan-3-yl)-N-methylacrylamide,
N-(1-(7-(8-ethynyl-7-fluoronaphthalen-1-yl)-8-fluoro-2-(((S)-1-methylpiperidin-2-yl)methoxy)pyrido[4,3-d]pyrimidin-4-yl)-4,4-dimethylazepan-3-yl)-N-methylacrylamide,
N-(1-(7-(3-cyano-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4,4-dimethylazepan-3-yl)-N-methylacrylamide,
N-(1-(7-(3-(2-cyanopropan-2-yl)-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-4,4-dimethylazepan-3-yl)-N-methylacrylamide,
N-(1-(7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)-4,4-dimethylazepan-3-yl)-N-methylacrylamide,
N-(1-(6-cyano-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4,4-dimethylazepan-3-yl)-N-methylacrylamide,
N-(1-(6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4,4-dimethylazepan-3-yl)-N-methylacrylamide,
N-(1-(7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)-4,4-dimethylazepan-3-yl)-N-methylacrylamide,
N-(1-(6-(2-cyanopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4,4-dimethylazepan-3-yl)-N-methylacrylamide,
N-(1-(6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-4,4-dimethylazepan-3-yl)-N-methylacrylamide,
N-(1-(7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)-4,4-dimethylazepan-3-yl)-N-methylacrylamide,
N-(4-(7-(8-ethynyl-7-fluoro-3-hydroxynaphthalen-1-yl)-8-fluoro-2-(((R)-1-methylpiperidin-2-yl)methoxy)pyrido[4,3-d]pyrimidin-4-yl)-1,4-oxazepan-6-yl)acrylamide,
N-(4-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-1,4-oxazepan-6-yl)acrylamide,
N-(4-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)acrylamide,
N-(4-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)acrylamide,
N-(4-(7-(8-ethynyl-7-fluoronaphthalen-1-yl)-8-fluoro-2-(((S)-1-methylpyrrolidin-2-yl)methoxy)pyrido[4,3-d]pyrimidin-4-yl)-1,4-oxazepan-6-yl)acrylamide,
N-(4-(7-(8-ethynyl-7-fluoronaphthalen-1-yl)-8-fluoro-2-(((S)-1-methylpiperidin-2-yl)methoxy)pyrido[4,3-d]pyrimidin-4-yl)-1,4-oxazepan-6-yl)acrylamide,
N-(4-(7-(3-cyano-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)acrylamide,
N-(4-(7-(3-(2-cyanopropan-2-yl)-8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)acrylamide,
N-(4-(7-(8-ethynyl-7-fluoro-3-(2-hydroxypropan-2-yl)naphthalen-1-yl)-8-fluoro-2-((tetrahydro-1H-pyrrolizin-7a(5H)-yl)methoxy)pyrido[4,3-d]pyrimidin-4-yl)-1,4-oxazepan-6-yl)acrylamide,
N-(4-(6-cyano-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-1,4-oxazepan-6-yl)acrylamide,
N-(4-(6-(2-aminopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-1,4-oxazepan-6-yl)acrylamide,
N-(4-(7-(8-ethynyl-7-fluoro-3-hydroxynaphthalen-1-yl)-8-fluoro-6-(2-hydroxypropan-2-yl)-2-((tetrahydro-1H-pyrrolizin-7a(5H)-yl)methoxy)quinazolin-4-yl)-1,4-oxazepan-6-yl)acrylamide,
N-(4-(6-(2-cyanopropan-2-yl)-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-1,4-oxazepan-6-yl)acrylamide,
N-(4-(6-chloro-7-(8-ethynyl-7-fluoro-3-hydroxynaphthalen-1-yl)-8-fluoro-2-((tetrahydro-1H-pyrrolizin-7a(5H)-yl)methoxy)quinazolin-4-yl)-1,4-oxazepan-6-yl)acrylamide,
N-(4-(7-(8-ethynyl-7-fluoronaphthalen-1-yl)-8-(methoxy-d3)-2-((tetrahydro-1H-pyrrolizin-7a(5H)-yl)methoxy)pyrido[4,3-d]pyrimidin-4-yl)-1,4-oxazepan-6-yl)acrylamide

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPpCD and SBEpCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl.

The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

Spirocycloalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalikyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups;

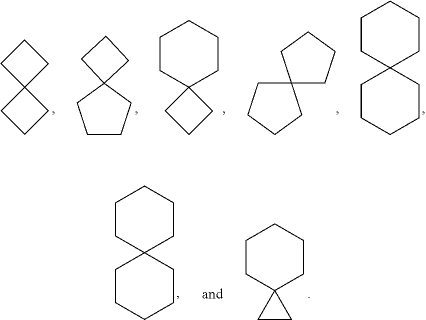

The term “fused-carbocyclic” refers to a polycyclic cyclyl group, wherein each ring in the group shares an adjacent pair of carbon atoms with another ring in the group, wherein one or more rings can contain one or more double bonds. In certain embodiments, the fused heterocyclyl is bicyclic. In certain embodiments, the fused-carbocyclic contains more than two rings, at least two of which share an adjacent pair of atoms. In one embodiment, the fused-carbocyclic is a 5 to 20 membered, 5 to 16 membered, or 5 to 10 membered polycyclic cyclyl group. Representative examples of fused-carbocyclic include, but are not limited to the following groups:

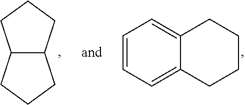 [SMILES: C1CC2CCCC2C1 ; c1ccc2c(c1)CCCC2]

The term “bridged-carbocyclic” refers to a group having at least two rings sharing three or more common ring atoms, separating the two bridgehead atoms by a bridge containing at least one atom. The bridgehead atoms are the atoms from which three bonds radiate and where the rings meet. The rings of the bridged carbocyclyl can have one or more double bonds. In one embodiment, the bridged carbocyclyl is bicyclic. In one embodiment, the bridged carbocyclyl is a 5 to 20 membered, 5 to 16 membered, or 5 to 10 membered polycyclic carbocyclyl group. Representative examples of bridged carbocyclyl include, but are not limited to the following groups:

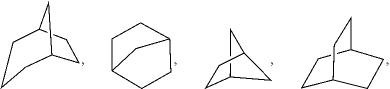

The term “heterocycloalkyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se) and one or more double bonds.

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

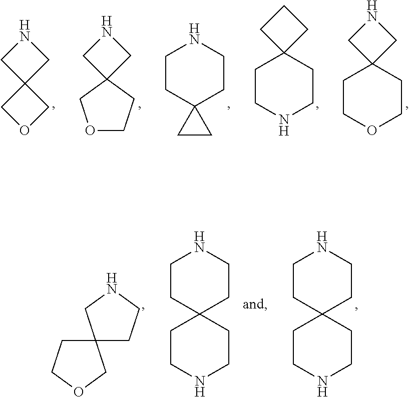

Fused heterocyclyl refers to a polycyclic heterocyclyl group, wherein each ring in the group shares an adjacent pair of atoms (such as carbon atoms) with another ring in the group, wherein one or more rings can contain one or more double bonds, and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic. In certain embodiments, the fused heterocyclyl contains more than two rings, at least two of which share an adjacent pair of atoms. In one embodiment, the fused heterocyclyl is a 5 to 20 membered, 5 to 16 membered, or 5 to 10 membered polycyclic heterocyclyl group. Representative examples of fused heterocyclyl include, but are not limited to the following groups:

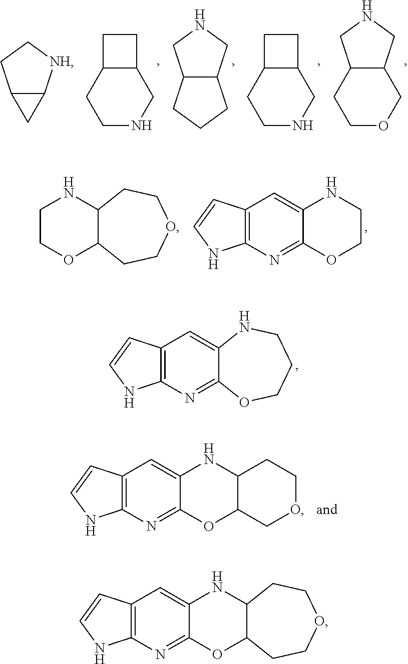

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 16 membered, or 5 to 10 membered polycyclic heterocyclyl group. Representative examples of bridged heterocyclyl include, but are not limited to the following groups:

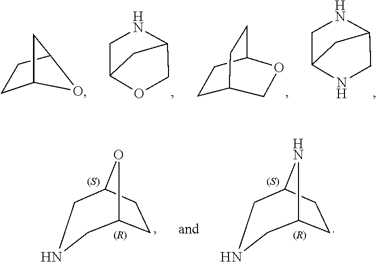

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group.

Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR_{1B}, ALK5/TGFBR1, ALK6/BMPR_{1B}, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), P3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q252H), ABL1(T3151), ABL1(Y253F), ALK (C1156Y), ALK(L1196M), ALK (F1174L), ALK (R1275Q), BRAF(V599E), BTK(E41K), CHK2(1157T), c-Kit(A829P), c-KIT(D816H), c-KIT(D816V), c-Kit(D820E), c-Kit(N822K), C-Kit (T6701), c-Kit(V559D), c-Kit(V559D/V654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g.HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate (e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARP inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry.

The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

An approach to synthesize compounds of

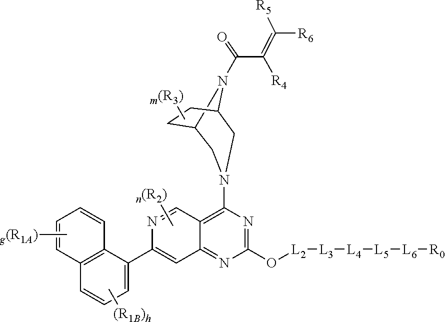

is described in Scheme (1-B).

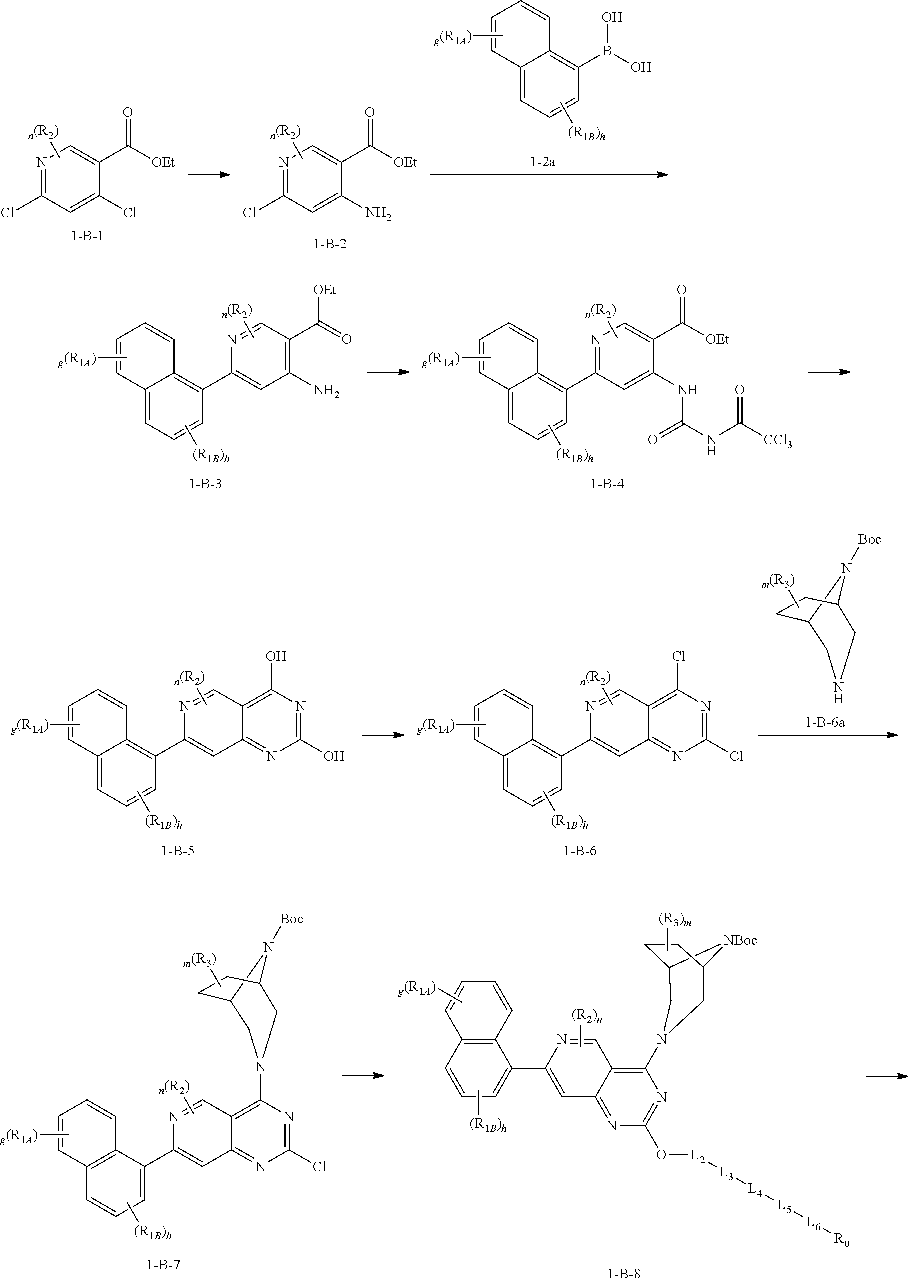

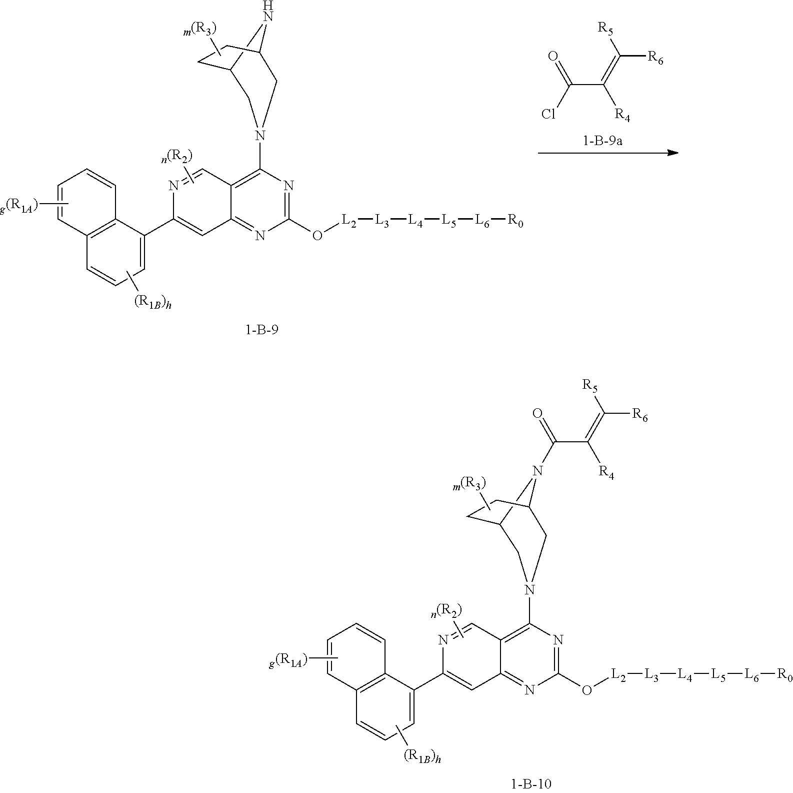

In Scheme (1-B), the starting material 1-B-1 can be prepared by conventional procedures using appropriate compounds and reagent. The starting material 1-B-1 is converted to intermediate 1-B-2 through a SNAr substitution reaction, which can be converted to the intermediate 1-B-3 readily. After that, 1-B-3 is transformed to 1-B-6 via a literate known method. Next, the intermediate 1-B-6 react with 1-B-6a to give 1-B-7, which can couple with a suitable alcohol derivatives to generate 1-B-8. Finally, 1-B-8 is deprotected to afford 1-B-9, which can react with 1-B-9a to yield the target compound 1-B-10.

Also, the target compounds can be synthesized by alternative methods but not limited to the above procedures.

An approach to synthesize compounds of

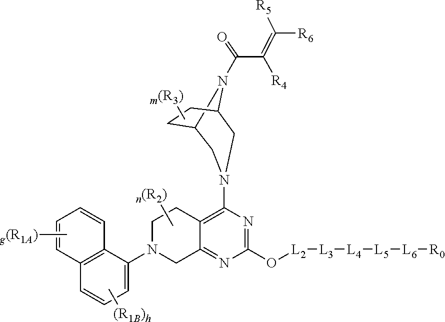

is described in Scheme (1-C).

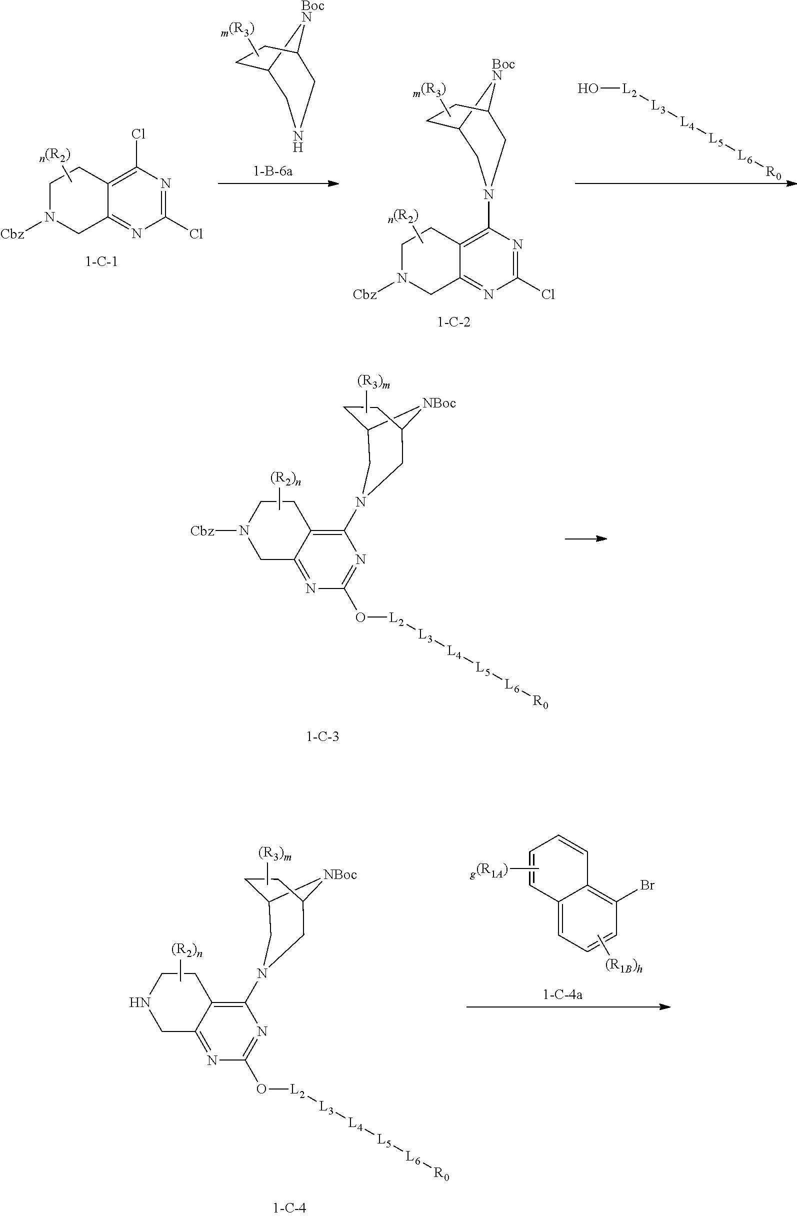

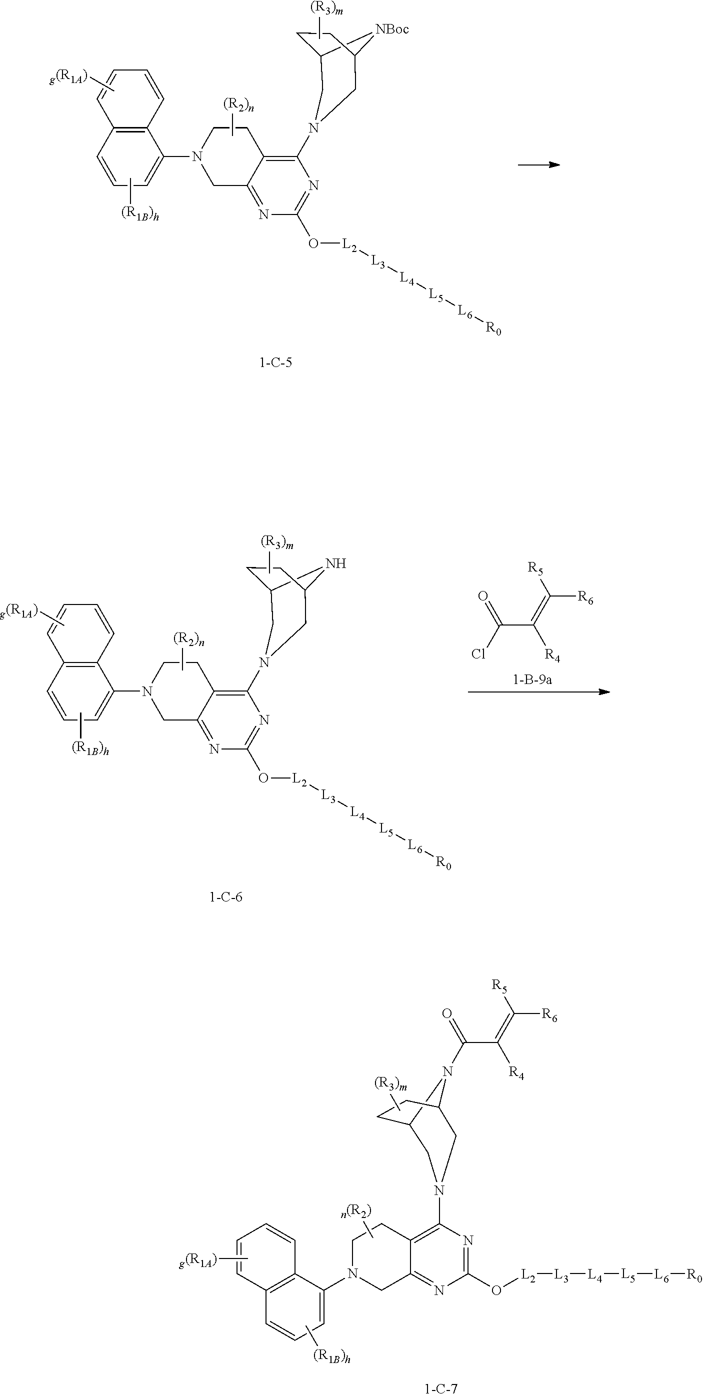

In Scheme (1-C), the starting material 1-C-1 can be prepared by conventional procedures using appropriate compounds and reagent. The starting material 1-C-1 can react with 1-B-6a to give intermediate 1-C-2, which can be converted to the intermediate 1-B-3 via a coupling reaction. After that, 1-C-3 is deprotected to afford 1-C-4, which can go through a Buchwald coupling reaction with 1-C-4a to give 1-C-5. Finally, 1-C-5 is deprotected to afford 1-C-6, which can react with 1-B-9a to yield the target compound 1-C-7.

Also, the target compounds can be synthesized by alternative methods but not limited to the above procedures.

An approach to synthesize compounds of

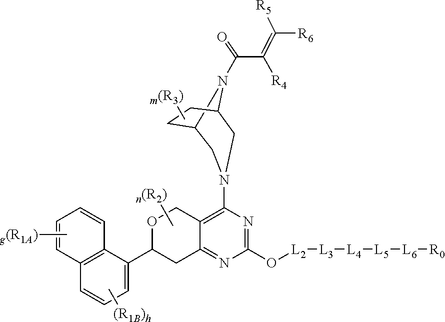

is described in Scheme (1-D)

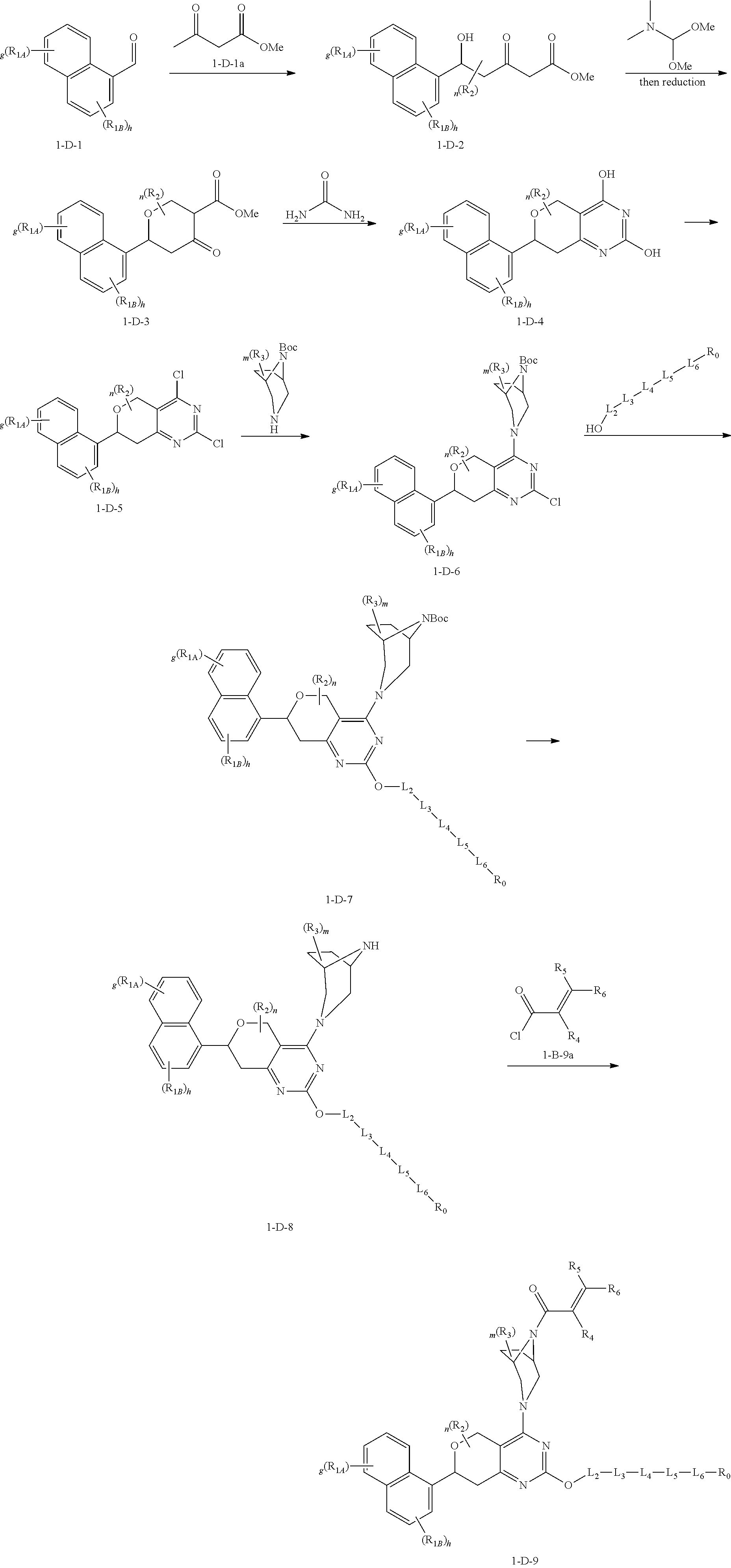

In Scheme (1-D), the starting material 1-D-1 can be prepared by conventional procedures using appropriate compounds and reagent. The starting material 1-D-1 can react with 1-D-1a in the present of a base to provide intermediate 1-D-2, which can be converted to 1-D-2 via a literate known method. The intermediate 1-D-4 can be formed through cyclization of 1-D-3 with urea. After that, 1-D-4 can be converted to 1-D-5 readily, which can go through a sequence of two-step coupling reaction to give 1-D-7. Finally, 1-D-7 is deprotected to afford 1-D-8, which can react with 1-B-9a to yield the target compound 1-D-9.

Also, the target compounds can be synthesized by alternative methods but not limited to the above procedures.

An approach to synthesize compounds of

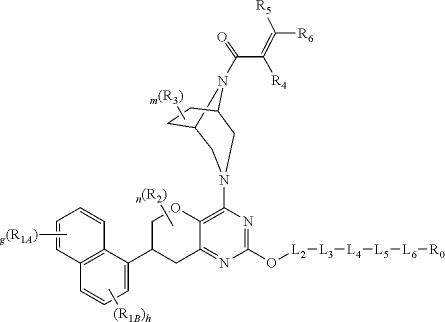

is described in Scheme (1-E).

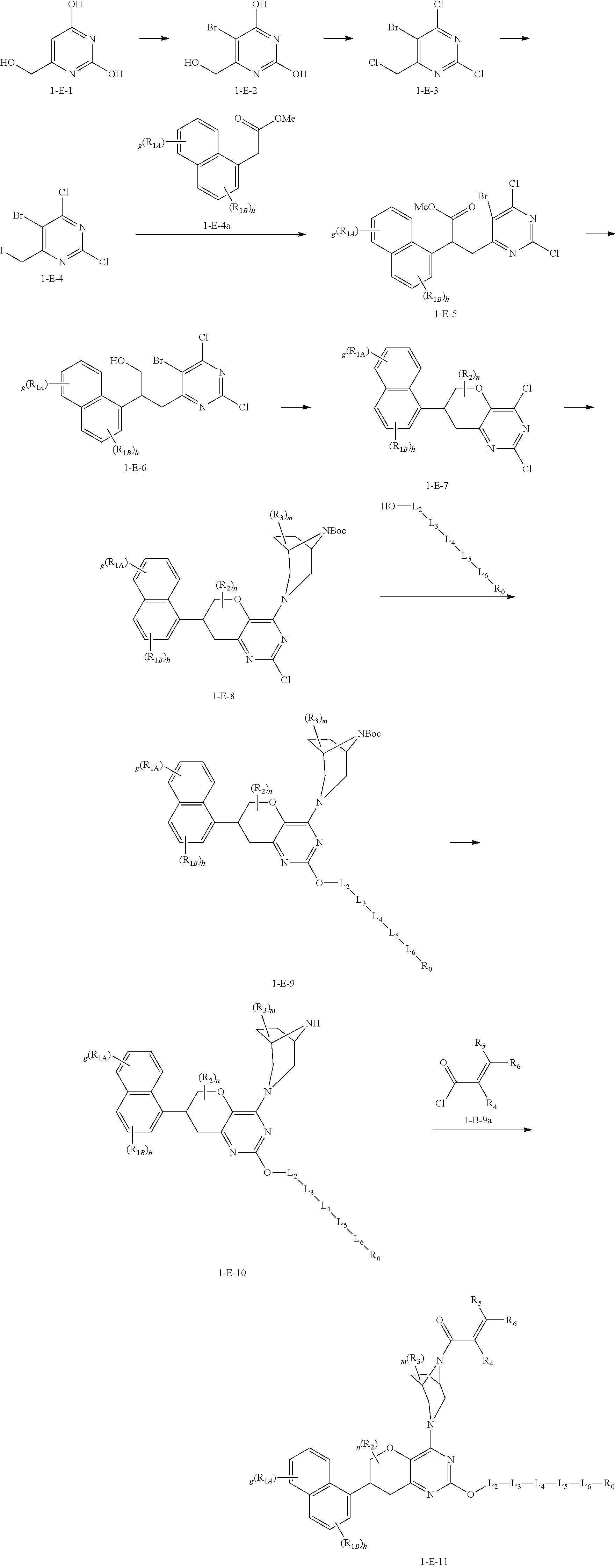

In Scheme (1-E), the starting material 1-E-1 can be prepared by conventional procedures using appropriate compounds and reagent. The starting material 1-E-1 can be converted to 1-E-4 via a literate known method. The intermediate 1-E-4 can react with 1-E-4a to give 1-E-5, which is reduced to give 1-E-6. After that, 1-E-6 undergoes an intermolecular cyclization to afford 1-E-7. Next, the intermediate 1-E-7 can go through a sequence of two-step coupling reaction to provide 1-E-9. Finally, 1-E-9 is deprotected to afford 1-E-10, which can react with 1-B-9a to yield the target compound 1-E-11.

Also, the target compounds can be synthesized by alternative methods but not limited to the above procedures.

An approach to synthesize compounds of

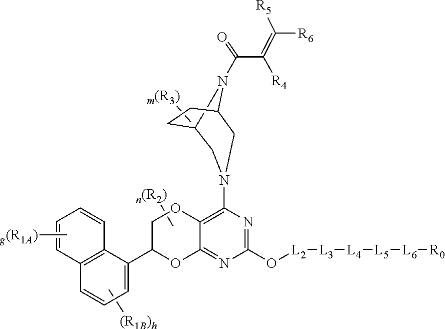

is described in Scheme (1-F).

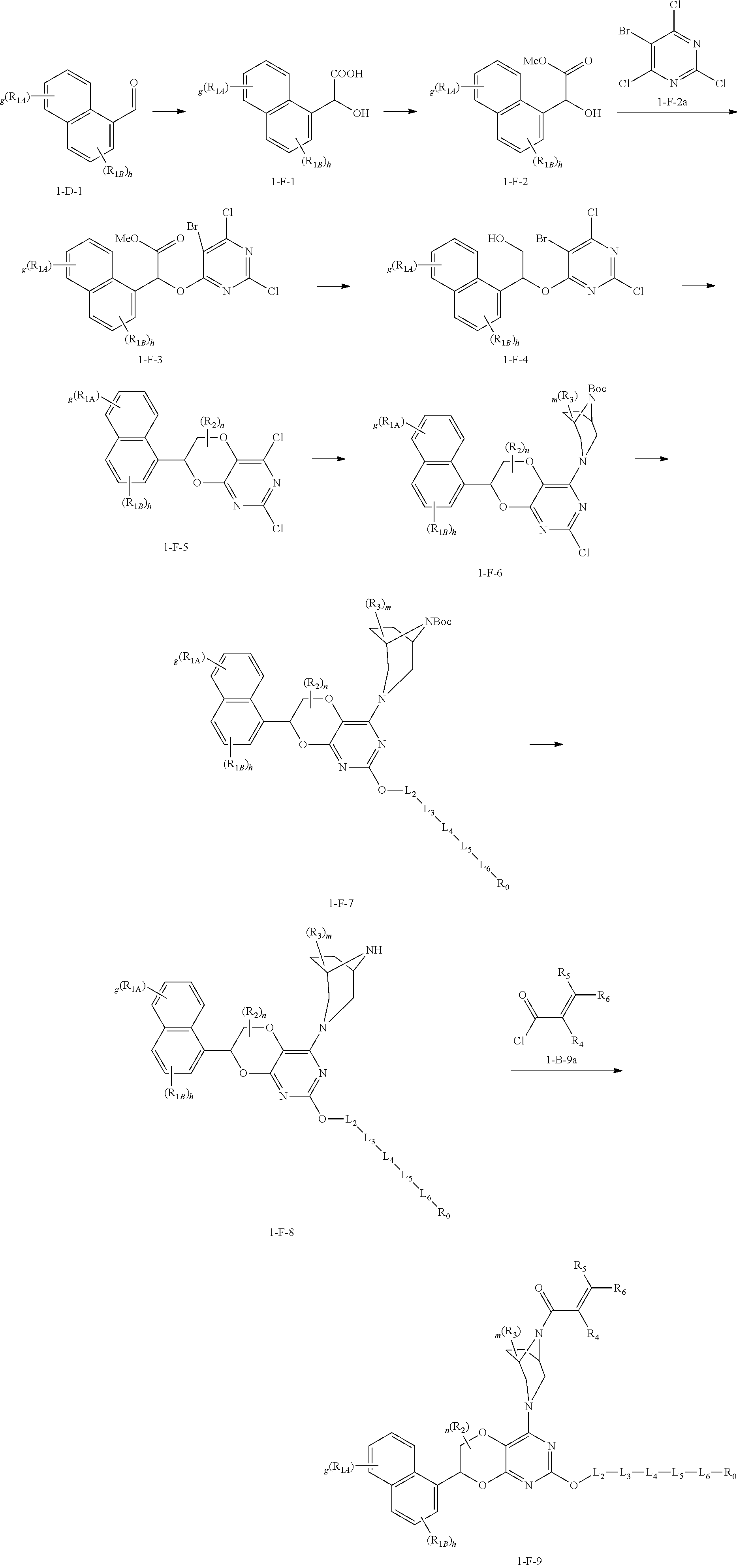

In Scheme (1-F), the starting material 1-D-1 can be prepared by conventional procedures using appropriate compounds and reagent. The starting material 1-D-1 can be converted to 1-F-2 via a literate known method. The intermediate 1-F-2 can react with 1-F-2a to give 1-F-3, which is reduced to give 1-F-4. After that, 1-F-4 undergoes an intermolecular cyclization to afford 1-F-5. Next, the intermediate 1-F-5 can go through a sequence of two-step coupling reaction to provide 1-F-7. Finally, 1-F-7 is deprotected to afford 1-F-8, which can react with 1-B-8a to yield the target compound 1-F-9.

Also, the target compounds can be synthesized by alternative methods but not limited to the above procedures.

An approach to synthesize compounds of

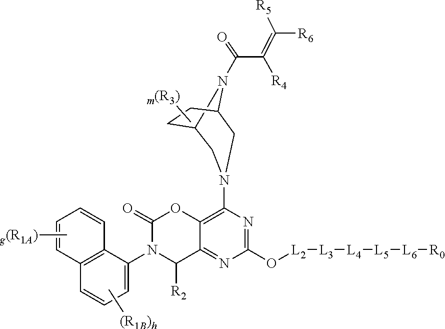

is described in Scheme (1-G).

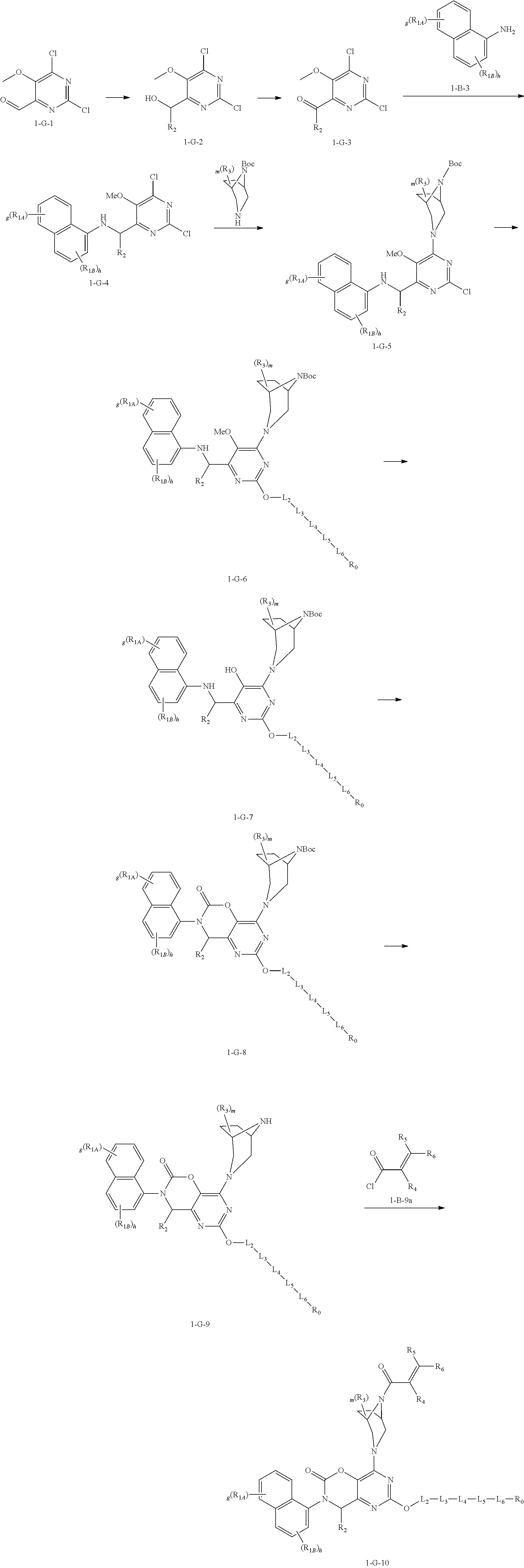

In Scheme (1-G), the starting material 1-G-1 can be prepared by conventional procedures using appropriate compounds and reagent. The starting material 1-G-1 can be converted to 1-G-3 via a literate known method. The intermediate 1-G-3 can be converted to 1-G-4 via a reductive amination reaction. Next, the intermediate 1-G-4 can go through a sequence of two-step coupling reaction to provide 1-G-6. The demethylation of 1-G-6 can afford 1-G-7, which can be further transform to 1-G-8. Finally, 1-G-8 is deprotected to afford 1-G-9, which can react with 1-B-9a to yield the target compound 1-G-10.

Also, the target compounds can be synthesized by alternative methods but not limited to the above procedures.

An approach to synthesize compounds of

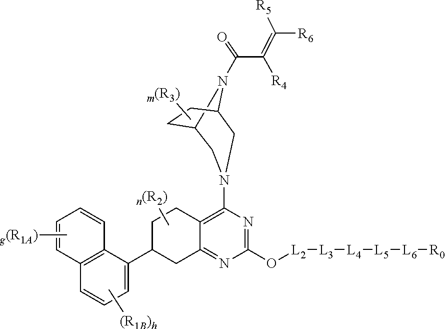

is described in Scheme (1-H).

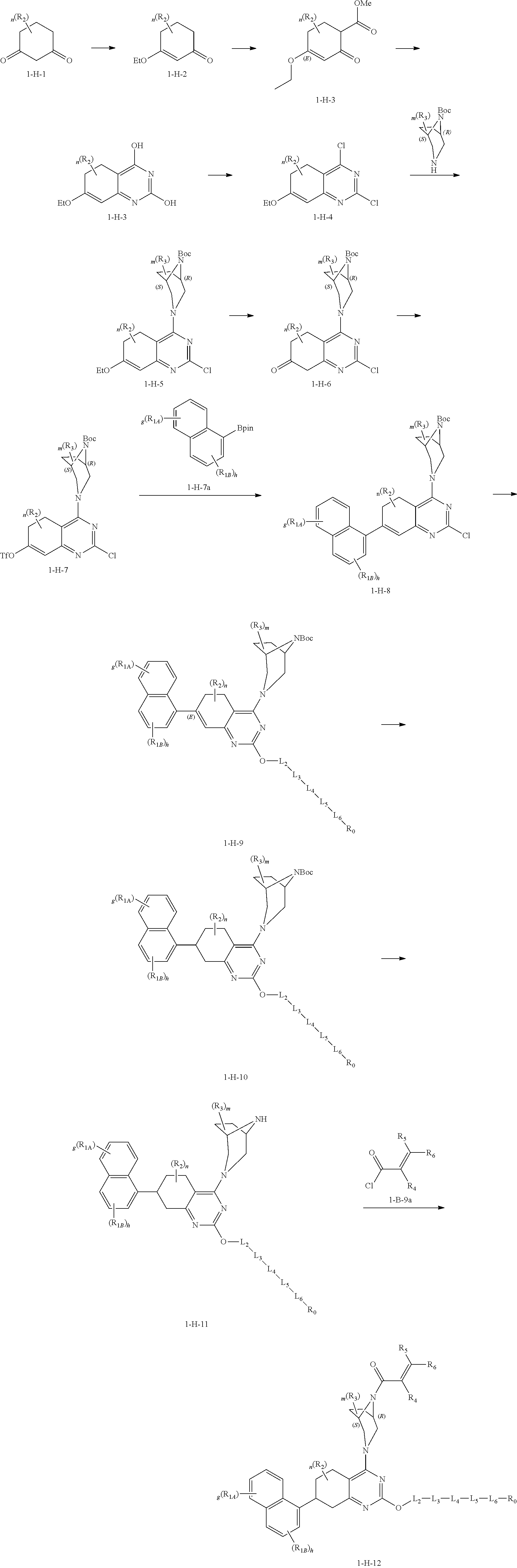

In Scheme (1-H), the starting material 1-H-1 can be prepared by conventional procedures using appropriate compounds and reagent. The starting material 1-H-1 can be converted to 1-H-4 via a literate known method. The intermediate 1-H-4 can be converted to 1-H-5 via a SNAr substitution reaction. After that, the intermediate 1-H-5 is deprotected to give 1-H-6, which can be converted to 1-H-7. The intermediate 1-H-7 can go through a Suzuki coupling reaction with 1-H-7a to provide 1-H-8, which can be transform to 1-H-9. Next, 1-H-9 can be reduced to 1-H-10 readily, which is deprotected to give 1-H-11. Finally, the intermediate 1-H-11 can react with 1-B-9a to yield the target compound 1-H-12.

Also, the target compounds can be synthesized by alternative methods but not limited to the above procedures.

An approach to synthesize compounds of

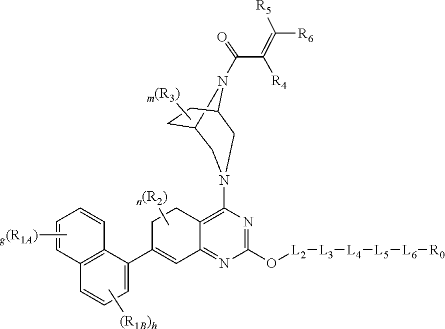

is described in Scheme (1-I)

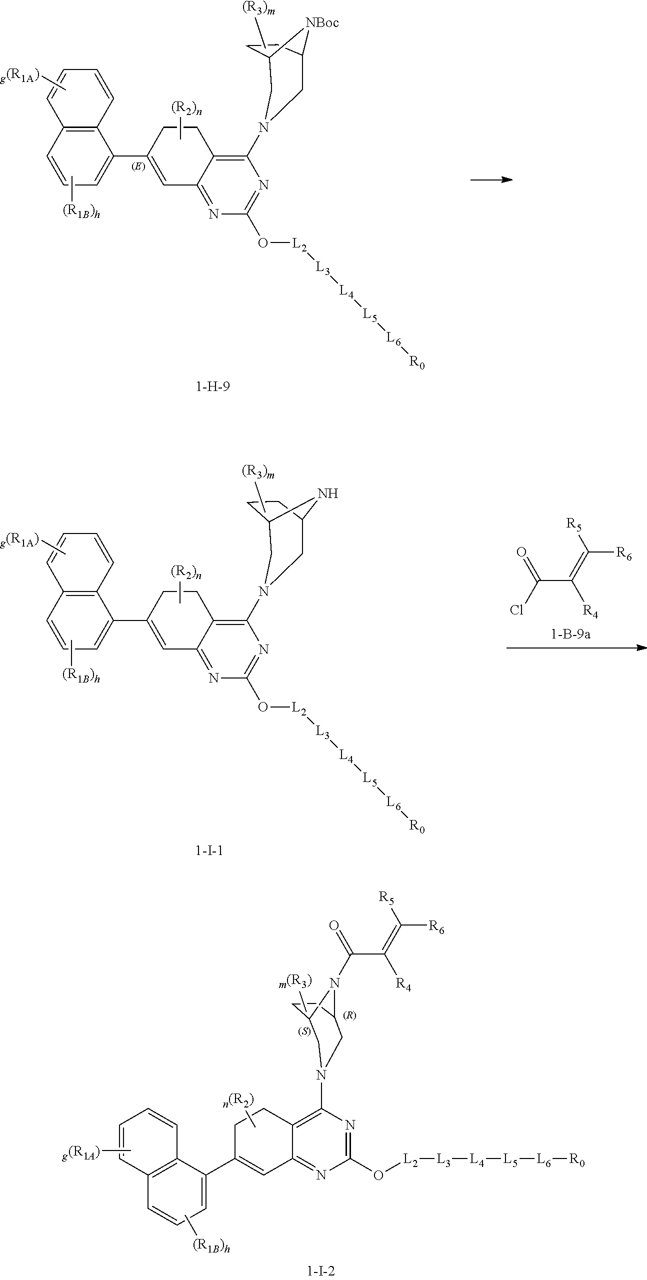

In Scheme (1-H), the starting material 1-H-9 can be prepared by conventional procedures using appropriate compounds and reagent. The intermediate 1-H-9 is deprotected to afford 1-I-1, which can react with 1-B-9a to yield the target compound 1-I-2.

Also, the target compounds can be synthesized by alternative methods but not limited to the above procedures.

An approach to synthesize compounds of

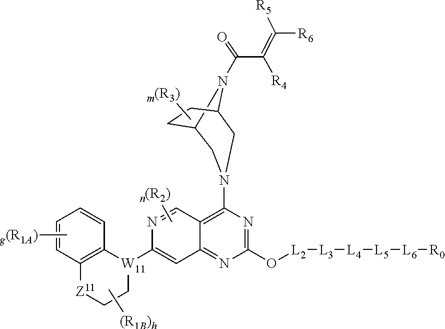

is described in Scheme (1-J).

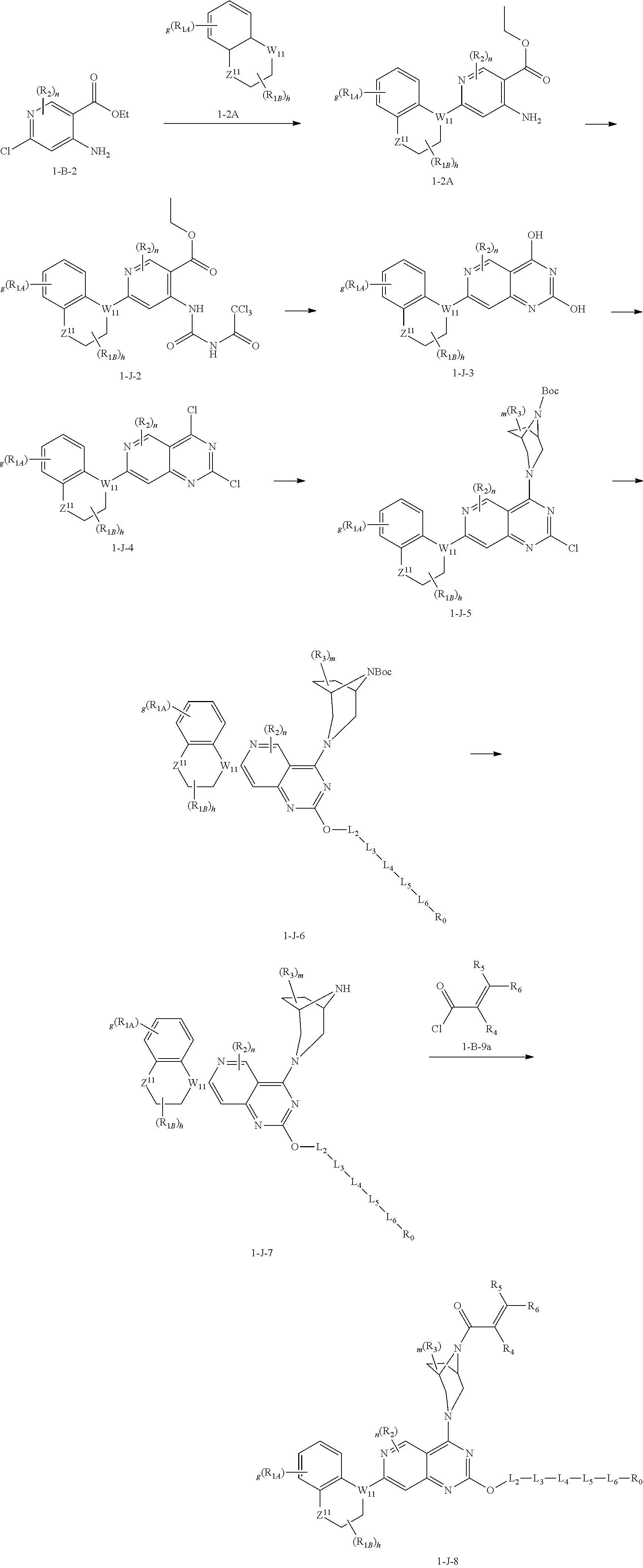

In Scheme (1-J), the starting material 1-B-2 can be prepared by conventional procedures using appropriate compounds and reagent. The starting material 1-B-2 can be converted to the intermediate 1-J-1 readily. After that, 1-J-1 is transformed to 1-J-4 via a known method, and the intermediate 1-J-4 can be converted to 1-J-6 through a sequence of two-step coupling reaction. Finally, the intermediate 1-J-6 is deprotected to afford 1-J-7, which can react with 1-B-9a to yield the target compound 1-J-8.

Also, the target compounds can be synthesized by alternative methods but not limited to the above procedures.

An approach to synthesize compounds of

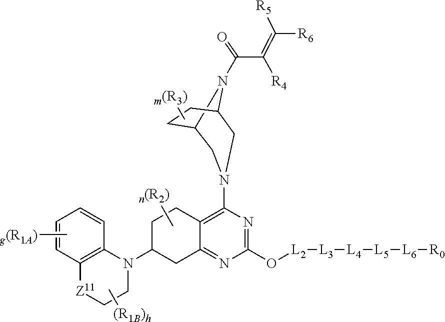

is described in Scheme (1-K).

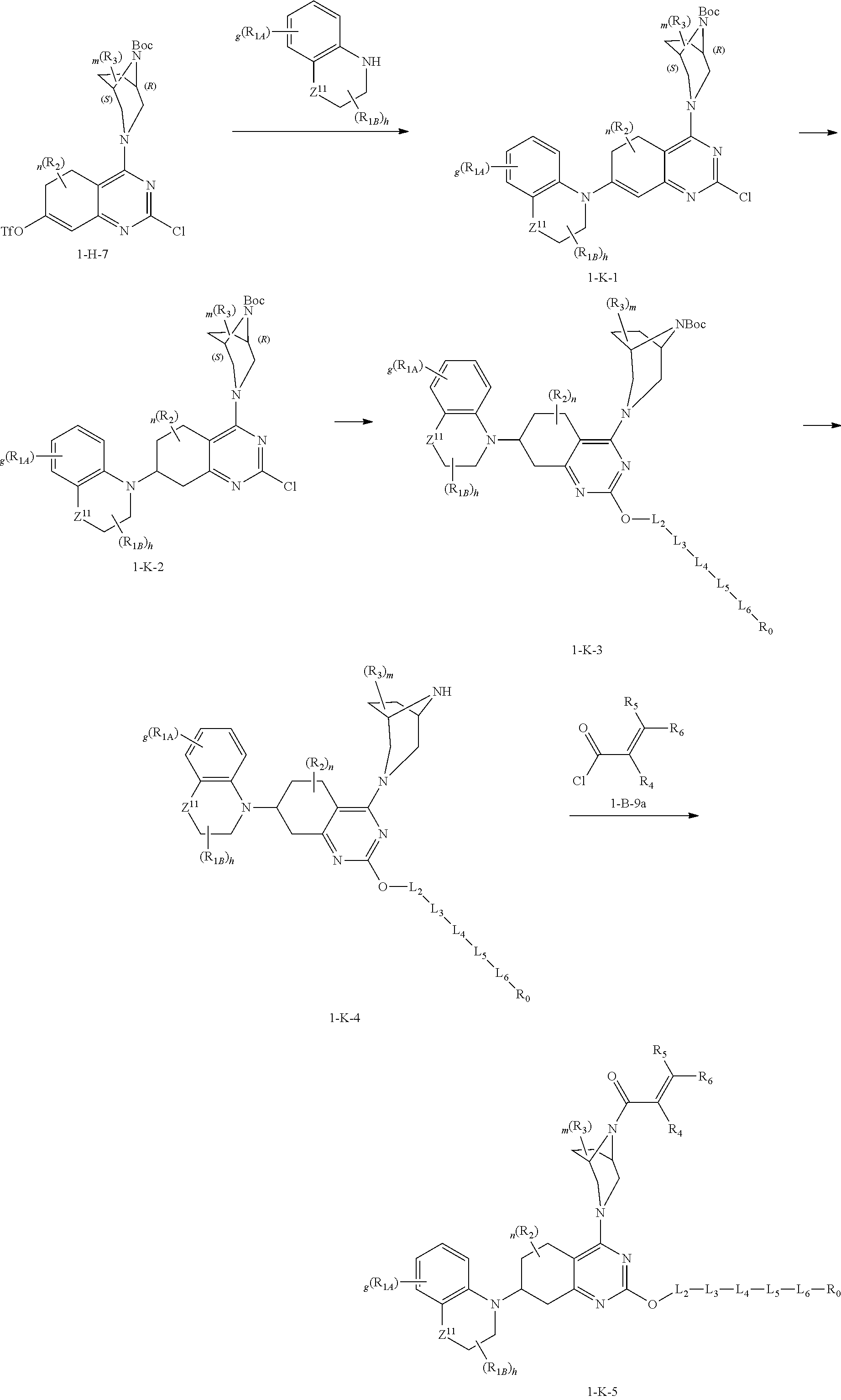

In Scheme (1-K), the starting material 1-H-7 can be prepared by conventional procedures using appropriate compounds and reagent. The starting material 1-H-7 can be converted to the intermediate 1-K-1. After that, 1-K-1 is reduced to 1-K-2, which can be further converted to 1-K-3. After that, the intermediate 1-K-3 is deprotected to afford 1-K-4, which can react with 1-B-9a to yield the target compound 1-K-5.

Also, the target compounds can be synthesized by alternative methods but not limited to the above procedures.

An approach to synthesize compounds of

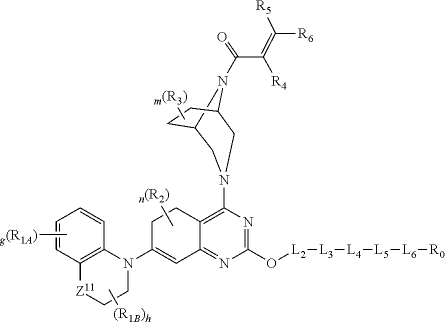

is described in Scheme (1-L)

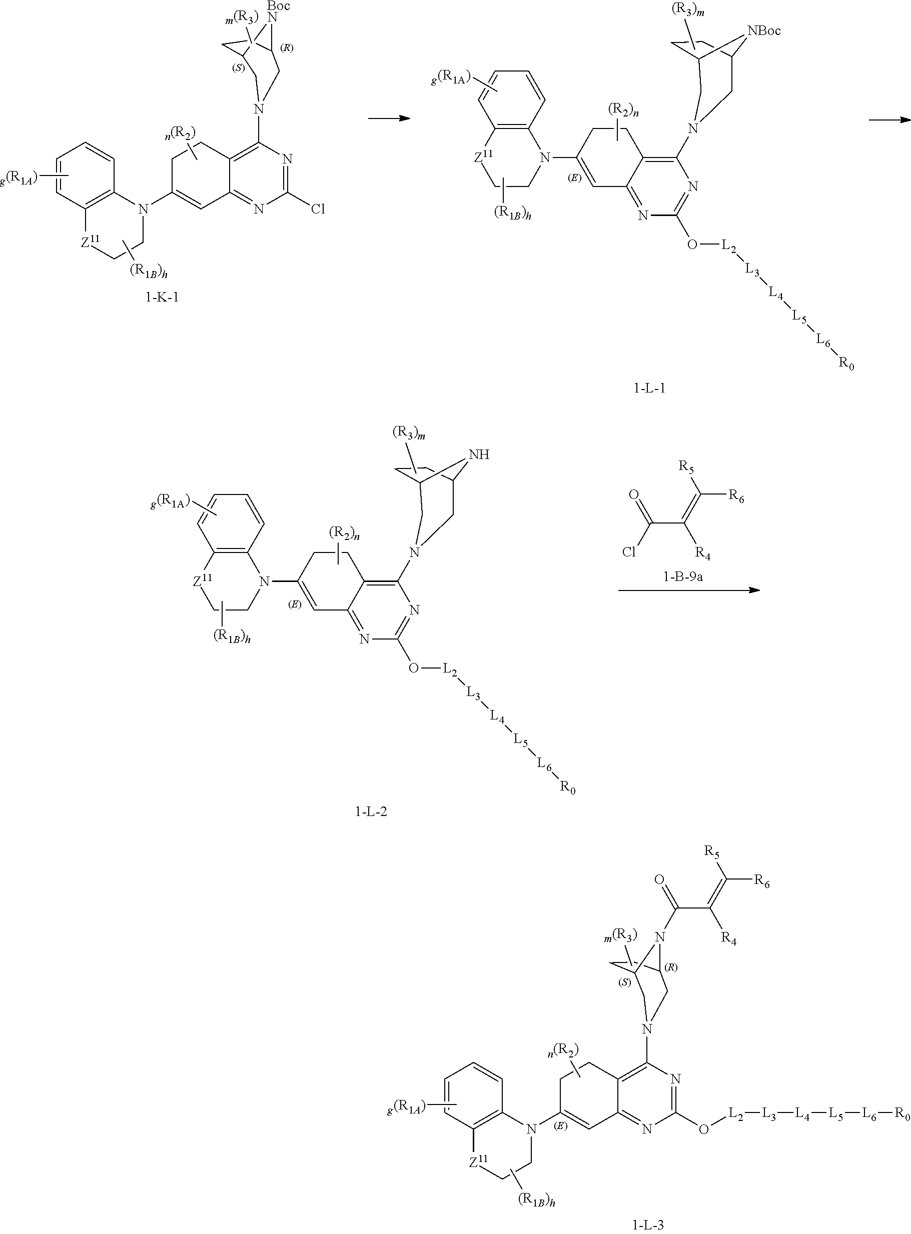

In Scheme (1-L), the starting material 1-K-1 can be prepared by conventional procedures using appropriate compounds and reagent. The starting material 1-K-1 can be converted to the intermediate 1-L-1. After that, the intermediate 1-L-1 is deprotected to afford 1-L-2, which can react with 1-B-9a to yield the target compound 1-L-3.

Also, the target compounds can be synthesized by alternative methods but not limited to the above procedures.

An approach to synthesize compounds of

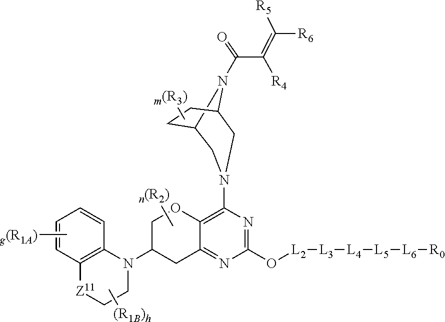

is described in Scheme (1-M).

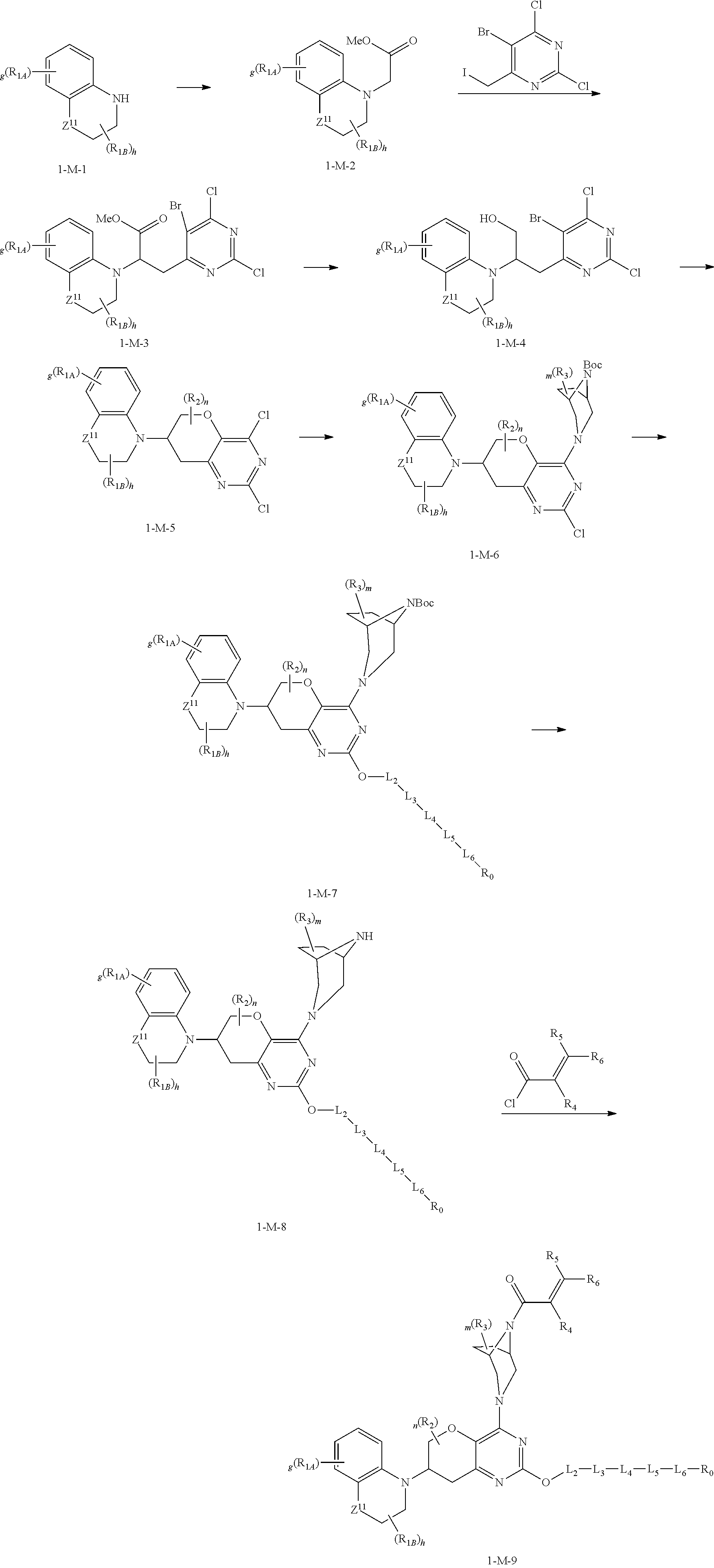

In Scheme (1-M), the starting material 1-M-1 can be prepared by conventional procedures using appropriate compounds and reagent. The starting material 1-M-1 can be converted to 1-M-2 readily, which can be transform to 1-M-5 with the similar method described in Scheme (1-E). The intermediate 1-M-5 can go through a sequence of two-step coupling reaction to provide 1-M-7. Finally, 1-M-7 is deprotected to afford 1-M-8, which can react with 1-B-9a to yield the target compound 1-M-9.

Also, the target compounds can be synthesized by alternative methods but not limited to the above procedures.

An approach to synthesize compounds of

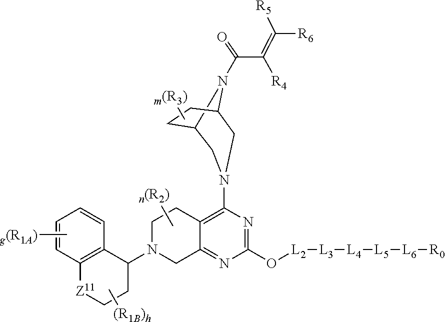

is described in Scheme (1-N).

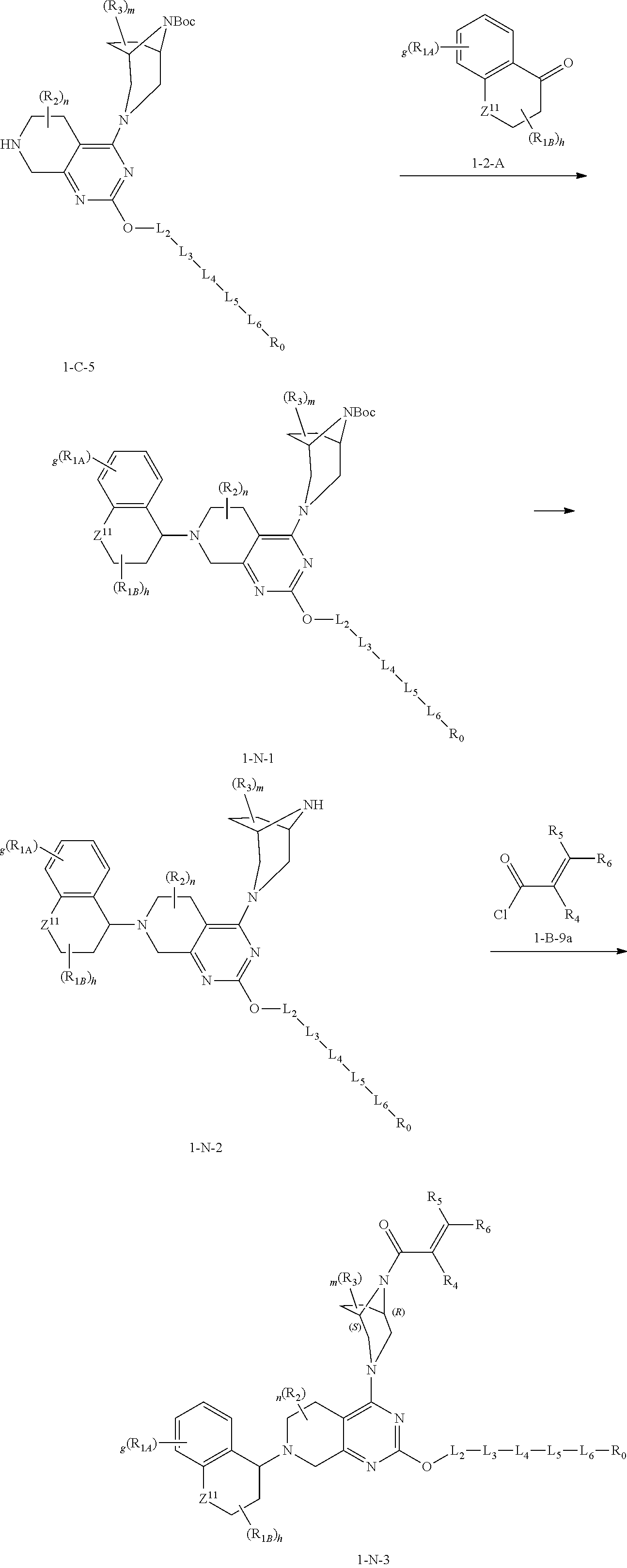

In Scheme (1-N), the starting material 1-C-5 can be prepared by conventional procedures using appropriate compounds and reagent. The starting material 1-C-5 can be converted to 1-N-1 via a reductive amination reaction. After that, 1-N-1 is deprotected to afford 1-N-2, which can react with 1-B-9a to yield the target compound 1-N-3.

Also, the target compounds can be synthesized by alternative methods but not limited to the above procedures.

The compounds

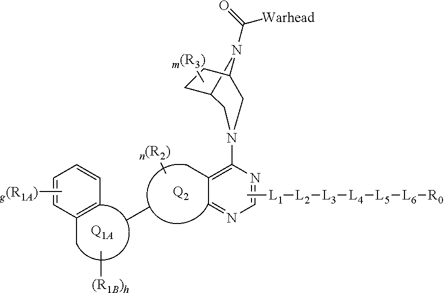

can be made by the method referred to Scheme 1-B to 1-N, by using different starting material and reagents, or by the conventional organic reactions.

The compound

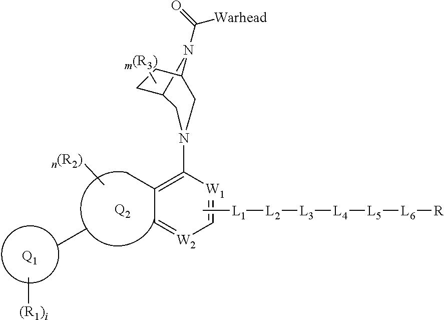

can be made by the method referred to Scheme 1-B to 1-N, by using different starting material and reagents, or by the conventional organic reactions.

An approach to synthesize compounds of

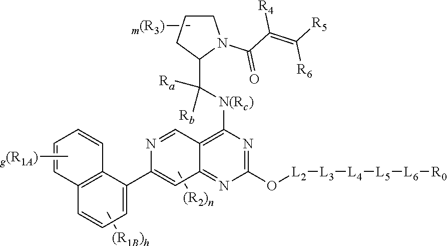

is described in Scheme (2-C).

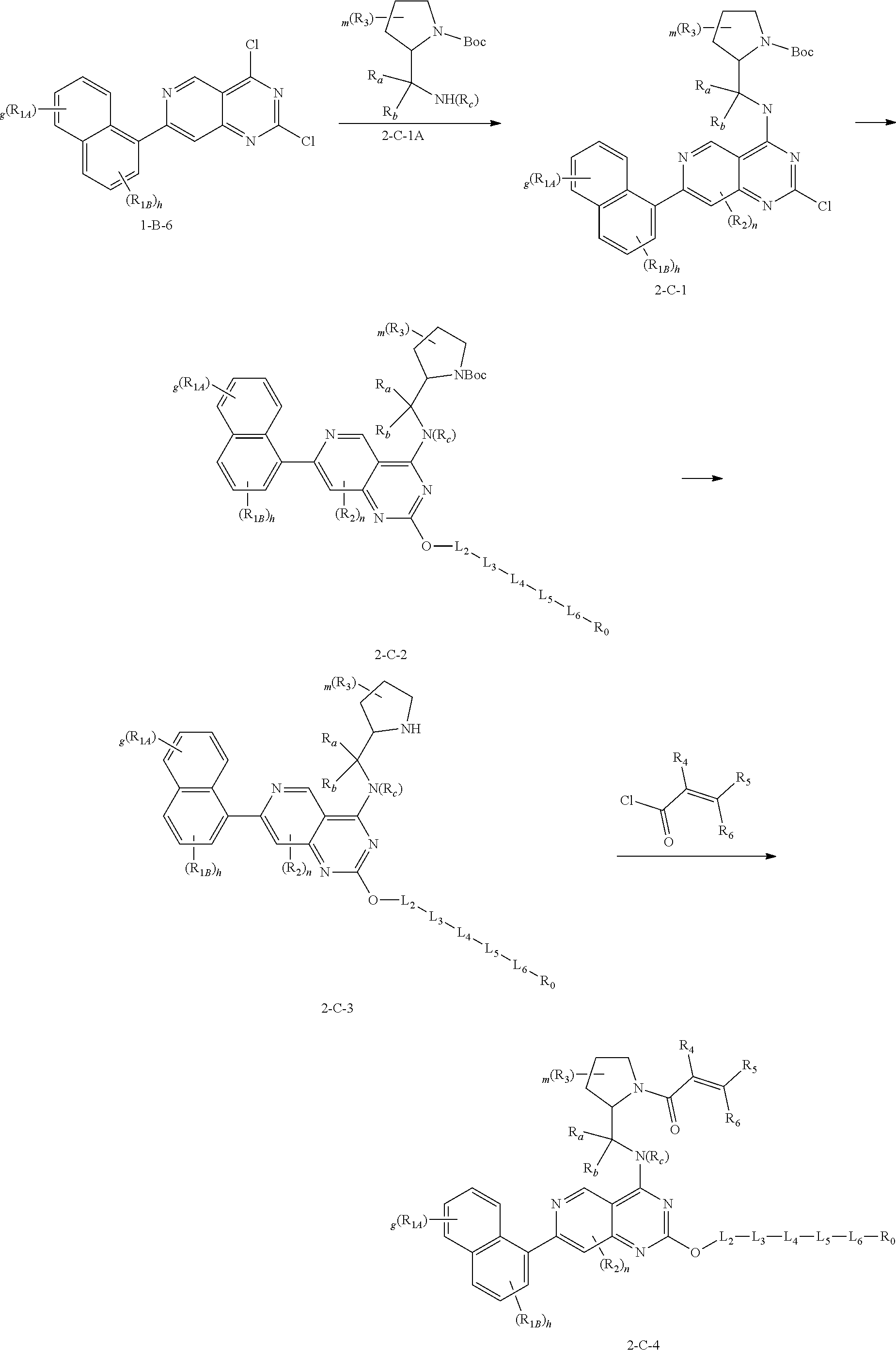

In Scheme (2-C), the starting material 1-B-6 can be prepared by conventional procedures using appropriate compounds and reagent. The starting material 1-B-6 is converted to intermediate 2-C-1 through a SNAr substitution reaction, which can be converted to the intermediate 2-C-2 readily. Finally, 2-C-2 is deprotected to afford 2-C-3, which can react with 1-B-9a to yield the target compound 2-C-4.

Also, the target compounds can be synthesized by alternative methods but not limited to the above procedures.

An approach to synthesize compounds of

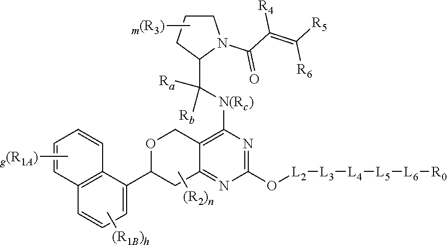

is described in Scheme (2-D).

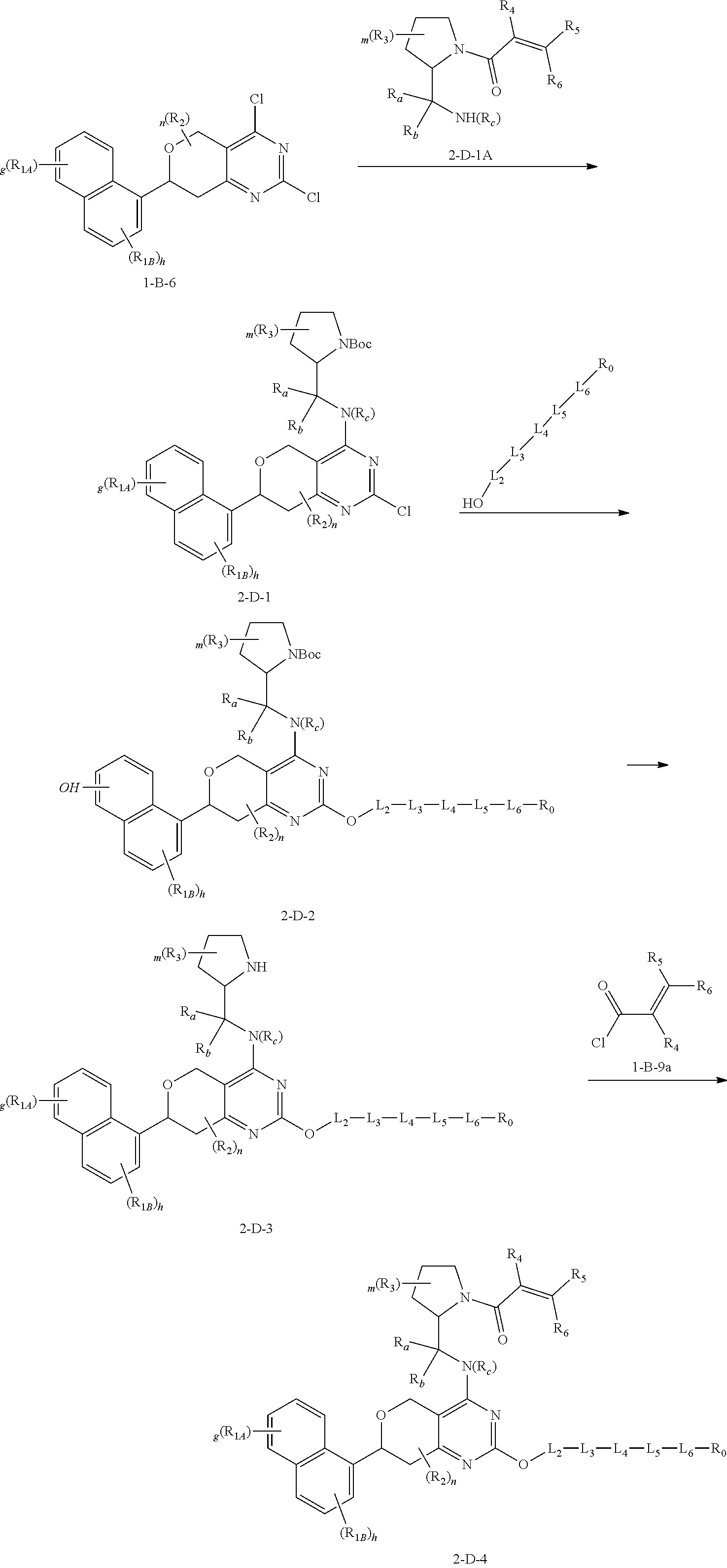

In Scheme (2-0), the starting material 1-D-5 can be prepared by conventional procedures using appropriate compounds and reagent. The starting material 1-D-5 can go through a sequence of two-step coupling reaction to give 2-D-2. The intermediate 2-D-2 is deprotected to afford 2-D-3, which can react with 1-1B-9a to yield the target compound 2-D-4.

Also, the target compounds can be synthesized by alternative methods but not limited to the above procedures.

An approach to synthesize compounds of

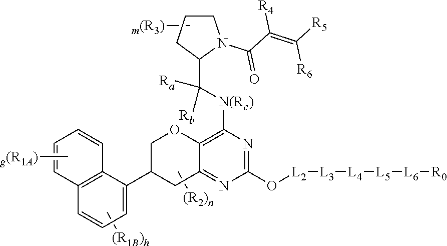

is described in Scheme (2-E).

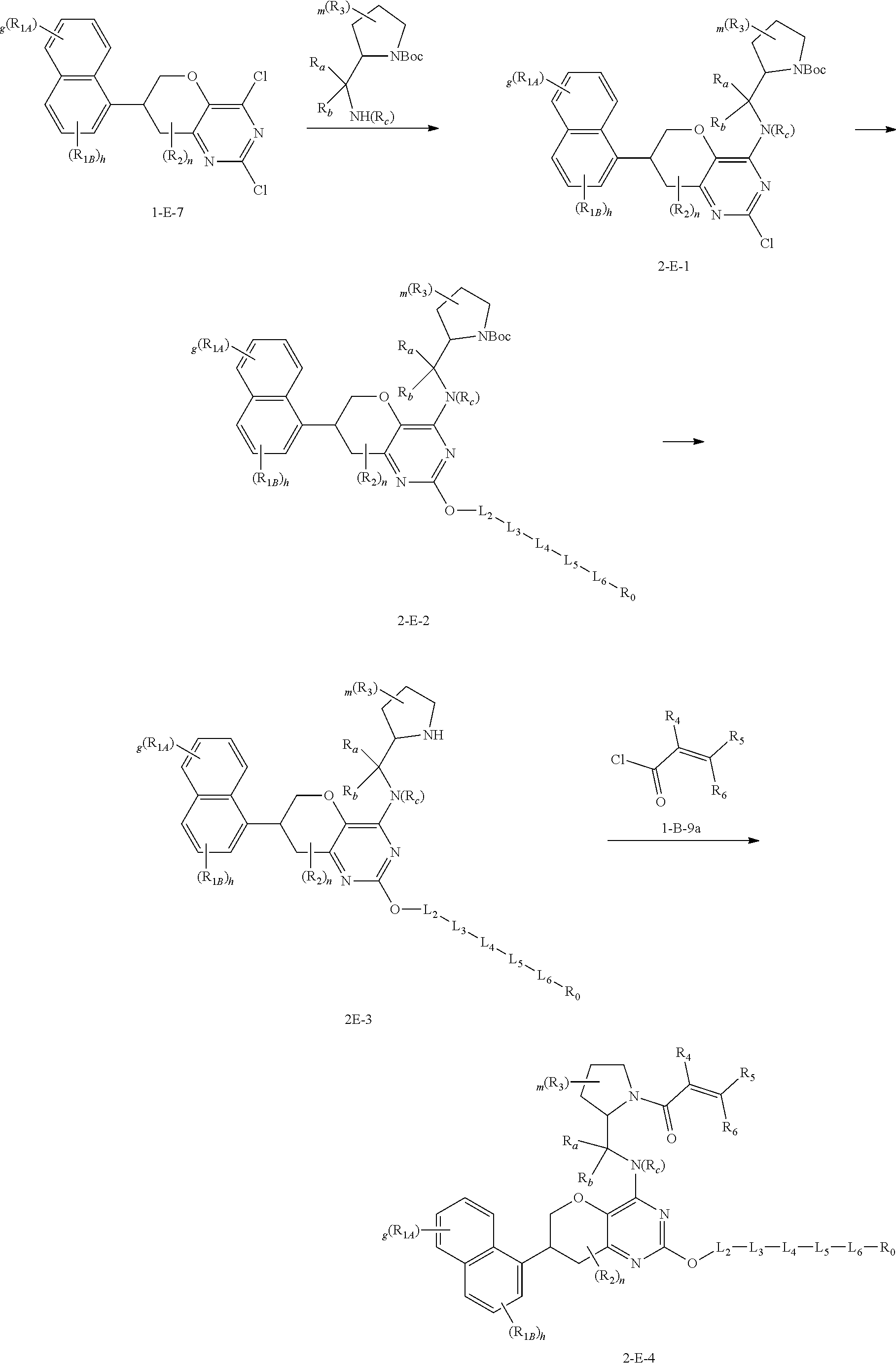

In Scheme (2-E), the starting material 1-E-7 can be prepared by conventional procedures using appropriate compounds and reagent. The starting material 1-E-7 can go through a sequence of two-step coupling reaction to provide 2-E-2. Finally, 2-E-2 is deprotected to afford 2-E-3, which can react with 11-1B-9a to yield the target compound 2-E-4.

Also, the target compounds can be synthesized by alternative methods but not limited to the above procedures.

An approach to synthesize compounds of

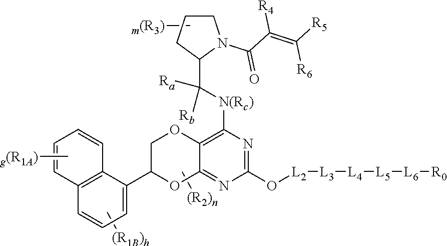

is described in Scheme (2-F).

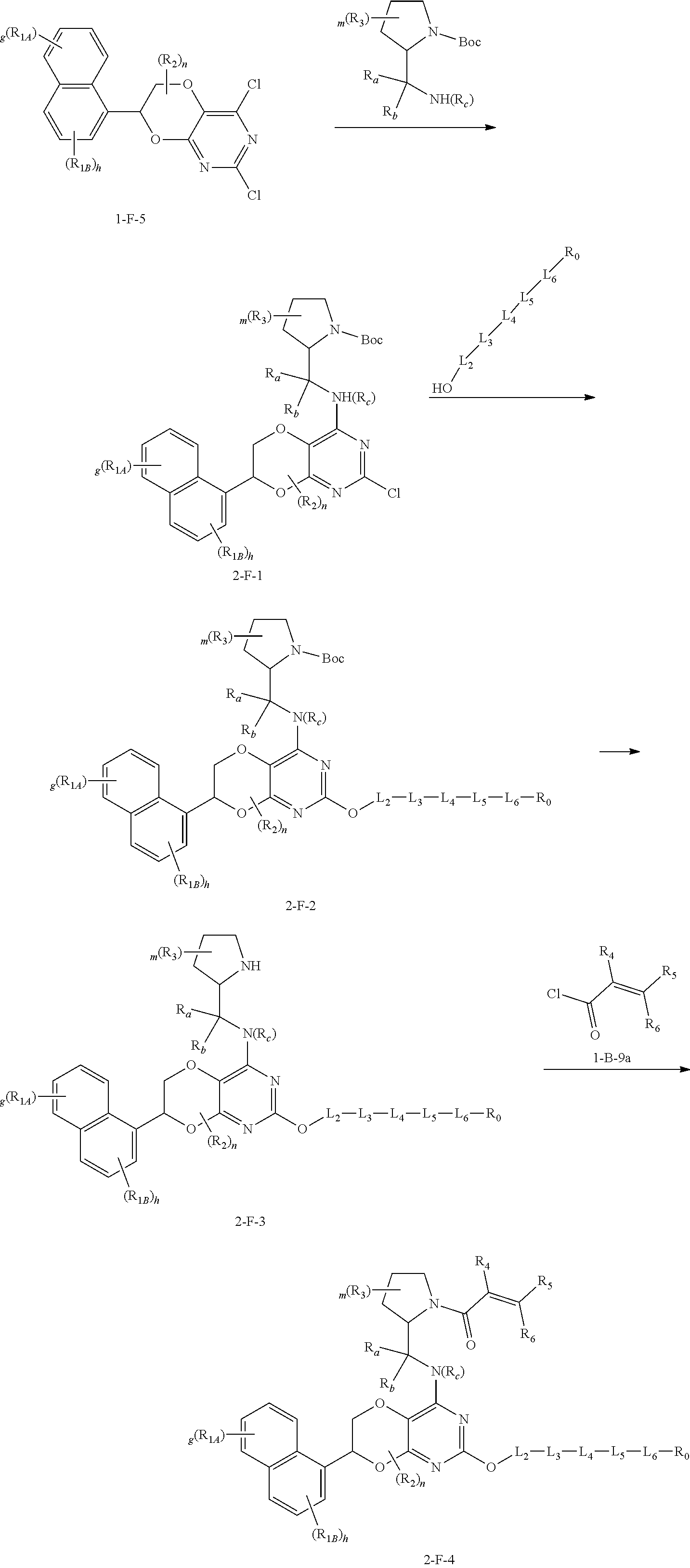

In Scheme (2-F), the starting material 1-F-5 can be prepared by conventional procedures using appropriate compounds and reagent. The starting material 1-F-5 can go through a sequence of two-step coupling reaction to provide 2-F-2. Finally, 2-F-2 is deprotected to afford 2-F-3, which can react with 1-B-9a to yield the target compound 2-F-4.

Also, the target compounds can be synthesized by alternative methods but not limited to the above procedures.

An approach to synthesize compounds of

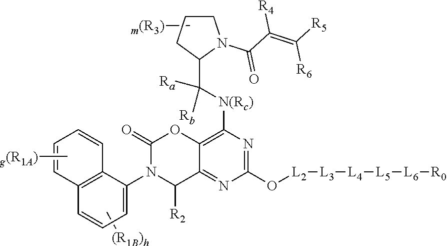

is described in Scheme (2-G).

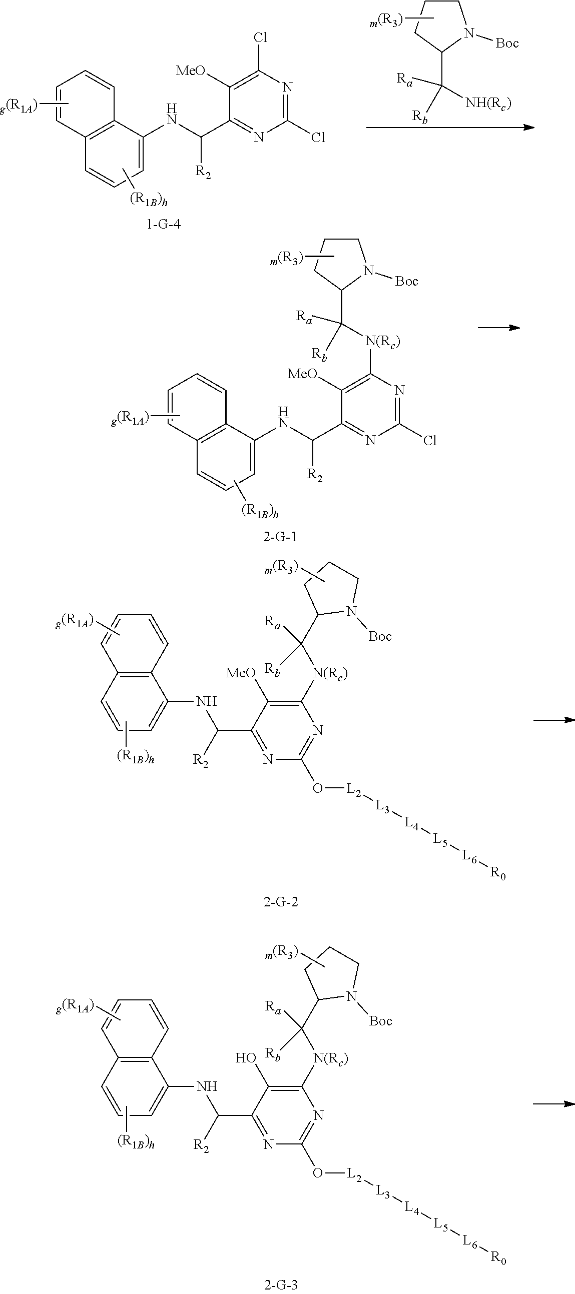

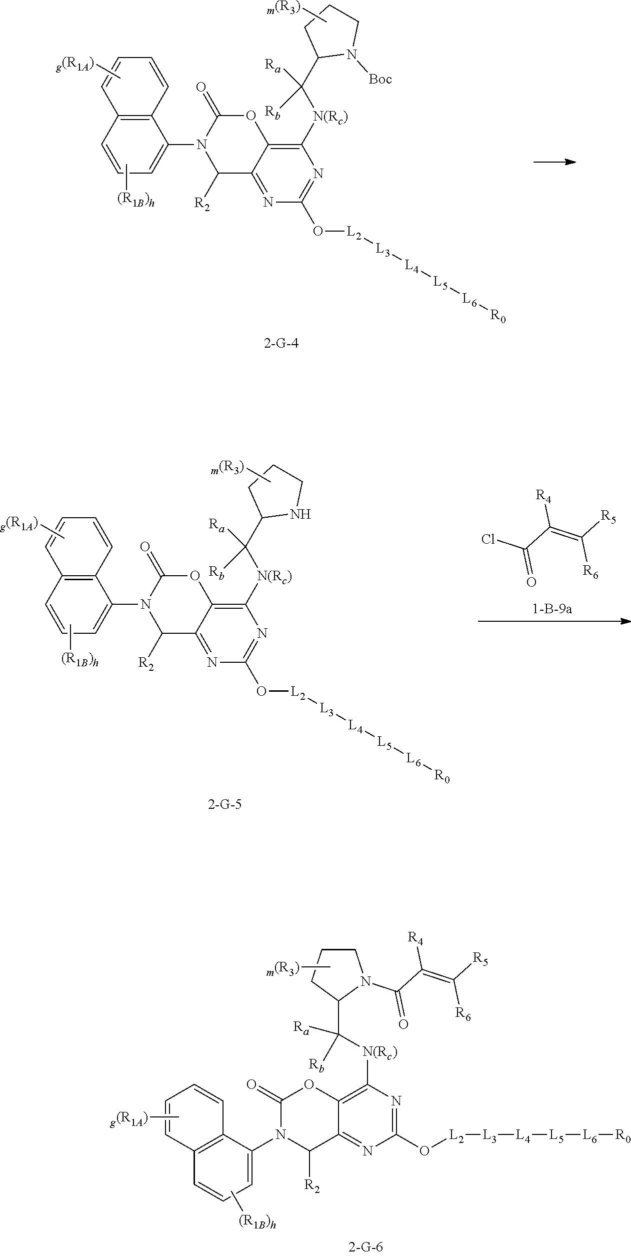

In Scheme (2-G), the starting material 1-G-4 can be prepared by conventional procedures using appropriate compounds and reagent. The starting material 1-G-4 can go through a sequence of two-step coupling reaction to provide 2-G-2. The demethylation of 2-G-2 can afford 2-G-3, which can be further transform to 2-G-4. Finally, 2-G-4 is deprotected to afford 2-G-5, which can react with 1-1B-9a to yield the target compound 2-G-6.

Also, the target compounds can be synthesized by alternative methods but not limited to the above procedures.

An approach to synthesize compounds of

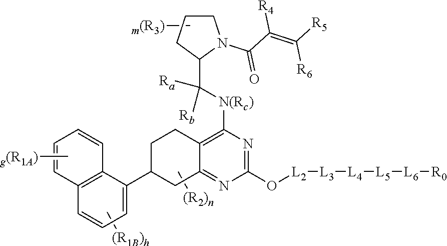

is described in Scheme (2-H).

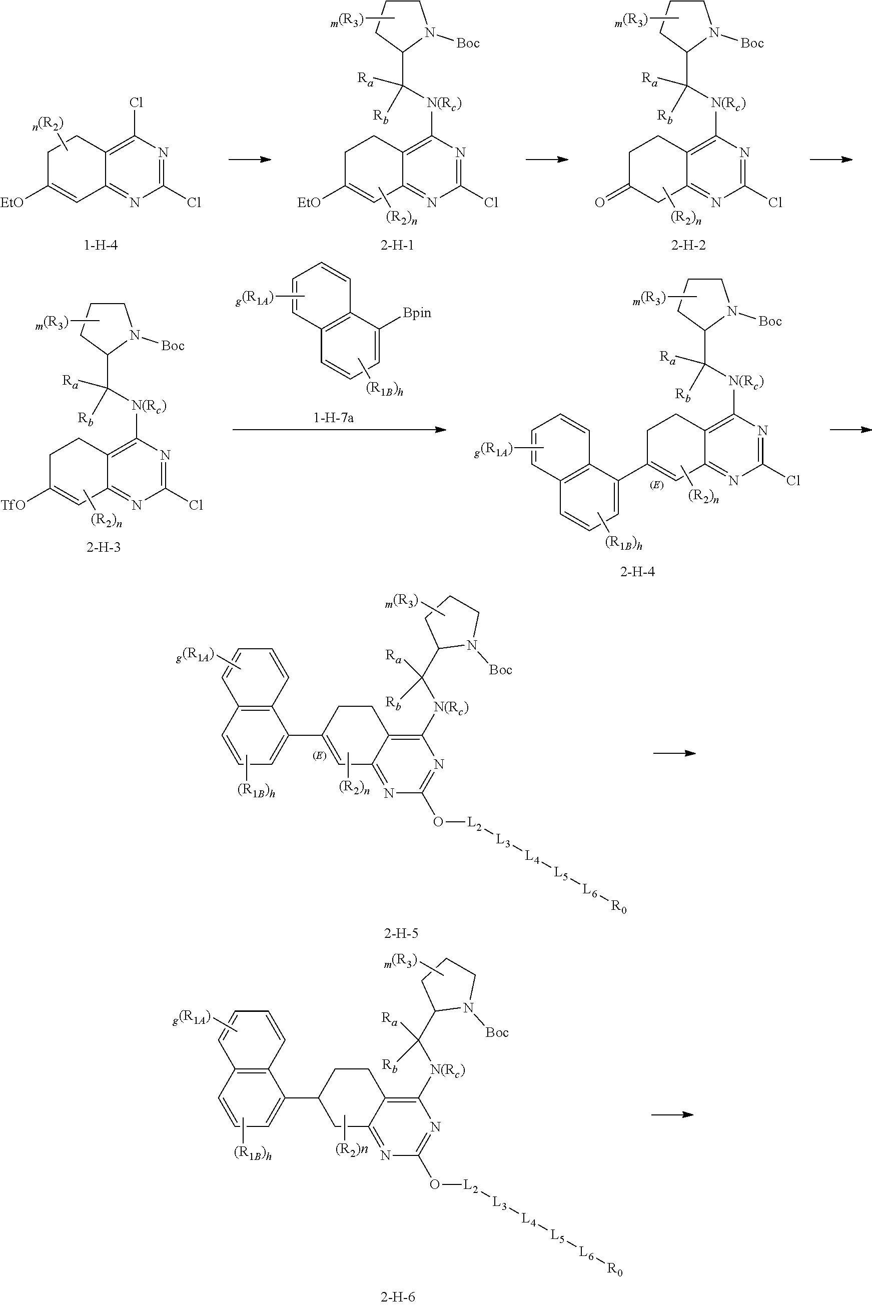

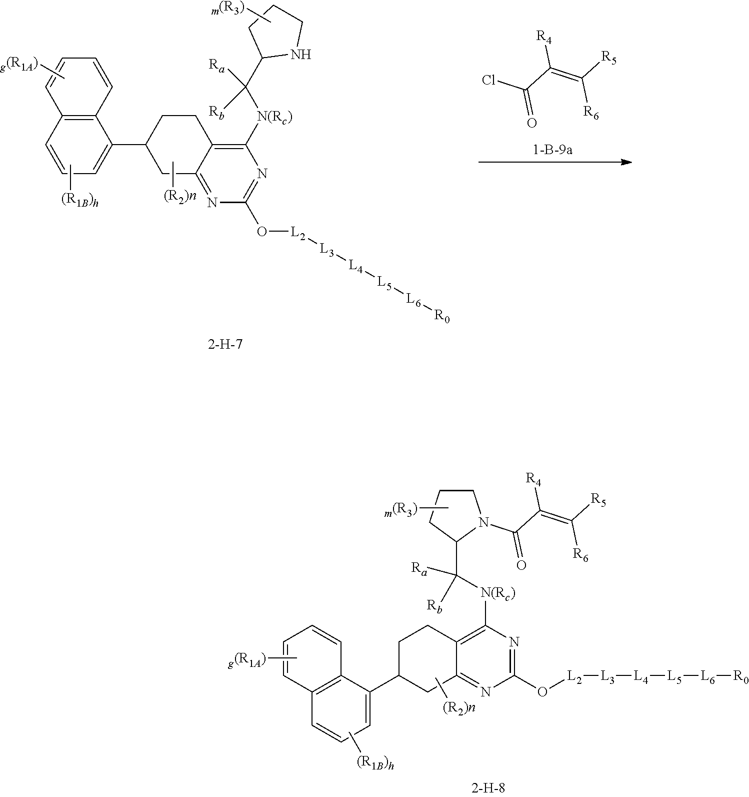

In Scheme (2-H), the starting material 1-H-4 can be prepared by conventional procedures using appropriate compounds and reagent. The starting material 1-H-4 can be converted to 2-H-1 via a SNAr substitution reaction. After that, the intermediate 2-H-1 is deprotected to give 2-H-2, which can be converted to 2—H-3. The intermediate 2-H-3 can go through a Suzuki coupling reaction with 1-H-7a to provide 2-H-4, which can be transform to 2-H-5. Next, 2-H-5 can be reduced to 2-H-6 readily, which is deprotected to give 2-H-7. Finally, the intermediate 2-H-7 can react with 1-B-9a to yield the target compound 2-H-8.

Also, the target compounds can be synthesized by alternative methods but not limited to the above procedures.

An approach to synthesize compounds of

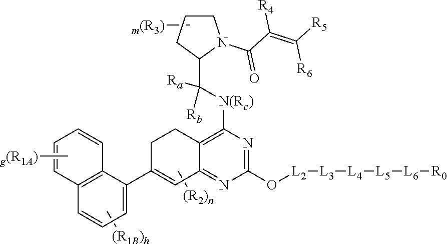

is described in Scheme (2-I).

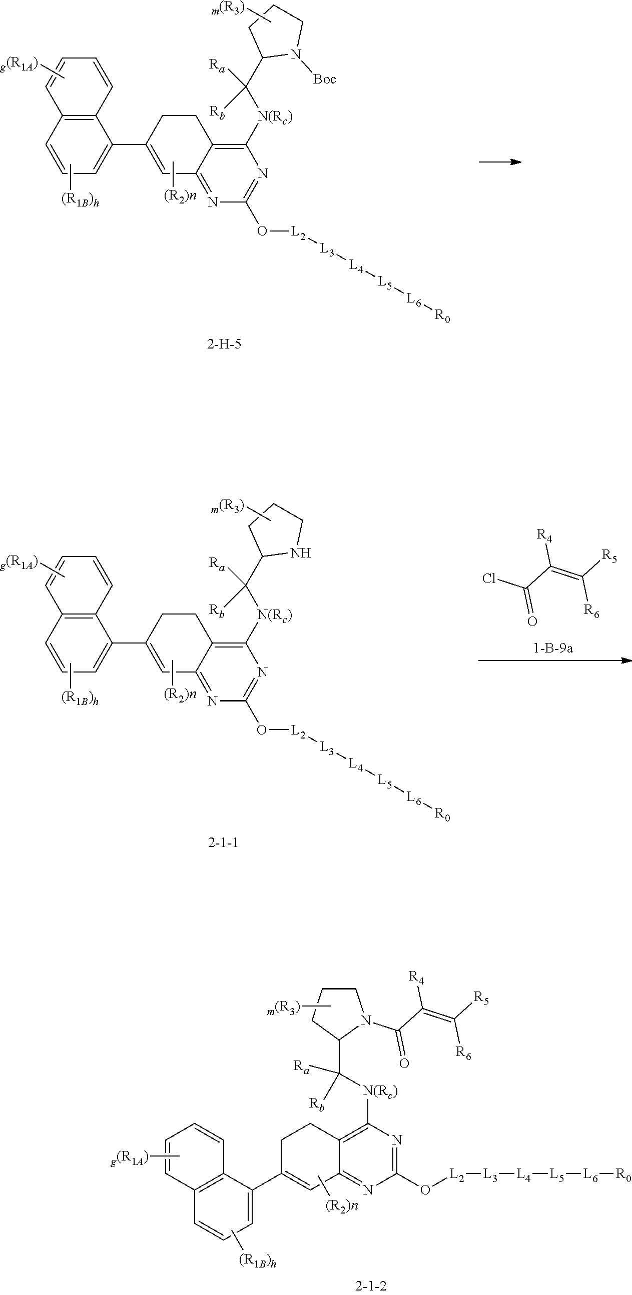

In Scheme (2-I), the starting material 2-H-5 can be prepared by conventional procedures using appropriate compounds and reagent. The intermediate 2-H-5 is deprotected to afford 2-I-1, which can react with 1-B-9a to yield the target compound 2-I-2.

Also, the target compounds can be synthesized by alternative methods but not limited to the above procedures.

An approach to synthesize compounds of

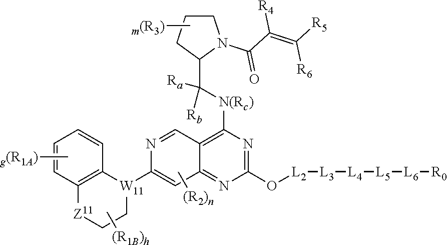

is described in Scheme (2-J)

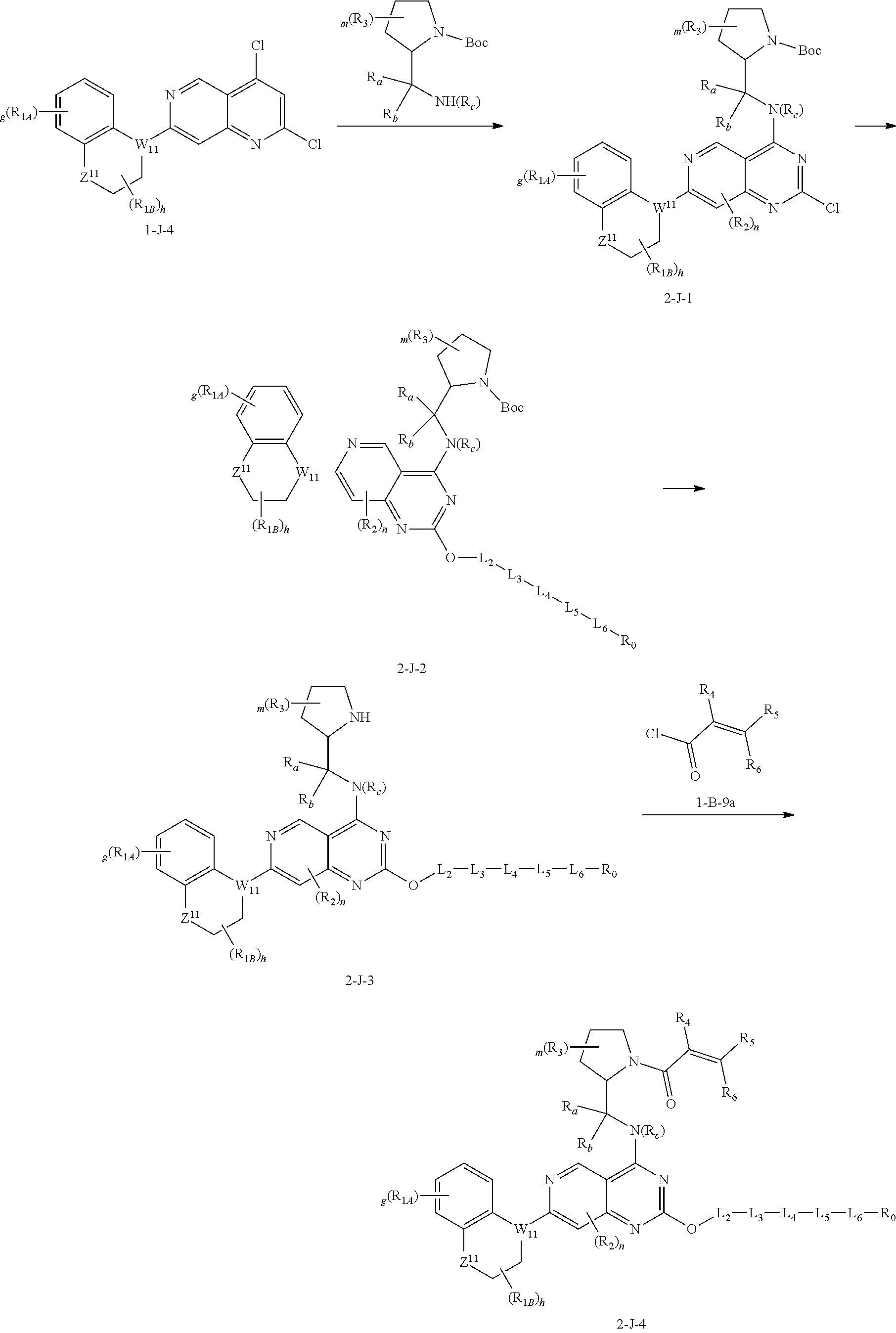

In Scheme (2-J), the starting material 1-J-4 can be prepared by conventional procedures using appropriate compounds and reagent. The starting material 1-J-4 can be converted to 2-J-2 through a sequence of two-step reaction. Finally, the intermediate 2-J-2 is deprotected to afford 2-J-3, which can react with 1B-9a to yield the target compound 2-J-4.

Also, the target compounds can be synthesized by alternative methods but not limited to the above procedures.

An approach to synthesize compounds of

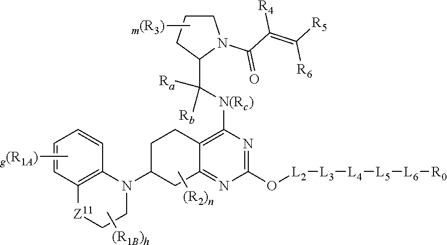

is described in Scheme (2-K).

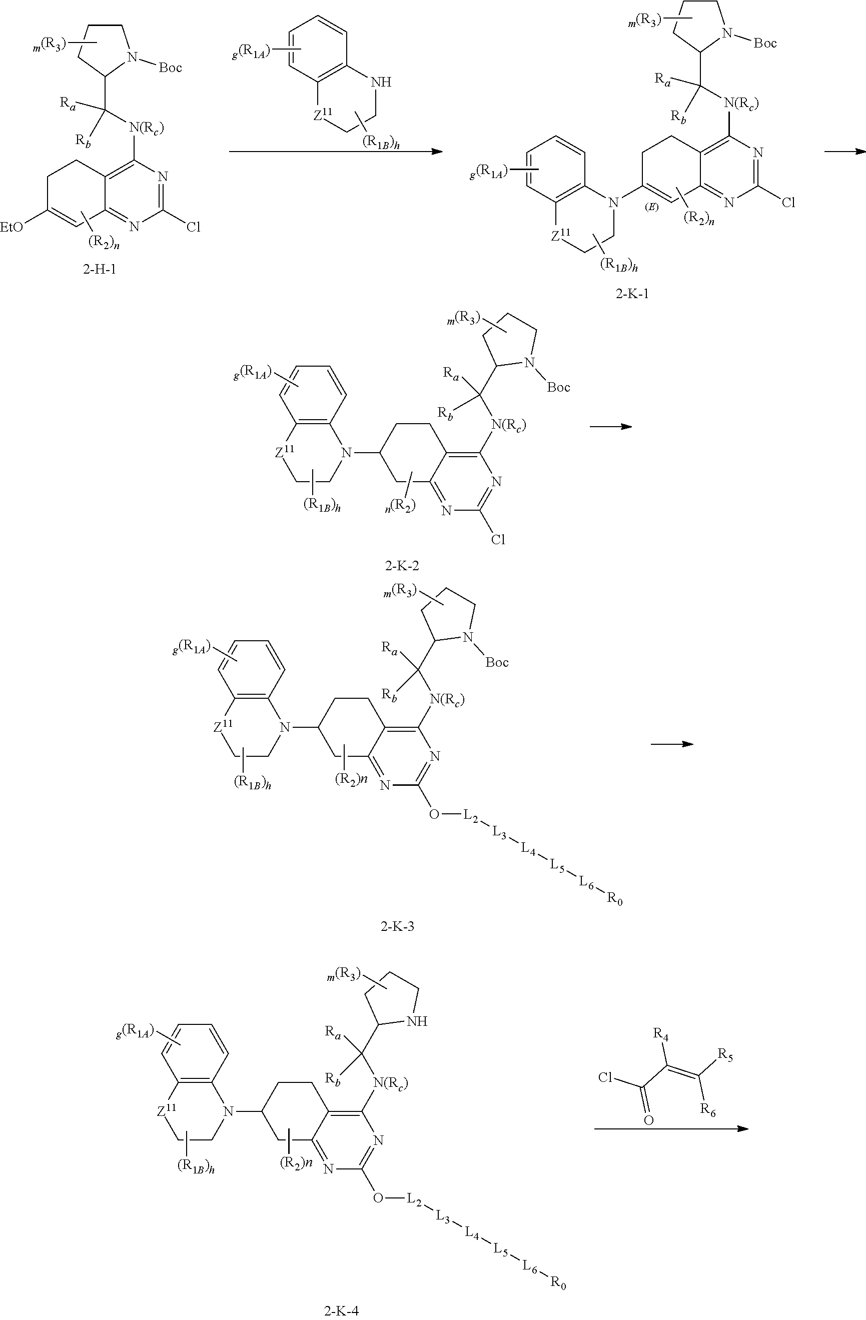

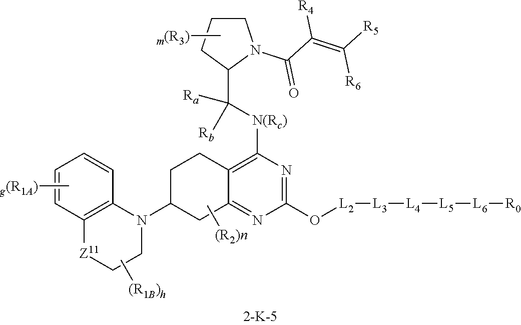

In Scheme (2-K), the starting material 2-H-1 can be prepared by conventional procedures using appropriate compounds and reagent. The starting material 2-H-1 can be converted to the intermediate 2-K-1. After that, 2-K-1 is reduced to 2-K-2, which can be converted to 2-K-3 via a general coupling reaction. Finally, the intermediate 2-K-3 is deprotected to afford 2-K-4, which can react with 1-B-9a to yield the target compound 2-K-5.

Also, the target compounds can be synthesized by alternative methods but not limited to the above procedures.

An approach to synthesize compounds of

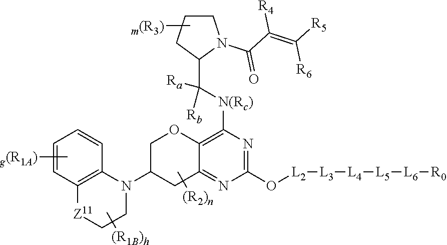

is described in Scheme (2-L).

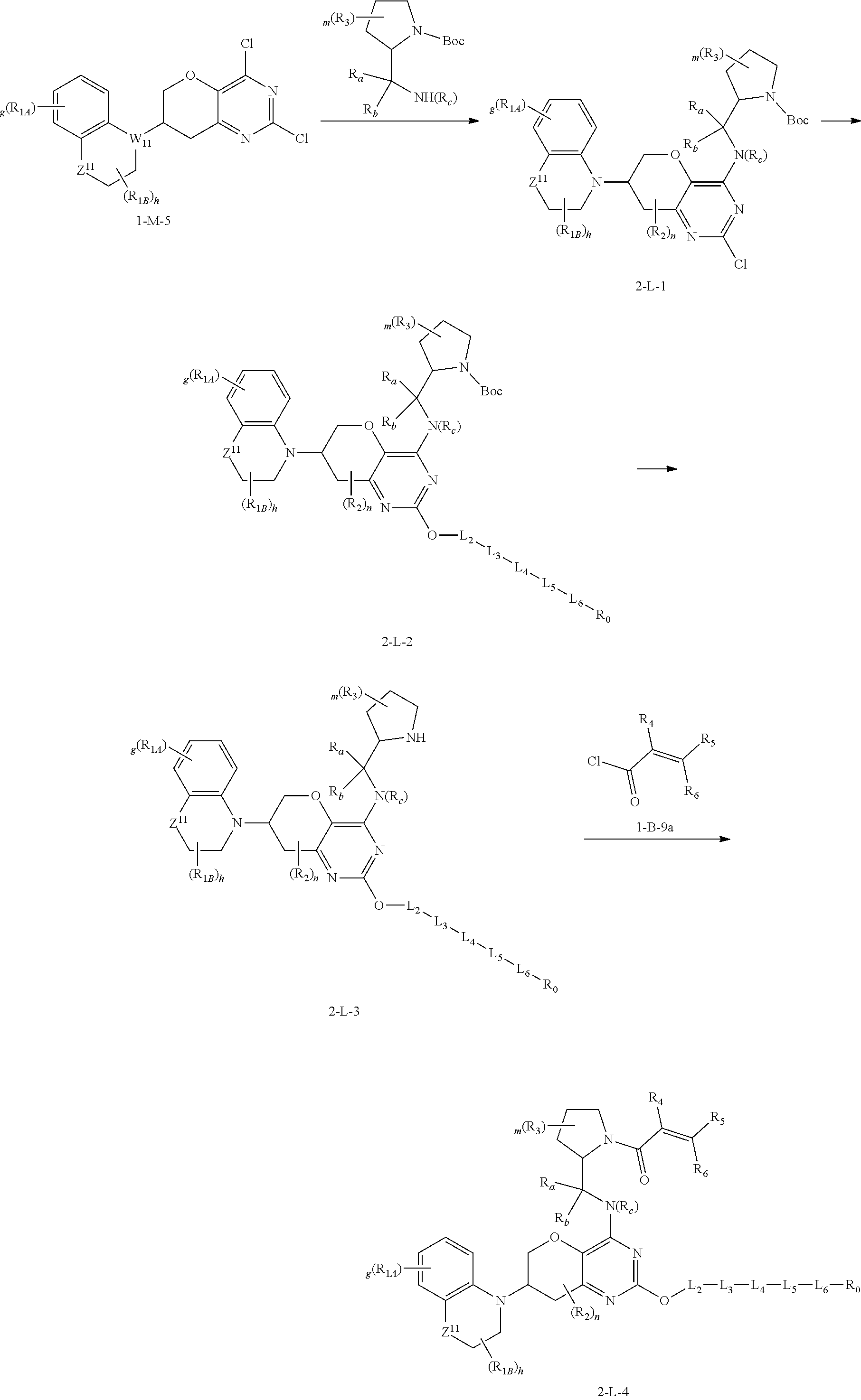

In Scheme (2-L), the starting material 1-M-5 can be prepared by conventional procedures using appropriate compounds and reagent. The starting material 1-M-5 can go through a sequence of two-step coupling reaction to provide 2-L-2. Finally, 2-L-2 is deprotected to afford 2-L-3, which can react with 1-B-9a to yield the target compound 2-L-4.

Also, the target compounds can be synthesized by alternative methods but not limited to the above procedures.

An approach to synthesize compounds of

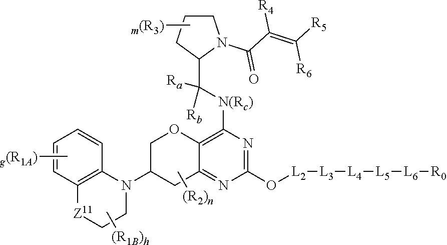

is described in Scheme (2-M).

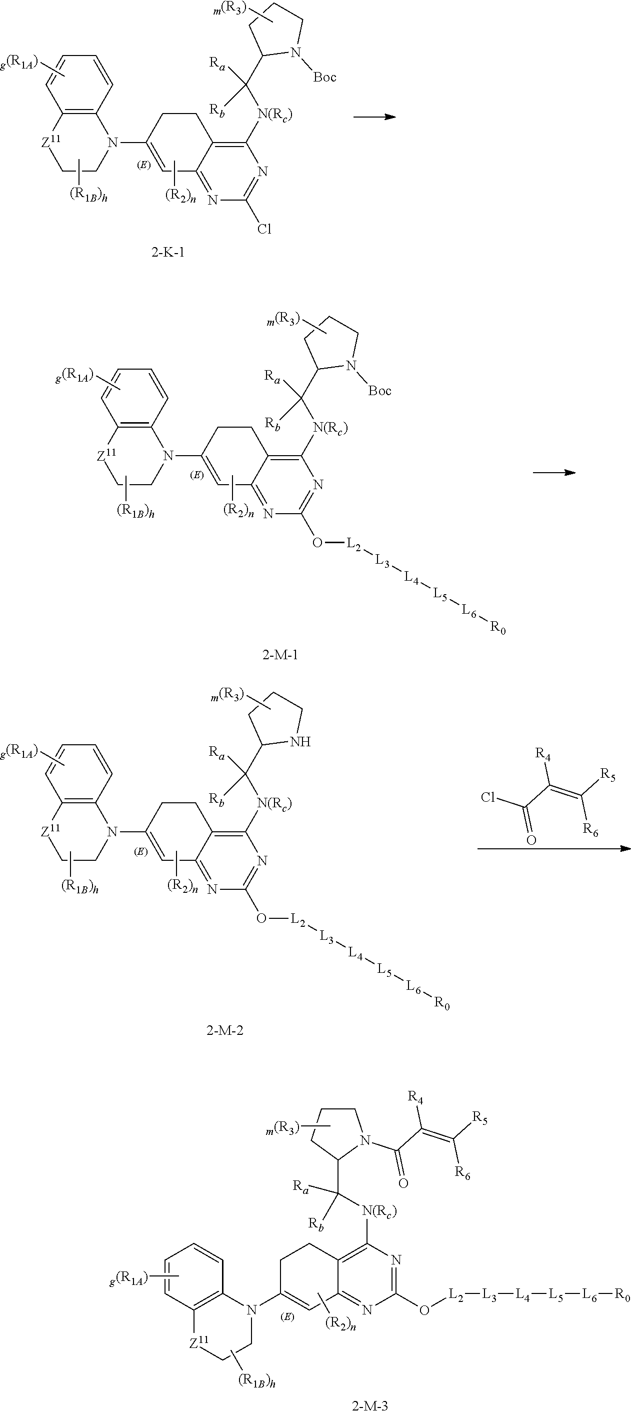

In Scheme (2-M), the starting material 2-K-1 can be prepared by conventional procedures using appropriate compounds and reagent. The starting material 2-K-1 can be converted to 2-M-1 readily. After that, the intermediate 2-M-1 is deprotected to afford 2-M-2, which can react with 1-B-9a to yield the target compound 2-M-3.

Also, the target compounds can be synthesized by alternative methods but not limited to the above procedures.

An approach to synthesize compounds of

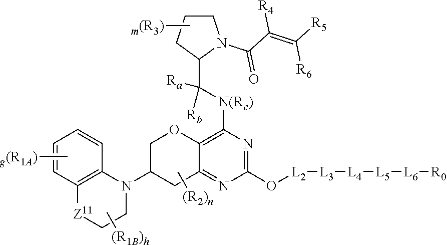

is described in Scheme (2-N).

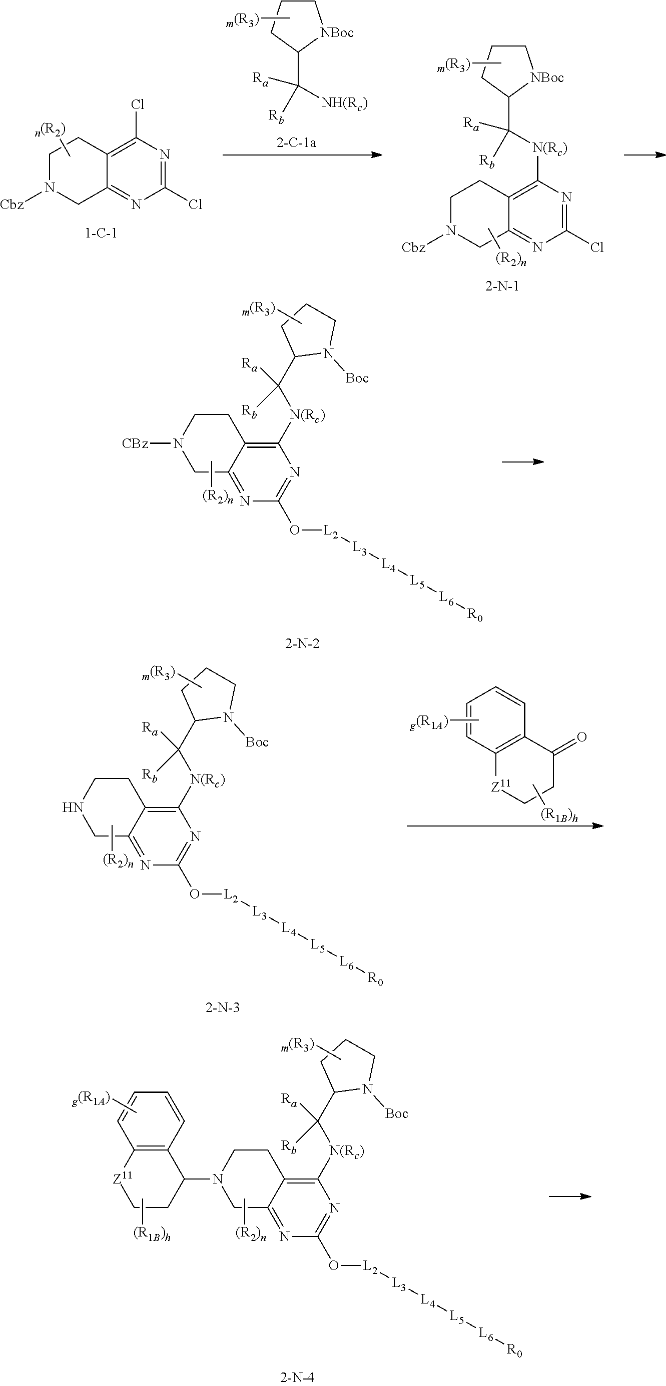

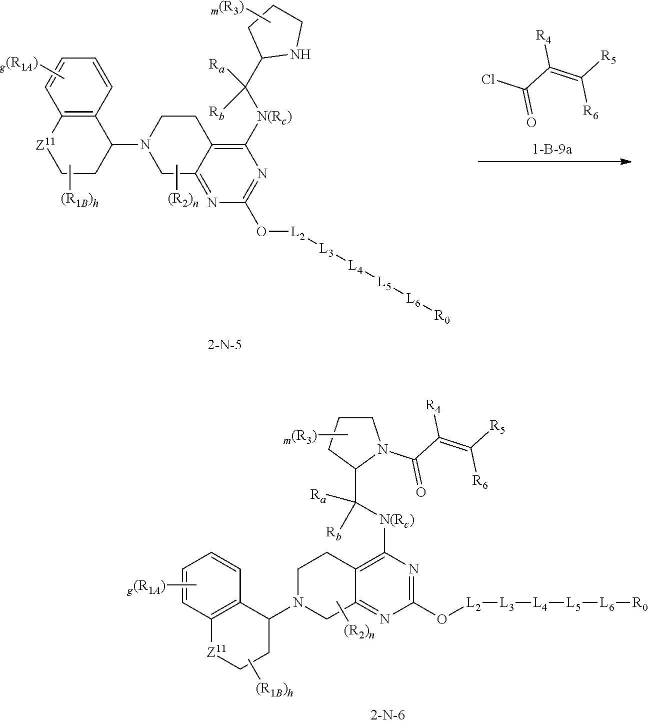

In Scheme (2-N), the starting material 1-C-1 can be prepared by conventional procedures using appropriate compounds and reagent. The starting material 1-C-1 can react with 2-C-1a to give intermediate 2-N-1, which can be converted to the intermediate 2-N-2 via a coupling reaction. After that, 2-N-2 is deprotected to afford 2-N-3, which can go through a reductive amination reaction to give 2-N-4. Finally, 2-N-4 is deprotected to afford 2-N-5, which can react with 1-B-9a to yield the target compound 2-N-6.

Also, the target compounds can be synthesized by alternative methods but not limited to the above procedures.

An approach to synthesize compounds of

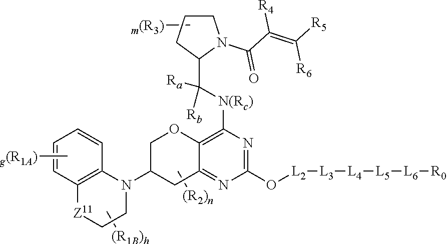

is described in Scheme (2-O).

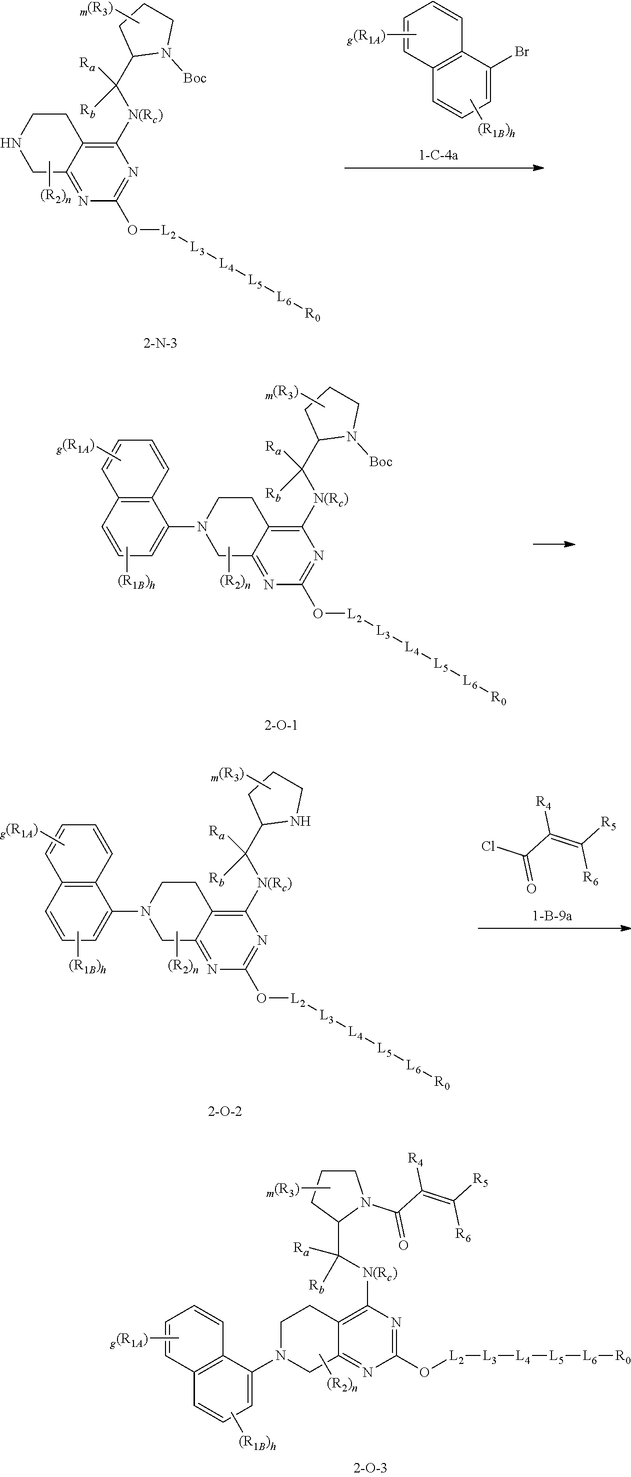

In Scheme (2-O), the starting material 2-N-3 can be prepared by conventional procedures using appropriate compounds and reagent. The starting material 2-N-3 can go through a Buchwald coupling reaction with 1-C-4a to give 2-O-1. Finally, 2-O-1 is deprotected to afford 2-O-2, which can react with 1-B-9a to yield the target compound 2-O-3.

Also, the target compounds can be synthesized by alternative methods but not limited to the above procedures.

The compounds

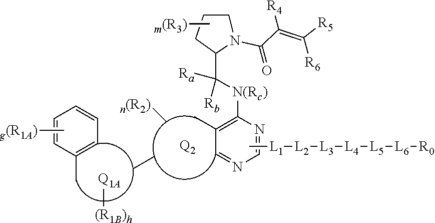

can be made by the method referred to Scheme 2-C to 2-O, by using different starting material and reagents, or by the conventional organic reactions.

The compounds

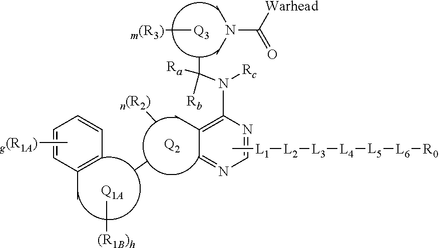

can be made by the method referred to Scheme 2-C to 2-O, by using different starting material and reagents, or by the conventional organic reactions.

The compounds

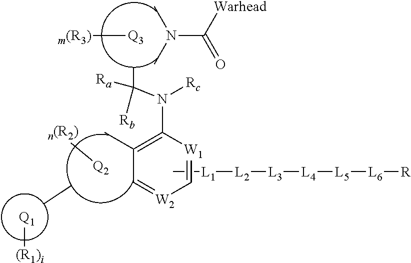

can be made by the method referred to Scheme 2-C to 2-O, by using different starting material and reagents, or by the conventional organic reactions.

An approach to synthesize compounds of

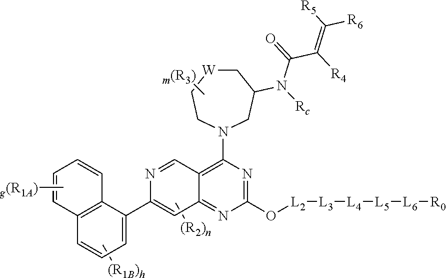

is described in Scheme (3-C).

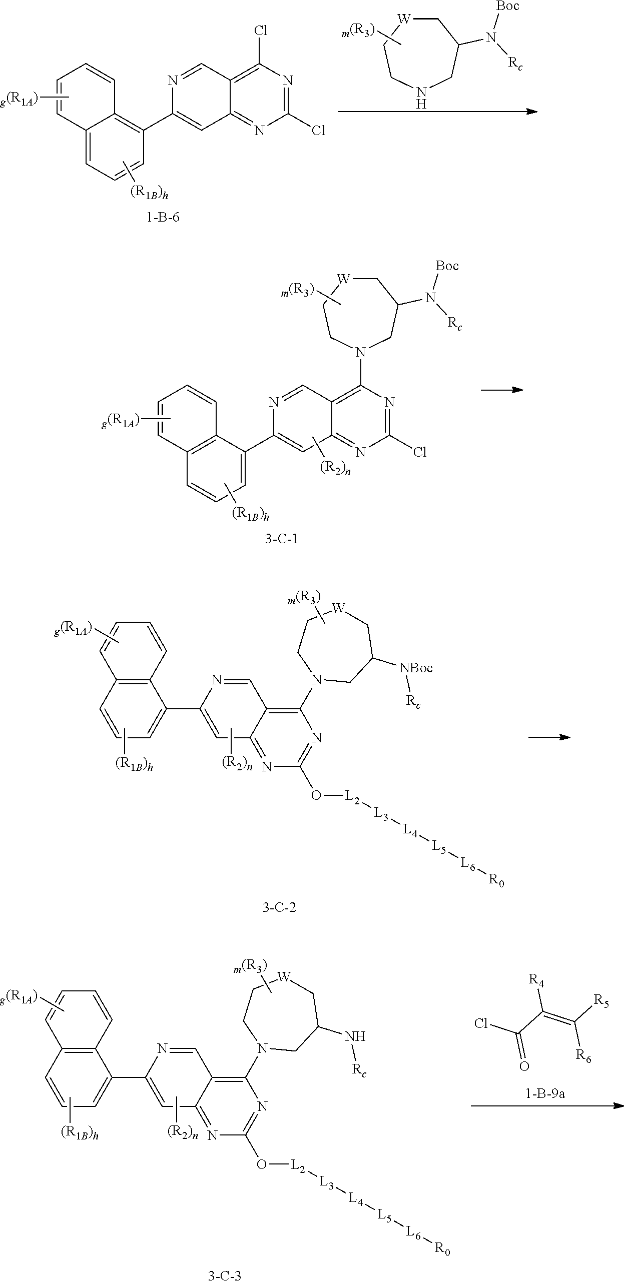

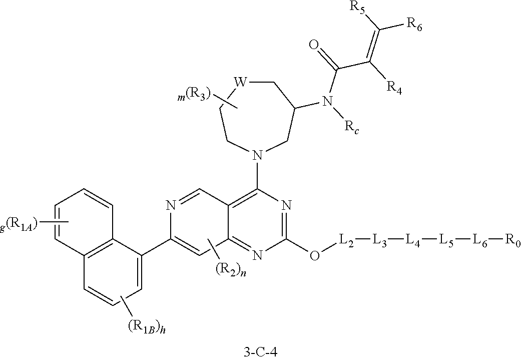

In Scheme (3-C), the starting material 1-B-6 can be prepared by conventional procedures using appropriate compounds and reagent. The starting material 1-B-6 is converted to intermediate 3-C-1 through a SNAr substitution reaction, which can be converted to the intermediate 3-C-2 readily. Finally, 3-C-2 is deprotected to afford 3-C-3, which can react with 1-B-9a to yield the target compound 3-C-4.

Also, the target compounds can be synthesized by alternative methods but not limited to the above procedures.

An approach to synthesize compounds of

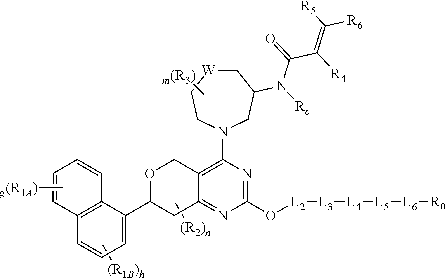

is described in Scheme (3-D).

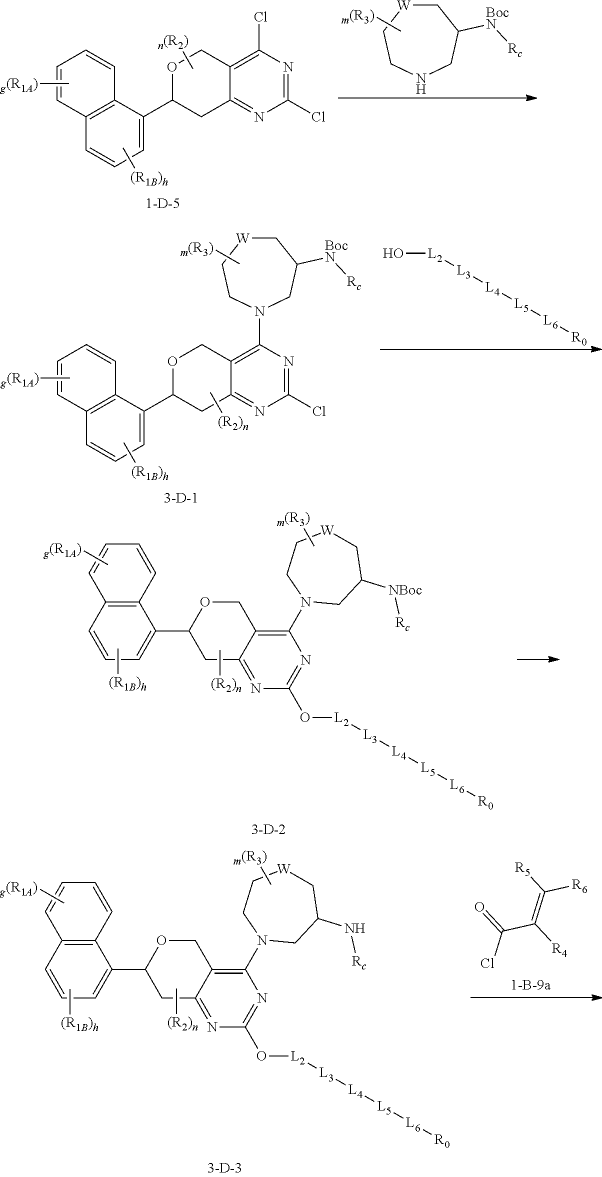

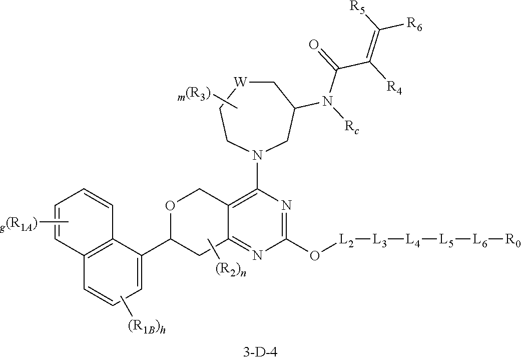

In Scheme (3-D), the starting material 1-D-5 can be prepared by conventional procedures using appropriate compounds and reagent. The starting material 1-D-5 can go through a sequence of two-step coupling reaction to give 3-D-2. The intermediate 3-D-2 is deprotected to afford 3-D-3, which can react with 1-B-9a to yield the target compound 3-D-4.

Also, the target compounds can be synthesized by alternative methods but not limited to the above procedures.

An approach to synthesize compounds of

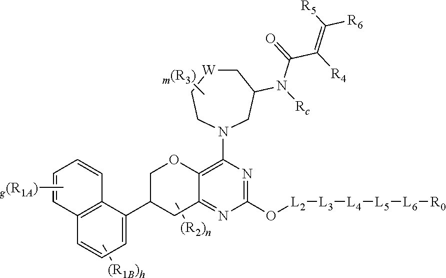

is described in Scheme (3-E).

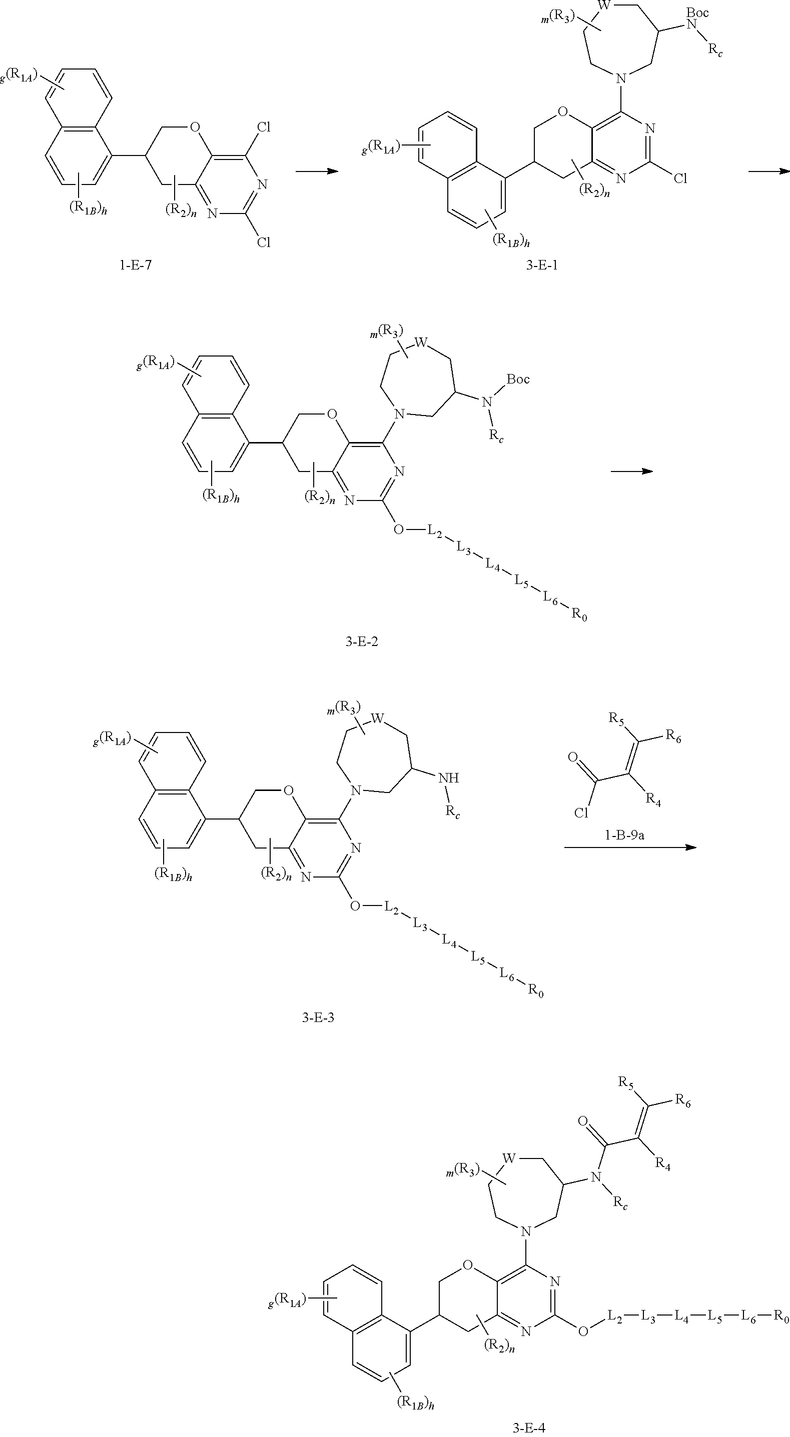

In Scheme (3-E), the starting material 1-E-7 can be prepared by conventional procedures using appropriate compounds and reagent. The starting material 1-E-7 can go through a sequence of two-step coupling reaction to provide 3-E-2. After that, 3-E-2 is deprotected to afford 3-E-3, which can react with 1-B-9a to yield the target compound 3-E-4.

Also, the target compounds can be synthesized by alternative methods but not limited to the above procedures.

An approach to synthesize compounds of

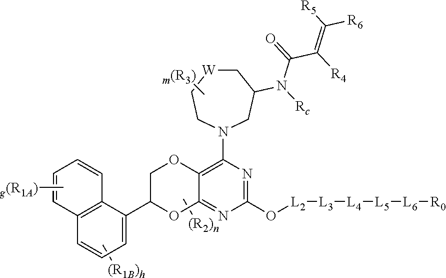

is described in Scheme (3-F).

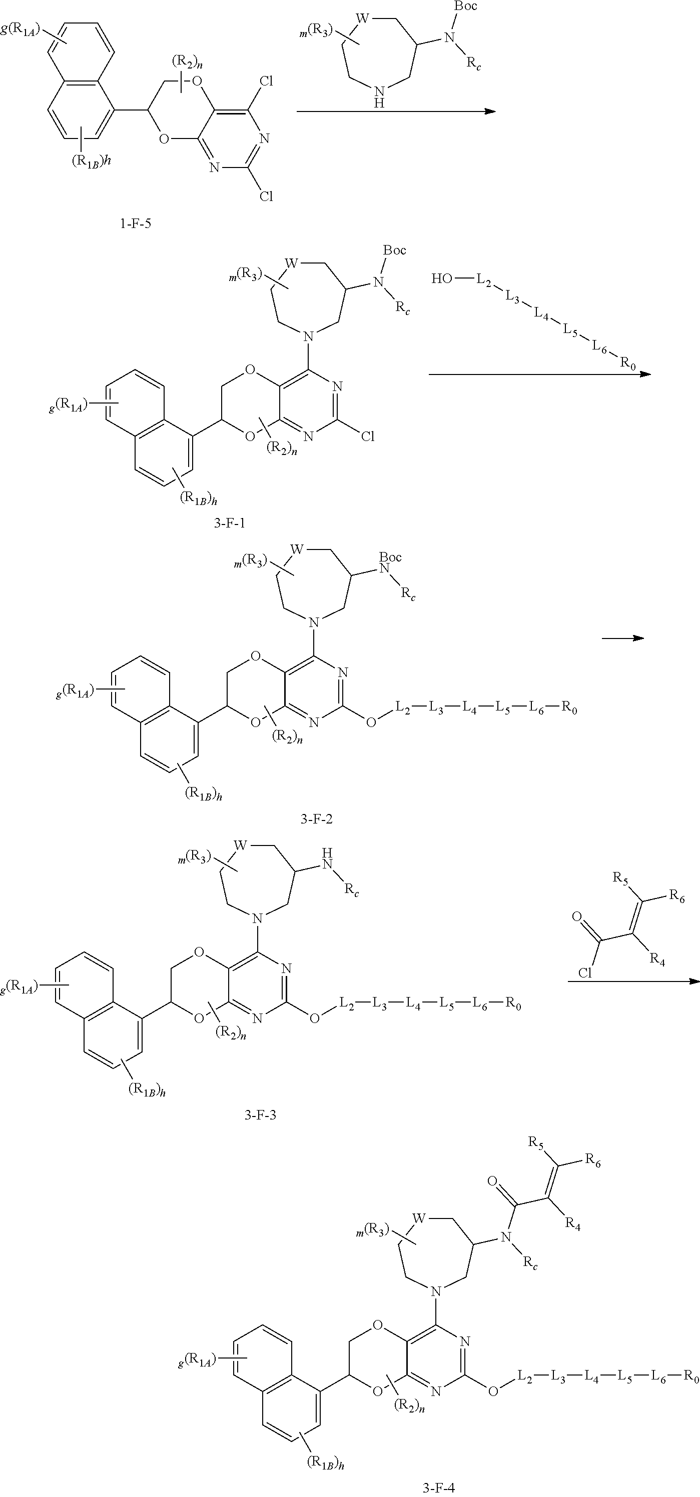

In Scheme (3-F), the starting material 1-F-5 can be prepared by conventional procedures using appropriate compounds and reagent. The starting material 1-F-5 can go through a sequence of two-step coupling reaction to provide 3-F-2. Finally, 3-F-2 is deprotected to afford 3-F-3, which can react with 1-B-9a to yield the target compound 3-F-4.

Also, the target compounds can be synthesized by alternative methods but not limited to the above procedures.

An approach to synthesize compounds of

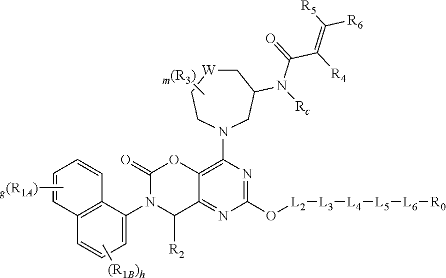

is described in Scheme (3-G).

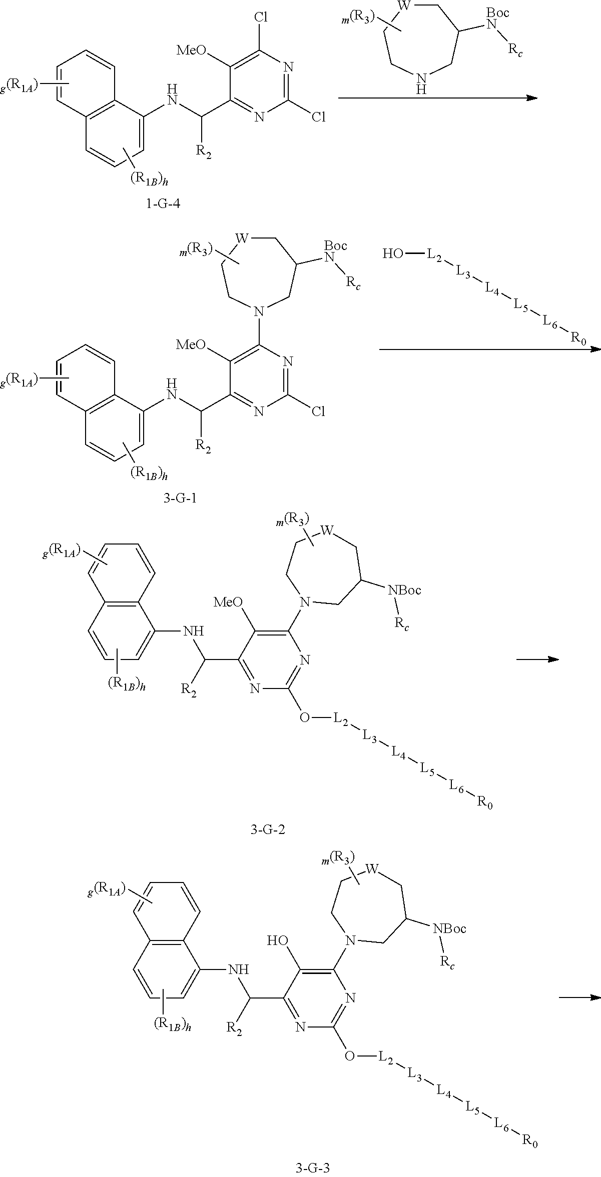

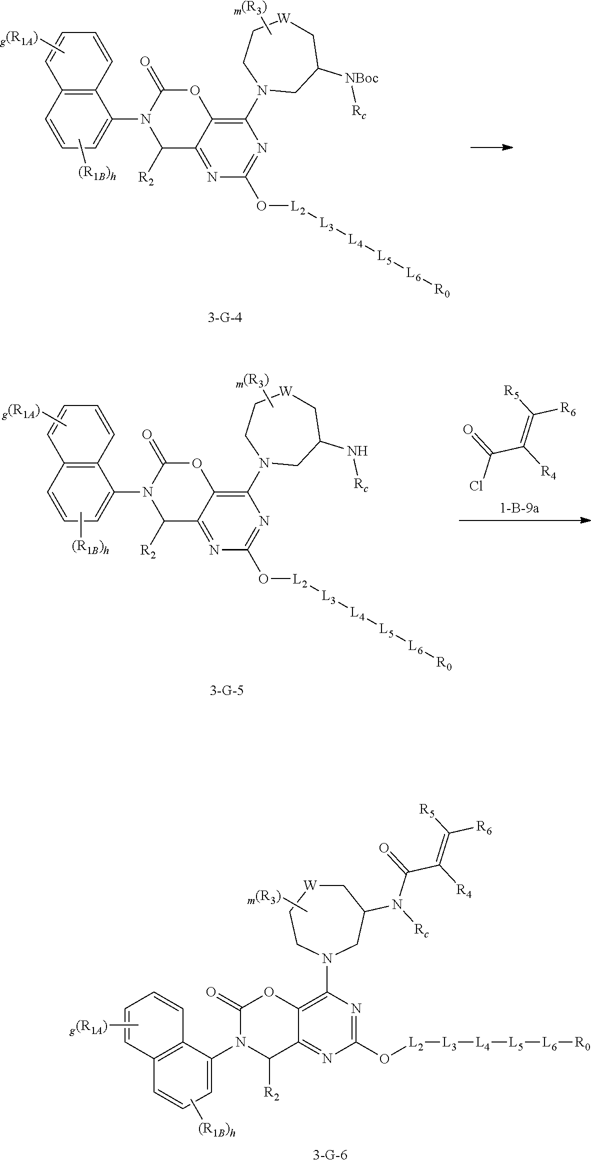

In Scheme (3-G), the starting material 1-G-4 can be prepared by conventional procedures using appropriate compounds and reagent. The starting material 1-G-4 can go through a sequence of two-step coupling reaction to provide 3-G-2. The demethylation of 3-G-2 can afford 3-G-3, which can be further transform to 3-G-4. Finally, the intermediate 3-G-4 is deprotected to afford 3-G-5, which can react with 1-B-9a to yield the target compound 3-G-6.

Also, the target compounds can be synthesized by alternative methods but not limited to the above procedures.

An approach to synthesize compounds of

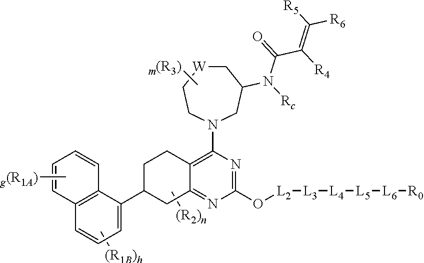

is described in Scheme (3-H).

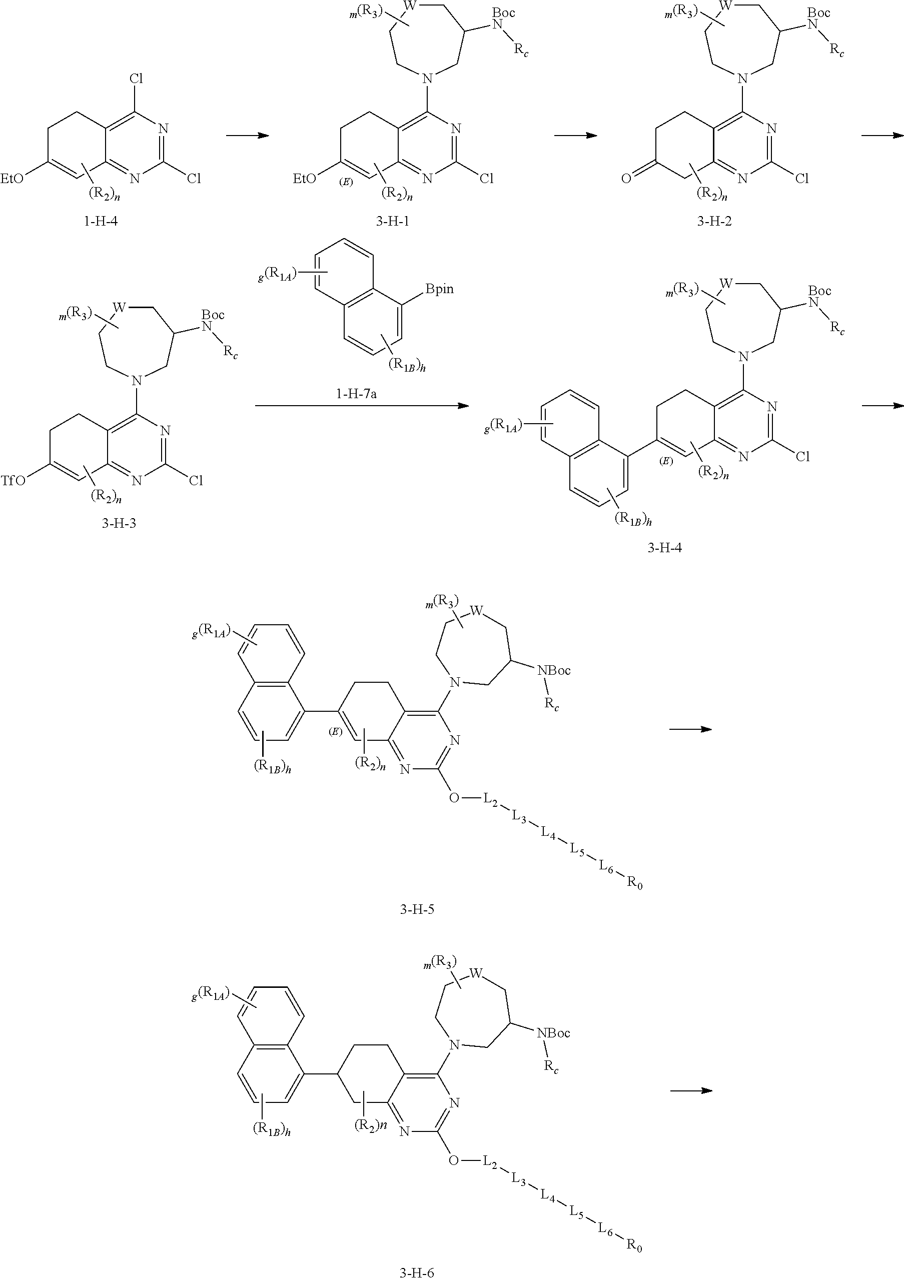

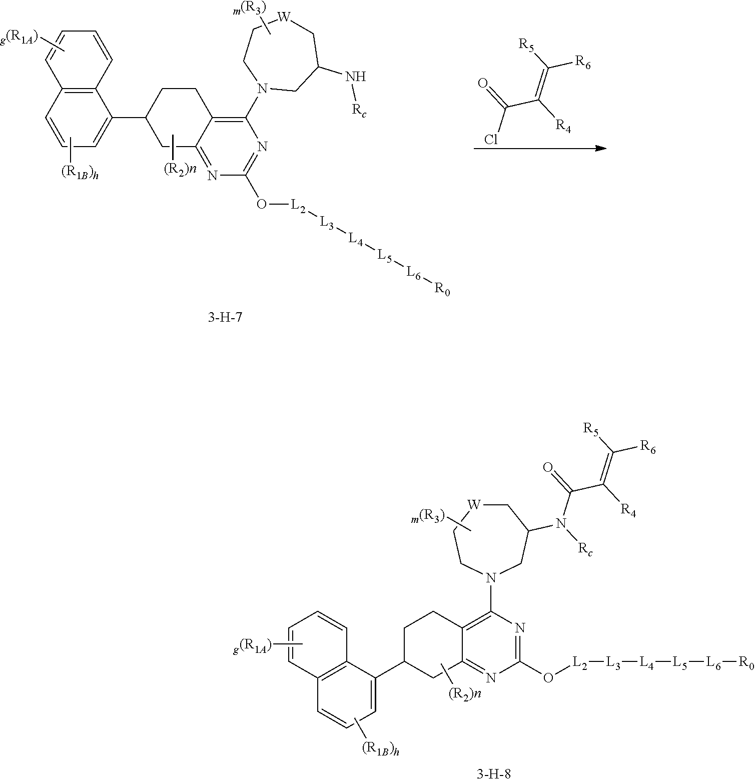

In Scheme (3-H), the starting material 1-H-4 can be prepared by conventional procedures using appropriate compounds and reagent. The starting material 1-H-4 can be converted to 3-H-1 via a SNAr substitution reaction. After that, the intermediate 3-H-1 is deprotected to give 3-H-2, which can be converted to 3-H-3 readily. The intermediate 3-H-3 can go through a Suzuki coupling reaction with 1-H-7a to provide 3-H-4, which can be converted to 3-H-5. Next, the intermediate 3-H-5 can be hydrogenated to 3-H-6, which can be deprotected to give 3-H-7. Finally, the intermediate 3-H-7 can react with 1-B-9a to yield the target compound 3-H-8.

Also, the target compounds can be synthesized by alternative methods but not limited to the above procedures.

An approach to synthesize compounds of

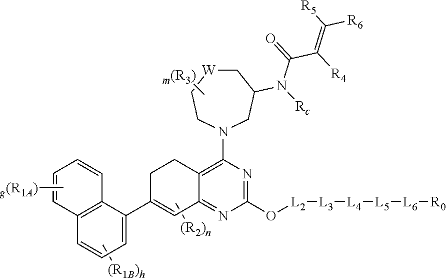

is described in Scheme (3-I).

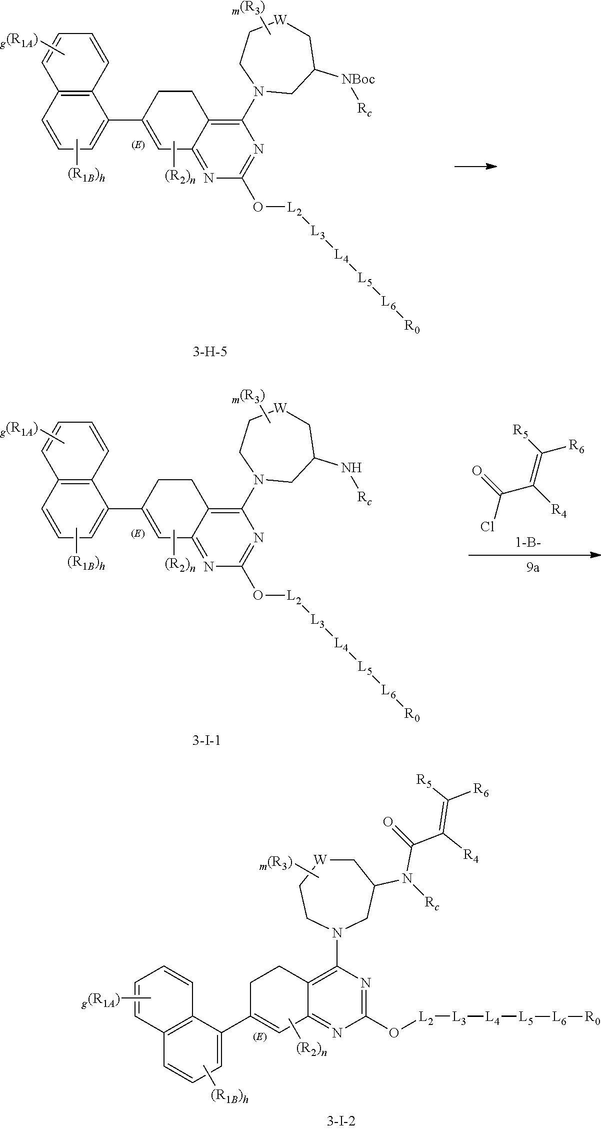

In Scheme (3-I), the starting material 3-H-5 can be prepared by conventional procedures using appropriate compounds and reagent. The intermediate 3-H-5 is deprotected to afford 3-I-1, which can react with 1-B-9a to yield the target compound 3-I-2.

Also, the target compounds can be synthesized by alternative methods but not limited to the above procedures.

An approach to synthesize compounds of

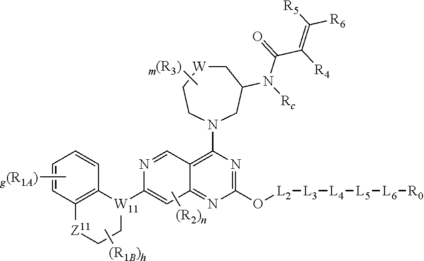

is described in Scheme (3-J).

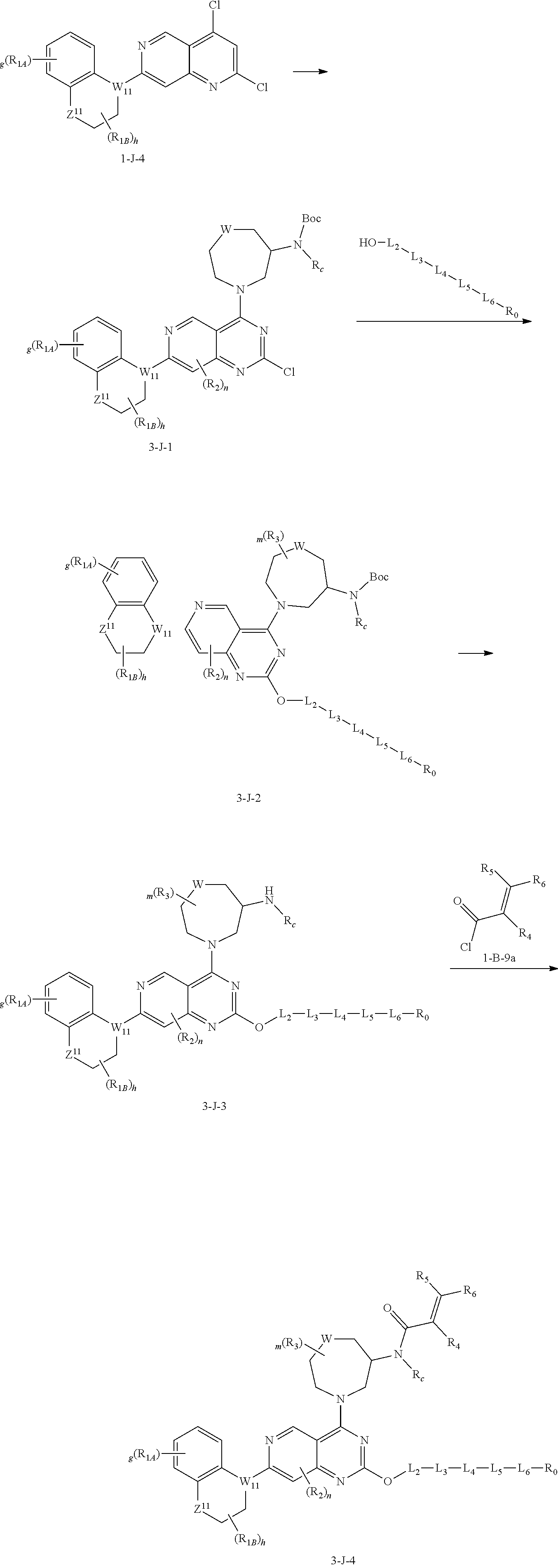

In Scheme (3-J), the starting material 1-J-4 can be prepared by conventional procedures using appropriate compounds and reagent. The starting material 1-J-4 can be converted to 3-J-2 through a sequence of two-step reaction. After that, the intermediate 3-J-2 is deprotected to afford 3-J-3, which can react with 1-B-9a to yield the target compound 3-J-4.

Also, the target compounds can be synthesized by alternative methods but not limited to the above procedures.

An approach to synthesize compounds of

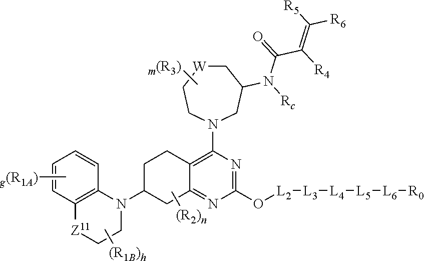

is described in Scheme (3-K).

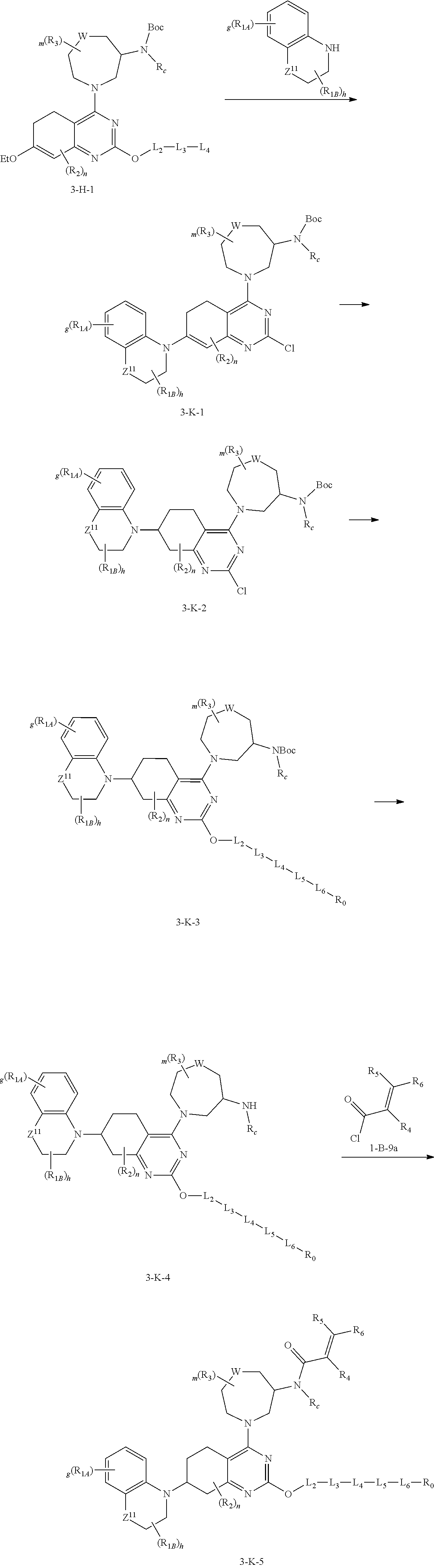

In Scheme (3-K), the starting material 3-H-1 can be prepared by conventional procedures using appropriate compounds and reagent. The starting material 3-H-1 can be converted to the intermediate 3-K-1. After that, 3-K-1 is reduced to 3-K-2, which can be converted to 3-K-3 via a general coupling reaction. Finally, the intermediate 3-K-3 is deprotected to afford 3-K-4, which can react with 1-B-9a to yield the target compound 3-K-5.

Also, the target compounds can be synthesized by alternative methods but not limited to the above procedures.

An approach to synthesize compounds of

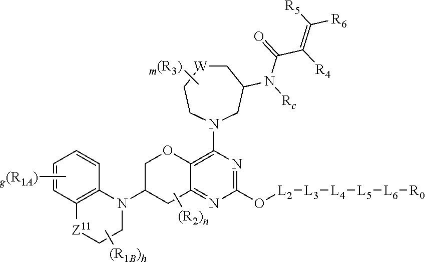

is described in Scheme (3-L).

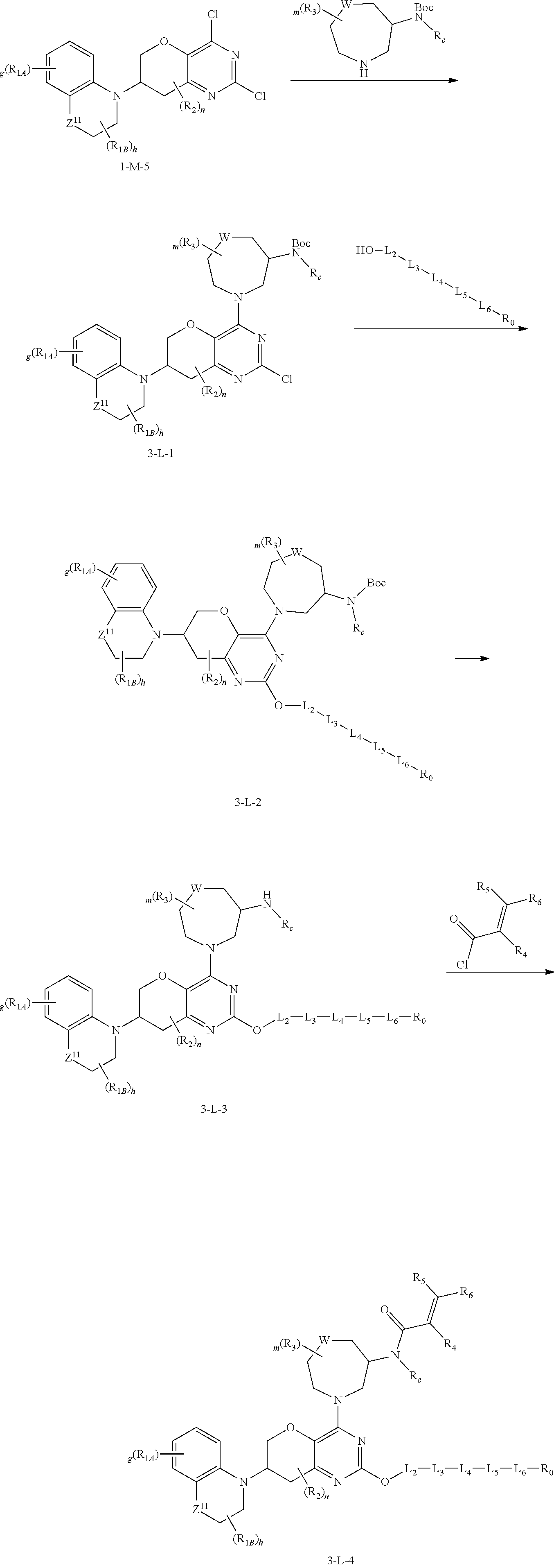

In Scheme (3-L), the starting material 1-M-5 can be prepared by conventional procedures using appropriate compounds and reagent. The starting material 1-M-5 can go through a sequence of two-step coupling reaction to provide 3-L-2. After that, 3-L-2 is deprotected to afford 3-L-3, which can react with 1-B-9a to yield the target compound 3-L-4.

Also, the target compounds can be synthesized by alternative methods but not limited to the above procedures.

An approach to synthesize compounds of

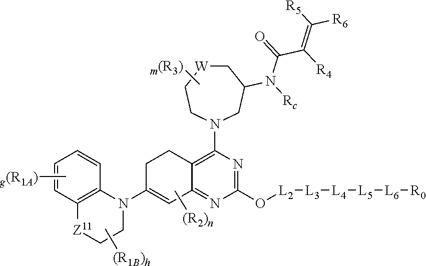

is described in Scheme (3-M).

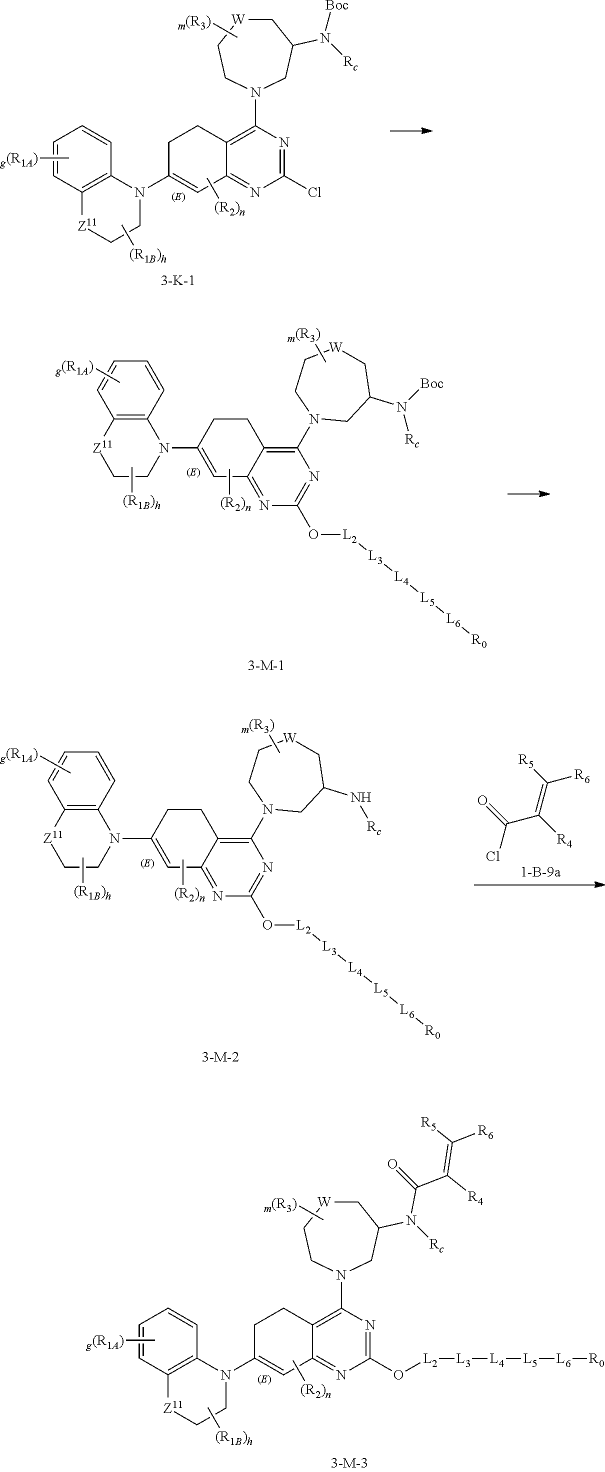

In Scheme (3-M), the starting material 3-K-1 can be prepared by conventional procedures using appropriate compounds and reagent. The starting material 3-K-1 can be converted to 3-M-1. After that, the intermediate 3-M-1 is deprotected to afford 3-M-2, which can react with 1-B-9a to yield the target compound 3-M-3.

Also, the target compounds can be synthesized by alternative methods but not limited to the above procedures.

An approach to synthesize compounds of

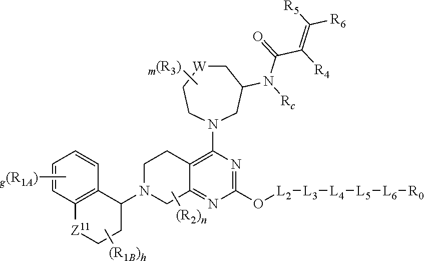

is described in Scheme (3-N).

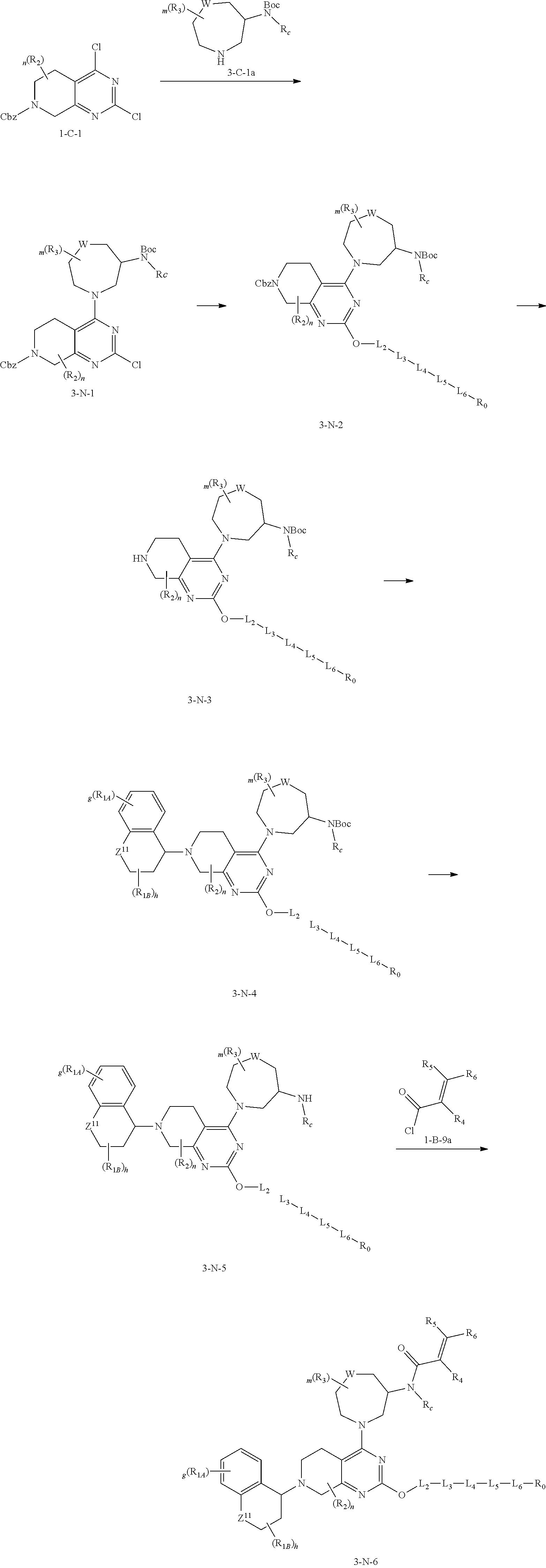

In Scheme (3-N), the starting material 1-C-1 can be prepared by conventional procedures using appropriate compounds and reagent. The starting material 1-C-1 can react with 3-C-1a to give intermediate 3-N-1, which can be converted to the intermediate 3-N-2 via a coupling reaction. After that, 3-N-2 is deprotected to afford 3-N-3, which can go through a reductive amination reaction to give 3-N-4. Finally, 3-N-4 is deprotected to afford 3-N-5, which can react with 1-B-9a to yield the target compound 3-N-6.

Also, the target compounds can be synthesized by alternative methods but not limited to the above procedures.

An approach to synthesize compounds of

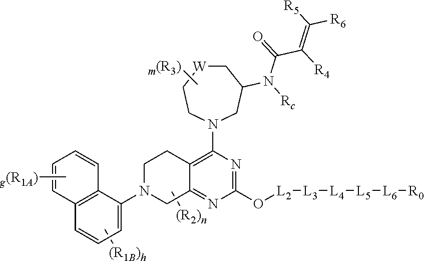

is described in Scheme (3-O)

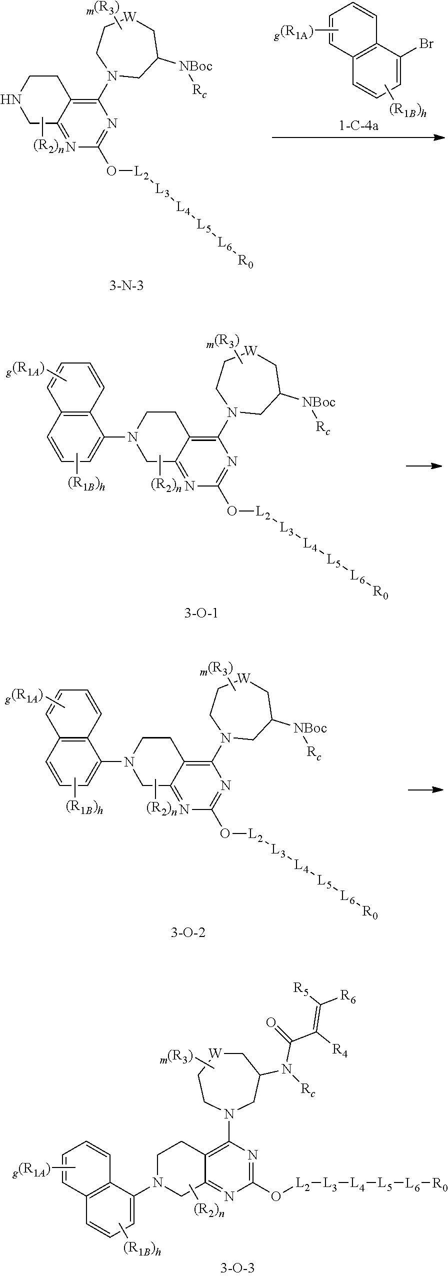

In Scheme (3-O), the starting material 3-N-3 can be prepared by conventional procedures using appropriate compounds and reagent. The starting material 3-N-3 can go through a Buchwald coupling reaction with 1-C-4a to give 3-O-1. Finally, 3-O-1 is deprotected to afford 3-O-2, which can react with 1-B-9a to yield the target compound 3-O-3.

Also, the target compounds can be synthesized by alternative methods but not limited to the above procedures.

The compounds

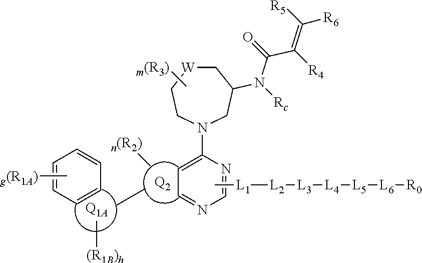

can be made by the method referred to Scheme 3-C to 3-O, by using different starting material and reagents, or by the conventional organic reactions.

The compounds

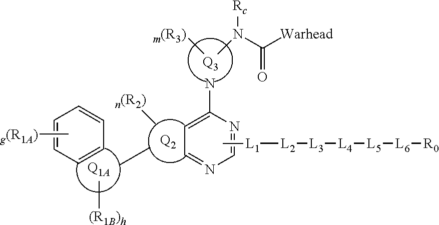

can be made by the method referred to Scheme 3-C to 3-O, by using different starting material and reagents, or by the conventional organic reactions.

The compounds

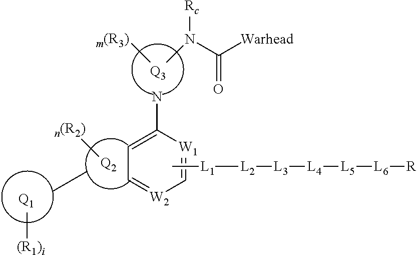

can be made by the method referred to Scheme 3-C to 3-O, by using different starting material and reagents, or by the conventional organic reactions.

An approach to synthesize compounds of

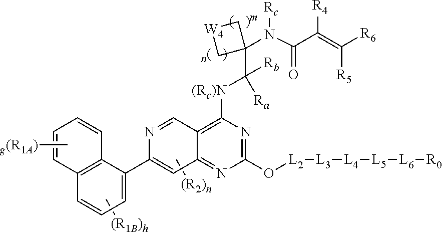

is described in Scheme (4-C).

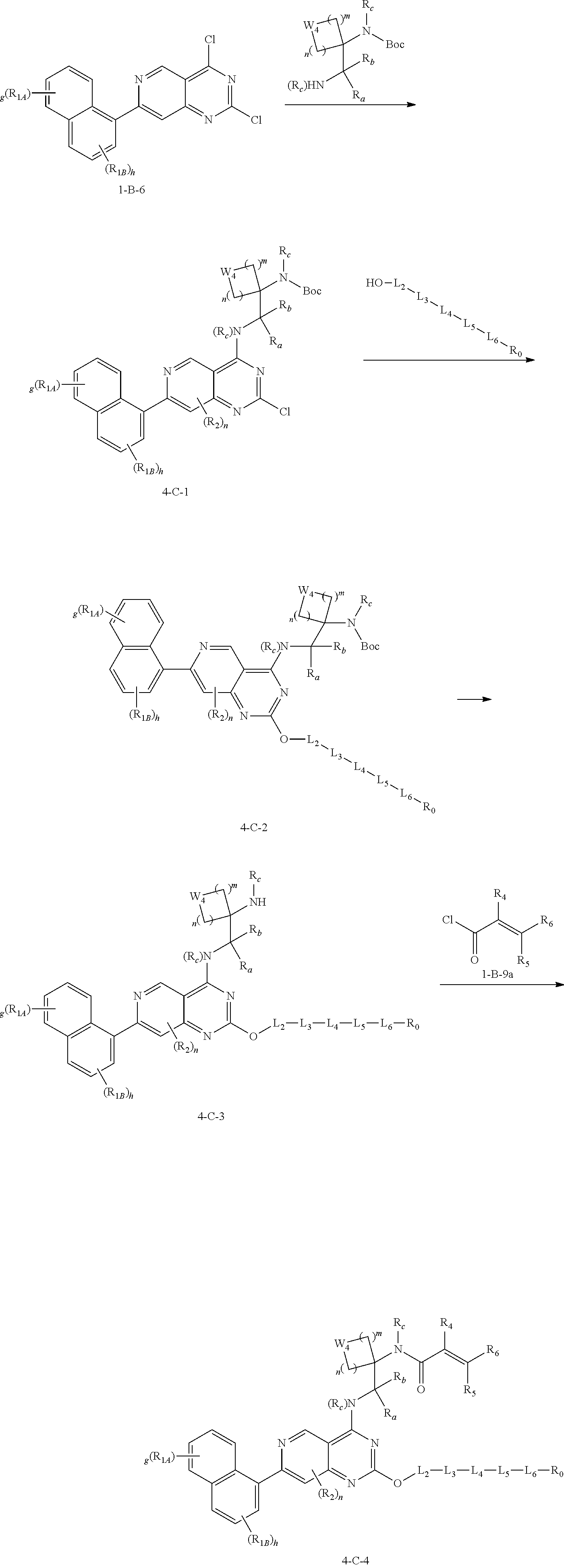

In Scheme (4-C), the starting material 1-1B-6 can be prepared by conventional procedures using appropriate compounds and reagent. The starting material 1-1B-6 is converted to intermediate 4-C-1 through a SNAr substitution reaction, which can be converted to the intermediate 4-C-2 readily. After that, 4-C-2 is deprotected to afford 4-C-3, which can react with 1-B-9a to yield the target compound 4-C-4.

Also, the target compounds can be synthesized by alternative methods but not limited to the above procedures.

An approach to synthesize compounds of

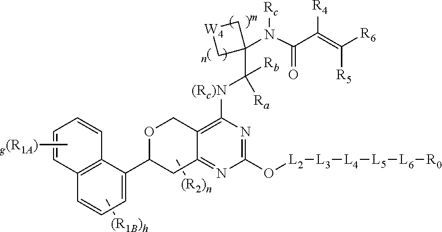

is described in Scheme (4-D).

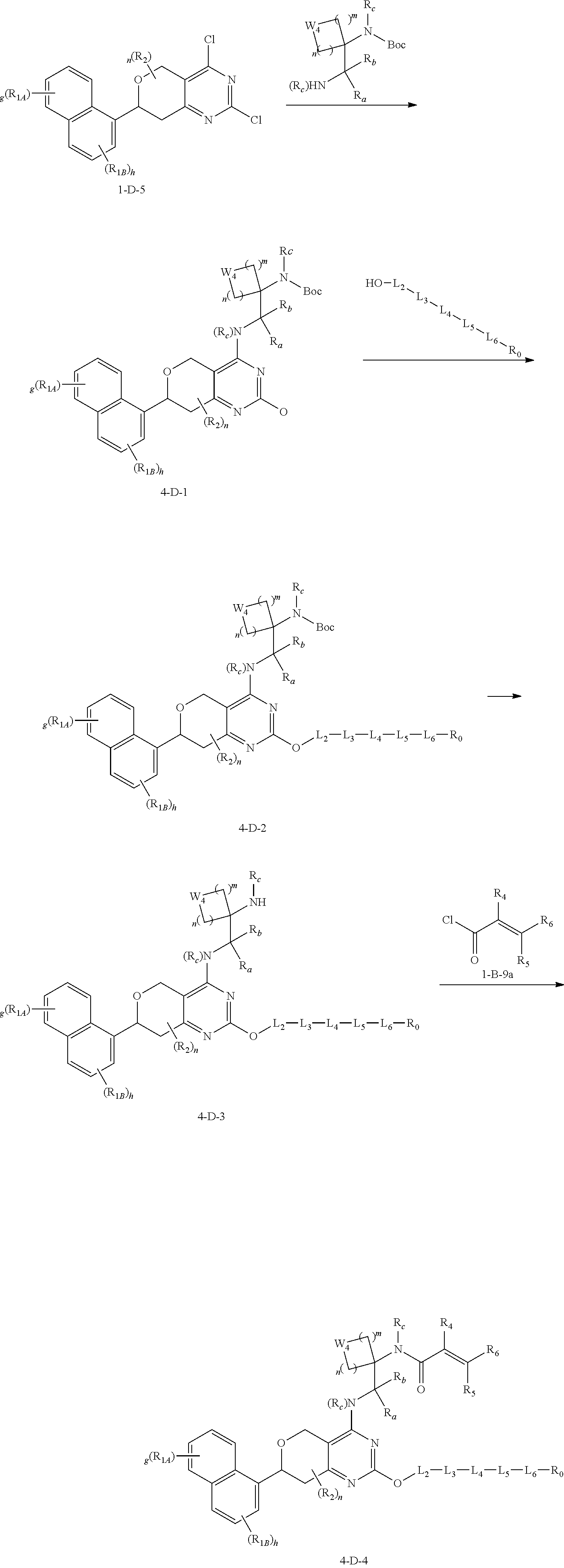

In Scheme (4-D), the starting material 1-D-5 can be prepared by conventional procedures using appropriate compounds and reagent. The starting material 1-D-5 can go through a sequence of two-step coupling reaction to give 4-D-2. The intermediate 4-D-2 is deprotected to afford 4-D-3, which can react with 1-B-9a to yield the target compound 4-D-4.

Also, the target compounds can be synthesized by alternative methods but not limited to the above procedures.

An approach to synthesize compounds of

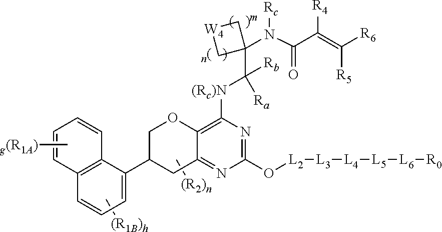

is described in Scheme (4-E).

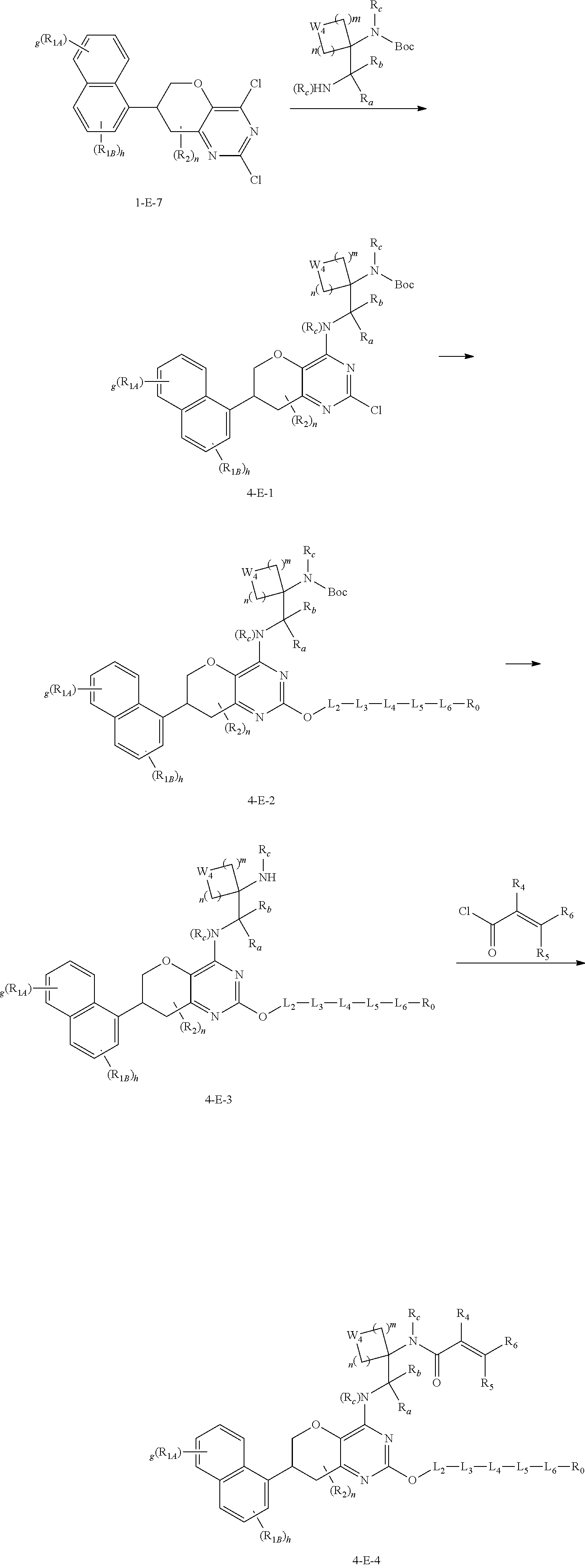

In Scheme (4-E), the starting material 1-E-7 can be prepared by conventional procedures using appropriate compounds and reagent. The starting material 1-E-7 can go through a sequence of two-step coupling reaction to provide 4-E-2. Finally, 4-E-2 is deprotected to afford 4-E-3, which can react with 1-B-9a to yield the target compound 4-E-4.

Also, the target compounds can be synthesized by alternative methods but not limited to the above procedures.

An approach to synthesize compounds of

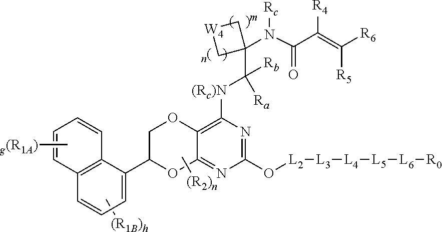

is described in Scheme (4-F).

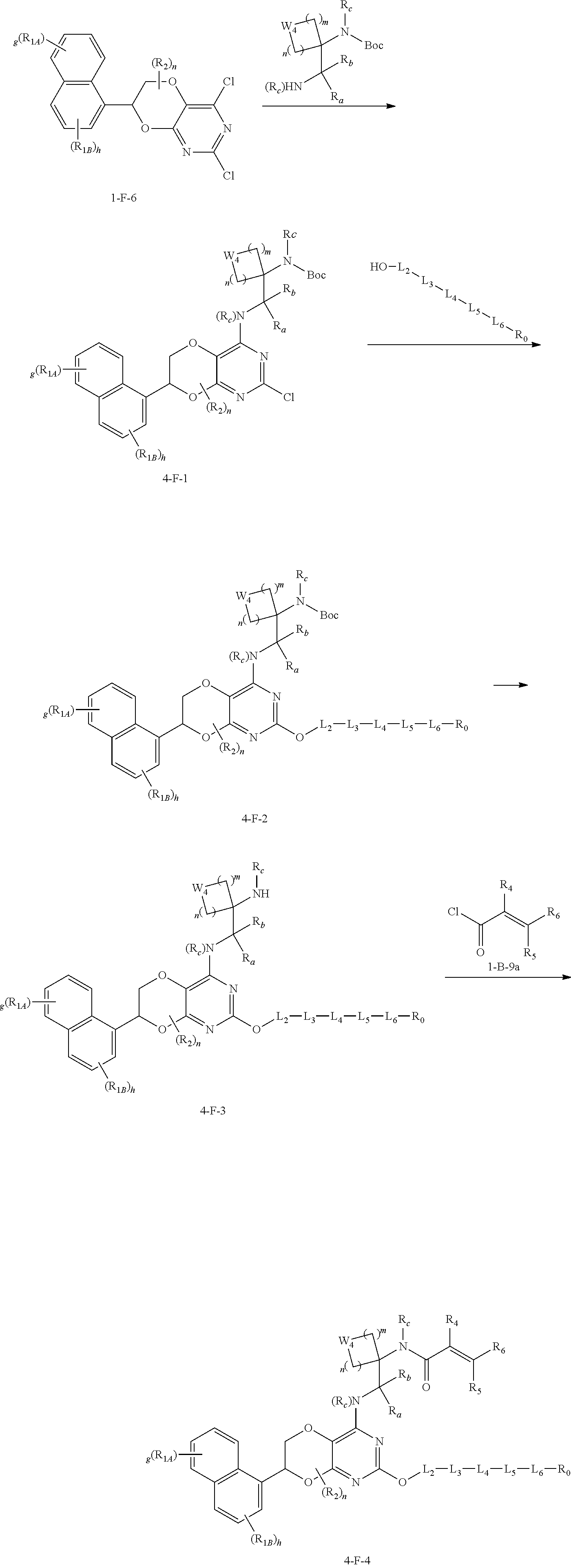

In Scheme (4-F), the starting material 1-F-6 can be prepared by conventional procedures using appropriate compounds and reagent. The starting material 1-F-6 can go through a sequence of two-step coupling reaction to provide 4-F-2. Finally, 4-F-2 is deprotected to afford 4-F-3, which can react with 1-B-9a to yield the target compound 4-F-4.

Also, the target compounds can be synthesized by alternative methods but not limited to the above procedures.

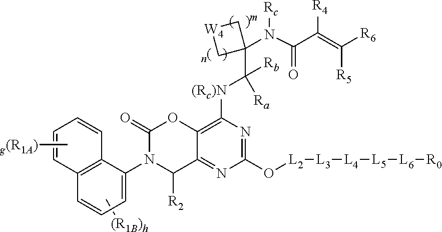

is described in Scheme (4-G).

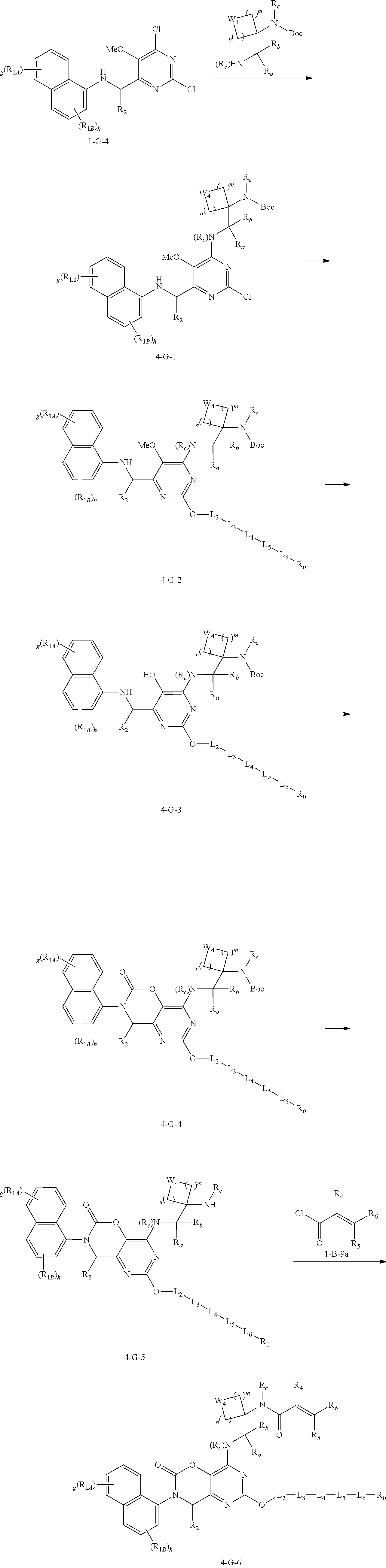

In Scheme (4-G), the starting material 1-G-4 can be prepared by conventional procedures using appropriate compounds and reagent. The starting material 1-G-4 can go through a sequence of two-step coupling reaction to provide 4-G-2. The demethylation of 4-G-2 can afford 4-G-3, which can be further transform to 4-G-4. Finally, the intermediate 4-G-4 is deprotected to afford 4-G-5, which can react with 1-B-9a to yield the target compound 4-G-6.

Also, the target compounds can be synthesized by alternative methods but not limited to the above procedures.

An approach to synthesize compounds of

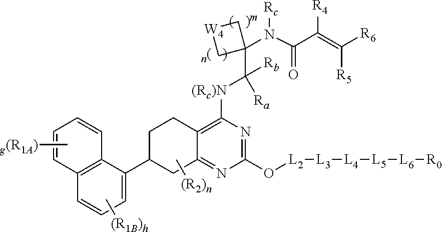

is described in Scheme (4-H).

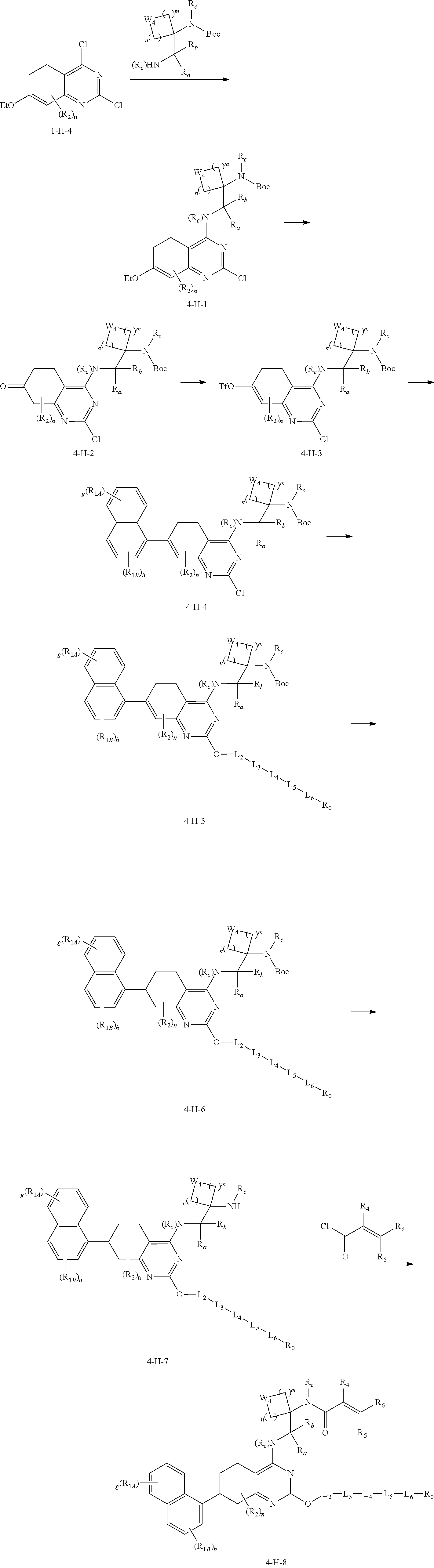

In Scheme (4-H), the starting material 1-H-4 can be prepared by conventional procedures using appropriate compounds and reagent. The starting material 1-H-4 can be converted to 4-H-1 via a SNAr substitution reaction. After that, the intermediate 4-H-1 is deprotected to give 4-H-2, which can be converted to 4-H-3. The intermediate 4-H-3 can go through a Suzuki coupling reaction with 1-H-7a to provide 4-H-4, which can be transform to 4-H-5. Next, 4-H-5 can be hydrogenated to 4-H-6, which is deprotected to give 4-H-7. Finally, the intermediate 4-H-7 can react with 1-B-9a to yield the target compound 4-H-8.

Also, the target compounds can be synthesized by alternative methods but not limited to the above procedures.

An approach to synthesize compounds of

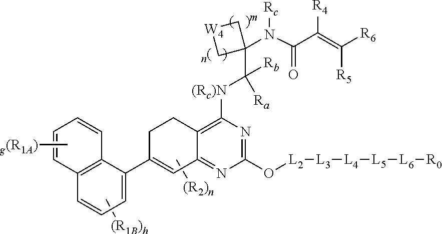

is described in Scheme (4-I).

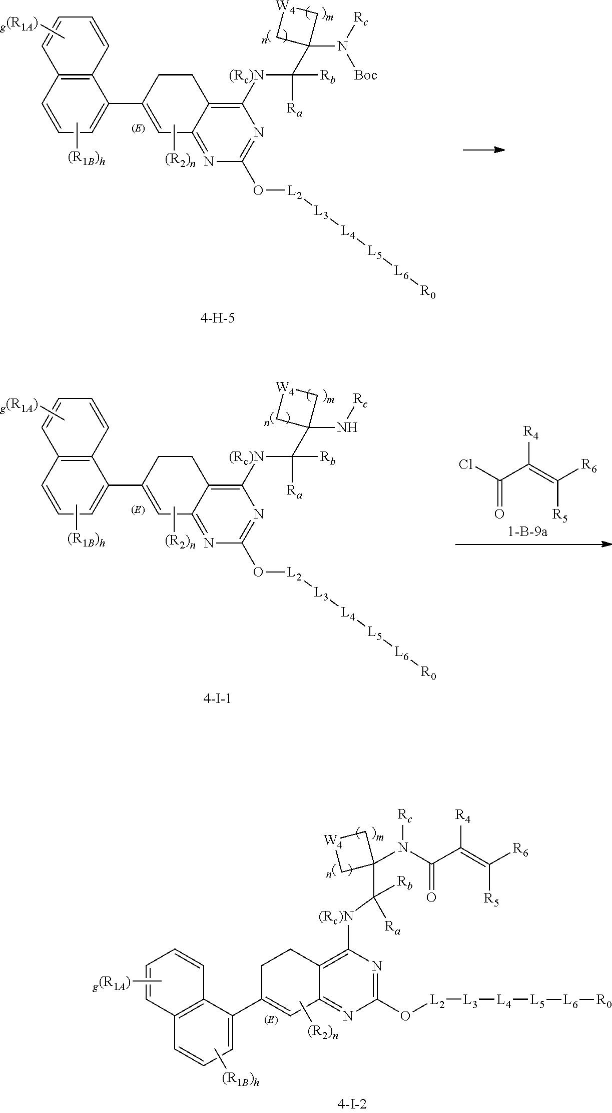

In Scheme (4-I), the starting material 4-H-5 can be prepared by conventional procedures using appropriate compounds and reagent. The intermediate 4-H-5 is deprotected to afford 4-I-1, which can react with 1-B-9a to yield the target compound 4-I-2.

Also, the target compounds can be synthesized by alternative methods but not limited to the above procedures.

An approach to synthesize compounds of

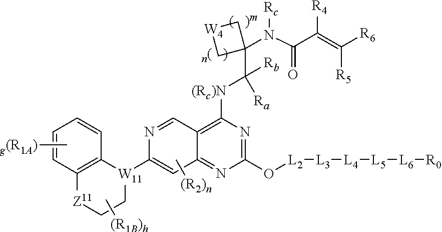

is described in Scheme (4-J).

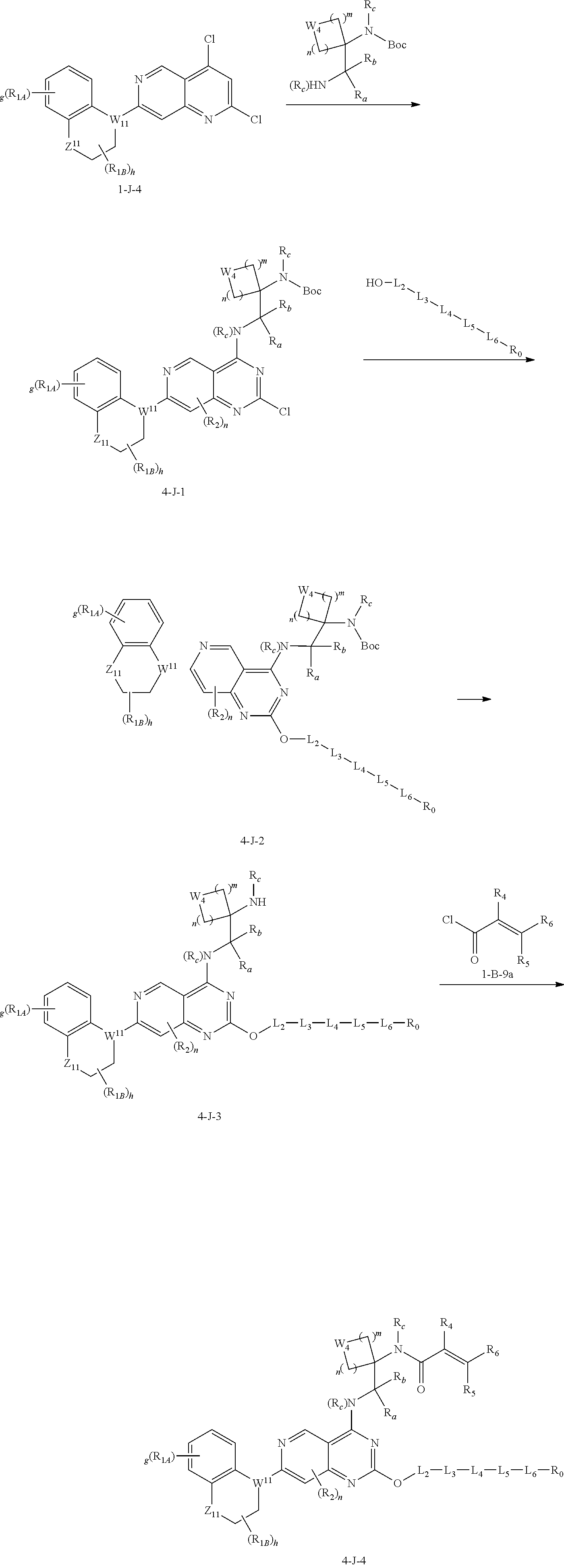

In Scheme (4-J), the starting material 1-J-4 can be prepared by conventional procedures using appropriate compounds and reagent. The starting material 1-J-4 can be converted to 4-J-2 through a sequence of two-step reaction. Finally, the intermediate 4-J-2 is deprotected to afford 4-J-3, which can react with 1-B-9a to yield the target compound 4-J-4.

Also, the target compounds can be synthesized by alternative methods but not limited to the above procedures.

An approach to synthesize compounds of

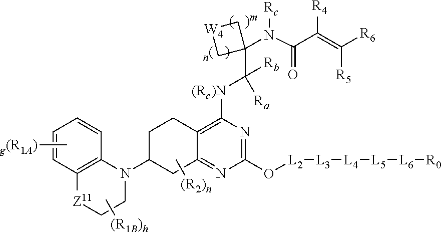

is described in Scheme (4-K).

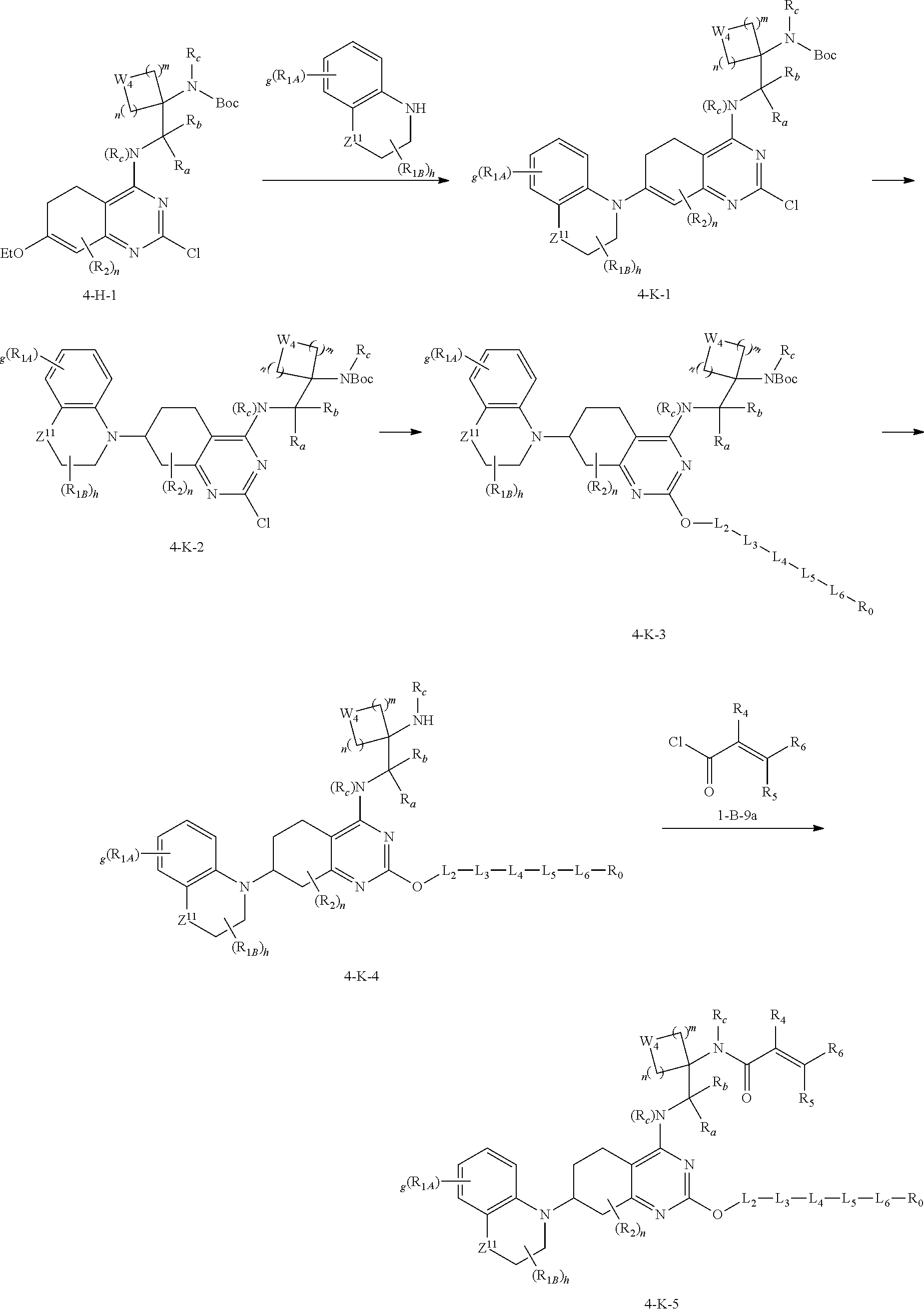

In Scheme (4-K), the starting material 4-H-1 can be prepared by conventional procedures using appropriate compounds and reagent. The starting material 4-H-1 can be converted to the intermediate 4-K-1. After that, 4-K-1 is reduced to 4-K-2, which can be converted to 4-K-3 via a general coupling reaction. Finally, the intermediate 4-K-3 is deprotected to afford 4-K-4, which can react with 1-B-9a to yield the target compound 4-K-5.

Also, the target compounds can be synthesized by alternative methods but not limited to the above procedures.

An approach to synthesize compounds of

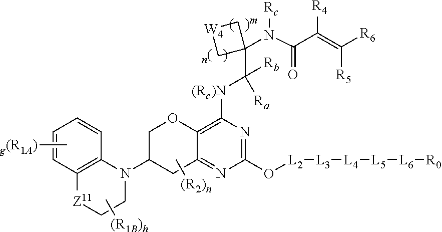

is described in Scheme (4-L).

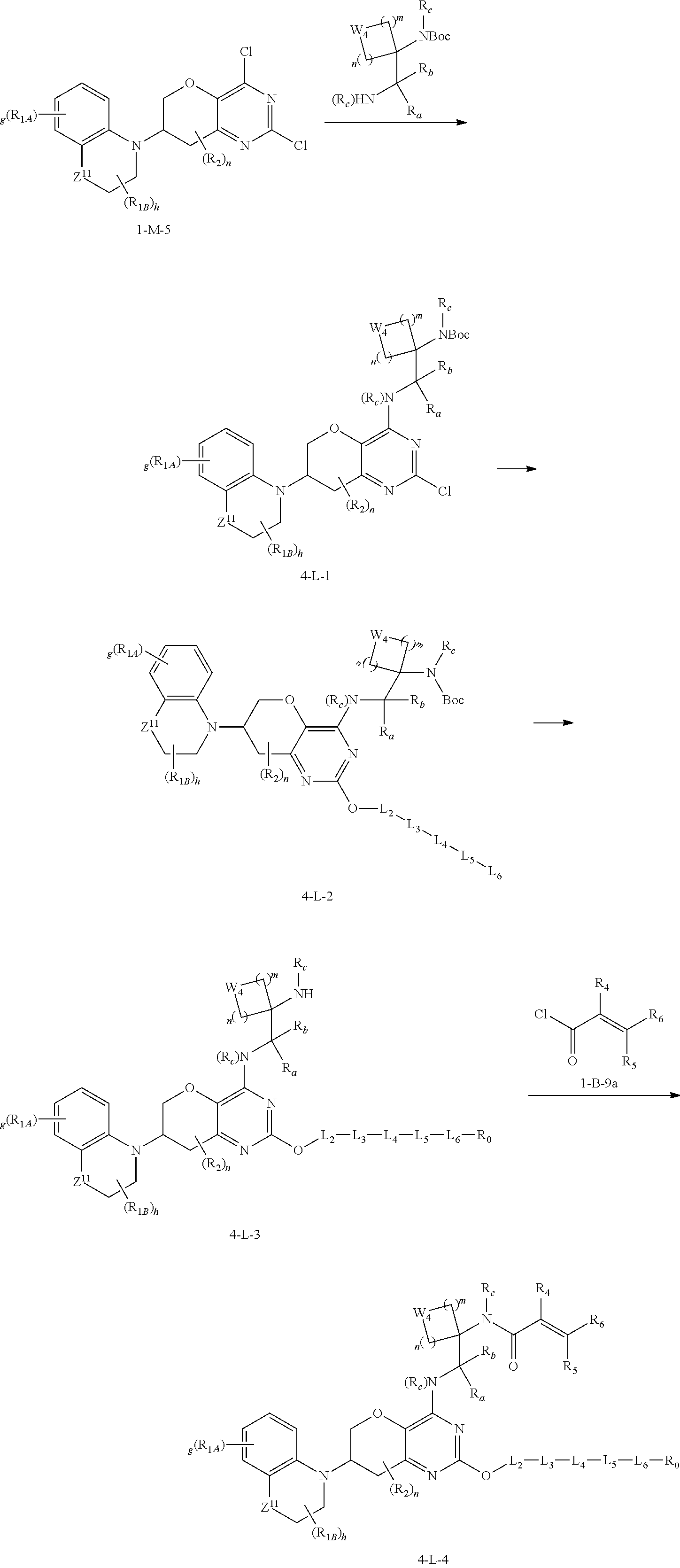

In Scheme (4-L), the starting material 1-M-5 can be prepared by conventional procedures using appropriate compounds and reagent. The starting material 1-M-5 can go through a sequence of two-step coupling reaction to provide 4-L-2. Finally, 4-L-2 is deprotected to afford 4-L-3, which can react with 1-B-9a to yield the target compound 4-L-4.

Also, the target compounds can be synthesized by alternative methods but not limited to the above procedures.

An approach to synthesize compounds of

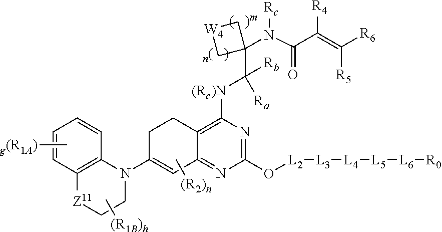

is described in Scheme (4-M).

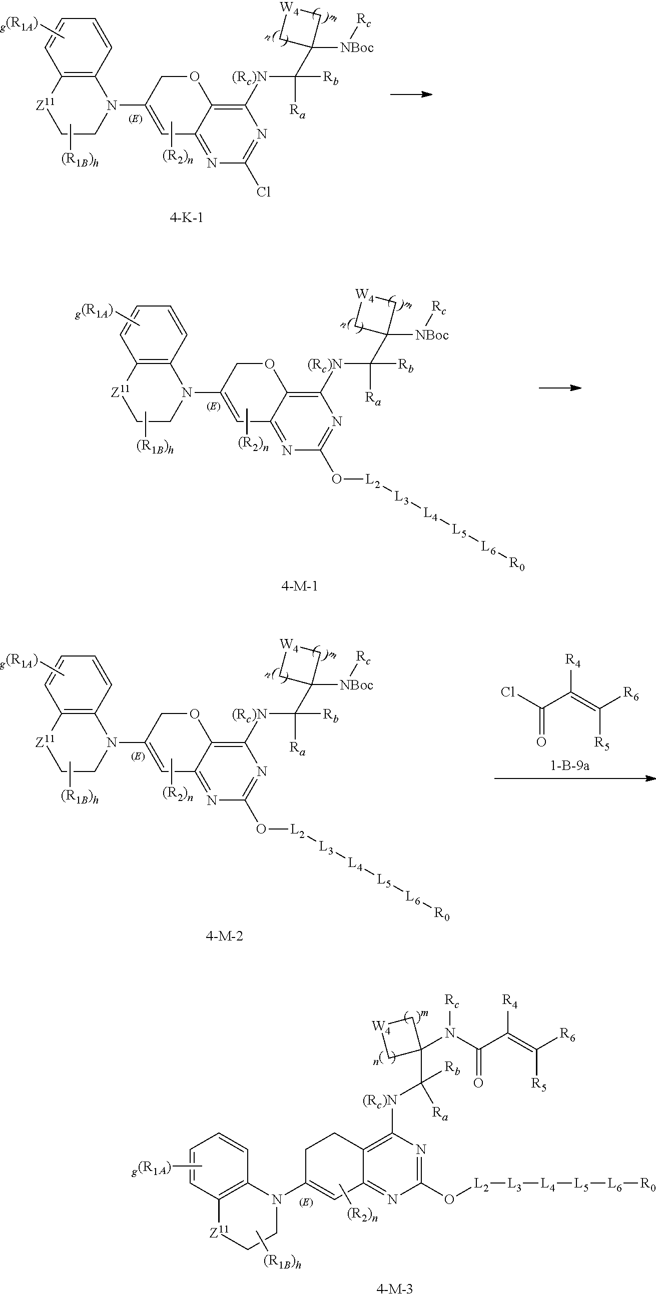

In Scheme (4-M), the starting material 4-K-1 can be prepared by conventional procedures using appropriate compounds and reagent. The starting material 4-K-2 can be converted to 4-M-1. After that, the intermediate 4-M-1 is deprotected to afford 4-M-2, which can react with 1-B-9a to yield the target compound 4-M-3.

Also, the target compounds can be synthesized by alternative methods but not limited to the above procedures.

An approach to synthesize compounds of

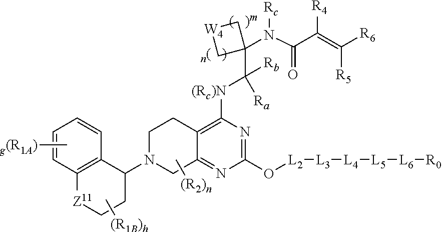

is described in Scheme (4-N).

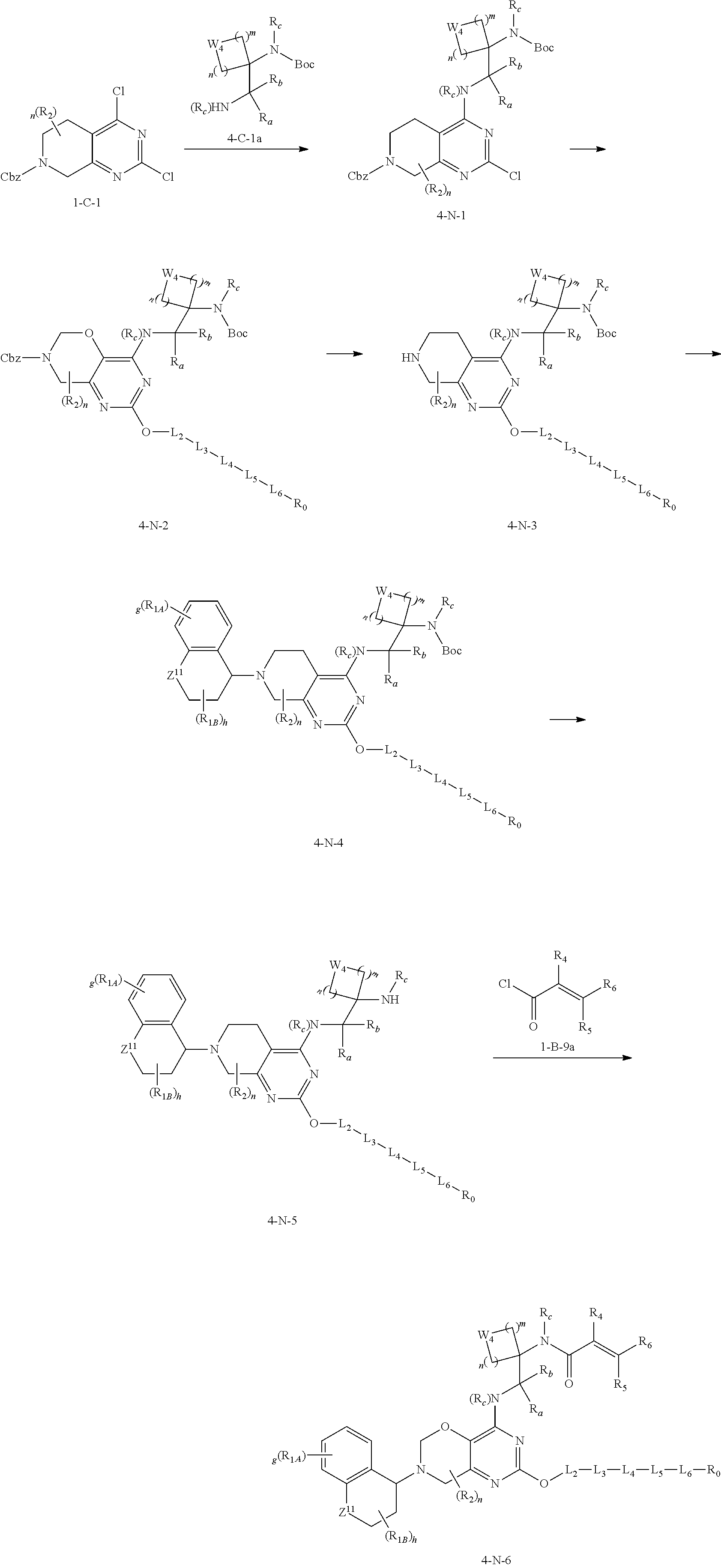

In Scheme (4-N), the starting material 1-C-1 can be prepared by conventional procedures using appropriate compounds and reagent. The starting material 1-C-1 can react with 4-C-1a to give intermediate 4-N-1, which can be converted to the intermediate 4-N-2 readily. After that, 4-N-2 is deprotected to afford 4-N-3, which can go through a reductive amination reaction to give 4-N-4. Finally, 4-N-4 is deprotected to afford 4-N-5, which can react with 1-B-9a to yield the target compound 4-N-6.

Also, the target compounds can be synthesized by alternative methods but not limited to the above procedures.

An approach to synthesize compounds of

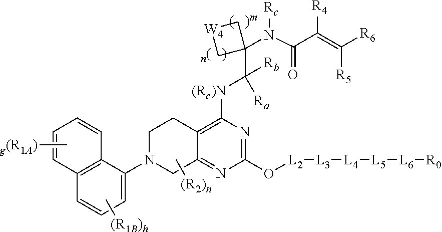

is described in Scheme (4-O).

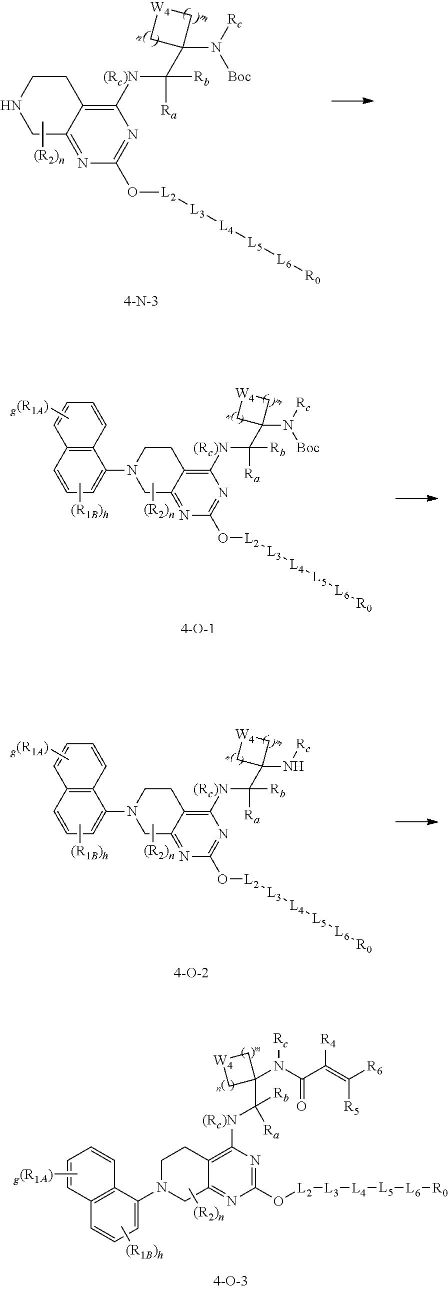

In Scheme (4-O), the starting material 4-N-3 can be prepared by conventional procedures using appropriate compounds and reagent. The starting material 4-N-3 can go through a Buchwald coupling reaction to give 4-O-1. After that, 4-O-1 is deprotected to afford 4-O-2, which can react with 1-B-9a to yield the target compound 4-O-3.

Also, the target compounds can be synthesized by alternative methods but not limited to the above procedures.

The compounds

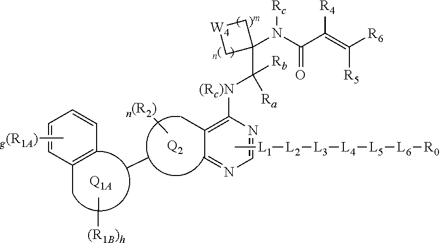

can be made by the method referred to Scheme 4-C to 4-O, by using different starting material and reagents, or by the conventional organic reactions.

The compounds

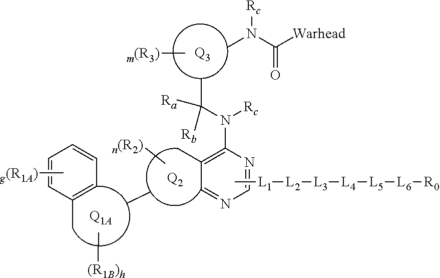

can be made by the method referred to Scheme 4-C to 4-O, by using different starting material and reagents, or by the conventional organic reactions.

The compounds

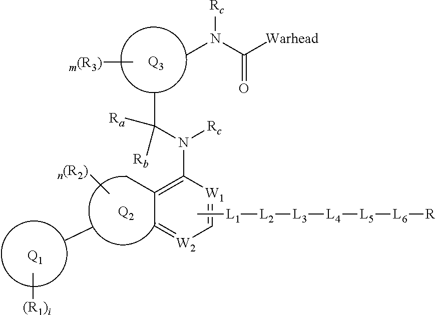

can be made by the method referred to Scheme 4-C to 4-O, by using different starting material and reagents, or by the conventional organic reactions.

An approach to synthesize compounds of

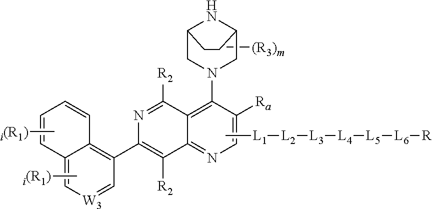

is described in Scheme A.

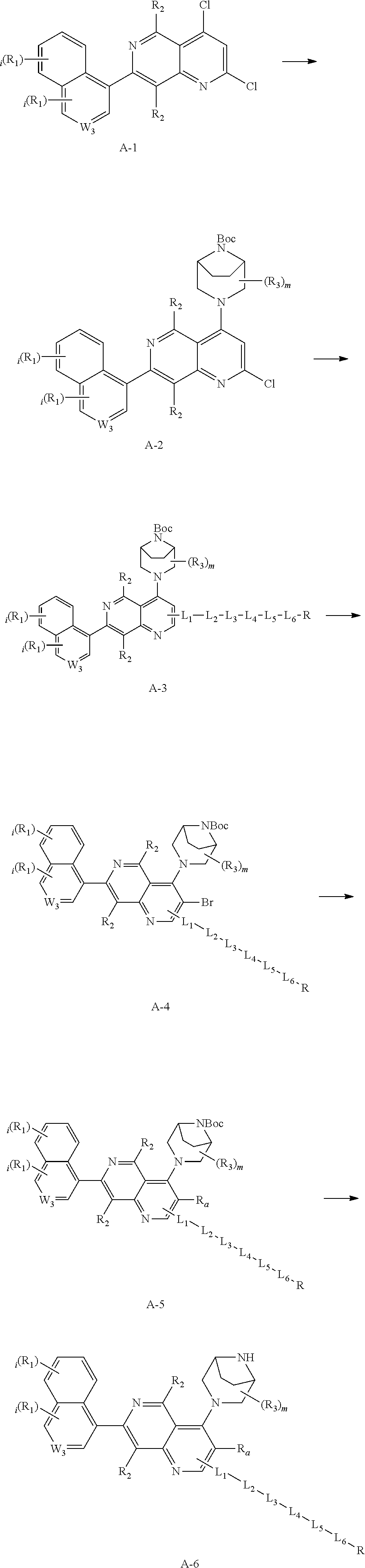

In Scheme A, the starting material A-1 can be prepared by conventional procedures using appropriate compounds and reagents. The starting material A-1 is converted to intermediate A-2 via Buchwald coupling reaction with high yield. After that, the intermediate A-2 can be converted to A-3, which can further react with a suitable bromination reagent to afford the bromide A-4. Finally, the intermediate A-4 can be transformed to intermediate A-5 readily, which is deprotected to give the target compound A-6.

Also, the target compounds can be synthesized by alternative methods but not limited to the above procedures.

An approach to synthesize compounds of

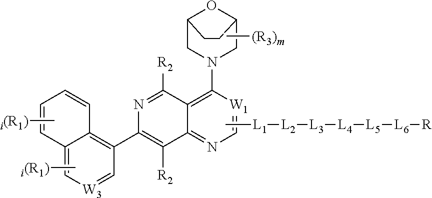

is described in Scheme B.

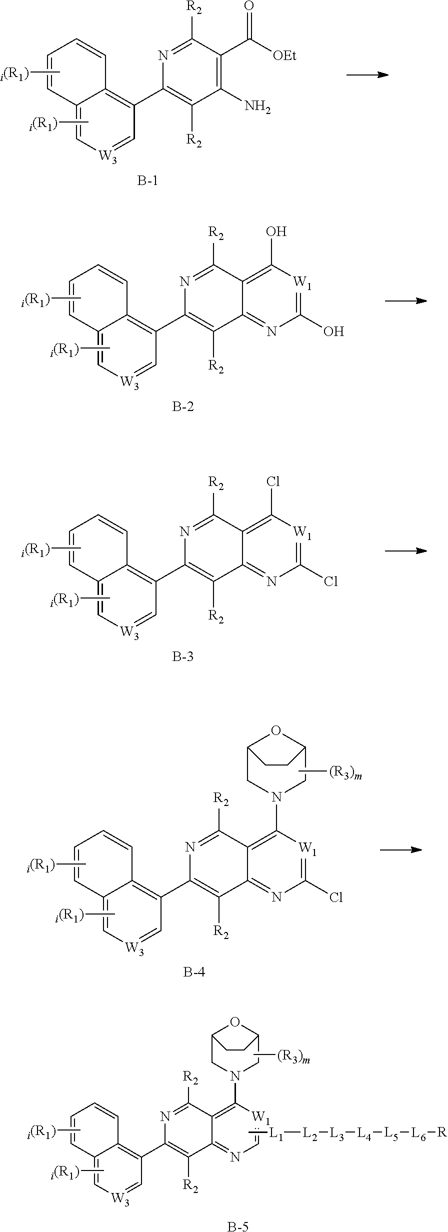

In Scheme B, the starting material B-1 can be prepared by conventional procedures using appropriate compounds and reagent. The starting material B-1 is converted to intermediate B-2 by conventional organic reaction, which can be converted to di-choloride B-3 readily. Finally, B-3 is transformed to the intermediate B-4, which undergoes a coupling reaction to yield the target compound B-5.

Also, the target compounds can be synthesized by alternative methods but not limited to the above procedures.

An approach to synthesize compounds of

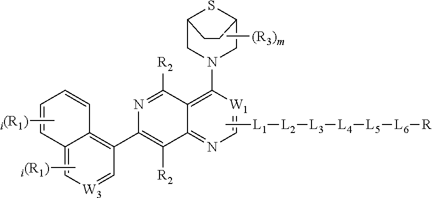

is described in Scheme C.

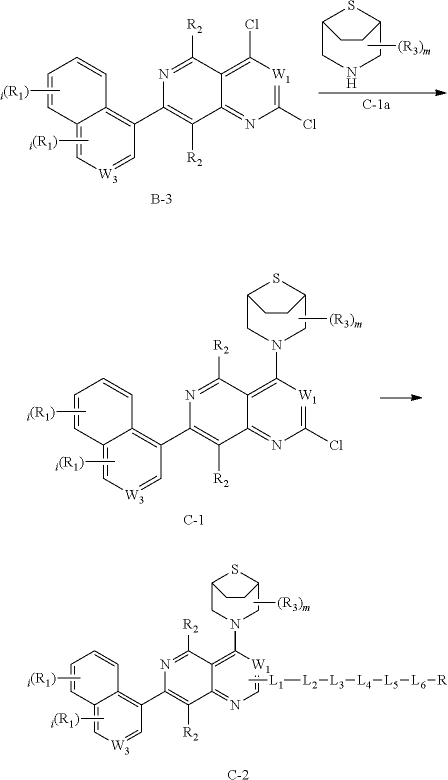

In Scheme C, the starting material B-3 can be prepared by conventional procedures using appropriate compounds and reagent. The starting material B-3 can react with C-1a to afford C-1, which undergoes a coupling reaction to yield the target compound C-2.

Also, the target compounds can be synthesized by alternative methods but not limited to the above procedures.

An approach to synthesize compounds of

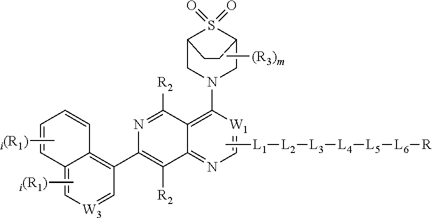

is described in Scheme D.

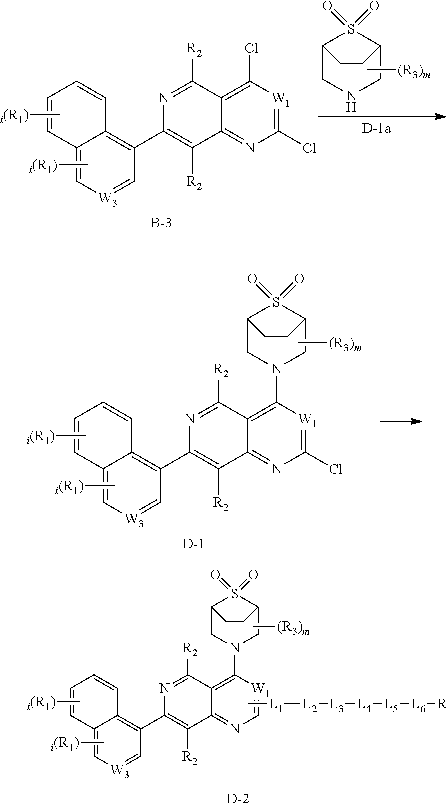

In Scheme D, the starting material B-3 can be prepared by conventional procedures using appropriate compounds and reagent. The starting material B-3 can react with D-1a to afford D-1, which undergoes a coupling reaction to yield the target compound D-2.

Also, the target compounds can be synthesized by alternative methods but not limited to the above procedures.

An approach to synthesize compounds of

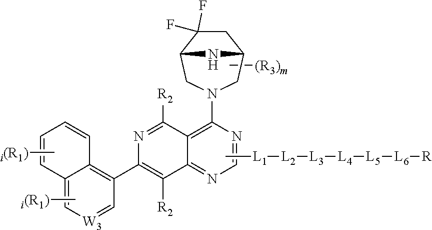

is described in Scheme E.

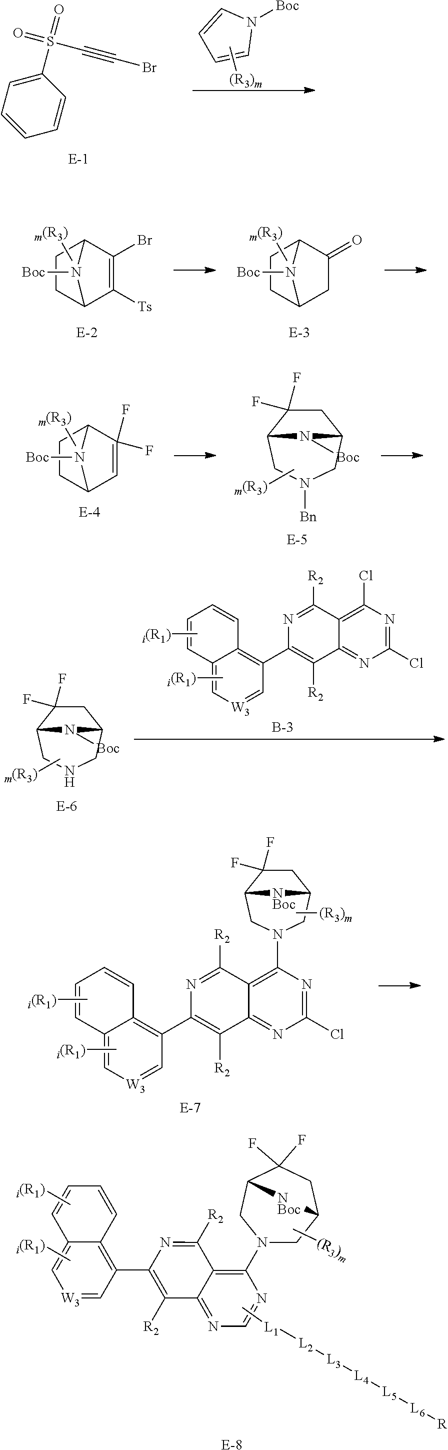

In Scheme E, the starting material E-1 can be prepared by conventional procedures using appropriate compounds and reagent. The starting material E-1 is converted to E-3 through a literate-known conditions, which can be converted to the di-fluoride E-4. Ozonolysis of the intermediate E-4, followed by reductive amination with benzylamine can give the intermediate E-5 readily. After that, the intermediate E-5 is deprotected to give E-6, which can react with intermediate B-3 to afford E-7. Finally, the intermediate E-7 undergoes a two-step sequence of coupling and deprotection reactions to yield the target compound E-8.

Also, the target compounds can be synthesized by alternative methods but not limited to the above procedures.

An approach to synthesize compounds of

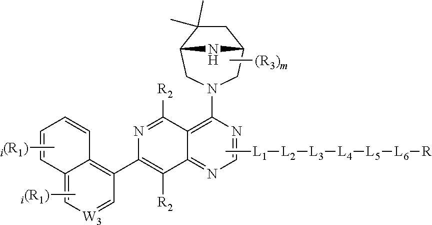

is described in Scheme F.

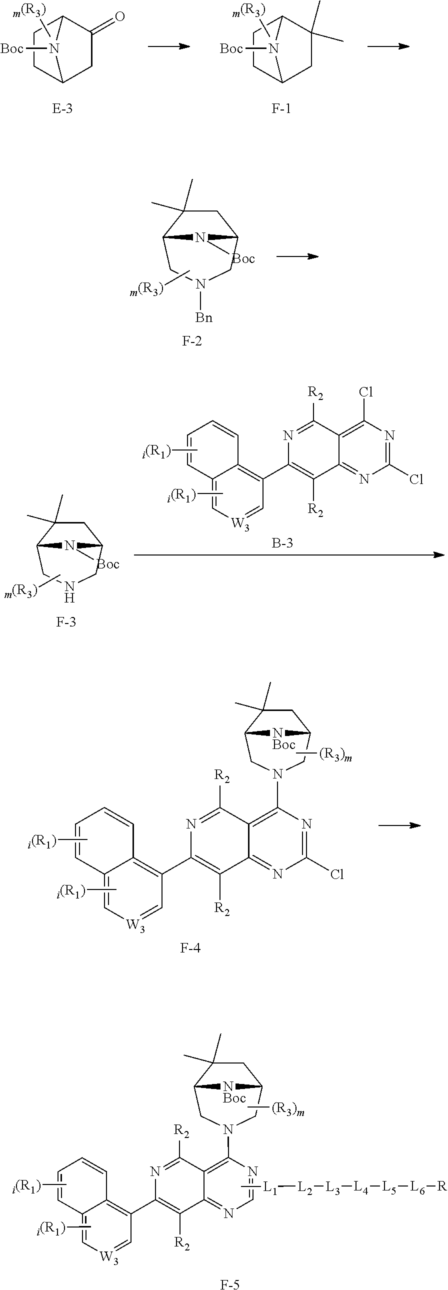

In Scheme F, the starting material E-3 can be prepared by conventional procedures using appropriate compounds and reagent. The starting material E-3 can be converted to F-1 via a literate-known conditions. After that, ozonolysis of the intermediate F-1, followed by reductive amination with benzylamine can give the intermediate F-2 readily. Next, the intermediate F-2 is deprotected to give F-3, which can react with intermediate B-3 to afford F-4. Finally, the intermediate F-4 undergoes a two-step sequence of coupling and deprotection reactions to yield the target compound F-5.

Also, the target compounds can be synthesized by alternative methods but not limited to the above procedures. An approach to synthesize compounds of

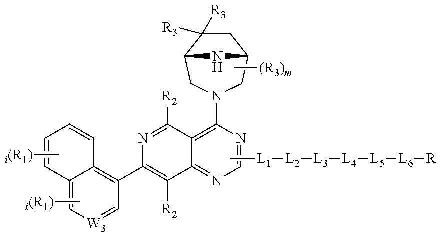

is described in Scheme G.

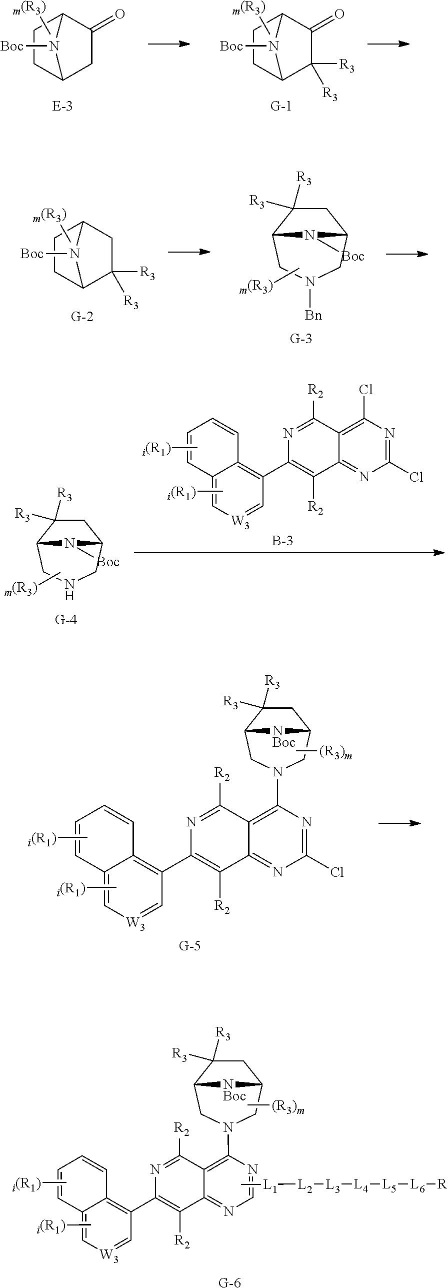

In Scheme G, the starting material E-3 can be prepared by conventional procedures using appropriate compounds and reagent. The starting material E-3 can be converted to G-1, which is reduced to afford the intermediate G-2. After that, ozonolysis of the intermediate G-2, followed by reductive amination with benzylamine can give the intermediate 10-3. Next, the intermediate G-3 is deprotected to give G-4, which can react with intermediate B-3 to afford G-5. Finally, the intermediate G-5 undergoes a two-step sequence of coupling and deprotection reaction to yield the target compound G-6.

Also, the target compounds can be synthesized by alternative methods but not limited to the above procedures.

An approach to synthesize compounds of

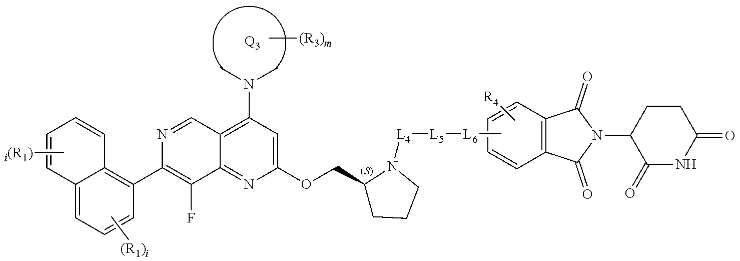

is described in Scheme H.

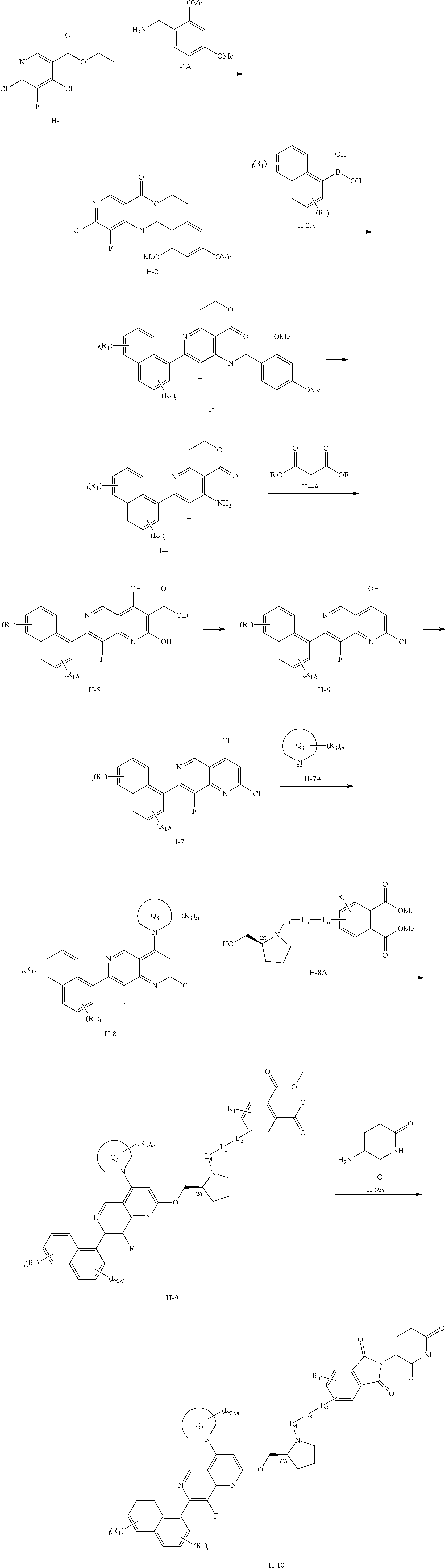

In Scheme H, the starting material H-1 can be prepared by conventional procedures using appropriate compounds and reagents. The starting material H-1 can react with H-1A to afford H-2 by a conventional SNAr reaction, which further undergoes Suzuki coupling reaction with H-2A to give H-3. The deprotection of H-3 can generate H-4, which can react with H-4A to give intermediate H-5. After that, the decarboxylation of H-5 can give H-6, which is converted to chloride H-7 readily. Next. H-7 can undergo a SNAr reaction with H-7A give the key intermediate H-8. Finally, H-8 can react with H-8A to give H-9, which is treated with H-9A to afford the target compounds H-10.

Also, the target compounds can be synthesized by alternative methods but not limited to the above procedures.

The compounds

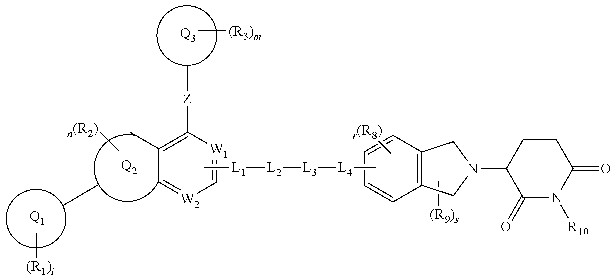

can be made by the method referred to Scheme H, by using different starting material and reagents, or by the conventional organic reactions.

The compounds

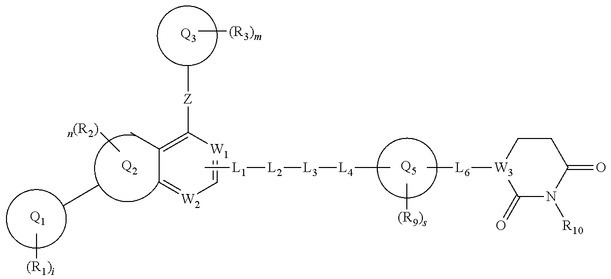

can be made by the method referred to Scheme H, by using different starting material and reagents, or by the conventional organic reactions.

An approach to synthesize compounds of

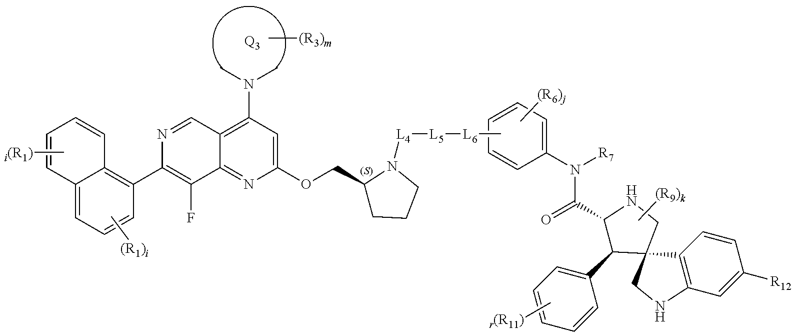

is described in Scheme I.

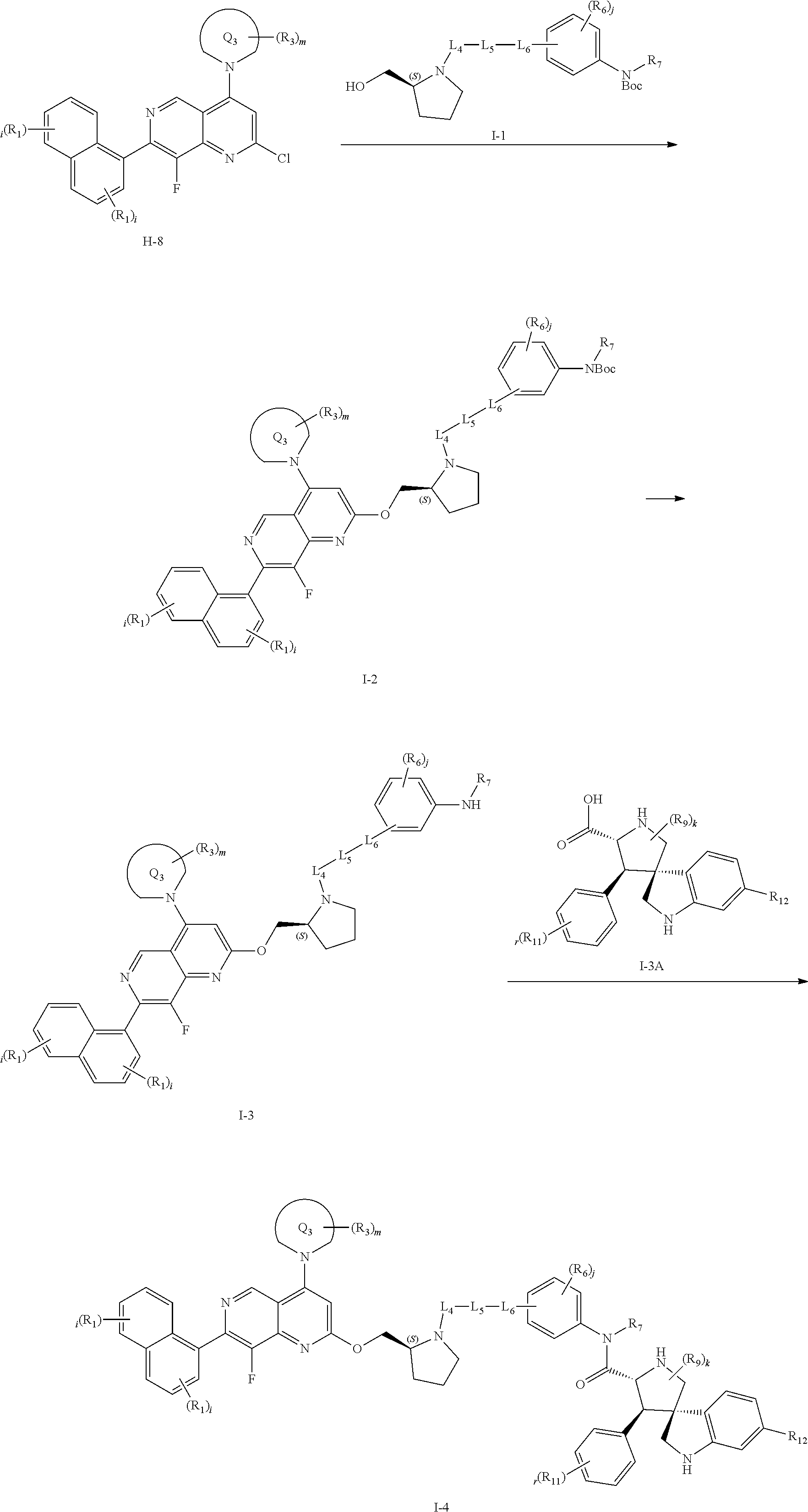

In Scheme I, the starting material H-8 can react with I-1 to afford I-2 through an SNAr reaction, which can further be deprotected to give intermediate I-3. Finally, the condensation of I-3 and I-3A under a suitable condition can yield the target compounds I-4.

Also, the target compounds can be synthesized by alternative methods but not limited to the above procedures.

An approach to synthesize intermediate of I-3

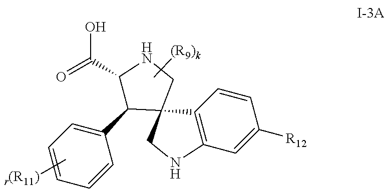

in the Scheme I is described in Scheme J. R₉, R₁₁, R₁₂, k, and r, in general Scheme J is the same as those described in the Summary section above.

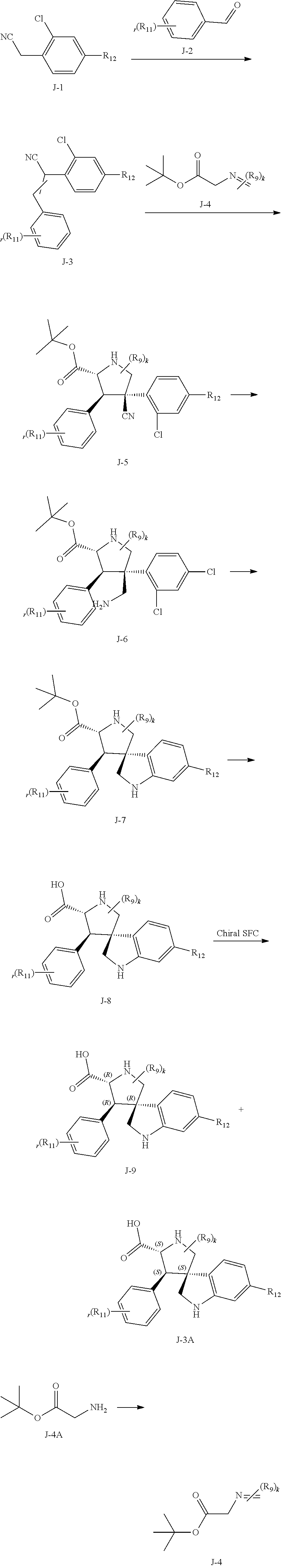

In Scheme J, the starting material J-1 can be prepared by conventional procedures using appropriate compounds and reagents. J-1 can react with J-2 to afford J-3, which can undergo an intermolecular [3+2]cyclization with J-4 derived from J-4A, to give J-5. After that, J-5 can be reduced to J-6, and then J-6 can go through an intramolecular coupling reaction to give J-7. The intermediate J-7 can be transformed to J-8. Finally, J-9 can be obtained from J-8 by chiral separation.

Also, the target compounds can be synthesized by alternative methods but not limited to the above procedures.

The compounds

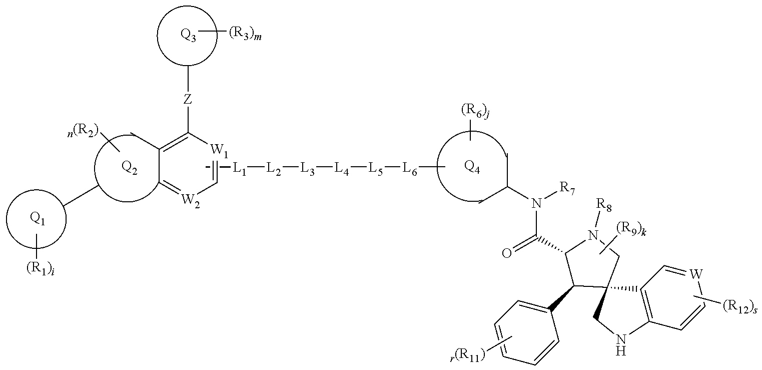

can be made by the method referred to Scheme I, by using different starting material and reagents, or by the conventional organic reactions.

The compounds

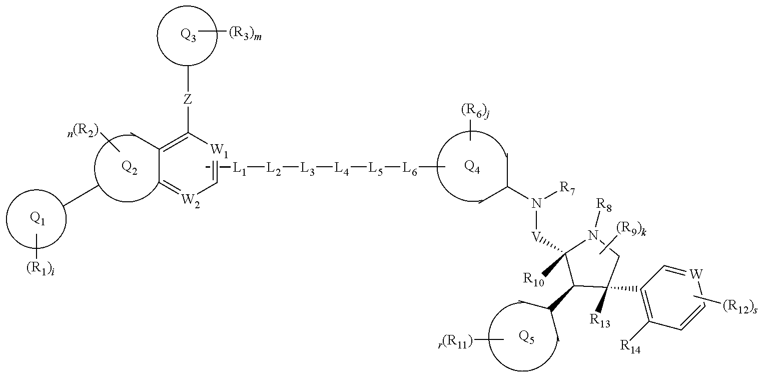

can be made by the method referred to Scheme I, by using different starting material and reagents, or by the conventional organic reactions.

The compounds and processes of the present invention will be better understood in connection with the following examples, which are intended as an illustration only and not limiting of the scope of the invention.

Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example INT_1: Preparation of 2,4,7-trichloro-8-fluoropyrido[4,3-d]pyrimidine

Synthesis of 2-chloro-3-fluoropyridine-4-carboxylic acid: Into a 2000 mL round-bottom flask, were placed methyl 2-chloro-3-fluoropyridine-4-carboxylate (100.0 g, 527.5 mmol, 1.0 eq), THE (500 mL), water (500 mL) and LiOH (50.5 g, 2110.0 mmol, 4.0 eq). The reaction mixture was stirred for 3 hours at 25° C. The resulting mixture was then quenched by the addition of water (400 mL) and adjust to pH=5.0 with HCl (1 M). The resulting mixture was extracted with EtOAc (2×500 mL). The combined organic layers were washed with brine (1000 mL), dried over anhydrous Na₂SO₄. After filtration, the filtrate was concentrated under vacuum to give 2-chloro-3-fluoropyridine-4-carboxylic acid as a white solid (85.0 g, 91.8%). LC-MS (ESI, m/z) M+1: 176.

Synthesis of tert-butyl (2-chloro-3-fluoropyridin-4-yl)carbamate: Into a 2000 mL round-bottom flask, were placed 2-chloro-3-fluoroisonicotinic acid (75.0 g, 427.2 mmol, 1.0 eq), toluene (550 mL), t-BuOH (500 mL), 4A molecular sieve (300 g) and Et₃N (129.7 g, 1281.8 mmol, 3.0 eq). The resulting mixture was stirred at 110° C. for 0.5 hour under nitrogen, then the mixture was cooled to 25° C. After that, diphenylphosphoryl azide (176.0 g, 640.9 mmol, 1.5 eq) was added at 25° C. The reaction mixture was stirred for 5 hours at 110° C. The resulting mixture was quenched by the addition of water (400 mL) and then extracted with EtOAc (3×500 mL). The combined organic layers were washed with brine (1 L), dried over anhydrous Na₂SO₄. After filtration, the filtrate was concentrated under vacuum. The crude residue was applied onto a silica gel column and eluted with ethyl acetate/petroleum ether=1:5 to give tert-butyl (2-chloro-3-fluoropyridin-4-yl)carbamate as a white solid (85.0 g, 80.7%). LC-MS (ESI, m/z) M+1: 247.

Synthesis of 2-chloro-3-fluoropyridin-4-amine: Into a 1000 mL round-bottom flask, were placed tert-butyl (2-chloro-3-fluoropyridin-4-yl)carbamate (85.0 g, 344.6 mmol, 1.0 eq), MeCN (110 mL) and HCl in dioxane (350 mL, 4 M) at 5° C. The reaction mixture was stirred at 25° C. for 4 hours. The resulting mixture was filtered, the filter cake was diluted with saturated NaHCO₃ solution and extracted with EtOAc (2×500 mL). The combined organic layers were washed with brine (500 mL), dried over anhydrous Na₂SO₄. After filtration, the filtrate was concentrated under vacuum to give 2-chloro-3-fluoropyridin-4-amine as a white solid (42.0 g, 83.2%). LC-MS (ESI, m/z) M+1: 147.

Synthesis of 2-chloro-3-fluoro-5-iodopyridin-4-amine: Into a 1000 mL round-bottom flask, were placed 2-chloro-3-fluoropyridin-4-amine (42.0 g, 286.6 mmol, 1.0 eq), NIS (77.4 g, 343.9 mmol, 1.2 eq), MeCN (300 mL) and TsOH·H₂O (2.5 g, 14.3 mmol, 0.05 eq) at 25° C. The resulting mixture was stirred for 16 hours at 70° C. The reaction mixture was quenched by the addition of water (300 mL) and then extracted with EtOAc (2×500 mL). The combined organic layers were washed with saturated aq. NaHCO₃ (500 mL), saturated aq. Na₂SO₃ (500 mL) and brine (500 mL), dried over anhydrous Na₂SO₄. After filtration, the filtrate was concentrated under vacuum to give 2-chloro-3-fluoro-5-iodopyridin-4-amine as a yellow solid (90.0 g, crude). LC-MS (ESI, m/z) M+1: 273.

Synthesis of ethyl 4-amino-6-chloro-5-fluoronicotinate: Into a 2000 mL pressure tank reactor, were placed 2-chloro-3-fluoro-5-iodopyridin-4-amine (90.0 g, 330.3 mmol, 1.0 eq), EtOH (1700 mL), Pd(PPh₃)₂Cl₂ (23.0 g, 33.0 mmol, 0.1 eq) and TEA (120.0 g, 1189.2 mmol, 3.6 eq). The reaction mixture was stirred for 16 hours at 80° C. under CO (5 atm) atmosphere. The reaction mixture was filtered, the filtrate was concentrated under vacuum to give ethyl 4-amino-6-chloro-5-fluoronicotinate as a yellow solid (68.0 g, 94.2%). LC-MS (ESI, m/z) M+1: 219.

Synthesis of ethyl 6-chloro-5-fluoro-4-(3-(2,2,2-trichloroacetyl)ureido)nicotinate: Into a 500 mL round-bottom flask, were placed ethyl ethyl 4-amino-6-chloro-5-fluoronicotinate (68.0 g, 311.1 mmol, 1.0 eq), THE (190 mL) and 2,2,2-trichloroacetyl isocyanate (87.9 g, 466.6 mmol, 1.5 eq). The reaction mixture was stirred for 20 min at 25° C. The resulting mixture was concentrated under vacuum. The residue was triturated with MTBE to give ethyl 6-chloro-5-fluoro-4-(3-(2,2,2-trichloroacetyl)ureido)nicotinate as a white solid (77.0 g, 60.8%). LC-MS (ESI, m/z) M+1: 406/408.

Synthesis of 7-chloro-8-fluoropyrido[4,3-d]pyrimidine-2,4-diol: Into a 500 mL round-bottom flask, were placed ethyl 6-chloro-5-fluoro-4-(3-(2,2,2-trichloroacetyl)ureido)nicotinate (77.0 g, 189.2 mmol, 1.0 eq) and NH₃ (g) in MeOH (77 mL, 20%). The reaction mixture was stirred for 1 hour at 25° C. The resulting mixture was concentrated under vacuum. The residue was triturated with MTBE to give 7-chloro-8-fluoropyrido[4,3-d]pyrimidine-2,4-diol as a white solid (35.0 g, 85.8%). LC-MS (ESI, m/z) M-1: 214.

Synthesis of 2,4,7-trichloro-8-fluoropyrido[4,3-d]pyrimidine: To a stirred solution of POCl₃ (406.0 g, 2647.8 mmol, 22.8 eq) was added DIEA (75.0 g, 580.3 mmol, 5.0 eq) dropwise at 5° C. After that, 7-chloro-8-fluoropyrido[4,3-d]pyrimidine-2,4-diol (25.0 g, 116.0 mmol, 1.0 eq) was added in portions at 5° C. The resulting mixture was stirred for additional 3 hours at 100° C. The resulting mixture was concentrated under vacuum and poured onto ice-water (300 mL), and then extracted with EtOAc (2×300 mL). The combined organic layers were washed with brine (300 mL), dried over anhydrous Na₂SO₄. After filtration, the filtrate was concentrated under vacuum. The crude residue was applied onto a silica gel column and eluted with ethyl acetate/petroleum ether=1:5 to give 2,4,7-trichloro-8-fluoropyrido[4,3-d]pyrimidine as a yellow solid (16.5 g, 56.3%). ¹HNMR (300 MHz, DMSO-d₆) 38.92 (s, 1H).

### Example INT_2: Preparation of triisopropyl({2-[6-(methoxymethoxy)-8-(4,4,5,5-tetramethyl-1,3,2-dioxaborolan-2-yl)naphthalen-1-yl]ethynyl})silane

Synthesis of 8-[2-(triisopropylsilyl)ethynyl]naphthalene-1,3-diol: Into a 250 mL round-bottom flask purged, were placed naphthalene-1,3-diol (5.0 g, 31.2 mmol, 1.0 eq), (2-bromoethynyl)triisopropylsilane (9.8 g, 37.4 mmol, 1.2 eq), dioxane (60 mL), AcOK (6.1 g, 62.4 mmol, 2.0 eq) and [Ru(p-Cymene)Cl₂]₂(1.9 g, 3.1 mmol, 0.1 eq). The reaction mixture was stirred for 12 hours at 110° C. under a nitrogen atmosphere. The resulting mixture was concentrated under vacuum. The crude residue was applied onto a silica gel column and eluted with ethyl acetate/petroleum ether=1:4 to give 8-[2-(triisopropylsilyl)ethynyl]naphthalene-1,3-diol as a light yellow solid (6.0 g, 56.4%). LC-MS (ESI, m/z) M-1: 339.

Synthesis of 3-(methoxymethoxy)-8-((triisopropylsilyl)ethynyl)naphthalen-1-ol: Into a 200-mL round bottom flask, were placed 8-[2-(triisopropylsilyl)ethynyl]naphthalene-1,3-diol (6.0 g, 17.6 mmol, 1.0 eq), DIEA (6.8 g, 52.9 mmol, 3.0 eq) and CH₂Cl₂ (100 mL). After that, MOMBr (3.3 g, 26.4 mmol, 1.5 eq) was added dropwise at −40° C. under a nitrogen atmosphere. The reaction mixture was stirred at −40° C. for 30 mins. The reaction mixture was quenched by the addition of water (20 mL) and then extracted with CH₂Cl₂ (2×200 mL). The combined organic layers were washed with brine (500 mL), dried over anhydrous Na₂SO₄, and concentrated under vacuum. The crude residue was applied onto a silica gel column and eluted with ethyl acetate/petroleum ether=1:3 to give 3-(methoxymethoxy)-8-[2-(triisopropylsilyl)ethynyl]naphthalen-1-ol as a light yellow solid (5.0 g, 73.8%). LC-MS (ESI, m/z) M+1: 385.

Synthesis of 3-(methoxymethoxy)-8-[2-(triisopropylsilyl)ethynyl]naphthalen-1-yl trifluoromethanesulfonate: Into a 250 mL round-bottom flask were placed 3-(methoxymethoxy)-8-[2-(triisopropylsilyl)ethynyl]naphthalen-1-ol (5.0 g, 13.0 mmol, 1.0 eq), DIEA (5.0 g, 39.0 mmol, 3.0 eq) and CH₂Cl₂ (100 mL). After that, Tf₂O (5.5 g, 19.5 mmol, 1.5 eq) was added dropwise at −40° C. under nitrogen atmosphere. The resulting mixture was stirred for 1 hour at −40° C. under nitrogen atmosphere. The reaction was quenched by the addition of water (10 mL) at −40° C. and then extracted with CH₂Cl₂ (2×100 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:5 to give 3-(methoxymethoxy)-8-[2-(triisopropylsilyl)ethynyl]naphthalen-1-yl trifluoromethanesulfonate as a light yellow solid (4.5 g, 67.0%). LC-MS (ESI, m/z) M-CF₃SO₂+1: 384. ¹HNMR (400 MHz, Chloroform-d) δ 7.75-7.73 (m, 2H), 7.50-7.41 (m, 2H), 7.33 (d, J=2.4 Hz, 1H), 5.31 (s, 2H), 3.54 (s, 3H), 1.19 (d, J=5.8 Hz, 18H), 0.94-0.82 (m, 3H).

Synthesis of triisopropyl({2-[6-(methoxymethoxy)-8-(4,4,5,5-tetramethyl-1,3,2-dioxaborolan-2-yl)naphthalen-1-yl]ethynyl})silane: Into a 250 mL round-bottom flask, were placed 3-(methoxymethoxy)-8-[2-(triisopropylsilyl)ethynyl]naphthalen-1-yl trifluoromethanesulfonate (4.5 g, 8.7 mmol, 1.0 eq), bis(pinacolato)diboron (4.4 g, 17.4 mmol, 2.0 eq), toluene (100 mL), Pd(dppf)Cl₂·CH₂Cl₂ (0.7 g, 0.9 mmol, 0.1 eq) and KOAc (3.0 g, 30.5 mmol, 3.5 eq). The resulting mixture was stirred for 16 hours at 110° C. under nitrogen atmosphere. The reaction mixture was quenched by the addition of water (100 mL) and then extracted with EtOAc (2×100 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:20 to give triisopropyl({2-[6-(methoxymethoxy)-8-(4,4,5,5-tetramethyl-1,3,2-dioxaborolan-2-yl)naphthalen-1-yl]ethynyl})silane as a yellow solid (3.0 g, 69.7%). LC-MS (ESI, m/z) M+1: 495. ¹HNMR (400 MHz, Chloroform-d) δ 7.74-7.66 (m, 2H), 7.48 (d, J=2.6 Hz, 1H), 7.42-7.32 (m, 2H), 5.30 (s, 2H), 3.52 (s, 3H), 1.45 (s, 12H), 1.18-1.17 (m, 18H), 1.17-1.16 (m, 3H).

### Example INT_3: Preparation of ((2R,7aS)-2-fluorotetrahydro-1H-pyrrolizin-7a(5H)-yl)methanol

Synthesis of ethyl 2-methylene-5-oxotetrahydro-1H-pyrrolizine-7a(5H)-carboxylate: To a stirred mixture of ethyl 5-oxopyrrolidine-2-carboxylate (100.0 g, 636.2 mmol, 1.0 eq) and 3-chloro-2-(chloromethyl)prop-1-ene (318.1 g, 2544.8 mmol, 4.0 eq) in THE (600 mL) was added LiHMDS (1336 mL, 1336 mmol, 2.1 eq) dropwise at −40° C. under nitrogen atmosphere. The resulting mixture was stirred overnight at 25° C. under nitrogen atmosphere. The reaction was quenched by the addition of sat. NH₄Cl (aq.) (100 mL) at 0-5° C. The mixture was neutralized to pH=7 with HCl (1 M), and then extracted with EtOAc (2×800 mL). The combined organic layers were washed with water (1 L) and brine (1 L), dried over anhydrous Na₂SO₄. After filtration, the filtrate was concentrated under vacuum. The crude residue was applied onto a silica gel column and eluted with ethyl acetate/petroleum ether=2:1 to give ethyl 2-methylene-5-oxotetrahydro-1H-pyrrolizine-7a(5H)-carboxylate as a yellow oil (70.0 g, 52.6%). ¹HNMR (400 MHz, DMSO-d₆) δ 5.04 (ddq, J=18.2, 3.3, 2.0 Hz, 2H), 4.15 (d, J=7.1 Hz, 1H), 4.15-4.04 (m, 2H), 3.63-3.51 (m, 1H), 2.91 (dd, J=15.4, 1.6 Hz, 1H), 2.67-2.52 (m, 2H), 2.41 (ddd, J=12.7, 9.1, 1.8 Hz, 1H), 2.35-2.12 (m, 2H), 1.19 (t, J=7.1 Hz, 3H).

Synthesis of ethyl 2,5-dioxotetrahydro-1H-pyrrolizine-7a(5H)-carboxylate: Into a 2000 mL round-bottom flask purged, were placed ethyl 2-methylidene-5-oxo-tetrahydropyrrolizine-7a-carboxylate (70.0 g, 334.8 mmol, 1.0 eq), MeCN (700 mL), CH₂Cl₂ (700 mL), water (1050 mL) and trichlororuthenium hydrate (3.8 g, 16.7 mmol, 0.05 eq). After that, sodium periodate (286.4 g, 1339 mmol, 4.0 eq) was added in portions at 5° C. The reaction mixture was stirred for additional 6 hours at 25° C. The resulting mixture was basified to pH=9 with saturated NaHCO₃ (aq.) and then extracted with EtOAc (2×1500 mL). The combined organic layers were washed with brine (1500 mL), dried over anhydrous Na₂SO₄. After filtration, the filtrate was concentrated under vacuum. The crude residue was applied onto a silica gel column and eluted with ethyl acetate/petroleum ether=2:1 to give ethyl 2,5-dioxotetrahydro-1H-pyrrolizine-7a(5H)-carboxylate as a colorless oil (40.0 g, 56.6%). LC-MS (ESI, m/z) M+1: 212. ¹HNMR (400 MHz, Chloroform-d) δ 4.25 (q, J=7.1 Hz, 2H), 4.13 (dd, J=18.5, 1.0 Hz, 1H), 3.57 (dd, J=18.5, 1.4 Hz, 1H), 3.05-2.92 (m, 2H), 2.92-2.75 (m, 1H), 2.47 (ddd, J=16.8, 9.5, 1.4 Hz, 2H), 2.19 (ddd, J=13.0, 11.2, 9.5 Hz, 1H), 1.30 (t, J=7.1 Hz, 3H).

Synthesis of ethyl (2S,7aS)-2-hydroxy-5-oxotetrahydro-1H-pyrrolizine-7a(5H)-carboxylate: To a stirred solution of ethyl 2,5-dioxotetrahydro-1H-pyrrolizine-7a(5H)-carboxylate (20 g, 94.7 mmol, 1.0 eq) in EtOH (100 mL) was added NaBH₄ (1.1 g, 28.4 mmol, 0.3 eq) in portions at 5° C. The resulting mixture was stirred at 5° C. for 20 min. The reaction mixture was then quenched by the addition of sat. NH₄Cl (aq.) (20 mL) at 5° C. and stirred at this temperature for 30 min. The resulting mixture was concentrated under vacuum. The crude residue was applied onto a silica gel column and eluted with ethyl acetate/petroleum ether=2:1 to give ethyl (2S,7aS)-2-hydroxy-5-oxotetrahydro-1H-pyrrolizine-7a(5H)-carboxylate as a yellow oil (10.0 g, 49.5%). LC-MS (ESI, m/z) M+1: 214. ¹HNMR (400 MHz, Chloroform-d) δ 4.63 (d, J=2.3 Hz, 1H), 4.22 (q, J=7.1 Hz, 2H), 3.87 (dt, J=12.6, 1.7 Hz, 1H), 3.22 (ddd, J=12.5, 4.3, 1.2 Hz, 1H), 2.87-2.74 (m, 1H), 2.74-2.64 (m, 1H), 2.56 (dd, J=14.0, 5.5 Hz, 1H), 2.44 (dd, J=16.1, 9.1 Hz, 1H), 2.24 (td, J=12.1, 9.0 Hz, 1H), 1.98 (ddd, J=14.0, 2.8, 1.5 Hz, 1H), 1.30 (t, J=7.1 Hz, 3H).

Synthesis of ethyl (2R,7aS)-2-fluoro-5-oxotetrahydro-1H-pyrrolizine-7a(5H)-carboxylate: To a stirred solution of ethyl (2S,7aS)-2-hydroxy-5-oxotetrahydro-1H-pyrrolizine-7a(5H)-carboxylate (5.0 g, 23.4 mmol, 1.0 eq) in CH₂Cl₂ (50 mL) was added DAST (4.9 g, 30.4 mmol, 1.3 eq) dropwise at −78° C. under nitrogen atmosphere. The reaction mixture was stirred at 25° C. for 16 hours. The reaction mixture was quenched by the addition of MeOH (2.0 mL) and water (60 mL) at 5° C., and then extracted with CH₂Cl₂ (3×80 mL). The combined organic layers were washed with brine (150 mL), dried over anhydrous Na₂SO₄. After filtration, the filtrate was concentrated under vacuum. The crude residue was applied onto a silica gel column and eluted with ethyl acetate/petroleum ether=2:1 to give ethyl (2R,7aS)-2-fluoro-5-oxotetrahydro-1H-pyrrolizine-7a(5H)-carboxylate as a light yellow oil (3.5 g, 69.4%). LC-MS (ESI, m/z) M+1: 216.

Synthesis of ethyl (2R,7aS)-2-fluoro-5-oxotetrahydro-1H-pyrrolizine-7a(5H)-carboxylate: 1.5 g of ethyl (2R,7aS)-2-fluoro-5-oxotetrahydro-1H-pyrrolizine-7a(5H)-carboxylate was separated by SFC using the following conditions: Column, 5: IC-3 50*3.0 mm, 3.0 um; mobile phase B: EtOH; Flow rate: 3.0 ml/min; Gradient: 10% B to 55% B in 3 min, RT: 0.766 min; Detector, 220 nm. Finally, ethyl (2R,7aS)-2-fluoro-5-oxotetrahydro-1H-pyrrolizine-7a(5H)-carboxylate was obtained as a colorless oil (450 mg, 30%). T_{R}=0.766 min in CHIRAL-SFC, Column: CHIRALPACK IC-3 50*3.0 mm, 3.0 um. mobile phase A: CO₂; mobile phase B: EtOH. Method Set: 10% to 50%_B1_2_2200, Oven Temperature: 35° C. ¹HNMR (300 MHz, Chloroform-d) δ 5.4-5.19 (m, 1H), 4.26-4.18 (m, 3H), 4.20-4.15 (m, 1H), 3.18 (ddd, J=33.6, 13.5, 3.4 Hz, 1H), 2.75-2.62 (m, 2H), 2.44 (ddd, J=17.6, 8.9, 4.7 Hz, 1H), 2.33-2.07 (m, 2H), 1.30 (t, J=7.1 Hz, 3H).

Synthesis of ((2R,7aS)-2-fluorotetrahydro-1H-pyrrolizin-7a(5H)-yl)methanol: To a stirred solution of ethyl (2R,7aS)-2-fluoro-5-oxotetrahydro-1H-pyrrolizine-7a(5H)-carboxylate (300 mg, 1.4 mmol, 1.0 eq) in THF (5.0 mL) was added LiAlH₄ (80 mg, 2.1 mmol, 1.5 eq) in portions at 5° C. and stirred at 70° C. for 3 hours. The reaction was quenched with Na₂SO₄·10 H₂O (2.0 g). The resulting mixture was filtered, the filter cake was washed with EtOAc (2×10 mL). The filtrate was concentrated under reduced pressure to afford ((2R,7aS)-2-fluorotetrahydro-1H-pyrrolizin-7a(5H)-yl)methanol as colorless oil (200 mg, 90.1%). LC-MS (ESI, m/z) M+1: 160.

### Example INT_4: Preparation of {2-[2-fluoro-6-(methoxymethoxy)-8-(4,4,5,5-tetramethyl-1,3,2-dioxaborolan-2-yl)naphthalen-1-yl]ethynyl}triisopropylsilane

Synthesis of 7-fluoro-8-[2-(triisopropylsilyl)ethynyl]naphthalene-1,3-diol: To a solution of 7-fluoronaphthalene-1,3-diol (5.0 g, 28.1 mmol, 1.0 eq) and (2-bromoethynyl)triisopropylsilane (8.8 g, 33.7 mmol, 1.2 eq) in dioxane (60 mL) were added AcOK (3.4 g, 56.1 mmol, 2.0 eq) and [Ru(p-Cymene)Cl₂]₂(1.7 g, 2.8 mmol, 0.1 eq). The resulting mixture was stirred for 12 hours at 110° C. under a nitrogen atmosphere. The resulting mixture was concentrated under vacuum. The crude residue was applied onto a silica gel column and eluted with ethyl acetate/petroleum ether=4:1 to give 7-fluoro-8-[2-(triisopropylsilyl)ethynyl]naphthalene-1,3-diol as a light yellow solid (7.0 g, 69.6%). LC-MS (ESI, m/z) M-1: 357. ¹HNMR (400 MHz, DMSO-d₆) δ 10.04 (s, 1H), 9.58 (s, 1H), 7.63 (dd, J=9.1, 5.7 Hz, 1H), 7.25 (t, J=9.0 Hz, 1H), 6.62 (dd, J=18.3, 2.4 Hz, 2H), 1.17-1.13 (m, 21H).

Synthesis of 7-fluoro-3-(methoxymethoxy)-8-[2-(triisopropylsilyl)ethynyl]naphthalen-1-ol: To a solution of 7-fluoro-8-[2-(triisopropylsilyl)ethynyl]naphthalene-1,3-diol (7.0 g, 19.5 mmol, 1.0 eq) and DIEA (7.6 g, 58.6 mmol, 3.0 eq) in CH₂Cl₂ (100 mL) was added dropwise bromomethoxymethane (3.7 g, 29.3 mmol, 1.5 eq) at −40° C. under N₂ atmosphere. The reaction mixture was stirred at −40° C. for 30 min. The reaction mixture was quenched by the addition of water (20 mL) and then extracted with CH₂Cl₂ (2×200 mL). The combined organic layers were washed with brine (500 mL), dried over anhydrous Na₂SO₄, and concentrated under vacuum. The crude residue was applied onto a silica gel column and eluted with ethyl acetate/petroleum ether=1:3 to give 7-fluoro-3-(methoxymethoxy)-8-[2-(triisopropylsilyl)ethynyl]naphthalen-1-ol as a light yellow solid (4.0 g, 50.9%). LC-MS (ESI, m/z) M+1: 403.

Synthesis of 7-fluoro-3-(methoxymethoxy)-8-[2-(triisopropylsilyl)ethynyl]naphthalen-1-yl trifluoromethanesulfonate: To a stirred mixture of 7-fluoro-3-(methoxymethoxy)-8-[2-(triisopropylsilyl)ethynyl]naphthalen-1-ol (4.0 g, 9.9 mmol, 1.0 eq) and DIEA (3.9 g, 29.8 mmol, 3.0 eq) in CH₂Cl₂ (60 mL) were added Tf₂O (4.2 g, 14.9 mmol, 1.5 eq) dropwise at −40° C. under nitrogen atmosphere. The resulting mixture was stirred for 1 hour at −40° C. under nitrogen atmosphere. The reaction was quenched by the addition of water (10 mL) at −40° C. and then extracted with CH₂Cl₂ (2×100 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:5 to give 7-fluoro-3-(methoxymethoxy)-8-[2-(triisopropylsilyl)ethynyl]naphthalen-1-yl trifluoromethanesulfonate as a light yellow solid (5.0 g, 94.1%). ¹HNMR (400 MHz, Chloroform-d) δ 7.72 (dd, J=9.1, 5.4 Hz, 1H), 7.44 (d, J=2.4 Hz, 1H), 7.40-7.29 (m, 2H), 5.30 (s, 2H), 3.54 (s, 3H), 1.31-1.23 (m, 3H), 1.20-1.18 (m, 18H).

Synthesis of {2-[2-fluoro-6-(methoxymethoxy)-8-(4,4,5,5-tetramethyl-1,3,2-dioxaborolan-2-yl)naphthalen-1-yl]ethynyl}triisopropylsilane: Into a 250 mL round-bottom flask were added 7-fluoro-3-(methoxymethoxy)-8-[2-(triisopropylsilyl)ethynyl]naphthalen-1-yl trifluoromethanesulfonate (5.0 g, 9.4 mmol, 1.0 eq), bis(pinacolato)diboron (3.6 g, 14.0 mmol, 1.5 eq), toluene (100 mL), Pd(dppf)Cl₂ (760 mg, 0.9 mmol, 0.1 eq) and KOAc (3.2 g, 32.7 mmol, 3.5 eq) at 25° C. The resulting mixture was stirred for 16 hours at 100° C. under nitrogen atmosphere. The reaction mixture was quenched by the addition of water (100 mL) and then extracted with EtOAc (2×100 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:20 to give {2-[2-fluoro-6-(methoxymethoxy)-8-(4,4,5,5-tetramethyl-1,3,2-dioxaborolan-2-yl)naphthalen-1-yl]ethynyl}triisopropylsilane as a yellow solid (2.0 g, 41.7%). LC-MS (ESI, m/z) M+1: 513. ¹HNMR (400 MHz, Chloroform-d) δ 7.68 (dd, J=9.0, 5.6 Hz, 1H), 7.52 (d, J=2.6 Hz, 1H), 7.39 (d, J=2.6 Hz, 1H), 7.24 (t, J=8.8 Hz, 1H), 5.29 (s, 2H), 3.52 (s, 3H), 1.45 (s, 12H), 1.29 (s, 3H), 1.19-1.17 (m, 18H).

### Example INT_5: Preparation of ((2-fluoro-8-(4,4,5,5-tetramethyl-1,3,2-dioxaborolan-2-yl)naphthalen-1-yl)ethynyl)triisopropylsilane

Synthesis of 7-fluoronaphthalen-1-ol: Into a 100 mL 3-necked round-bottom flask, were added 7-fluoro-3,4-dihydronaphthalen-1(2H)-one (2.0 g, 12.2 mmol, 1.0 eq), AcOH (40 mL), HBr in AcOH (0.2 mL). After that, Br₂ (2.1 g, 13.4 mmol, 1.1 eq) was added dropwise at 0° C. The resulting mixture was stirred for additional 3 hours at 25° C. The reaction was quenched with water (40 mL) and extracted with CH₂Cl₂ (3×40 mL). The combined organic layers were dried over anhydrous Na₂SO₄. After filtration, the filtrate was concentrated under vacuum to afford a brown oil, which was dissolved in DMF (15 mL). To the above mixture was added LiBr (1.8 g, 20.7 mmol, 1.7 eq) and Li₂CO₃ (1.5 g, 20.7 mmol, 1.7 eq). The resulting mixture was stirred for additional 3.5 hours at 160° C. The reaction was quenched with water (15 mL) and extracted with EtOAc (3×15 mL). The combined organic layers were dried over anhydrous Na₂SO₄. After filtration, the filtrate was concentrated under vacuum. The crude residue was applied onto a silica gel column and eluted with ethyl acetate/petroleum ether=1:5 to give 7-fluoronaphthalen-1-ol as a light yellow solid (1.3 g, 65.8%). ¹HNMR (300 MHz, Chloroform-d) δ 7.92 (dd, J=10.4, 2.7 Hz, 1H), 7.34 (dd, J=9.3, 5.7 Hz, 1H), 7.31-7.21 (m, 2H), 7.09 (ddd, J=9.3, 8.1, 2.7 Hz, 1H), 6.95 (d, J=7.6 Hz, 1H).

Synthesis of 7-fluoro-8-((triisopropylsilyl)ethynyl)naphthalen-1-ol: Into a 250 mL round-bottom flask, were placed 7-fluoronaphthalen-1-ol (5.0 g, 28.1 mmol, 1.0 eq), (2-bromoethynyl)triisopropylsilane (8.8 g, 33.7 mmol, 1.2 eq), dioxane (60 mL), AcOK (5.5 g, 56.1 mmol, 2.0 eq), [Ru(p-Cymene)Cl₂]₂(1.7 g, 2.8 mmol, 0.1 eq). The resulting mixture was stirred for 12 hours at 110° C. under a nitrogen atmosphere. The resulting mixture was concentrated under vacuum. The crude residue was applied onto a silica gel column and eluted with ethyl acetate/petroleum ether=4:1 to give 7-fluoro-8-((triisopropylsilyl)ethynyl)naphthalen-1-ol as a light yellow solid (7.0 g, 69.6%).

Synthesis of 7-fluoro-8-((triisopropylsilyl)ethynyl)naphthalen-1-yl trifluoromethanesulfonate: Into a 250 mL round bottom flask, were placed 7-fluoro-8-((triisopropylsilyl)ethynyl)naphthalen-1-ol (4.0 g, 9.9 mmol, 1.0 eq), DIEA (3.9 g, 29.8 mmol, 3.0 eq), CH₂Cl₂ (60 mL). After that, Tf₂O (4.2 g, 14.9 mmol, 1.5 eq) was added dropwise at −40° C. under nitrogen atmosphere. The resulting mixture was stirred for 1 hour at −40° C. under nitrogen atmosphere. The reaction was quenched with water (100 mL) and extracted with CH₂Cl₂ (2×100 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:5 to give 7-fluoro-8-((triisopropylsilyl)ethynyl)naphthalen-1-yl trifluoromethanesulfonate as a light yellow solid (5.0 g, 94.1%).

Synthesis of ((2-fluoro-8-(4,4,5,5-tetramethyl-1,3,2-dioxaborolan-2-yl)naphthalen-1-yl)ethynyl)triisopropylsilane: Into a 250 mL round-bottom flask, were placed 7-fluoro-3-(methoxymethoxy)-8-((triisopropylsilyl)ethynyl)naphthalen-1-yl trifluoromethanesulfonate (5.0 g, 9.4 mmol, 1.0 eq), bis(pinacolato)diboron (3.6 g, 14.0 mmol, 1.5 eq), Toluene (100 mL), Pd(dppf)Cl₂ (760 mg, 0.9 mmol, 0.1 eq), KOAc (3.2 g, 32.7 mmol, 3.5 eq) at 25° C. The resulting mixture was stirred for 16 hours at 100° C. under nitrogen atmosphere. The reaction was quenched with water (100 mL) and extracted with ethyl acetate (2×100 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:20 to give ((2-fluoro-8-(4,4,5,5-tetramethyl-1,3,2-dioxaborolan-2-yl)naphthalen-1-yl)ethynyl)triisopropylsilane as a yellow solid (2.0 g, 41.7%). ¹HNMR (300 MHz, DMSO-d₆) δ 8.11-7.97 (m, 2H), 7.79-7.66 (m, 1H), 7.62-7.52 (m, 2H), 1.35 (s, 12H), 1.13 (d, J=4.8 Hz, 21H).

### Example INT_6: Preparation of tert-butyl (1R,5S)-3-(2,7-dichloro-8-fluoropyrido[4,3-d]pyrimidin-4-yl)-3,8-diazabicyclo[3.2.1]octane-8-carboxylate

Synthesis of tert-butyl (1R,5S)-3-(2,7-dichloro-8-fluoropyrido[4,3-d]pyrimidin-4-yl)-3,8-diazabicyclo[3.2.1]octane-8-carboxylate: Into a 1000 mL round-bottom flask, were placed 2,4,7-trichloro-8-fluoropyrido[4,3-d]pyrimidine (10.0 g, 39.6 mmol, 1.0 eq) and CH₂Cl₂ (160 mL). After that, DIEA (12.8 g, 99.0 mmol, 2.5 eq) was added dropwise at −40° C. The reaction mixture was stirred at −40° C. for 15 min. Then to the mixture was added a solution of tert-butyl (1R,5S)-3,8-diazabicyclo[3.2.1]octane-8-carboxylate (8.4 g, 39.6 mmol, 1.0 eq) in CH₂Cl₂ (35 mL) dropwise at −40° C. The resulting mixture was stirred for additional 15 min at −40° C. The reaction mixture was quenched by the addition of water (200 mL), and then extracted with EtOAc (2×300 mL). The combined organic layers were washed with brine (300 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1R,5S)-3-(2,7-dichloro-8-fluoropyrido[4,3-d]pyrimidin-4-yl)-3,8-diazabicyclo[3.2.1]octane-8-carboxylate as a yellow solid (13.6 g, 80.2%). LC-MS (ESI, m/z) M+1: 428.

### Example INT_7: Preparation of tert-butyl N-[(3R)-1-[7-chloro-8-fluoro-2-(hexahydropyrrolizin-7a-ylmethoxy)pyrido[4,3-d]pyrimidin-4-yl]azepan-3-yl]carbamate (assumed) & tert-butyl N-[(3S)-1-[7-chloro-8-fluoro-2-(hexahydropyrrolizin-7a-ylmethoxy)pyrido[4,3-d]pyrimidin-4-yl]azepan-3-yl]carbamate (assumed)

Synthesis of tert-butyl N-(1-{2,7-dichloro-8-fluoropyrido[4,3-d]pyrimidin-4-yl}azepan-3-yl)carbamate: Into a 100 mL 3-necked round-bottom flask, were placed 2,4,7-trichloro-8-fluoropyrido[4,3-d]pyrimidine (1.0 g, 4.0 mmol, 1.0 eq), CH₂Cl₂ (20 mL). After that, DIEA (1.0 g, 7.9 mmol, 2.0 eq) was added dropwised at −40° C. Then the reaction mixture was stirred at −40° C. for 15 min, this was followed by the addition of a solution of tert-butyl N-(azepan-3-yl)carbamate (850 mg, 4.0 mmol, 1.0 eq) in CH₂Cl₂ (5 mL) at −40° C. The resulting mixture was stirred for additional 45 min at −40° C. The reaction was quenched by the addition of water (50 mL) and extracted with ethyl acetate (2×50 mL). The combined organic layers were washed with brine (2×50 mL), and dried over anhydrous Na₂SO₄. After filtration, the filtrate was concentrated under vacuum. The crude residue was purified onto a silica gel column and eluted with ethyl acetate/petroleum ether=1:2 to give tert-butyl N-(1-{2,7-dichloro-8-fluoropyrido[4,3-d]pyrimidin-4-yl}azepan-3-yl)carbamate as a light yellow solid (1.5 g, 88.0%). ¹HNMR (300 MHz, DMSO-d₆) δ 9.14 (s, 1H), 7.05 (d, J=7.2, 1H), 4.25, (d, J=12.9, 1H), 3.91-3.73 (m, 4H), 2.06-1.70 (m, 4H), 1.63-1.45 (m, 2H), 1.41 (s, 9H).

Synthesis of tert-butyl N-{1-[7-chloro-8-fluoro-2-(hexahydropyrrolizin-7a-ylmethoxy)pyrido[4,3-d]pyrimidin-4-yl]azepan-3-yl}carbamate: Into a 100-mL 3-necked round-bottom flask, were placed tert-butyl N-(1-{2,7-dichloro-8-fluoropyrido[4,3-d]pyrimidin-4-yl}azepan-3-yl)carbamate (1.4 g, 3.3 mmol, 1.0 eq), hexahydropyrrolizin-7a-ylmethanol (460 mg, 3.3 mmol, 1.0 eq) and THE (20 mL). After that, NaH (267 mg, 6.5 mmol, 2.0 eq., 60%) was added at 0° C. The resulting solution was stirred for 4 hours at 25° C. The reaction was quenched by the addition of water (40 mL) and extracted with ethyl acetate (2×40 mL). The combined organic layers were washed with brine (2×40 mL) and dried over anhydrous Na₂SO₄. After filtration, the filtrate was concentrated under vacuum. The crude residue was purified onto a silica gel column and eluted with dichloromethane/methanol=5:1 to give tert-butyl N-{1-[7-chloro-8-fluoro-2-(hexahydropyrrolizin-7a-ylmethoxy)pyrido[4,3-d]pyrimidin-4-yl]azepan-3-yl}carbamate as a light yellow solid (1.2 g, 68.9%). LC-MS (ESI, m/z) M+1: 535/537.

Synthesis of tert-butyl N-[(3R)-1-[7-chloro-8-fluoro-2-(hexahydropyrrolizin-7a-ylmethoxy)pyrido[4,3-d]pyrimidin-4-yl]azepan-3-yl]carbamate (assumed) & tert-butyl N-[(3S)-1-[7-chloro-8-fluoro-2-(hexahydropyrrolizin-7a-ylmethoxy)pyrido[4,3-d]pyrimidin-4-yl]azepan-3-yl]carbamate (assumed): 1.0 g of tert-butyl N-{1-[7-chloro-8-fluoro-2-(hexahydropyrrolizin-7a-ylmethoxy)pyrido[4,3-d]pyrimidin-4-yl]azepan-3-yl}carbamate was purified by Chiral-Prep-HPLC using the following conditions: Column: CHIRALPAK IE, 3*25 cm, 5 μm; Mobile Phase A: MTBE (0.1% DEA)-HPLC, Mobile Phase B: IPA-HPLC; Flow rate: 30 mL/min; Gradient: 20% B to 20% B in 16 min; Wave Length: 220/254 nm. Finally, tert-butyl N-[(3R)-1-[7-chloro-8-fluoro-2-(hexahydropyrrolizin-7a-ylmethoxy)pyrido[4,3-d]pyrimidin-4-yl]azepan-3-yl]carbamate (assumed) was obtained as a light yellow oil (430 mg, 43.0%) and tert-butyl N-[(3S)-1-[7-chloro-8-fluoro-2-(hexahydropyrrolizin-7a-ylmethoxy)pyrido[4,3-d]pyrimidin-4-yl]azepan-3-yl]carbamate (assumed) was obtained as a light yellow oil (440 mg, 44.0%). 2A, T_{R}=3.311 min in CHIRAL-HPLC, Column: CHIRALPAK IE-3, 100*4.6 mm, 3 um IE30CS—XF004, phase A: MTBE (0.5% DEA); mobile phase B: IPA, Conc. of Pump B: 20.0%, Oven Temperature: 25° C. 2B, T_{R}=3.982 min in CHIRAL-HPLC, Column: CHIRALPAK IE-3, 100*4.6 mm, 3 um IE30CS—XF004, phase A: MTBE(0.5% DEA); mobile phase B: IPA, Conc. of Pump B: 20.0%, Oven Temperature: 25° C.

### Example INT_8: Preparation of tert-butyl (1-(aminomethyl)cyclobutyl)(methyl)carbamate

Synthesis of 1-((tert-butoxycarbonyl)(methyl)amino)cyclobutane-1-carboxylic acid: Into a 500 mL 3-necked round-bottom flask, were added 1-[(tert-butoxycarbonyl)amino]cyclobutane-1-carboxylic acid (10.0 g, 46.4 mmol, 1.0 eq) and tetrahydrofuran (200 mL) at 25° C. After that, NaH (2.8 g, 60% in mineral oil, 116.6 mmol, 2.5 eq) was added in portions at 0° C. The resulting mixture was stirred for additional 30 min at 0° C. Then CH₃I (9.9 g, 69.7 mmol, 1.5 eq) was added dropwise at 0° C. The resulting mixture was stirred for additional 16 hours at 25° C. The reaction was quenched by the addition of water (200 mL) and acidified to pH=4 with conc. HCl. The resulting mixture was extracted with EtOAc (3×200 mL). The combined organic layers were washed with brine (2×200 mL), dried over anhydrous Na₂SO₄. After filtration, the filtrate was concentrated under vacuum to afford 1-((tert-butoxycarbonyl)(methyl)amino)cyclobutane-1-carboxylic acid as a colorless oil (10.0 g, 93.8%). ¹HNMR (300 MHz, DMSO-d₆) δ 12.40 (s, 1H), 2.72 (s, 3H), 2.47-2.22 (m, 4H), 2.12-1.84 (m, 1H), 1.82-1.67 (m, 1H), 1.35 (s, 9H).

Synthesis of tert-butyl (1-carbamoylcyclobutyl)(methyl)carbamate: Into a 500 mL 3-necked round-bottom flask, were added 1-((tert-butoxycarbonyl)(methyl)amino)cyclobutane-1-carboxylic acid (10.0 g, 43.6 mmol, 1.0 eq), DCM (200 mL), NH₄Cl (7.0 g, 130.8 mmol, 3.0 eq), DIEA (6.7 g, 52.3 mmol, 1.2 eq) and HATU (19.9 g, 52.3 mmol, 1.2 eq) at 25° C. The resulting mixture was stirred for 16 hours at 30° C. The reaction was quenched by the addition of water (150 mL) and then extracted with CH₂Cl₂ (3×150 mL). The combined organic layers were washed with brine (2×200 mL), dried over anhydrous Na₂SO₄. After filtration, the filtrate was concentrated under vacuum to give tert-butyl (1-carbamoylcyclobutyl)(methyl)carbamate as a brown yellow oil (20.0 g, crude). ¹HNMR (300 MHz, DMSO-d₆) δ 6.91 (d, J=34.1 Hz, 1H), 3.62 (pd, J=6.6, 3.8 Hz, 1H), 3.20-3.06 (m, 1H), 2.77 (s, 2H), 2.70 (s, 3H), 2.48-2.09 (m, 2H), 1.95-1.55 (m, 1H), 1.45-1.16 (m, 9H).

Synthesis of tert-butyl (1-(aminomethyl)cyclobutyl)(methyl)carbamate: Into a 500 mL 3-necked round-bottom flask, were added tert-butyl (1-carbamoylcyclobutyl)(methyl)carbamate (20.0 g, 87.6 mmol, 1.0 eq) and BH₃-Me₂S (35.0 mL, 350.0 mmol, 4.0 eq) at 25° C. The resulting mixture was stirred for 12 hours at 25° C. The reaction was quenched by the addition of MeOH (50 mL) at 25° C. The resulting mixture was concentrated under vacuum to give tert-butyl (1-(aminomethyl)cyclobutyl)(methyl)carbamate as a brown oil (25.0 g, crude).

Synthesis of tert-butyl (1-((((benzyloxy)carbonyl)amino)methyl)cyclobutyl)(methyl)carbamate: Into a 500 mL 3-necked round-bottom flask, were added tert-butyl (1-(aminomethyl)cyclobutyl)(methyl)carbamate (25.0 g, 116.6 mmol, 1.0 eq), CH₂Cl₂ (250 mL) and TEA (23.6 g, 233.3 mmol, 2.0 eq) at 25° C. After that, Cbz-Cl (13.9 g, 81.6 mmol, 0.7 eq) was added dropwise at 0° C. The resulting mixture was stirred for additional 4 hours at 25° C. The reaction was quenched by the addition of water (100 mL) and extracted with CH₂Cl₂ (3×100 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to afford tert-butyl (1-((((benzyloxy)carbonyl)amino)methyl)cyclobutyl)(methyl)carbamate as a colorless oil (800 mg, 1.9%). ¹HNMR (300 MHz, DMSO-d₆) δ 7.44-7.20 (m, 5H), 5.02 (s, 2H), 3.36 (d, J=6.6 Hz, 2H), 2.57 (s, 3H), 2.06 (h, J=10.5 Hz, 4H), 1.70-1.54 (m, 3H), 1.37 (s, 9H).

Synthesis of tert-butyl (1-(aminomethyl)cyclobutyl)(methyl)carbamate: Into a 100 mL round-bottom flask, were added tert-butyl (1-((((benzyloxy)carbonyl)amino)methyl)cyclobutyl)(methyl)carbamate (800 mg, 2.2 mmol, 1.0 eq), MeOH (10 mL) and Pd/C (50 mg, 0.5 mmol, 0.2 eq) at 25° C. The resulting mixture was stirred for 12 hours at 25° C. under H₂ (3 atm) atmosphere. The reaction mixture was filtered, the filter cake was washed with MeOH (2×10 mL). The filtrate was concentrated under vacuum to afford tert-butyl (1-(aminomethyl)cyclobutyl)(methyl)carbamate as a colorless oil (400 mg, 81.2%). ¹HNMR (300 MHz, DMSO-d₆) δ 2.77 (s, 2H), 2.66 (s, 3H), 2.16-1.91 (m, 4H), 1.67-1.51 (m, 2H), 1.37 (s, 9H).

### Example INT_9: Preparation of tert-butyl (1-(aminomethyl)cyclopentyl)(methyl)carbamate

Synthesis of 1-((tert-butoxycarbonyl)(methyl)amino)cyclopentane-1-carboxylic acid: Into a 500 mL 3-necked round-bottom flask, were added 1-((tert-butoxycarbonyl)amino)cyclopentane-1-carboxylic acid (10.0 g, 43.6 mmol, 1.0 eq) and tetrahydrofuran (200 mL) at 25° C. After that, NaH (2.6 g, 60% in mineral oil, 109.0 mmol, 2.5 eq) was added in portions at 0° C. The resulting mixture was stirred for an additional 30 min at 0° C. Then CH₃I (9.3 g, 65.4 mmol, 1.5 eq) was added dropwise at 0° C. The resulting mixture was stirred for additional 16 hours at 25° C. The reaction was quenched by the addition of water (200 mL) and then acidified to pH=4 with conc. HCl. The resulting mixture was extracted with ethyl acetate (3×200 mL). The combined organic layers were washed with brine (2×200 mL), dried over anhydrous Na₂SO₄. After filtration, the filtrate was concentrated under vacuum to afford 1-((tert-butoxycarbonyl)(methyl)amino)cyclopentane-1-carboxylic acid as a light brown oil (11.0 g, crude). ¹HNMR (300 MHz, DMSO-d₆) δ 2.86 (s, 3H), 2.28-2.07 (m, 2H), 1.96-1.80 (m, 2H), 1.74-1.52 (m, 4H), 1.36 (s, 9H).

Synthesis of tert-butyl (1-carbamoylcyclopentyl)(methyl)carbamate: Into a 500 mL 3-necked round-bottom flask, were added 1-((tert-butoxycarbonyl)(methyl)amino)cyclopentane-1-carboxylic acid (11.0 g, 45.2 mmol, 1.0 eq), CH₂Cl₂ (200 mL), NH₄Cl (7.2 g, 135.6 mmol, 3.0 eq), DIEA (7.0 g, 54.2 mmol, 1.2 eq) and HATU (20.6 g, 54.2 mmol, 1.2 eq) at 25° C. The resulting mixture was stirred for 16 hours at 30° C. The reaction was quenched by the addition of water (150 mL) and then extracted with CH₂Cl₂ (3×150 mL). The combined organic layers were washed with brine (2×200 mL), dried over anhydrous Na₂SO₄. After filtration, the filtrate was concentrated under vacuum to afford tert-butyl (1-carbamoylcyclopentyl)(methyl)carbamate as a brown oil (25.0 g, crude).

Synthesis of tert-butyl (1-(aminomethyl)cyclopentyl)(methyl)carbamate: Into a 500 mL 3-necked round-bottom flask, were added tert-butyl (1-carbamoylcyclopentyl)(methyl)carbamate (25.0 g, 103.1 mmol, 1.0 eq), tetrahydrofuran (100 mL), and BH₃-tetrahydrofuran (40 mL, 400.0 mmol, 3.8 eq) at 25° C. The resulting mixture was stirred for 12 hours at 25° C. The reaction was quenched by the addition of MeOH (50 mL). The resulting mixture was concentrated under vacuum to give tert-butyl (1-(aminomethyl)cyclopentyl)(methyl)carbamate as a brown oil (20.0 g, crude).

Synthesis of tert-butyl (1-((((benzyloxy)carbonyl)amino)methyl)cyclopentyl)(methyl)carbamate: Into a 500 mL 3-necked round-bottom flask, were added tert-butyl (1-(aminomethyl)cyclopentyl)(methyl)carbamate (20.0 g, 87.5 mmol, 1.0 eq), CH₂Cl₂ (200 mL), and TEA (17.7 g, 175.1 mmol, 2.0 eq) at 25° C. After that, Cbz-Cl (10.4 g, 61.3 mmol, 0.7 eq) was added dropwise at 0° C. The resulting mixture was stirred for additional 4 hours at 25° C. The reaction was quenched by the addition of water (100 mL) and then extracted with CH₂Cl₂ (3×100 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to afford tert-butyl (1-((((benzyloxy)carbonyl)amino)methyl)cyclopentyl)(methyl)carbamate as a colorless oil (1.4 g, 4.4%). ¹HNMR (300 MHz, DMSO-d₆) δ 7.35 (s, 5H), 5.02 (s, 2H), 3.27 (d, J=6.2 Hz, 2H), 2.77 (s, 3H), 1.96 (d, J=12.6 Hz, 2H), 1.72 (d, J=11.3 Hz, 2H), 1.67-1.48 (m, 4H), 1.39 (s, 9H).

Synthesis of tert-butyl (1-(aminomethyl)cyclopentyl)(methyl)carbamate: Into a 100 mL round-bottom flask, were added tert-butyl (1-((((benzyloxy)carbonyl)amino)methyl)cyclopentyl)(methyl)carbamate (1.5 g, 4.1 mmol, 1.0 eq), MeOH (30 mL) and Pd/C (0.1 g, 0.8 mmol, 0.2 eq) at 25° C. The resulting mixture was stirred for 12 hours at 25° C. under H₂(3 atm) atmosphere. The resulting mixture was filtered, the filter cake was washed with MeOH (2×10 mL). The filtrate was concentrated under vacuum to afford tert-butyl (1-(aminomethyl)cyclopentyl)(methyl)carbamate as a colorless oil (800 mg, 84.6%). ¹HNMR (300 MHz, DMSO-d₆) δ 2.87 (s, 3H), 2.68 (s, 2H), 2.03-1.87 (m, 2H), 1.84-1.66 (m, 2H), 1.65-1.43 (m, 2H), 1.39 (s, 9H).

### Example INT_10: Preparation of tert-butyl (1R,5S)-3-(7-chloro-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate and tert-butyl (1R,5S)-3-(7-chloro-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate

Synthesis of 1-tert-butyl 2-methyl (4R)-4-fluoro-2-methylpyrrolidine-1,2-dicarboxylate: Into a 500 mL 3-necked round-bottom flask were added 1-tert-butyl 2-methyl (2S,4R)-4-fluoropyrrolidine-1,2-dicarboxylate (10.0 g, 40.4 mmol, 1.0 eq) and THE (200 mL) at 25° C. After that, LiHMDS (40 ml, 80.6 mmol, 2.0 eq) was added dropwise at −78° C. The resulting mixture was stirred for additional 1 h at −78° C. Then to the above mixture was added MeI (11.5 g, 81.0 mmol, 2.0 eq) dropwiser at −78° C. The resulting mixture was stirred for additional 3 h at 25° C. The reaction was quenched by the addition of water (200 mL) and then extracted with EtOAc (3×200 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to afford 1-tert-butyl 2-methyl (4R)-4-fluoro-2-methylpyrrolidine-1,2-dicarboxylate as a colorless oil (10.0 g, 94.6%). ¹HNMR (400 MHz, DMSO-d₆) δ 5.47-5.01 (m, 1H), 3.80-3.70 (m, 2H), 3.70-3.57 (m, 3H), 2.49-2.13 (m, 3H), 1.59-1.49 (m, 2H), 1.44-1.27 (m, 9H).

Synthesis of ((4R)-4-fluoro-1,2-dimethylpyrrolidin-2-yl)methanol: Into a 500 mL 3-necked round-bottom flask were added 1-tert-butyl 2-methyl (4R)-4-fluoro-2-methylpyrrolidine-1,2-dicarboxylate (10.0 g, 38.2 mmol, 1.0 eq) and THE (200 mL) at 25° C. After that, LiAlH₄ (2.9 g, 76.5 mmol, 2.0 eq) was added in portions at 0° C. The resulting mixture was stirred for additional 3 hours at 0° C. The reaction was quenched by the addition of water (3 mL), 15% NaOH (3 mL) and water (9 mL) at 0° C. in this order, and then stirred for 15 min. The resulting mixture was filtered, the filter cake was washed with THE (2×30 mL). The filtrate was concentrated under vacuum. The crude product was purified by distillation under 30 mmHg and the fraction was collected at 60° C. to afford ((4R)-4-fluoro-1,2-dimethylpyrrolidin-2-yl)methanol as a colorless oil (3.5 g, 62.1%). ¹HNMR (400 MHz, DMSO-d₆) δ 5.29-4.93 (m, 1H), 4.35 (d, J=35.1 Hz, 1H), 3.4-3.12 (m, 2H), 3.09-2.89 (m, 1H), 2.87-2.63 (m, 1H), 2.35-2.08 (m, 4H), 2.05-1.75 (m, 1H), 0.96 (s, 1H), 0.84 (s, 2H).

Synthesis of tert-butyl (1R,5S)-3-(7-chloro-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate and tert-butyl (1R,5S)-3-(7-chloro-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate: Into a 40 mL vial were added ((4R)-4-fluoro-1,2-dimethylpyrrolidin-2-yl)methanol (516 mg, 3.5 mmol, 1.5 eq) and tetrahydrofuran (20 mL) at 25° C. After that, NaH (112 mg, 4.7 mmol, 2.0 eq) was added in portions over 5 min at 0° C. The resulting mixture was stirred for additional 30 min at 25° C. Next, tert-butyl (1R,5S)-3-(2,7-dichloro-8-fluoropyrido[4,3-d]pyrimidin-4-yl)-3,8-diazabicyclo[3.2.1]octane-8-carboxylate (1 g, 2.3 mmol, 1.0 eq) was added in portions over 5 min at 25° C. The resulting mixture was stirred for additional 3 hours at 25° C. The reaction was quenched with sat. NH₄Cl (20 mL) and extracted with ethyl acetate (3×20 mL). The combined organic layers were washed with brine (20 mL), dried over anhydrous Na₂SO₄. After filtration, the filtrate was concentrated under vacuum. The crude residue was applied onto a silica gel column and eluted with dichloromethane/methanol=10:1. The crude product was purified by Prep-SFC using the following conditions. Finally, tert-butyl (1R,5S)-3-(7-chloro-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate was obtained as a white solid (180 mg, 14.3%) and tert-butyl (1R,5S)-3-(7-chloro-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate was obtained as a white solid (500 mg, 39.7%). A, T_{R}=1.29 min in CHIRAL-SFC, Column: (R, R)-WHELK-01-Kromasil, 3*25 cm, 5 μm; Mobile Phase A: CO₂, Mobile Phase B: CH₃OH (0.1% 2M NH₃—CH₃OH); Flow rate: 80 mL/min; Gradient: isocratic 50% B; Column Temperature(25° C.): 35; Back Pressure(bar): 100; Wave Length: 220 nm. B, T_{R}=1.47 min in CHIRAL-SFC, Column: (R, R)-WHELK-01-Kromasil, 3*25 cm, 5 μm; Mobile Phase A: CO₂, Mobile Phase B: CH₃OH (0.1% 2M NH₃—CH₃OH); Flow rate: 80 mL/min; Gradient: isocratic 50% B; Column Temperature(25° C.): 35; Back Pressure(bar): 100; Wave Length: 220 nm. LC-MS (ESI, m/z) M+1: 539/541.

### Example 1: Preparation of 1-[3-(2-{[(2R,7aS)-2-fluoro-hexahydropyrrolizin-7a-yl]methoxy}-7-(8-ethynyl-3-hydroxynaphthalen-1-yl)-8-fluoropyrido[4,3-d]pyrimidin-4-yl)-3,8-diazabicyclo[3.2.1]octan-8-yl]prop-2-en-1-one

Synthesis of tert-butyl (1R,5S)-3-(2,7-dichloro-8-fluoropyrido[4,3-d]pyrimidin-4-yl)-3,8-diazabicyclo[3.2.1]octane-8-carboxylate: To a stirred solution of 2,4,7-trichloro-8-fluoropyrido[4,3-d]pyrimidine (10.0 g, 39.6 mmol, 1.0 eq) in CH₂Cl₂ (160 mL) was added DIEA (12.8 g, 99.0 mmol, 2.5 eq) dropwise at −40° C. The reaction mixture was stirred at −40° C. for 15 min. After that, a solution of tert-butyl (1R,5S)-3,8-diazabicyclo[3.2.1]octane-8-carboxylate (8.4 g, 39.6 mmol, 1.0 eq) in CH₂Cl₂ (35 mL) was added dropwise at −40° C. The resulting mixture was stirred for an additional 15 min at −40° C. The reaction mixture was quenched by the addition of water (200 mL) and then extracted with EtOAc (2×300 mL). The combined organic layers were washed with brine (300 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1R,5S)-3-(2,7-dichloro-8-fluoropyrido[4,3-d]pyrimidin-4-yl)-3,8-diazabicyclo[3.2.1]octane-8-carboxylate as a yellow solid (13.6 g, 80.2%). LC-MS (ESI, m/z) M+1: 428.

Synthesis of tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate: Into a 50 mL round-bottom flask, were placed tert-butyl (1R,5S)-3-{2,7-dichloro-8-fluoropyrido[4,3-d]pyrimidin-4-yl}-3,8-diazabicyclo[3.2.1]octane-8-carboxylate (511 mg, 1.2 mmol, 1.0 eq), [(2R,7aS)-2-fluoro-hexahydropyrrolizin-7a-yl]methanol (190 mg, 1.2 mmol, 1.0 eq) and THF (10 mL). After that, NaH (95 mg, 2.4 mmol, 2.0 eq, 60%) was added in portions at 0° C. under nitrogen atmosphere. The resulting mixture was stirred for 1 hour at 25° C. The reaction mixture was quenched by the addition of water/ice (2 mL) and then extracted with EtOAc (2×20 mL). The combined organic layers were washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to afford tert-butyl (1R,5S)-3-(2-{[(2R,7aS)-2-fluoro-hexahydropyrrolizin-7a-yl]methoxy}-7-chloro-8-fluoropyrido[4,3-d]pyrimidin-4-yl)-3,8-diazabicyclo[3.2.1]octane-8-carboxylate as a colorless oil (160 mg, 24.3%). LC-MS (ESI, m/z) M+1: 551.

Synthesis of tert-butyl (1R,5S)-3-(2-{[(2R,7aS)-2-fluoro-hexahydropyrrolizin-7a-yl]methoxy}-8-fluoro-7-[3-(methoxymethoxy)-8-[2-(triisopropylsilyl)ethynyl]naphthalen-1-yl]pyrido[4,3-d]pyrimidin-4-yl)-3,8-diazabicyclo[3.2.1]octane-8-carboxylate: Into a 50 mL round-bottom flask, were placed tert-butyl (1R,5S)-3-(2-{[(2R,7aS)-2-fluoro-hexahydropyrrolizin-7a-yl]methoxy}-7-chloro-8-fluoropyrido[4,3-d]pyrimidin-4-yl)-3,8-diazabicyclo[3.2.1]octane-8-carboxylate (160 mg, 0.3 mmol, 1.0 eq), triisopropyl((6-(methoxymethoxy)-8-(4,4,5,5-tetramethyl-1,3,2-dioxaborolan-2-yl)naphthalen-1-yl)ethynyl)silane (230 mg, 0.5 mmol, 1.6 eq), K₂CO₃ (120 mg, 0.9 mmol, 3.0 eq), CataCXium Pd G3 (21 mg, 0.03 mmol, 0.1 eq) and Solvents (DME/water=10:1, 4 mL) under nitrogen atmosphere. The resulting mixture was stirred for 4 hours at 80° C. under nitrogen atmosphere. The reaction mixture was quenched by the addition of water (10 mL) and then extracted with EtOAc (2×15 mL). The combined organic layers were washed with brine (15 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1R,5S)-3-(2-{[(2R,7aS)-2-fluoro-hexahydropyrrolizin-7a-yl]methoxy}-8-fluoro-7-[3-(methoxymethoxy)-8-[2-(triisopropylsilyl)ethynyl]naphthalen-1-yl]pyrido[4,3-d]pyrimidin-4-yl)-3,8-diazabicyclo[3.2.1]octane-8-carboxylate as a yellow solid (150 mg, 58.5%). LC-MS (ESI, m/z) M+1: 883.

Synthesis of tert-butyl (1R,5S)-3-(2-{[(2R,7aS)-2-fluoro-hexahydropyrrolizin-7a-yl]methoxy}-7-[8-ethynyl-3-(methoxymethoxy)naphthalen-1-yl]-8-fluoropyrido[4,3-d]pyrimidin-4-yl)-3,8-diazabicyclo[3.2.1]octane-8-carboxylate: Into an 8 mL sealed tube, were placed tert-butyl (1R,5S)-3-(2-{[(2R,7aS)-2-fluoro-hexahydropyrrolizin-7a-yl]methoxy}-8-fluoro-7-[3-(methoxymethoxy)-8-[2-(triisopropylsilyl)ethynyl]naphthalen-1-yl]pyrido[4,3-d]pyrimidin-4-yl)-3,8-diazabicyclo[3.2.1]octane-8-carboxylate (150 mg, 0.17 mmol, 1.0 eq), CsF (258 mg, 1.7 mmol, 10.0 eq) and DMF (3 mL). The resulting mixture was stirred for 2 hours at 25° C. The resulting mixture was quenched by the addition of water (10 mL) and then extracted with EtOAc (2×10 mL). The combined organic layers were washed with brine (20 mL), dried over anhydrous Na₂SO₄. After filtration, the filtrate was concentrated under vacuum. Finally, tert-butyl (1R,5S)-3-(2-{[(2R,7aS)-2-fluoro-hexahydropyrrolizin-7a-yl]methoxy}-7-[8-ethynyl-3-(methoxymethoxy)naphthalen-1-yl]-8-fluoropyrido[4,3-d]pyrimidin-4-yl)-3,8-diazabicyclo[3.2.1]octane-8-carboxylate was obtained as a yellow solid (100 mg, 81.0%). LC-MS (ESI, m/z) M+1: 727.

Synthesis of 4-(2-{[(2R,7aS)-2-fluoro-hexahydropyrrolizin-7a-yl]methoxy}-4-[(1R,5S)-3,8-diazabicyclo[3.2.1]octan-3-yl]-8-fluoropyrido[4,3-d]pyrimidin-7-yl)-5-ethynylnaphthalen-2-ol: Into an 8 mL sealed tube, were placed tert-butyl (1R,5S)-3-(2-{[(2R,7aS)-2-fluoro-hexahydropyrrolizin-7a-yl]methoxy}-7-[8-ethynyl-3-(methoxymethoxy)naphthalen-1-yl]-8-fluoropyrido[4,3-d]pyrimidin-4-yl)-3,8-diazabicyclo[3.2.1]octane-8-carboxylate (100 mg, 0.14 mmol, 1.0 eq) and CH₂Cl₂ (2 mL). After that, HCl (gas) in 1,4-dioxane (4 M, 0.5 mL, 2.0 mmol, 14.5 eq) was added dropwise at 0° C. The resulting mixture was stirred for 1 hour at 0° C. The mixture was neutralized to pH=8 with NH₃ in MeOH (2 M). The resulting mixture was concentrated under vacuum at 0° C. The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min) detector, UV 254 nm to afford 4-(2-{[(2R,7aS)-2-fluoro-hexahydropyrrolizin-7a-yl]methoxy}-4-[(1R,5S)-3,8-diazabicyclo[3.2.1]octan-3-yl]-8-fluoropyrido[4,3-d]pyrimidin-7-yl)-5-ethynylnaphthalen-2-ol as a yellow solid (20 mg, 25.0%). LC-MS (ESI, m/z) M+1: 583. ¹HNMR (400 MHz, DMSO-d₆) δ 10.14 (s, 1H), 9.03 (s, 1H), 7.88 (d, J=7.9 Hz, 1H), 7.44 (dt, J=15.0, 7.1 Hz, 2H), 7.34 (d, J=2.6 Hz, 1H), 7.12 (d, J=2.5 Hz, 1H), 5.28 (d, J=54.2 Hz, 1H), 4.48 (d, J=12.2 Hz, 1H), 4.30 (d, J=12.2 Hz, 1H), 4.11 (dd, J=10.2, 2.6 Hz, 1H), 4.01 (d, J=10.4 Hz, 1H), 3.65 (s, 1H), 3.57 (s, 1H), 3.55 (s, 3H), 3.09 (d, J=9.8 Hz, 2H), 3.02 (s, 1H), 2.84 (s, 1H), 2.14 (d, J=5.1 Hz, 1H), 2.05 (s, 1H), 2.01 (s, 1H), 1.84 (d, J=13.8 Hz, 1H), 1.78 (d, J=9.8 Hz, 2H), 1.66 (s, 4H).

Synthesis of 1-[3-(2-{[(2R,7aS)-2-fluoro-hexahydropyrrolizin-7a-yl]methoxy}-7-(8-ethynyl-3-hydroxynaphthalen-1-yl)-8-fluoropyrido[4,3-d]pyrimidin-4-yl)-3,8-diazabicyclo[3.2.1]octan-8-yl]prop-2-en-1-one: Into an 8 mL sealed tube were added 4-(2-{[(2R,7aS)-2-fluoro-hexahydropyrrolizin-7a-yl]methoxy}-4-[(1R,5S)-3,8-diazabicyclo[3.2.1]octan-3-yl]-8-fluoropyrido[4,3-d]pyrimidin-7-yl)-5-ethynylnaphthalen-2-ol (20 mg, 0.03 mmol, 1.0 eq), CH₂Cl₂ (1 mL) and DIEA (13 mg, 0.09 mmol, 3.0 eq) at 25° C. After that, acryloyl chloride (3 mg, 0.03 mmol, 1.0 eq) was added dropwise at 0° C. The resulting mixture was stirred for 2 hours at 25° C. The reaction mixture was quenched by the addition of water (0.5 mL) and then extracted with CH₂Cl₂ (2×10 mL). The combined organic layers were washed with brine (10 mL), dried over anhydrous Na₂SO₄. After filtration, the filtrate was concentrated under reduced pressure. The residue was dissolved in THE (0.5 mL) and water (0.5 mL) and then LiOH (3 mg, 0.14 mmol, 4.0 eq) was added at 25° C. The reaction mixture was stirred for an additional 3 hours at 25° C. The resulting mixture was concentrated under reduced pressure. The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min) detector, UV 254 nm to afford 1-[3-(2-{[(2R,7aS)-2-fluoro-hexahydropyrrolizin-7a-yl]methoxy}-7-(8-ethynyl-3-hydroxynaphthalen-1-yl)-8-fluoropyrido[4,3-d]pyrimidin-4-yl)-3,8-diazabicyclo[3.2.1]octan-8-yl]prop-2-en-1-one as a yellow solid (5.0 mg, 22.88%). LC-MS (ESI, m/z) M+1: 637. ¹HNMR (400 MHz, DMSO-d₆) δ 10.15 (s, 1H), 9.05 (s, 1H), 7.89 (d, J=8.0 Hz, 1H), 7.45-7.43 (m, 2H), 7.35 (s, 1H), 7.13 (s, 1H), 6.82 (dd, J=16.7, 10.3 Hz, 1H), 6.26 (d, J=16.6 Hz, 1H), 5.79 (d, J=10.4 Hz, 1H), 5.36-5.22 (m, 1H), 4.76 (s, 2H), 4.65 (d, J=12.7 Hz, 1H), 4.44 (s, 1H), 4.14 (d, J=10.4 Hz, 1H), 4.04 (d, J=10.5 Hz, 1H), 3.69 (d, J=12.4 Hz, 1H), 3.60 (s, 2H), 3.10 (d, J=9.5 Hz, 2H), 3.02 (s, 1H), 2.84 (s, 1H), 2.14-2.01 (m, 4H), 1.89-1.75 (m, 6H).

### Example 2: Preparation of 1-[(2S)-2-{[(2-{[(2R,7aS)-2-fluoro-hexahydropyrrolizin-7a-yl]methoxy}-7-(8-ethynyl-7-fluoro-3-hydroxynaphthalen-1-yl)-8-fluoropyrido[4,3-d]pyrimidin-4-yl)(methyl)amino]methyl}pyrrolidin-1-yl]prop-2-en-1-one

Synthesis of 1-[(2S)-2-{[(2-{[(2R,7aS)-2-fluoro-hexahydropyrrolizin-7a-yl]methoxy}-7-(8-ethynyl-7-fluoro-3-hydroxynaphthalen-1-yl)-8-fluoropyrido[4,3-d]pyrimidin-4-yl)(methyl)amino]methyl}pyrrolidin-1-yl]prop-2-en-1-one: Into an 8 mL sealed tube were added 4-(2-{[(2R,7aS)-2-fluoro-hexahydropyrrolizin-7a-yl]methoxy}-8-fluoro-4-{methyl[(2S)-pyrrolidin-2-ylmethyl]amino}pyrido[4,3-d]pyrimidin-7-yl)-5-ethynyl-6-fluoronaphthalen-2-ol (30 mg, 0.05 mmol, 1.0 eq) and DIEA (16 mg, 0.1 mmol, 2.5 eq) at 25° C. After that, acryloyl chloride (3 mg, 0.03 mmol, 0.7 eq) was added dropwise at 0° C. The reaction mixture was stirred for 1 hour at 0° C. The resulting mixture was quenched by the addition of water (5 mL) and then extracted with CH₂Cl₂ (2×10 mL). The combined organic layers were washed with brine (10 mL), dried over anhydrous Na₂SO₄. After filtration, the filtrate was concentrated under reduced pressure. The residue was purified by Prep-HPLC using the following conditions ((Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min) detector, UV 254 nm.) to afford 1-[(2S)-2-{[(2-{[(2R,7aS)-2-fluoro-hexahydropyrrolizin-7a-yl]methoxy}-7-(8-ethynyl-7-fluoro-3-hydroxynaphthalen-1-yl)-8-fluoropyrido[4,3-d]pyrimidin-4-yl)(methyl)amino]methyl}pyrrolidin-1-yl]prop-2-en-1-one as a yellow solid (10 mg, 30.6%). LC-MS (ESI, m/z) M+1: 657. ¹HNMR (400 MHz, DMSO-d₆) δ 10.15 (s, 1H), 9.34-9.03 (m, 1H), 7.98 (dd, J=9.8, 6.0 Hz, 1H), 7.57-7.32 (m, 2H), 7.32-6.61 (m, 2H), 6.14-6.03 (m, 1H), 5.69 (q, J=9.9, 9.1 Hz, 1H), 5.29 (m, 1H), 5.09-4.75 (m, 1H), 4.36-3.91 (m, 5H), 3.85 (d, J=24.1 Hz, 1H), 3.62 (d, J=13.4 Hz, 1H), 3.22 (d, J=11.8 Hz, 1H), 3.11 (d, J=11.9 Hz, 2H), 3.04 (s, 3H), 2.84 (d, J=8.5 Hz, 1H), 2.27-1.93 (m, 6H), 1.92-1.71 (m, 4H).

### Example 3: Preparation of 1-((1R,5S)-3-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one

Synthesis of tert-butyl (1R,5S)-3-(7-chloro-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate: Into a 40 mL vial, were added tert-butyl (1R,5S)-3-(2,7-dichloro-8-fluoropyrido[4,3-d]pyrimidin-4-yl)-3,8-diazabicyclo[3.2.1]octane-8-carboxylate (600 mg, 1.4 mmol, 1.0 eq), dioxane (6 mL), (tetrahydro-1H-pyrrolizin-7a(5H)-yl)methanol (297 mg, 2.1 mmol, 1.5 eq), Cs₂CO₃ (1369 mg, 4.2 mmol, 3.0 eq). The resulting mixture was stirred for additional 16 hours at 90° C. The reaction was quenched with water (6 mL) and extracted with EtOAc (3×6 mL). The combined organic layers were dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(7-chloro-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate as a white solid (370 mg, 49.6%). LC-MS (ESI, m/z) M+1: 533/535.

Synthesis of tert-butyl (1R,5S)-3-(8-fluoro-7-(7-fluoro-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)-3,8-diazabicyclo[3.2.1]octane-8-carboxylate: Into an 8 mL vial, were added tert-butyl (1R,5S)-3-(7-chloro-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate (145 mg, 0.3 mmol, 1.0 eq), ((2-fluoro-8-(4,4,5,5-tetramethyl-1,3,2-dioxaborolan-2-yl)naphthalen-1-yl)ethynyl)triisopropylsilane (115 mg, 0.4 mmol, 1.3 eq), DME (2 mL), H₂O (0.2 mL), cata CXium A Pd G₃ (20 mg, 0.02 mmol, 0.1 eq), K₂CO₃ (113 mg, 0.8 mmol, 3.0 eq) under nitrogen atmosphere. The resulting mixture was stirred for 3 hours at 80° C. under nitrogen atmosphere. The reaction was quenched with water (2 mL) and extracted with EtOAc (3×2 mL). The combined organic layers were dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(8-fluoro-7-(7-fluoro-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)-3,8-diazabicyclo[3.2.1]octane-8-carboxylate as a light yellow solid (150 mg, 66.9%). LC-MS (ESI, m/z) M+1: 823.

Synthesis of tert-butyl (1R,5S)-3-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate: Into an 8 mL vial, were added tert-butyl (1R,5S)-3-(8-fluoro-7-(7-fluoro-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)-3,8-diazabicyclo[3.2.1]octane-8-carboxylate (140 mg, 0.2 mmol, 1.0 eq), CsF (259 mg, 1.7 mmol, 10.0 eq), DMF (3 mL). The resulting mixture was stirred for additional 2 hours at 30° C. The reaction was quenched with water (3 mL) and extracted with EtOAc (3×3 mL). The combined organic layers were dried over anhydrous Na₂SO₄. After filtration, the filtrate was concentrated under vacuum to afford tert-butyl 3-[7-(8-ethynyl-7-fluoronaphthalen-1-yl)-8-fluoro-2-(hexahydropyrrolizin-7a-ylmethoxy)pyrido[4,3-d]pyrimidin-4-yl]-3,8-diazabicyclo[3.2.1]octane-8-carboxylate as a brown solid (95 mg, crude). LC-MS (ESI, m/z) M+1: 667.

Synthesis of 4-((1R,5S)-3,8-diazabicyclo[3.2.1]octan-3-yl)-7-(8-ethynyl-7-fluoronaphthalen-1-yl)-8-fluoro-2-((tetrahydro-1H-pyrrolizin-7a(5H)-yl)methoxy)pyrido[4,3-d]pyrimidine HCl salt: Into an 8 mL vial, were added tert-butyl (1R,5S)-3-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate (80 mg, 0.1 mmol, 1.0 eq), HCl(gas) in 1,4-dioxane (2 mL), CH₂Cl₂ (0.5 mL). The resulting mixture was stirred for additional 1 hours at 25° C. The resulting mixture was concentrated under vacuum. Finally, 4-((1R,5S)-3,8-diazabicyclo[3.2.1]octan-3-yl)-7-(8-ethynyl-7-fluoronaphthalen-1-yl)-8-fluoro-2-((tetrahydro-1H-pyrrolizin-7a(5H)-yl)methoxy)pyrido[4,3-d]pyrimidine HCl salt was obtained as a brown solid (60 mg, crude). LC-MS (ESI, m/z) M+1: 567.

Synthesis of 1-((1R,5S)-3-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one: Into an 8 mL vial, were added 4-((1R,5S)-3,8-diazabicyclo[3.2.1]octan-3-yl)-7-(8-ethynyl-7-fluoronaphthalen-1-yl)-8-fluoro-2-((tetrahydro-1H-pyrrolizin-7a(5H)-yl)methoxy)pyrido[4,3-d]pyrimidine (50 mg, 0.1 mmol, 1.0 eq), CH₂Cl₂ (1 mL), acryloyl chloride (6 mg, 0.1 mmol, 0.7 eq), DIEA (28 mg, 0.2 mmol, 2.5 eq). The resulting mixture was stirred for additional 1 h at 0° C. The reaction was quenched with water (1 mL) and extracted with CH₂Cl₂ (3×1 mL). The combined organic layers were dried over anhydrous Na₂SO₄. After filtration, the filtrate was concentrated under vacuum. The residue was purified by reverse flash chromatography using the following conditions: column, C18 silica gel; mobile phase, MeCN in water (0.1% NH₃·H₂O), 10% to 50% gradient in 10 min; detector, UV 254 nm. Finally, 1-((1R,5S)-3-(7-(8-ethynyl-7-fluoronaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one was obtained as a light yellow solid (8 mg, 14.6%). LC-MS (ESI, m/z) M+1: 621. ¹HNMR (400 MHz, DMSO-d₆) δ 9.07 (s, 1H), 8.30-8.16 (m, 2H), 7.76-7.56 (m, 3H), 6.85-6.74 (m, 1H), 6.27 (d, J=15.9 Hz, 1H), 5.82-5.73 (m, 1H), 4.76 (s, 2H), 4.64 (d, J=12.7 Hz, 1H), 4.46 (t, J=13.3 Hz, 1H), 4.05 (d, J=9.1 Hz, 3H), 3.76-3.53 (m, 2H), 2.94 (dt, J=10.4, 5.5 Hz, 2H), 2.57 (d, J=7.3 Hz, 2H), 1.90 (dt, J=11.9, 5.9 Hz, 4H), 1.79-157 (m, 8H).

### Example 4: Preparation of 1-((1R,5S)-3-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one

Synthesis of tert-butyl (1R,5S)-3-(7-chloro-2-(3-(dimethylamino)azetidin-1-yl)-8-fluoropyrido[4,3-d]pyrimidin-4-yl)-3,8-diazabicyclo[3.2.1]octane-8-carboxylate: Into a 40 mL vial, were placed tert-butyl (1R,5S)-3-(2,7-dichloro-8-fluoropyrido[4,3-d]pyrimidin-4-yl)-3,8-diazabicyclo[3.2.1]octane-8-carboxylate (500 mg, 1.2 mmol, 1.0 eq), N,N-dimethylazetidin-3-amine (117 mg, 1.2 mmol, 1.0 eq), acetonitrile (10 mL) and Cs₂CO₃ (761 mg, 2.3 mmol, 2.0 eq) at 25° C. The resulting mixture was stirred for additional 4 hours at 80° C. The resulting mixture was diluted with H₂O (30 mL) and extracted with CH₂Cl₂ (4×10 mL). The combined organic layers were washed with brine (10 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(7-chloro-2-(3-(dimethylamino)azetidin-1-yl)-8-fluoropyrido[4,3-d]pyrimidin-4-yl)-3,8-diazabicyclo[3.2.1]octane-8-carboxylate as a white solid (640 mg, 90%). LC-MS (ESI, m/z) M+1: 492/494.

Synthesis of tert-butyl (1R,5S)-3-(2-(3-(dimethylamino)azetidin-1-yl)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate: Into an 8 mL vial, were placed tert-butyl (1R,5S)-3-(7-chloro-2-(3-(dimethylamino)azetidin-1-yl)-8-fluoropyrido[4,3-d]pyrimidin-4-yl)-3,8-diazabicyclo[3.2.1]octane-8-carboxylate (200 mg, 0.4 mmol, 1.0 eq), ((2-fluoro-6-(methoxymethoxy)-8-(4,4,5,5-tetramethyl-1,3,2-dioxaborolan-2-yl)naphthalen-1-yl)ethynyl)triisopropylsilane (250 mg, 0.5 mmol, 1.2 eq), dimethoxyethane (4 mL), H₂O (0.4 mL), K₂CO₃ (169 mg, 1.2 mmol, 3.0 eq) and CataCXium A Pd G3 (30 mg, 0.04 mmol, 0.1 eq) at 25° C. under nitrogen atmosphere. The resulting mixture was stirred for 4 hours at 80° C. under nitrogen atmosphere. The resulting mixture was diluted with H₂O (5 mL) and extracted with CH₂Cl₂ (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(2-(3-(dimethylamino)azetidin-1-yl)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate as a white solid (170 mg, 53.4%). LC-MS (ESI, m/z) M+1: 842.

Synthesis of tert-butyl (1R,5S)-3-(2-(3-(dimethylamino)azetidin-1-yl)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate: Into an 8 mL vial, were placed tert-butyl (1R,5S)-3-(2-(3-(dimethylamino)azetidin-1-yl)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate (160 mg, 0.2 mmol, 1.0 eq), CsF (289 mg, 1.9 mmol, 10.0 eq) and DMF (3 mL). The resulting mixture was stirred for 2 hours at 25° C. The resulting mixture was then quenched by the addition of water (5 mL) and extracted with ethyl acetate (4×5 mL). The combined organic layers were washed with brine (5 mL), dried over anhydrous Na₂SO₄. After filtration, the filtrate was concentrated under vacuum to give tert-butyl (1R,5S)-3-(2-(3-(dimethylamino)azetidin-1-yl)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate as a yellow solid (150 mg, crude). LC-MS (ESI, m/z) M+1: 686.

Synthesis of 4-(4-((1R,5S)-3,8-diazabicyclo[3.2.1]octan-3-yl)-2-(3-(dimethylamino)azetidin-1-yl)-8-fluoropyrido[4,3-d]pyrimidin-7-yl)-5-ethynyl-6-fluoronaphthalen-2-ol: Into an 8 mL sealed tube, were placed tert-butyl (1R,5S)-3-(2-(3-(dimethylamino)azetidin-1-yl)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (140 mg, 0.2 mmol, 1.0 eq) and CH₂Cl₂ (2 mL). After that, HCl(gas) in 1,4-dioxane (1 mL) was added dropwise at 0° C. The resulting mixture was stirred for 1 hour at 0° C. The mixture was neutralized to pH=8 with NH₃ in methanol (2 M). The resulting mixture was diluted water (5 mL) and extracted with CH₂Cl₂ (4×5 mL). The combined organic layers were washed with brine (5 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-2-(3-(dimethylamino)azetidin-1-yl)-8-fluoropyrido[4,3-d]pyrimidin-7-yl)-5-ethynyl-6-fluoronaphthalen-2-ol as a yellow solid (100 mg, 90.4%). LC-MS (ESI, m/z) M+1: 542.

Synthesis of 1-((1R,5S)-3-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one: Into an 8 mL sealed tube, were placed 4-(4-((1R,5S)-3,8-diazabicyclo[3.2.1]octan-3-yl)-2-(3-(dimethylamino)azetidin-1-yl)-8-fluoropyrido[4,3-d]pyrimidin-7-yl)-5-ethynyl-6-fluoronaphthalen-2-ol (90 mg, 0.2 mmol, 1.0 eq), CH₂Cl₂ (2 mL) and DIEA (54 mg, 0.4 mmol, 2.5 eq) at 25° C. After that, acryloyl chloride (12 mg, 0.1 mmol, 0.8 eq) was added dropwise at 0° C. The resulting mixture was stirred for 1 hour at 0° C. The reaction was quenched by the addition of water (0.5 mL) at 25° C. The resulting mixture was extracted with CH₂Cl₂ (2×10 mL). The combined organic layers were washed with brine (10 mL), dried over anhydrous Na₂SO₄. After filtration, the filtrate was concentrated under reduced pressure. The residue was dissolved in tetrahydrofuran (2 mL) and H₂O (2 mL). To the above mixture was added LiOH·H₂O (28 mg, 0.7 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, H₂O (10 mmol/L NH₄HCO₃+0.1% NH₃·H₂O) and CH₃CN (45% CH₃CN up to 85% in 10 min), Flow rate: 60 mL/min; Detector, 254/220 nm. Finally, 1-((1R,5S)-3-(2-(3-(dimethylamino)azetidin-1-yl)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one as a yellow solid (10 mg, 10.3%). LC-MS (ESI, m/z) M+1: 596. ¹HNMR (300 MHz, Chloroform-d) δ 8.72 (s, 1H), 7.61 (dd, J=8.1, 5.7 Hz, 1H), 7.24-7.04 (m, 3H), 6.65-6.33 (m, 2H), 5.81 (dd, J=8.7, 2.4 Hz, 1H), 4.93 (s, 1H), 4.67-3.99 (m, 6H), 3.61 (dt, J=9.0, 8.4 Hz, 2H), 3.22 (s, 1H), 2.82 (s, 1H), 2.26 (s, 6H), 1.98 (s, 3H), 1.85-1.69 (m, 2H), 1.28 (s, 1H).

### Example 5: Preparation of 1-((1R,5S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one

Synthesis of tert-butyl (1R,5S)-3-(7-chloro-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate: Into a 100 mL round-bottom flask, were placed tert-butyl 3-{2,7-dichloro-8-fluoropyrido[4,3-d]pyrimidin-4-yl}-3,8-diazabicyclo[3.2.1]octane-8-carboxylate (700 mg, 1.6 mmol, 1.0 eq), hexahydropyrrolizin-7a-ylmethanol (346 mg, 2.5 mmol, 1.5 eq), dioxane (20 mL) and Cs₂CO₃ (1598 mg, 4.9 mmol, 3.0 eq) at 25° C. The resulting mixture was stirred for 16 hours at 90° C. under nitrogen atmosphere. The reaction was quenched by the addition of water (10 mL) at 25° C. The resulting mixture was extracted with EtOAc (2×20 mL). The combined organic layers were washed with brine (30 mL), dried over anhydrous Na₂SO₄. After filtration, the filtrate was concentrated under reduced pressure. The crude residue was applied onto a silica gel column and eluted with CH₂Cl₂/MeOH=10:1 to afford tert-butyl (1R,5S)-3-(7-chloro-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate as a white solid (600 mg, 68.9%). LC-MS (ESI, m/z) M+1: 533.

Synthesis of tert-butyl (1R,5S)-3-(8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)-3,8-diazabicyclo[3.2.1]octane-8-carboxylate: Into a 50 mL round-bottom flask, were placed tert-butyl (1R,5S)-3-(7-chloro-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate (300 mg, 0.4 mmol, 1.0 eq), {2-[2-fluoro-6-(methoxymethoxy)-8-(4,4,5,5-tetramethyl-1,3,2-dioxaborolan-2-yl)naphthalen-1-yl]ethynyl}triisopropylsilane (275 mg, 0.5 mmol, 1.3 eq), DME (5 mL), H₂O (0.5 mL), K₂CO₃ (171 mg, 1.2 mmol, 3.0 eq) and CataCXium A Pd G3 (26 mg, 0.04 mmol, 0.1 eq) at 25° C. under nitrogen atmosphere. The resulting mixture was stirred for 4 hours at 80° C. under nitrogen atmosphere. The reaction mixture was then quenched by the addition of H₂O (5 mL). The resulting mixture was extracted with EtOAc (2×15 mL). The combined organic layers were washed with brine (15 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1R,5S)-3-(8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)-3,8-diazabicyclo[3.2.1]octane-8-carboxylate as an off-white solid (300 mg, 82.3%). LC-MS (ESI, m/z) M+1: 883.

Synthesis of tert-butyl (1R,5S)-3-(7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate: Into a 40 mL sealed tube, were placed tert-butyl (1R,5S)-3-(8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)-3,8-diazabicyclo[3.2.1]octane-8-carboxylate (280 mg, 0.3 mmol, 1.0 eq), CsF (482 mg, 3.2 mmol, 10.0 eq) and DMF (5 mL). The resulting mixture was stirred for 2 hours at 25° C. The resulting mixture was quenched by the addition of water (10 mL) and then extracted with EtOAc (2×10 mL). The combined organic layers were washed with brine (20 mL), dried over anhydrous Na₂SO₄. After filtration, the filtrate was concentrated under vacuum to give tert-butyl (1R,5S)-3-(7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate as a yellow oil (200 mg, 86.8%). LC-MS (ESI, m/z) M+1: 727.

Synthesis of 4-(4-((1R,5S)-3,8-diazabicyclo[3.2.1]octan-3-yl)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-ol: Into an 8 mL sealed tube, were placed tert-butyl (1R,5S)-3-(7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-8-carboxylate (200 mg, 0.3 mmol, 1.0 eq) and CH₂Cl₂ (2 mL). After that, a solution of HCl (gas) in 1,4-dioxane (1 mL) was added dropwise at 0° C. The resulting mixture was stirred for 1 hour at 0° C. The mixture was neutralized to pH=8 with NH₃ in MeOH (2 M). The resulting mixture was diluted water (10 mL). The resulting mixture was extracted with CH₂Cl₂ (2×10 mL). The combined organic layers were washed with brine (10 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-ol as a yellow solid (100 mg, 62.4%). LC-MS (ESI, m/z) M+1: 583.

Synthesis of 1-((1R,5S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one: Into an 8 mL sealed tube, were placed 4-(4-((1R,5S)-3,8-diazabicyclo[3.2.1]octan-3-yl)-8-fluoro-2-((tetrahydro-1H-pyrrolizin-7a(5H)-yl)methoxy)pyrido[4,3-d]pyrimidin-7-yl)-5-ethynyl-6-fluoronaphthalen-2-ol (90 mg, 0.2 mmol, 1.0 eq), CH₂Cl₂ (3 mL) and DIEA (50 mg, 0.4 mmol, 2.5 eq) at 25° C. To the above mixture was added acryloyl chloride (14 mg, 0.2 mmol, 1.0 eq) dropwise at 0° C. The resulting mixture was stirred for 1 hour at 0° C. The reaction was quenched by the addition of water (0.5 mL) at 25° C. and then extracted with CH₂Cl₂ (2×10 mL). The combined organic layers were washed with brine (10 mL), dried over anhydrous Na₂SO₄. After filtration, the filtrate was concentrated under reduced pressure. The residue was dissolved in THE (2 mL) and H₂O (2 mL). To the above mixture was added LiOH·H₂O (15 mg, 0.6 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, H₂O (10 mmol/L NH₄HCO₃+0.1% NH₃·H₂O) and CH₃CN (45% CH₃CN up to 85% in 10 min), Flow rate: 60 mL/min; Detector, 254/220 nm. Finally, 1-((1R,5S)-3-(7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octan-8-yl)prop-2-en-1-one was obtained as a yellow solid (15 mg, 15.3%). LC-MS (ESI, m/z) M+1: 637. ¹HNMR (400 MHz, DMSO-d₆) δ 10.11 (s, 1H), 9.05 (s, 1H), 7.98 (dd, J=9.1, 5.8 Hz, 1H), 7.47 (t, J=9.0 Hz, 1H), 7.42-7.37 (m, 1H), 7.19 (s, 1H), 6.81 (dd, J=16.8, 10.4 Hz, 1H), 6.26 (d, J=16.7 Hz, 1H), 5.78 (d, J=10.5 Hz, 1H), 4.76 (s, 2H), 4.63 (s, 1H), 4.45 (t, J=13.6 Hz, 1H), 4.06 (s, 2H), 3.95 (s, 1H), 3.68 (d, J=11.4 Hz, 1H), 3.61 (s, 1H), 2.94 (dt, J=10.8, 5.6 Hz, 2H), 2.60-2.51 (m, 2H), 1.95-1.69 (m, 10H), 1.59 (dd, J=12.4, 7.1 Hz, 2H).

### Example 6: Preparation of 1-((1R,5S)-3-(7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octan-8-yl)prop-2-en-1-one

Synthesis of tert-butyl (1R,5S)-3-(7-chloro-8-fluoro-2-(((S)-1-methylpyrrolidin-2-yl)methoxy)pyrido[4,3-d]pyrimidin-4-yl)-3,8-diazabicyclo[3.2.1]octane-8-carboxylate: Into a 50 mL round-bottom flask, were placed tert-butyl (1R,5S)-3-(2,7-dichloro-8-fluoropyrido[4,3-d]pyrimidin-4-yl)-3,8-diazabicyclo[3.2.1]octane-8-carboxylate (600 mg, 1.4 mmol, 1.0 eq), (S)-(1-methylpyrrolidin-2-yl)methanol (161 mg, 1.4 mmol, 1.0 eq) and THE (15 mL) at 25° C. After that, NaH (67 mg, 2.8 mmol, 2.0 eq) was added in portions at 0° C. The resulting mixture was stirred for 4 hours at 25° C. The reaction was quenched by the addition of water (5 mL) at 25° C. and then extracted with EtOAc (2×20 mL). The combined organic layers were washed with brine (30 mL), dried over anhydrous Na₂SO₄. After filtration, the filtrate was concentrated under reduced pressure. The crude residue was applied onto a silica gel column and eluted with CH₂Cl₂/MeOH=10:1 to give tert-butyl (1R,5S)-3-(7-chloro-8-fluoro-2-(((S)-1-methylpyrrolidin-2-yl)methoxy)pyrido[4,3-d]pyrimidin-4-yl)-3,8-diazabicyclo[3.2.1]octane-8-carboxylate as a white solid (420 mg, 59.1%). LC-MS (ESI, m/z) M+1: 507.

Synthesis of tert-butyl (1R,5S)-3-(8-fluoro-7-(7-fluoro-3-(methoxymethoxy)-8-((triisopropylsilyl)ethynyl)naphthalen-1-yl)-2-(((S)-1-methylpyrrolidin-2-yl)methoxy)pyrido[4,3-d]pyrimidin-4-yl)-3,8-diazabicyclo[3.2.1]octane-8-carboxylate: Into a 50 mL round-bottom flask, were placed tert-butyl (1R,5S)-3-(7-chloro-8-fluoro-2-(((S)-1-methylpyrrolidin-2-yl)methoxy)pyrido[4,3-d]pyrimidin-4-yl)-3,8-diazabicyclo[3.2.1]octane-8-carboxylate (420 mg, 0.8 mmol, 1.0 eq), ((2-fluoro-6-(methoxymethoxy)-8-(4,4,5,5-tetramethyl-1,3,2-dioxaborolan-2-yl)naphthalen-1-yl)ethynyl)triisopropylsilane (552 mg, 1.0 mmol, 1.3 eq), K₂CO₃ (343 mg, 2.5 mmol, 3.0 eq), DME (10 mL), H₂O (1 mL) and CataCXium A Pd G3 (53 mg, 0.1 mmol, 0.1 eq) at 25° C. under nitrogen atmosphere. The resulting mixture was stirred for 4 hours at 80° C. under nitrogen atmosphere. The reaction mixture was quenched by the addition of H₂O (5 mL). The resulting mixture was extracted with EtOAc (2×15 mL). The combined organic layers were washed with brine (15 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1R,5S)-3-(8-fluoro-7-(7-fluoro-3-(methoxymethoxy)-8-((triisopropylsilyl)ethynyl)naphthalen-1-yl)-2-(((S)-1-methylpyrrolidin-2-yl)methoxy)pyrido[4,3-d]pyrimidin-4-yl)-3,8-diazabicyclo[3.2.1]octane-8-carboxylate as a colorless oil (250 mg, 35.2%). LC-MS (ESI, m/z) M+1: 858.

Synthesis of tert-butyl (1R,5S)-3-(7-(8-ethynyl-7-fluoro-3-(methoxymethoxy)naphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octane-8-carboxylate: Into a 40 mL sealed tube, were placed tert-butyl (1R,5S)-3-(8-fluoro-7-(7-fluoro-3-(methoxymethoxy)-8-((triisopropylsilyl)ethynyl)naphthalen-1-yl)-2-(((S)-1-methylpyrrolidin-2-yl)methoxy)pyrido[4,3-d]pyrimidin-4-yl)-3,8-diazabicyclo[3.2.1]octane-8-carboxylate (250 mg, 0.3 mmol, 1.0 eq), CsF (443 mg, 2.9 mmol, 10.0 eq) and DMF (5 mL). The resulting mixture was stirred for 2 hours at 25° C. The resulting mixture was quenched by the addition of water (10 mL) and then extracted with EtOAc (2×10 mL). The combined organic layers were washed with brine (20 mL), dried over anhydrous Na₂SO₄. After filtration, the filtrate was concentrated under vacuum to give tert-butyl (1R,5S)-3-(7-(8-ethynyl-7-fluoro-3-(methoxymethoxy)naphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octane-8-carboxylate a yellow oil (170 mg, 83.2%). LC-MS (ESI, m/z) M+1: 701.

Synthesis of 4-(4-((1R,5S)-3,8-diazabicyclo[3.2.1]octan-3-yl)-8-fluoro-2-(((S)-1-methylpyrrolidin-2-yl)methoxy)pyrido[4,3-d]pyrimidin-7-yl)-5-ethynyl-6-fluoronaphthalen-2-ol: Into an 8 mL sealed tube, were placed tert-butyl (1R,5S)-3-(7-(8-ethynyl-7-fluoro-3-(methoxymethoxy)naphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octane-8-carboxylate (170 mg, 0.2 mmol, 1.0 eq) and CH₂Cl₂ (2 mL). After that, HCl(gas) in 1,4-dioxane (1 mL) was added dropwise at 0° C. The resulting mixture was stirred for 1 hour at 0° C. The mixture was neutralized to pH=8 with NH₃ in MeOH (2 M). The resulting mixture was diluted water (10 mL). The resulting mixture was extracted with CH₂Cl₂ (2×10 mL). The combined organic layers were washed with brine (10 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-8-fluoro-2-(((S)-1-methylpyrrolidin-2-yl)methoxy)pyrido[4,3-d]pyrimidin-7-yl)-5-ethynyl-6-fluoronaphthalen-2-ol as a yellow solid (80 mg, 59.3%). LC-MS (ESI, m/z) M+1: 557.

Synthesis of 1-((1R,5S)-3-(7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octan-8-yl)prop-2-en-1-one: Into an 8 mL sealed tube, were placed 4-(4-{3,8-diazabicyclo[3.2.1]octan-3-yl}-8-fluoro-2-{[(2S)-1-methylpyrrolidin-2-yl]methoxy}pyrido[4,3-d]pyrimidin-7-yl)-5-ethynyl-6-fluoronaphthalen-2-ol (80 mg, 0.2 mmol, 1.0 eq), CH₂Cl₂ (3 mL) and DIEA (46 mg, 0.4 mmol, 2.5 eq) at 25° C. After that, acryloyl chloride (13 mg, 0.2 mmol, 1.0 eq) was added dropwise at 0° C. The resulting mixture was stirred for 1 hour at 0° C. The reaction was quenched by the addition of water (0.5 mL) at 25° C. and then extracted with CH₂Cl₂ (2×10 mL). The combined organic layers were washed with brine (10 mL), dried over anhydrous Na₂SO₄. After filtration, the filtrate was concentrated under reduced pressure. The residue was dissolved in THE (2 mL) and H₂O (2 mL). To the above mixture was added LiOH·H₂O (24 mg, 0.6 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, H₂O (10 mmol/L NH₄HCO₃+0.1% NH₃·H₂O) and CH₃CN (45% CH₃CN up to 85% in 10 min), Flow rate: 60 mL/min; Detector, 254/220 nm. Finally, 1-((1R,5S)-3-(7-(8-ethynyl-7-fluoro-3-hydroxynaphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octan-8-yl)prop-2-en-1-one was obtained as a yellow solid (20 mg, 22.79%). LC-MS (ESI, m/z) M+1: 611. ¹HNMR (400 MHz, DMSO-d₆) δ 10.16 (s, 1H), 9.06 (s, 1H), 7.99 (dd, J=9.2, 5.9 Hz, 1H), 7.47 (t, J=9.0 Hz, 1H), 7.40 (d, J=2.5 Hz, 1H), 7.19 (s, 1H), 6.82 (dd, J=16.7, 10.3 Hz, 1H), 6.26 (dd, J=16.8, 2.3 Hz, 1H), 5.79 (d, J=10.6 Hz, 1H), 4.76 (s, 2H), 4.64 (t, J=11.9 Hz, 1H), 4.43 (d, J=12.9 Hz, 2H), 4.23 (s, 1H), 3.96 (s, 1H), 3.69 (d, J=12.5 Hz, 1H), 3.62 (d, J=12.6 Hz, 1H), 3.03-2.95 (m, 1H), 2.60-2.55 (m, 1H), 2.40 (s, 3H), 2.25-2.19 (m, 1H), 2.03-1.92 (m, 2H), 1.84-1.79 (m, 4H), 1.73-1.68 (m, 3H).

### Example 7: Preparation of 1-((1R,5S)-3-(7-(8-ethynyl-7-fluoro-3-hydroxynaphthalen-1-yl)-8-fluoro-2-(((S)-1-methylpiperidin-2-yl)methoxy)pyrido[4,3-d]pyrimidin-4-yl)-3,8-diazabicyclo[3.2.1]octan-8-yl)prop-2-en-1-one

Synthesis of tert-butyl (1R,5S)-3-(7-chloro-8-fluoro-2-(((S)-1-methylpiperidin-2-yl)methoxy)pyrido[4,3-d]pyrimidin-4-yl)-3,8-diazabicyclo[3.2.1]octane-8-carboxylat: Into a 50 mL round-bottom flask, were placed tert-butyl (1R,5S)-3-(2,7-dichloro-8-fluoropyrido[4,3-d]pyrimidin-4-yl)-3,8-diazabicyclo[3.2.1]octane-8-carboxylate (750 mg, 1.8 mmol, 1.0 eq), (S)-(1-methylpiperidin-2-yl)methanol (226 mg, 1.8 mmol, 1.0 eq) and THE (15 mL) at 25° C. After that, NaH (84 mg, 3.6 mmol, 2.0 eq., 60%) was added in portions at 0° C. The resulting mixture was stirred for 4 hours at 25° C. The reaction was quenched by the addition of water (5 mL) at 25° C. The resulting mixture was extracted with EtOAc (2×20 mL). The combined organic layers were washed with brine (30 mL), dried over anhydrous Na₂SO₄. After filtration, the filtrate was concentrated under vacuum. The crude residue was applied onto a silica gel column and eluted with MeOH/CH₂Cl₂=1:10 to give tert-butyl (1R,5S)-3-(7-chloro-8-fluoro-2-(((S)-1-methylpiperidin-2-yl)methoxy)pyrido[4,3-d]pyrimidin-4-yl)-3,8-diazabicyclo[3.2.1]octane-8-carboxylate as a white solid (600 mg, 65.8%). LC-MS (ESI, m/z) M+1: 521.

Synthesis of tert-butyl (1R,5S)-3-(8-fluoro-7-(7-fluoro-3-(methoxymethoxy)-8-((triisopropylsilyl)ethynyl)naphthalen-1-yl)-2-(((S)-1-methylpiperidin-2-yl)methoxy)pyrido[4,3-d]pyrimidin-4-yl)-3,8-diazabicyclo[3.2.1]octane-8-carboxylate: Into a 50 mL round-bottom flask, were placed tert-butyl (1R,5S)-3-(7-chloro-8-fluoro-2-(((S)-1-methylpiperidin-2-yl)methoxy)pyrido[4,3-d]pyrimidin-4-yl)-3,8-diazabicyclo[3.2.1]octane-8-carboxylate (300 mg, 0.6 mmol, 1.0 eq), {2-[2-fluoro-6-(methoxymethoxy)-8-(4,4,5,5-tetramethyl-1,3,2-dioxaborolan-2-yl)naphthalen-1-yl]ethynyl}triisopropylsilane (354 mg, 0.7 mmol, 1.2 eq), K₂CO₃ (239 mg, 1.7 mmol, 3.0 eq), DME (10 mL), H₂O (1 mL) and CataCXium A Pd G3(37 mg, 0.06 mmol, 0.1 eq) at 25° C. under nitrogen atmosphere. The resulting mixture was stirred for 4 hours at 80° C. under nitrogen atmosphere. The reaction mixture was then quenched by the addition of water (5 mL). The resulting mixture was extracted with EtOAc (2×15 mL). The combined organic layers were washed with brine (15 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1R,5S)-3-(8-fluoro-7-(7-fluoro-3-(methoxymethoxy)-8-((triisopropylsilyl)ethynyl)naphthalen-1-yl)-2-(((S)-1-methylpiperidin-2-yl)methoxy)pyrido[4,3-d]pyrimidin-4-yl)-3,8-diazabicyclo[3.2.1]octane-8-carboxylate as a colorless oil (230 mg, 45.9%). LC-MS (ESI, m/z) M+1: 871.

Synthesis of tert-butyl (1R,5S)-3-(7-(8-ethynyl-7-fluoro-3-(methoxymethoxy)naphthalen-1-yl)-8-fluoro-2-(((S)-1-methylpiperidin-2-yl)methoxy)pyrido[4,3-d]pyrimidin-4-yl)-3,8-diazabicyclo[3.2.1]octane-8-carboxylate: Into a 40 mL sealed tube, were placed tert-butyl (1R,5S)-3-(8-fluoro-7-(7-fluoro-3-(methoxymethoxy)-8-((triisopropylsilyl)ethynyl)naphthalen-1-yl)-2-(((S)-1-methylpiperidin-2-yl)methoxy)pyrido[4,3-d]pyrimidin-4-yl)-3,8-diazabicyclo[3.2.1]octane-8-carboxylate (230 mg, 0.3 mmol, 1.0 eq), CsF (401 mg, 3.0 mmol, 10.0 eq) and DMF (5 mL). The resulting mixture was stirred for 2 hours at 25° C. The resulting mixture was then quenched by the addition of water (10 mL). The resulting mixture was extracted with EtOAc (2×10 mL). The combined organic layers were washed with brine (20 mL), dried over anhydrous Na₂SO₄. After filtration, the filtrate was concentrated under vacuum to give tert-butyl (1R,5S)-3-(7-(8-ethynyl-7-fluoro-3-(methoxymethoxy)naphthalen-1-yl)-8-fluoro-2-(((S)-1-methylpiperidin-2-yl)methoxy)pyrido[4,3-d]pyrimidin-4-yl)-3,8-diazabicyclo[3.2.1]octane-8-carboxylate as a yellow oil (120 mg, 63.6%). LC-MS (ESI, m/z) M+1: 715.

Synthesis of 4-(4-((1R,5S)-3,8-diazabicyclo[3.2.1]octan-3-yl)-8-fluoro-2-(((S)-1-methylpiperidin-2-yl)methoxy)pyrido[4,3-d]pyrimidin-7-yl)-5-ethynyl-6-fluoronaphthalen-2-ol: Into an 8 mL sealed tube, were placed t tert-butyl (1R,5S)-3-(7-(8-ethynyl-7-fluoro-3-(methoxymethoxy)naphthalen-1-yl)-8-fluoro-2-(((S)-1-methylpiperidin-2-yl)methoxy)pyrido[4,3-d]pyrimidin-4-yl)-3,8-diazabicyclo[3.2.1]octane-8-carboxylate (120 mg, 0.2 mmol, 1.0 eq) and CH₂Cl₂ (2 mL). After that, HCl(gas) in 1,4-dioxane (1 mL) was added dropwise at 0° C. The resulting mixture was stirred for 1 hour at 0° C. The mixture was neutralized to pH=8 with NH₃ in MeOH (2 M). The resulting mixture was diluted water (10 mL). The resulting mixture was extracted with CH₂Cl₂ (2×10 mL). The combined organic layers were washed with brine (10 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-8-fluoro-2-(((S)-1-methylpiperidin-2-yl)methoxy)pyrido[4,3-d]pyrimidin-7-yl)-5-ethynyl-6-fluoronaphthalen-2-ol as a yellow solid (60 mg, 62.6%). LC-MS (ESI, m/z) M+1: 571.

Synthesis of 1-((1R,5S)-3-(7-(8-ethynyl-7-fluoro-3-hydroxynaphthalen-1-yl)-8-fluoro-2-(((S)-1-methylpiperidin-2-yl)methoxy)pyrido[4,3-d]pyrimidin-4-yl)-3,8-diazabicyclo[3.2.1]octan-8-yl)prop-2-en-1-one: Into an 8 mL sealed tube, were placed 4-(4-((1R,5S)-3,8-diazabicyclo[3.2.1]octan-3-yl)-8-fluoro-2-(((S)-1-methylpiperidin-2-yl)methoxy)pyrido[4,3-d]pyrimidin-7-yl)-5-ethynyl-6-fluoronaphthalen-2-ol (60 mg, 0.105 mmol, 1 eq), CH₂Cl₂ (2 mL) and DIEA (34 mg, 0.3 mmol, 2.5 eq) at 25° C. After that, acryloyl chloride (10 mg, 0.1 mmol, 1.0 eq) was added dropwise at 0° C. The resulting mixture was stirred for 1 hour at 0° C. The reaction was quenched by the addition of water (5 mL) at 25° C. and then extracted with CH₂Cl₂ (2×10 mL). The combined organic layers were washed with brine (10 mL), dried over anhydrous Na₂SO₄. After filtration, the filtrate was concentrated under reduced pressure. The residue was dissolved in THE (1 mL) and H₂O (1 mL). After that, LiOH·H₂O (18 mg, 0.4 mmol, 4.0 eq) was added at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, H₂O (10 mmol/L NH₄HCO₃+0.1% NH₃·H₂O) and CH₃CN (45% CH₃CN up to 85% in 10 min), Flow rate: 60 mL/min; Detector, 254/220 nm. Finally, 1-((1R,5S)-3-(7-(8-ethynyl-7-fluoro-3-hydroxynaphthalen-1-yl)-8-fluoro-2-(((S)-1-methylpiperidin-2-yl)methoxy)pyrido[4,3-d]pyrimidin-4-yl)-3,8-diazabicyclo[3.2.1]octan-8-yl)prop-2-en-1-one was obtained as a yellow solid (20 mg, 30.5%). LC-MS (ESI, m/z) M+1: 625. ¹HNMR (400 MHz, DMSO-d₆) δ 10.16 (s, 1H), 9.06 (s, 1H), 7.99 (dd, J=9.1, 5.8 Hz, 1H), 7.47 (t, J=9.0 Hz, 1H), 7.40 (d, J=2.7 Hz, 1H), 7.19 (s, 1H), 6.82 (dd, J=16.7, 10.3 Hz, 1H), 6.27 (d, J=16.6 Hz, 1H), 5.79 (d, J=10.6 Hz, 1H), 4.76 (s, 2H), 4.67 (d, J=13.6 Hz, 1H), 4.57-4.28 (m, 3H), 3.96 (s, 1H), 3.70 (d, J=12.7 Hz, 1H), 3.62 (d, J=12.8 Hz, 1H), 2.80 (d, J=10.1 Hz, 1H), 2.28 (s, 3H), 2.11-2.07 (m, 1H), 1.92-1.68 (m, 7H), 1.63-1.34 (m, 3H), 1.28-1.24 (m, 1H).

### Example 8: Preparation of N-[(3R)-1-[7-(8-ethynyl-7-fluoro-3-hydroxynaphthalen-1-yl)-8-fluoro-2-(hexahydropyrrolizin-7a-ylmethoxy)pyrido[4,3-d]pyrimidin-4-yl]azepan-3-yl]prop-2-enamide (assumed)

Synthesis of tert-butyl N-[(3R)-1-{8-fluoro-7-[7-fluoro-3-(methoxymethoxy)-8-[2-(triisopropylsilyl)ethynyl]naphthalen-1-yl]-2-(hexahydropyrrolizin-7a-ylmethoxy)pyrido[4,3-d]pyrimidin-4-yl}azepan-3-yl]carbamate: Into a 40-mL sealed-tube purged and maintained with an inert atmosphere of nitrogen, were placed tert-butyl N-[(3R)-1-[7-chloro-8-fluoro-2-(hexahydropyrrolizin-7a-ylmethoxy)pyrido[4,3-d]pyrimidin-4-yl]azepan-3-yl]carbamate (assumed) (250 mg, 0.5 mmol, 1.0 eq), {2-[2-fluoro-6-(methoxymethoxy)-8-(4,4,5,5-tetramethyl-1,3,2-dioxaborolan-2-yl)naphthalen-1-yl]ethynyl}triisopropylsilane (216 mg, 0.6 mmol, 1.2 eq), K₂CO₃ (194 mg, 1.4 mmol, 3.0 eq), DME (5 mL), H₂O (0.5 mL), cataCXium A Pd G3 (34 mg, 0.05 mmol, 0.1 eq). The resulting solution was stirred for 2 hours at 80° C. The resulting mixture was quenched by the addition of water (40 mL) and then extracted with ethyl acetate (2×40 mL). The combined organics were dried over Na₂SO₄, filtered and concentrated under vacuum. The crude residue was purified onto a silica gel column and eluted with dichloromethane/methanol=10:1 to give tert-butyl N-[(3R)-1-{8-fluoro-7-[7-fluoro-3-(methoxymethoxy)-8-[2-(triisopropylsilyl)ethynyl]naphthalen-1-yl]-2-(hexahydropyrrolizin-7a-ylmethoxy)pyrido[4,3-d]pyrimidin-4-yl}azepan-3-yl]carbamate (assumed) as a light yellow solid (180 mg, 43.5%). LC-MS (ESI, m/z) M+1: 885.

Synthesis of tert-butyl N-[(3R)-1-{7-[8-ethynyl-7-fluoro-3-(methoxymethoxy)naphthalen-1-yl]-8-fluoro-2-(hexahydropyrrolizin-7a-ylmethoxy)pyrido[4,3-d]pyrimidin-4-yl}azepan-3-yl]carbamate (assumed): Into a 40-mL sealed tube, were placed tert-butyl N-[(3R)-1-{8-fluoro-7-[7-fluoro-3-(methoxymethoxy)-8-[2-(triisopropylsilyl)ethynyl]naphthalen-1-yl]-2-(hexahydropyrrolizin-7a-ylmethoxy)pyrido[4,3-d]pyrimidin-4-yl}azepan-3-yl]carbamate (assumed) (150 mg, 0.2 mmol, 1.0 eq), DMF (3 mL), CsF (257 mg, 1.7 mmol, 10.0 eq). The resulting solution was stirred for 2 hours at 25° C. The resulting mixture was quenched by the addition of water (40 mL) and then extracted with ethyl acetate (2×40 mL). The combined organics were dried over anhydrous Na₂SO₄ filtered and concentrated under vacuum. Finally, tert-butyl N-[(3R)-1-{7-[8-ethynyl-7-fluoro-3-(methoxymethoxy)naphthalen-1-yl]-8-fluoro-2-(hexahydropyrrolizin-7a-ylmethoxy)pyrido[4,3-d]pyrimidin-4-yl}azepan-3-yl]carbamate (assumed) was obtained as a light yellow solid (140 mg, crude). LC-MS (ESI, m/z) M+1: 729.

Synthesis of 4-{4-[(3R)-3-aminoazepan-1-yl]-8-fluoro-2-(hexahydropyrrolizin-7a-ylmethoxy)pyrido[4,3-d]pyrimidin-7-yl}-5-ethynyl-6-fluoronaphthalen-2-ol (assumed): Into a 50 mL 3-necked round-bottom, were placed tert-butyl N-[(3R)-1-{7-[8-ethynyl-7-fluoro-3-(methoxymethoxy)naphthalen-1-yl]-8-fluoro-2-(hexahydropyrrolizin-7a-ylmethoxy)pyrido[4,3-d]pyrimidin-4-yl}azepan-3-yl]carbamate (assumed) (130 mg, 0.2 mmol, 1.0 eq), CH₂Cl₂ (4 mL). After that, HCl(gas) in 1,4-dioxane (2 mL, 4 M) was added at 0° C. Then the mixture was stirred for 1 hour at 0° C. The mixture neutralized to pH=7-8 with NH₃ in MeOH (2 M). The resulting mixture was then quenched by the addition of water (40 mL) and extracted with CH₂Cl₂ (2×40 mL). The combined organics were dried over anhydrous Na₂SO₄. After filtration, the filtrate was concentrated under vacuum to give 4-{4-[(3R)-3-aminoazepan-1-yl]-8-fluoro-2-(hexahydropyrrolizin-7a-ylmethoxy)pyrido[4,3-d]pyrimidin-7-yl}-5-ethynyl-6-fluoronaphthalen-2-ol (assumed) as light yellow solid (110 mg, crude).

Synthesis of N-[(3R)-1-[7-(8-ethynyl-7-fluoro-3-hydroxynaphthalen-1-yl)-8-fluoro-2-(hexahydropyrrolizin-7a-ylmethoxy)pyrido[4,3-d]pyrimidin-4-yl]azepan-3-yl]prop-2-enamide (assumed): Into a 50 mL 3-necked round-bottom flask, were placed 4-{4-[(3R)-3-aminoazepan-1-yl]-8-fluoro-2-(hexahydropyrrolizin-7a-ylmethoxy)pyrido[4,3-d]pyrimidin-7-yl}-5-ethynyl-6-fluoronaphthalen-2-ol (assumed) (80 mg, 0.1 mmol, 1.0 eq), DIEA (44 mg, 0.3 mmol, 2.5 eq), CH₂Cl₂ (3 mL). This was followed by the addition of acryloyl chloride (15 mg, 0.15 mmol, 1.2 eq) at 0° C. Then the mixture was stirred for 1 hour at 0° C. The resulting mixture was then quenched by the addition of water (30 mL). The mixture was extracted with dichloromethane (2×30 mL) and washed with brine (30 mL). The mixture was dried over anhydrous Na₂SO₄. After filtration, the filtrate was concentrated under vacuum. To the above mixture was added THE (1 mL), H₂O (1 mL), LiOH (13 mg, 0.5 mmol, 4.0 eq). The resulting mixture was stirred for additional 1 hour at 25° C. The crude product was purified by Prep-HPLC using the following conditions: Column, SunFire Prep C18 OBD Column, 50*250 mm Sum 10 nm; mobile phase, Water (0.05% NH₄HCO₃) and CH₃CN (35% Phase B up to 45% in 7 min); Detector, UV 254/220 nm. Finally, N-[(3R)-1-[7-(8-ethynyl-7-fluoro-3-hydroxynaphthalen-1-yl)-8-fluoro-2-(hexahydropyrrolizin-7a-ylmethoxy)pyrido[4,3-d]pyrimidin-4-yl]azepan-3-yl]prop-2-enamide (assumed) was obtained as a light yellow solid (20 mg, 22.9%). LC-MS (ESI, m/z) M+1: 639. ¹HNMR (400 MHz, DMSO-d₆) δ 10.17 (s, 1H), 9.20 (d, J=4.0 Hz, 1H), 8.27 (dd, J=13.0, 7.6 Hz, 1H), 7.98 (dd, J=9.2, 6.0 Hz, 1H), 7.47 (t, J=9.0 Hz, 1H), 7.39 (d, J=2.6 Hz, 1H), 7.18 (dd, J=15.2, 2.6 Hz, 1H), 6.28-6.20 (m, 1H), 6.13-6.08 (m, 1H), 5.63-5.58 (m, 1H), 4.56-4.22 (m, 3H), 4.09-3.83 (m, 5H), 2.92 (dt, J=10.4, 5.6 Hz, 2H), 2.56-2.54 (m, 1H), 2.06 (br, 1H), 1.95-1.70 (m, 9H), 1.60-1.50 (m, 4H).

### Example 9: Preparation of N-[(3S)-1-[7-(8-ethynyl-7-fluoro-3-hydroxynaphthalen-1-yl)-8-fluoro-2-(hexahydropyrrolizin-7a-ylmethoxy)pyrido[4,3-d]pyrimidin-4-yl]azepan-3-yl]prop-2-enamide (assumed)

Synthesis of tert-butyl N-[(3S)-1-{8-fluoro-7-[7-fluoro-3-(methoxymethoxy)-8-[2-(triisopropylsilyl)ethynyl]naphthalen-1-yl]-2-(hexahydropyrrolizin-7a-ylmethoxy)pyrido[4,3-d]pyrimidin-4-yl}azepan-3-yl]carbamate (assumed): Into a 40-mL sealed-tube purged and maintained with an inert atmosphere of nitrogen, were placed tert-butyl N-[(3S)-1-[7-chloro-8-fluoro-2-(hexahydropyrrolizin-7a-ylmethoxy)pyrido[4,3-d]pyrimidin-4-yl]azepan-3-yl]carbamate (assumed) (250 mg, 0.5 mmol, 1.0 eq), {2-[2-fluoro-6-(methoxymethoxy)-8-(4,4,5,5-tetramethyl-1,3,2-dioxaborolan-2-yl)naphthalen-1-yl]ethynyl}triisopropylsilane (216 mg, 0.6 mmol, 1.2 eq), K₂CO₃ (194 mg, 1.4 mmol, 3.0 eq), DME (5 mL), H₂O (0.5 mL), cataCXium A Pd G3 (34 mg, 0.05 mmol, 0.1 eq). The resulting solution was stirred for 2 hours at 80° C. The resulting mixture was quenched by the addition of water (40 mL) and then extracted with ethyl acetate (2×40 mL) The combined organic layers were dried over anhydrous Na₂SO₄. After filtration, the filtrate was concentrated under vacuum. The crude residue was purified onto a silica gel column and eluted with dichloromethane/methanol=10:1 to give tert-butyl N-[(3S)-1-{8-fluoro-7-[7-fluoro-3-(methoxymethoxy)-8-[2-(triisopropylsilyl)ethynyl]naphthalen-1-yl]-2-(hexahydropyrrolizin-7a-ylmethoxy)pyrido[4,3-d]pyrimidin-4-yl}azepan-3-yl]carbamate (assumed) as a light yellow solid (190 mg, 45.9%). LC-MS (ESI, m/z) M+1: 885.

Synthesis of tert-butyl N-[(3S)-1-{7-[8-ethynyl-7-fluoro-3-(methoxymethoxy)naphthalen-1-yl]-8-fluoro-2-(hexahydropyrrolizin-7a-ylmethoxy)pyrido[4,3-d]pyrimidin-4-yl}azepan-3-yl]carbamate (assumed): Into a 40-mL sealed tube, were placed tert-butyl N-[(3S)-1-{8-fluoro-7-[7-fluoro-3-(methoxymethoxy)-8-[2-(triisopropylsilyl)ethynyl]naphthalen-1-yl]-2-(hexahydropyrrolizin-7a-ylmethoxy)pyrido[4,3-d]pyrimidin-4-yl}azepan-3-yl]carbamate (assumed) (160 mg, 0.2 mmol, 1.0 eq), DMF (3 mL), CsF (274 mg, 1.8 mmol, 10.0 eq). The resulting solution was stirred for 2 hours at 25° C. The resulting mixture was then quenched by the addition of water (40 mL) and then extracted with ethyl acetate (2×40 mL). The combined organic layers were dried over anhydrous Na₂SO₄. After filtration, the filtrate was concentrated under vacuum to give tert-butyl N-[(3S)-1-{7-[8-ethynyl-7-fluoro-3-(methoxymethoxy)naphthalen-1-yl]-8-fluoro-2-(hexahydropyrrolizin-7a-ylmethoxy)pyrido[4,3-d]pyrimidin-4-yl}azepan-3-yl]carbamate (assumed) was obtained as a light yellow solid (150 mg, crude). LC-MS (ESI, m/z) M+1: 729.

Synthesis of 4-{4-[(3S)-3-aminoazepan-1-yl]-8-fluoro-2-(hexahydropyrrolizin-7a-ylmethoxy)pyrido[4,3-d]pyrimidin-7-yl}-5-ethynyl-6-fluoronaphthalen-2-ol (assumed): Into a 50 mL 3-necked round-bottom, were placed tert-butyl N-[(3S)-1-{7-[8-ethynyl-7-fluoro-3-(methoxymethoxy)naphthalen-1-yl]-8-fluoro-2-(hexahydropyrrolizin-7a-ylmethoxy)pyrido[4,3-d]pyrimidin-4-yl}azepan-3-yl]carbamate (assumed) (130 mg, 0.2 mmol, 1.0 eq), CH₂Cl₂ (4 mL). This was followed by the addition of HCl(gas) in 1,4-dioxane (2 mL, 4 M) at 0° C. Then the mixture was stirred for 1 hour at 0° C. The mixture was neutralized to pH=7-8 with NH₃ in MeOH (2 M). The resulting mixture was quenched by the addition of water (40 mL) and then extracted with CH₂Cl₂ (2×40 mL). The combined organic layers were dried over anhydrous Na₂SO₄. After filtration, the filtrate was concentrated under vacuum to give 4-{4-[(3S)-3-aminoazepan-1-yl]-8-fluoro-2-(hexahydropyrrolizin-7a-ylmethoxy)pyrido[4,3-d]pyrimidin-7-yl}-5-ethynyl-6-fluoronaphthalen-2-ol (assumed) as a light yellow solid (110 mg, crude).

Synthesis of N-[(3S)-1-[7-(8-ethynyl-7-fluoro-3-hydroxynaphthalen-1-yl)-8-fluoro-2-(hexahydropyrrolizin-7a-ylmethoxy)pyrido[4,3-d]pyrimidin-4-yl]azepan-3-yl]prop-2-enamide (assumed): Into a 50 mL 3-necked round-bottom flask, were placed 4-{4-[(3S)-3-aminoazepan-1-yl]-8-fluoro-2-(hexahydropyrrolizin-7a-ylmethoxy)pyrido[4,3-d]pyrimidin-7-yl}-5-ethynyl-6-fluoronaphthalen-2-ol (assumed) (80 mg, 0.1 mmol, 1.0 eq), DIEA (44 mg, 0.3 mmol, 2.5 eq), CH₂Cl₂ (3 mL). This was followed by the addition of acryloyl chloride (15 mg, 0.2 mmol, 1.2 eq) at 0° C. Then the mixture was stirred for 1 hour at 0° C. The resulting mixture was then quenched by the addition of water (30 mL) and then extracted with dichloromethane (2×30 mL). The combined organic layers were dried over anhydrous Na₂SO₄. After filtration, the filtrate was concentrated under vacuum. To the above mixture was added THE (1 mL), H₂O (1 mL) and LiOH (13 mg, 0.5 mmol, 4.0 eq). The resulting mixture was stirred for additional 1 hour at 25° C. The crude product was purified by Prep-HPLC using the following conditions: Column, SunFire Prep C18 OBD Column, 50*250 mm Sum 10 nm; mobile phase, Water (0.05% NH₄HCO₃) and CH₃CN (35% Phase B up to 45% in 7 min); Detector, UV 254/220 nm. Finally, N-[(3S)-1-[7-(8-ethynyl-7-fluoro-3-hydroxynaphthalen-1-yl)-8-fluoro-2-(hexahydropyrrolizin-7a-ylmethoxy)pyrido[4,3-d]pyrimidin-4-yl]azepan-3-yl]prop-2-enamide (assumed) was obtained as a light yellow solid (20 mg, 22.9%). LC-MS (ESI, m/z) M+1: 639. ¹HNMR (400 MHz, DMSO-d₆) δ 10.22 (s, 1H), 9.20 (d, J=4.0 Hz, 1H), 8.27 (dd, J=13.2, 7.6 Hz, 1H), 7.97 (dd, J=9.2, 6.0 Hz, 1H), 7.46 (t, J=9.0 Hz, 1H), 7.39 (d, J=2.4 Hz, 1H), 7.18 (dd, J=15.2, 2.6 Hz, 1H), 6.27-6.20 (m, 1H), 6.13-6.08 (m, 1H), 5.63-5.59 (m, 1H), 4.55-4.19 (m, 3H), 4.09-3.58 (m, 5H), 2.91 (dt, J=10.6, 5.6 Hz, 2H), 2.56-2.53 (m, 1H), 2.07 (br, 1H), 1.95-1.69 (m, 9H), 1.59-1.50 (m, 4H).

### Example 10: Preparation of N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)-N-methylacrylamide

Synthesis of tert-butyl (1-(((2,7-dichloro-8-fluoropyrido[4,3-d]pyrimidin-4-yl)amino)methyl)cyclobutyl)(methyl)carbamate: Into a 40 mL vial, were added 2,4,7-trichloro-8-fluoropyrido[4,3-d]pyrimidine (400 mg, 1.6 mmol, 1.0 eq), CH₂Cl₂ (10 mL) and DIEA (512 mg, 4.0 mmol, 2.5 eq) at −40° C. The resulting mixture was stirred for additional 15 min at −40° C. After that, tert-butyl (1-(aminomethyl)cyclobutyl)(methyl)carbamate (340 mg, 1.6 mmol, 1.0 eq) was added in portions over 5 min at −40° C. The resulting mixture was stirred for additional 1 hour at −40° C. The reaction mixture was diluted with water (10 mL) and extracted with CH₂Cl₂ (3×10 mL). The combined organic layers were washed with brine (10 mL), dried over anhydrous Na₂SO₄. After filtration, the filtrate was concentrated under reduced pressure. The crude residue was applied onto a silica gel column and eluted with petroleum ether/ethyl acetate=1:1 to give tert-butyl (1-(((2,7-dichloro-8-fluoropyrido[4,3-d]pyrimidin-4-yl)amino)methyl)cyclobutyl)(methyl)carbamate as a yellow solid (500 mg, 76.5%). LC-MS (ESI, m/z) M+1: 430/432.

Synthesis of tert-butyl (1-(((7-chloro-8-fluoro-2-((tetrahydro-1H-pyrrolizin-7a(5H)-yl)methoxy)pyrido[4,3-d]pyrimidin-4-yl)amino)methyl)cyclobutyl)(methyl)carbamate: Into a 40 mL vial, were placed (tetrahydro-1H-pyrrolizin-7a(5H)-yl)methanol (148 g, 1.0 mmol, 1.5 eq) and tetrahydrofuran (6 mL) at 25° C. After that, NaH (34 mg, 60% in mineral oil, 1.4 mmol, 2.0 eq) was added in portions over 5 min at 0° C. The resulting mixture was stirred for additional 30 min at 25° C. Next, tert-butyl (1-(((2,7-dichloro-8-fluoropyrido[4,3-d]pyrimidin-4-yl)amino)methyl)cyclobutyl)(methyl)carbamate (300 mg, 0.7 mmol, 1.0 eq) was added in portions over 5 min at 25° C. The resulting mixture was stirred for additional 4 hours at 25° C. The reaction was quenched with sat. NH₄Cl (20 mL) and then extracted with ethyl acetate (3×10 mL). The combined organic layers were washed with brine (10 mL), dried over anhydrous Na₂SO₄. After filtration, the filtrate was concentrated under reduced pressure. The crude residue was applied onto a silica gel column and eluted with dichloromethane/methanol=10:1 to give tert-butyl (1-(((7-chloro-8-fluoro-2-((tetrahydro-1H-pyrrolizin-7a(5H)-yl)methoxy)pyrido[4,3-d]pyrimidin-4-yl)amino)methyl)cyclobutyl)(methyl)carbamate as a white solid (210 mg, 56.2%). LC-MS (ESI, m/z) M+1: 535/537.

Synthesis of tert-butyl (1-(((8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)amino)methyl)cyclobutyl)(methyl)carbamate: Into a 40 mL vial, were placed tert-butyl (1-(((7-chloro-8-fluoro-2-((tetrahydro-1H-pyrrolizin-7a(5H)-yl)methoxy)pyrido[4,3-d]pyrimidin-4-yl)amino)methyl)cyclobutyl)(methyl)carbamate (200 mg, 0.4 mmol, 1.0 eq), ((2-fluoro-6-(methoxymethoxy)-8-(4,4,5,5-tetramethyl-1,3,2-dioxaborolan-2-yl)naphthalen-1-yl)ethynyl)triisopropylsilane (230 mg, 0.4 mmol, 1.2 eq), dimethoxyethane (5 mL), water (0.5 mL), K₃PO₄ (159 mg, 0.7 mmol, 2.0 eq) and CataCXium A Pd G3 (27 mg, 0.04 mmol, 0.1 eq) at 25° C. under nitrogen atmosphere. The resulting mixture was stirred for 4 hours at 80° C. The reaction mixture was diluted with water (5 mL) and extracted with CH₂Cl₂ (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1-(((8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)amino)methyl)cyclobutyl)(methyl)carbamate as a brown solid (180 mg, 54.4%). LC-MS (ESI, m/z) M+1: 885/887.

Synthesis of tert-butyl (1-(((7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)(methyl)carbamate: Into an 8 mL vial, were placed tert-butyl (1-(((8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)amino)methyl)cyclobutyl)(methyl)carbamate (170 mg, 0.2 mmol, 1.0 eq), CsF (292 mg, 1.9 mmol, 10.0 eq) and DMF (4 mL). The resulting mixture was stirred for 2 hours at 25° C. The reaction was then quenched by the addition of water (5 mL) and extracted with ethyl acetate (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1-(((7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)(methyl)carbamate as a brown solid (110 mg, 78.5%). LC-MS (ESI, m/z) M+1: 729/731.

Synthesis of 5-ethynyl-6-fluoro-4-(8-fluoro-4-(((1-(methylamino)cyclobutyl)methyl)amino)-2-((tetrahydro-1H-pyrrolizin-7a(5H)-yl)methoxy)pyrido[4,3-d]pyrimidin-7-yl)naphthalen-2-ol: Into an 8 mL vial, were placed tert-butyl (1-(((7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)(methyl)carbamate (100 mg, 0.1 mmol, 1.0 eq) and CH₂Cl₂ (2 mL). After that, HCl (gas) in 1,4-dioxane (0.5 mL) was added dropwise at 0° C. The resulting mixture was stirred for 1 hour at 0° C. The mixture was neutralized to pH=8 with NH₃/Methanol (2 M). The reaction mixture was diluted water (5 mL) and extracted with CH₂Cl₂ (4×5 mL). The combined organic layers were washed with brine (5 mL), dried over anhydrous Na₂SO₄. After filtration, the filtrate was concentrated under vacuum at 0° C. to give 5-ethynyl-6-fluoro-4-(8-fluoro-4-(((1-(methylamino)cyclobutyl)methyl)amino)-2-((tetrahydro-1H-pyrrolizin-7a(5H)-yl)methoxy)pyrido[4,3-d]pyrimidin-7-yl)naphthalen-2-ol as a brown solid (80 mg, crude). LC-MS (ESI, m/z) M+1: 585.

Synthesis of N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)-N-methylacrylamide: Into an 8 mL sealed tube, were placed 5-ethynyl-6-fluoro-4-(8-fluoro-4-(((1-(methylamino)cyclobutyl)methyl)amino)-2-((tetrahydro-1H-pyrrolizin-7a(5H)-yl)methoxy)pyrido[4,3-d]pyrimidin-7-yl)naphthalen-2-ol (70 mg, 0.1 mmol, 1.0 eq), CH₂Cl₂ (2 mL) and DIEA (39 mg, 0.3 mmol, 2.5 eq) at 25° C. After that, acryloyl chloride (9 mg, 0.1 mmol, 0.8 eq) was added dropwise at 0° C. The resulting mixture was stirred for 1 hour at 0° C. The reaction was quenched by the addition of water (4 mL) and extracted with CH₂Cl₂ (3×5 mL). The combined organic layers were washed with brine (5 mL), dried over anhydrous Na₂SO₄. After filtration, the filtrate was concentrated under reduced pressure. The residue was dissolved in tetrahydrofuran (1 mL) and water (1 mL). To the above mixture was added LiOH·H₂O (20 mg, 0.5 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, H₂O (0.1% FA) and CH₃CN (5% CH₃CN up to 100% in 10 min), Flow rate: 60 mL/min; Detector, 254/220 nm. Finally, N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclobutyl)-N-methylacrylamide was obtained as a yellow solid (15 mg, 17.0%). LC-MS (ESI, m/z) M+1: 639. ¹HNMR (300 MHz, DMSO-d₆) δ 10.59 (s, 1H), 9.36 (d, J=2.4 Hz, 1H), 7.99 (dd, J=9.0, 5.7 Hz, 1H), 7.57-6.96 (m, 4H), 6.77-6.33 (m, 1H), 6.07-5.83 (m, 1H), 5.72-5.29 (m, 1H), 4.55 (s, 2H), 4.10 (d, J=9.6 Hz, 2H), 3.95 (s, 1H), 3.55-3.45 (m, 1H), 3.22 (s, 2H), 2.81 (d, J=4.2 Hz, 3H), 2.42-1.55 (m, 15H).

### Example 11: Preparation of N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)-N-methylacrylamide

Synthesis of tert-butyl (1-(((2,7-dichloro-8-fluoropyrido[4,3-d]pyrimidin-4-yl)amino)methyl)cyclopentyl)(methyl)carbamate: Into a 40 mL vial, were placed 2,4,7-trichloro-8-fluoropyrido[4,3-d]pyrimidine (400 mg, 1.6 mmol, 1.0 eq) and CH₂Cl₂ (5 mL). After that, DIEA (512 mg, 4.0 mmol, 2.5 eq) was added dropwise at −40° C. The resulting mixture was stirred at −40° C. for 15 min. To the above mixture was added a solution of tert-butyl (1-(aminomethyl)cyclopentyl)(methyl)carbamate (362 mg, 1.6 mmol, 1.0 eq) in CH₂Cl₂ (5 mL) dropwise at −40° C. The resulting mixture was stirred for additional 1 hour at −40° C. The reaction was quenched by the addition of water (10 mL) and extracted with ethyl acetate (3×20 mL). The combined organic layers were washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (1-(((2,7-dichloro-8-fluoropyrido[4,3-d]pyrimidin-4-yl)amino)methyl)cyclopentyl)(methyl)carbamate as a white solid (500 mg, 71.0%). LC-MS (ESI, m/z) M+1: 444/446.

Synthesis of tert-butyl (1-(((7-chloro-8-fluoro-2-((tetrahydro-1H-pyrrolizin-7a(5H)-yl)methoxy)pyrido[4,3-d]pyrimidin-4-yl)amino)methyl)cyclopentyl)(methyl)carbamate: Into a 40 mL vial, were placed (tetrahydro-1H-pyrrolizin-7a(5H)-yl)methanol (143 g, 1.0 mmol, 1.5 eq) and tetrahydrofuran (6 mL) at 25° C. After that, NaH (32 mg, 60% in mineral oil, 1.4 mmol, 2.0 eq) was added in portions over 5 min at 0° C. The resulting mixture was stirred for additional 30 min at 25° C. To the above mixture was added tert-butyl (1-(((2,7-dichloro-8-fluoropyrido[4,3-d]pyrimidin-4-yl)amino)methyl)cyclopentyl)(methyl)carbamate (300 mg, 0.7 mmol, 1.0 eq) in portions over 5 min at 25° C. The resulting mixture was stirred for additional 4 hours at 25° C. The reaction was quenched with sat. NH₄Cl (20 mL) and then extracted with ethyl acetate (3×10 mL). The combined organic layers were washed with brine (10 mL), dried over anhydrous Na₂SO₄. After filtration, the filtrate was concentrated under reduced pressure. The crude residue was applied onto a silica gel column and eluted with dichloromethane/methanol=10:1 to give tert-butyl (1-(((7-chloro-8-fluoro-2-((tetrahydro-1H-pyrrolizin-7a(5H)-yl)methoxy)pyrido[4,3-d]pyrimidin-4-yl)amino)methyl)cyclopentyl)(methyl)carbamate as a white solid (220 mg, 59.2%). LC-MS (ESI, m/z) M+1: 549.

Synthesis of tert-butyl (1-(((8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)amino)methyl)cyclopentyl)(methyl)carbamate: Into a 40 mL vial, were placed tert-butyl (1-(((7-chloro-8-fluoro-2-((tetrahydro-1H-pyrrolizin-7a(5H)-yl)methoxy)pyrido[4,3-d]pyrimidin-4-yl)amino)methyl)cyclopentyl)(methyl)carbamate (200 mg, 0.4 mmol, 1.0 eq), ((2-fluoro-6-(methoxymethoxy)-8-(4,4,5,5-tetramethyl-1,3,2-dioxaborolan-2-yl)naphthalen-1-yl)ethynyl)triisopropylsilane (224 mg, 0.4 mmol, 1.2 eq), dimethoxyethane (5 mL), water (0.5 mL), K₂CO₃ (101 mg, 0.7 mmol, 2.0 eq) and CataCXium A Pd G3 (27 mg, 0.04 mmol, 0.1 eq) at 25° C. under nitrogen atmosphere. The resulting mixture was stirred for 4 hours at 80° C. under nitrogen atmosphere. The reaction mixture was diluted with water (5 mL) and extracted with CH₂Cl₂ (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1-(((8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)amino)methyl)cyclopentyl)(methyl)carbamate as a brown solid (160 mg, 48.8%). LC-MS (ESI, m/z) M+1: 899.

Synthesis of tert-butyl (1-(((7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)(methyl)carbamate: Into an 8 mL vial, were placed tert-butyl (1-(((8-fluoro-7-(7-fluoro-3-(methoxymethoxy)-8-((triisopropylsilyl)ethynyl)naphthalen-1-yl)-2-((tetrahydro-1H-pyrrolizin-7a(5H)-yl)methoxy)pyrido[4,3-d]pyrimidin-4-yl)amino)methyl)cyclopentyl)(methyl)carbamate (150 mg, 0.2 mmol, 1.0 eq), CsF (253 mg, 1.7 mmol, 10.0 eq) and DMF (2 mL). The resulting mixture was stirred for 2 hours at 25° C. The resulting mixture was then quenched by the addition of water (5 mL) and extracted with ethyl acetate (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1-(((7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)(methyl)carbamate as a brown solid (100 mg, 80.7%). LC-MS (ESI, m/z) M+1: 743/745.

Synthesis of 5-ethynyl-6-fluoro-4-(8-fluoro-4-(((1-(methylamino)cyclopentyl)methyl)amino)-2-((tetrahydro-1H-pyrrolizin-7a(5H)-yl)methoxy)pyrido[4,3-d]pyrimidin-7-yl)naphthalen-2-ol: Into an 8 mL vial, were placed tert-butyl (1-(((7-(8-ethynyl-7-fluoro-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)(methyl)carbamate (100 mg, 0.1 mmol, 1.0 eq) and CH₂Cl₂ (2 mL). After that, HCl (gas) in 1,4-dioxane (0.5 mL) was added dropwise at 0° C. The resulting mixture was stirred for 1 hour at 0° C. The mixture was neutralized to pH=8 with NH₃/Methanol (2 M). The resulting mixture was diluted water (5 mL), and extracted with CH₂Cl₂ (2×5 mL). The combined organic layers were washed with brine (5 mL), dried over anhydrous Na₂SO₄. After filtration, the filtrate was concentrated under vacuum at 0° C. to give 5-ethynyl-6-fluoro-4-(8-fluoro-4-(((1-(methylamino)cyclopentyl)methyl)amino)-2-((tetrahydro-1H-pyrrolizin-7a(5H)-yl)methoxy)pyrido[4,3-d]pyrimidin-7-yl)naphthalen-2-ol as a brown solid (60 mg, crude). LC-MS (ESI, m/z) M+1: 599.

Synthesis of N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)-N-methylacrylamide: Into an 8 mL sealed tube, were placed 5-ethynyl-6-fluoro-4-(8-fluoro-4-(((1-(methylamino)cyclopentyl)methyl)amino)-2-((tetrahydro-1H-pyrrolizin-7a(5H)-yl)methoxy)pyrido[4,3-d]pyrimidin-7-yl)naphthalen-2-ol (50 mg, 0.08 mmol, 1.0 eq), CH₂Cl₂ (2 mL) and DIEA (27 mg, 0.2 mmol, 2.5 eq) at 25° C. After that, acryloyl chloride (6 mg, 0.07 mmol, 0.8 eq) was added dropwise at 0° C. The resulting mixture was stirred for 1 hour at 0° C. The reaction was quenched by the addition of water (4 mL) and then extracted with CH₂Cl₂ (3×5 mL). The combined organic layers were washed with brine (5 mL), dried over anhydrous Na₂SO₄. After filtration, the filtrate was concentrated under reduced pressure. The residue was dissolved in tetrahydrofuran (1 mL) and H₂O (1 mL). To the above mixture was added LiOH H₂O (14 mg, 0.3 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, water (0.1% FA) and CH₃CN (5% CH₃CN up to 100% in 10 min), Flow rate: 60 mL/min; Detector, 254/220 nm. Finally, N-(1-(((7-(8-ethynyl-7-fluoro-3-hydroxynaphthalen-1-yl)-8-fluoro-2-((tetrahydro-1H-pyrrolizin-7a(5H)-yl)methoxy)pyrido[4,3-d]pyrimidin-4-yl)amino)methyl)cyclopentyl)-N-methylacrylamide was obtained as a yellow solid (15 mg, 27.5%). LC-MS (ESI, m/z) M+1: 653. ¹HNMR (300 MHz, DMSO-d₆) δ 9.23 (s, 1H), 9.08 (s, 1H), 8.26 (s, 1H), 7.98 (dd, J=9.0, 6.0 Hz, 1H), 7.59-7.30 (m, 2H), 7.18 (d, J=2.4 Hz, 1H), 6.63 (dd, J=9.3, 6.6 Hz, 1H), 5.94 (dd, J=10.2, 3.6 Hz, 1H), 5.58 (dd, J=10.2, 2.4 Hz, 1H), 4.09 (s, 2H), 3.96 (d, J=9.6 Hz, 3H), 3.01 (d, J=8.1 Hz, 5H), 2.65 (t, J=7.2 Hz, 2H), 2.28 (s, 2H), 2.03-1.44 (m, 14H).

### Example 12: Preparation of 1-((1R,5S)-3-(2-(((S)-1,2-dimethylpyrrolidin-2-yl)methoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one

Synthesis of (S)-(1,2-dimethylpyrrolidin-2-yl)methanol: Into a 250 mL 3-necked round-bottom flask, were added (S)-1-(tert-butoxycarbonyl)-2-methylpyrrolidine-2-carboxylic acid (10 g, 43.6 mmol, 1.0 eq) and tetrahydrofuran (150 mL) at 25° C. After that, LiAlH₄ (5.0 g, 130.8 mmol, 3.0 eq) was added in portions over 10 min at 0° C. The resulting mixture was stirred for additional 4 hours at 25° C. The reaction was quenched by the addition of water (5 mL), 10% NaOH (5 mL) and water (15 mL) in this order at 0° C. The resulting mixture was filtered, the filter cake was washed with tetrahydrofuran (3×100 mL). The filtrate was concentrated under vacuum. The crude product was purified by distillation under vacuum and the fraction was collected at 80° C. to give (S)-(1,2-dimethylpyrrolidin-2-yl)methanol as a colorless oil (550 mg, 9.7%). ¹HNMR (400 MHz, Chloroform-d) δ 3.32 (d, J=10.2 Hz, 1H), 3.25 (d, J=9.8 Hz, 1H), 3.05 (dt, J=9.2, 5.2 Hz, 1H), 2.57 (q, J=8.7 Hz, 1H), 2.21 (s, 3H), 2.11-1.97 (m, 1H), 1.72 (qd, J=8.2, 5.2 Hz, 2H), 1.58-1.41 (m, 1H), 0.87 (s, 3H).

Synthesis of tert-butyl (1R,5S)-3-(7-chloro-2-(((S)-1,2-dimethylpyrrolidin-2-yl)methoxy)-8-fluoropyrido[4,3-d]pyrimidin-4-yl)-3,8-diazabicyclo[3.2.1]octane-8-carboxylate: Into a 40 mL vial, were placed (S)-(1,2-dimethylpyrrolidin-2-yl)methanol (181 mg, 1.4 mmol, 1.5 eq) and tetrahydrofuran (8 mL) at 25° C. After that, NaH (45 mg, 1.9 mmol, 2.0 eq) was added in portions over 5 min at 0° C. The resulting mixture was stirred for additional 30 min at 25° C. To the above mixture was added tert-butyl (1R,5S)-3-(2,7-dichloro-8-fluoropyrido[4,3-d]pyrimidin-4-yl)-3,8-diazabicyclo[3.2.1]octane-8-carboxylate (400 mg, 0.9 mmol, 1.0 eq) in portions over 5 min at 25° C. The resulting mixture was stirred for additional 3 hours at 25° C. The reaction was quenched by the addition of sat. NH₄Cl (20 mL) and then extracted with ethyl acetate (3×10 mL). The combined organic layers were washed with brine (10 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(7-chloro-2-(((S)-1,2-dimethylpyrrolidin-2-yl)methoxy)-8-fluoropyrido[4,3-d]pyrimidin-4-yl)-3,8-diazabicyclo[3.2.1]octane-8-carboxylate as a white solid (330 mg, 67.7%). LC-MS (ESI, m/z) M+1: 521.

Synthesis of tert-butyl (1R,5S)-3-(2-(((S)-1,2-dimethylpyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate: Into a 40 mL vial, were added tert-butyl (1R,5S)-3-(7-chloro-2-(((S)-1,2-dimethylpyrrolidin-2-yl)methoxy)-8-fluoropyrido[4,3-d]pyrimidin-4-yl)-3,8-diazabicyclo[3.2.1]octane-8-carboxylate (250 mg, 0.5 mmol, 1.0 eq), ((2-fluoro-6-(methoxymethoxy)-8-(4,4,5,5-tetramethyl-1,3,2-dioxaborolan-2-yl)naphthalen-1-yl)ethynyl)triisopropylsilane (295 mg, 0.6 mmol, 1.2 eq), K₃PO₄ (204 mg, 1.0 mmol, 2.0 eq), dimethoxyethane (10 mL), water (1 mL) and CataCXium A Pd G3 (35 mg, 0.05 mmol, 0.1 eq) at 25° C. The resulting mixture was stirred for 4 hours at 80° C. under nitrogen atmosphere. The reaction mixture was diluted with water (10 mL) and extracted with CH₂Cl₂ (4×10 mL). The combined organic layers were washed with brine (10 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(2-(((S)-1,2-dimethylpyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate as a brown solid (260 mg, 62.2%). LC-MS (ESI, m/z) M+1: 871.

Synthesis of tert-butyl (1R,5S)-3-(2-(((S)-1,2-dimethylpyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate: Into a 40 mL vial, were placed tert-butyl (1R,5S)-3-(2-(((S)-1,2-dimethylpyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate (250 mg, 0.3 mmol, 1.0 eq), CsF (436 mg, 3.0 mmol, 10.0 eq) and DMF (5 mL). The resulting mixture was stirred for 2 hours at 25° C. The resulting mixture was then quenched by the addition of water (5 mL) and extracted with ethyl acetate (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(2-(((S)-1,2-dimethylpyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate as a brown solid (180 mg, 87.7%). LC-MS (ESI, m/z) M+1: 715.

Synthesis of 4-(4-((1R,5S)-3,8-diazabicyclo[3.2.1]octan-3-yl)-2-(((S)-1,2-dimethylpyrrolidin-2-yl)methoxy)-8-fluoropyrido[4,3-d]pyrimidin-7-yl)-5-ethynyl-6-fluoronaphthalen-2-ol: Into an 8 mL sealed tube, were placed tert-butyl (1R,5S)-3-(2-(((S)-1,2-dimethylpyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (170 mg, 0.2 mmol, 1 eq) and CH₂Cl₂ (2 mL). After that, HCl (gas) in 1,4-dioxane (0.5 mL) was added dropwise at 0° C. The resulting mixture was stirred for 1 hour at 0° C. The reaction mixture was neutralized to pH=8 with NH₃/Methanol (2 M). The resulting mixture was diluted water (5 mL) and extracted with CH₂Cl₂ (4×5 mL). The combined organic layers were washed with brine (5 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-2-(((S)-1,2-dimethylpyrrolidin-2-yl)methoxy)-8-fluoropyrido[4,3-d]pyrimidin-7-yl)-5-ethynyl-6-fluoronaphthalen-2-ol as a white solid (170 mg, crude). LC-MS (ESI, m/z) M+1: 571.

Synthesis of 1-((1R,5S)-3-(2-(((S)-1,2-dimethylpyrrolidin-2-yl)methoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one: Into an 8 mL sealed tube, were placed 4-(4-((1R,5S)-3,8-diazabicyclo[3.2.1]octan-3-yl)-2-(((S)-1,2-dimethylpyrrolidin-2-yl)methoxy)-8-fluoropyrido[4,3-d]pyrimidin-7-yl)-5-ethynyl-6-fluoronaphthalen-2-ol (170 mg, 0.3 mmol, 1.0 eq), CH₂Cl₂ (4 mL) and DIEA (96 mg, 0.7 mmol, 2.5 eq) at 25° C. After that, acryloyl chloride (22 mg, 0.2 mmol, 0.8 eq) was added dropwise at 0° C. The resulting mixture was stirred for 1 hour at 0° C. The reaction was quenched by the addition of water (4 mL) and then extracted with CH₂Cl₂ (3×5 mL). The combined organic layers were washed with brine (5 mL), dried over anhydrous Na₂SO₄. After filtration, the filtrate was concentrated under vacuum. The residue was dissolved in tetrahydrofuran (2 mL) and water (2 mL). To the above mixture was added LiOH H₂O (50.0 mg, 1.2 mmol, 4.0 eq) at 25° C. The resulting mixture was stirred for additional 1 hour at 25° C. The reaction mixture was concentrated under vacuum at 0° C. The crude product was purified by Prep-HPLC using the following conditions: Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min), Flow rate: 60 mL/min; Detector, 254/220 nm. Finally, 1-((1R,5S)-3-(2-(((S)-1,2-dimethylpyrrolidin-2-yl)methoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl)prop-2-en-1-one was obtained as a yellow solid (13 mg, 6.9%). LC-MS (ESI, m/z) M+1: 625. ¹HNMR (300 MHz, Chloroform-d) δ 8.88 (d, J=11.8 Hz, 1H), 7.62 (t, J=4.5 Hz, 1H), 7.25-7.11 (m, 3H), 6.55-6.39 (m, 2H), 5.80 (dd, J=8.1, 3.9 Hz, 1H), 4.89 (s, 1H), 4.56 (m, 2H), 4.33 (m, 3H), 3.88-3.62 (m, 1H), 3.53 (m, 1H), 3.07 (s, 1H), 2.76 (d, J=7.2 Hz, 1H), 2.67 (t, J=8.4 Hz, 1H), 2.43 (m, 3H), 2.20-2.01 (m, 1H), 2.02-1.54 (m, 8H), 1.17 (s, 3H).

### Example 13: Preparation of 1-((1R,5S)-3-(7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octan-8-yl)prop-2-en-1-one

Synthesis of tert-butyl (1R,5S)-3-(8-fluoro-2-(((2S,4R)-4-fluoro-1,2-dimethylpyrrolidin-2-yl)methoxy)-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate: Into an 8 mL vial, were placed tert-butyl (1R,5S)-3-(7-chloro-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate (160 mg, 0.3 mmol, 1.0 eq), ((2-fluoro-6-(methoxymethoxy)-8-(4,4,5,5-tetramethyl-1,3,2-dioxaborolan-2-yl)naphthalen-1-yl)ethynyl)triisopropylsilane (183 mg, 0.4 mmol, 1.2 eq), dimethoxyethane (4 mL), water (0.4 mL), K₂CO₃ (82 mg, 0.6 mmol, 2.0 eq) and CataCXium A Pd G₃ (22 mg, 0.03 mmol, 0.1 eq) at 25° C. The resulting mixture was stirred for 16 hours at 80° C. under nitrogen atmosphere. The resulting mixture was diluted with water (5 mL) and extracted with CH₂Cl₂ (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(8-fluoro-2-(((2S,4R)-4-fluoro-1,2-dimethylpyrrolidin-2-yl)methoxy)-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate as a brown solid (100 mg, 37.8%). LC-MS (ESI, m/z) M+1: 889/891.

Synthesis of tert-butyl (1R,5S)-3-(7-(8-ethynyl-7-fluoro-3-(methoxymethoxy)naphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate: Into an 8 mL vial, were placed tert-butyl (1R,5S)-3-(8-fluoro-2-(((2S,4R)-4-fluoro-1,2-dimethylpyrrolidin-2-yl)methoxy)-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate (90 mg, 0.1 mmol, 1.0 eq), CsF (154 mg, 1.0 mmol, 10.0 eq) and DMF (2 mL). The resulting mixture was stirred for 2 hours at 25° C. The resulting mixture was quenched by the addition of water (5 mL) and extracted with ethyl acetate (4×5 mL). The combined organic layers were washed with brine (5 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(7-(8-ethynyl-7-fluoro-3-(methoxymethoxy)naphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate as a brown solid (60 mg, 80.8%). LC-MS (ESI, m/z) M+1: 733/735.

Synthesis of 4-(4-((1R,5S)-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-ol: Into an 8 mL vial, were placed tert-butyl (1R,5S)-3-(7-(8-ethynyl-7-fluoro-3-(methoxymethoxy)naphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate (55 mg, 0.08 mmol, 1.0 eq) and CH₂Cl₂ (2 mL). After that, HCl (gas) in 1,4-dioxane (0.2 mL) was added dropwise at 0° C. The resulting mixture was stirred for 1 hour at 0° C. The mixture was neutralized to pH=8 with NH₃/Methanol (2 M). The resulting mixture was diluted water (5 mL) and extracted with CH₂Cl₂ (4×5 mL). The combined organic layers were washed with brine (5 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-ol as a yellow solid (60 mg, crude). LC-MS (ESI, m/z) M+1: 589.

Synthesis of 1-((1R,5S)-3-(7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octan-8-yl)prop-2-en-1-one: Into an 8 mL sealed tube, were placed 4-(4-((1R,5S)-3,8-diazabicyclo[3.2.1]octan-3-yl)-8-fluoro-2-(((2S,4R)-4-fluoro-1,2-dimethylpyrrolidin-2-yl)methoxy)pyrido[4,3-d]pyrimidin-7-yl)-5-ethynyl-6-fluoronaphthalen-2-ol (50 mg, 0.1 mmol, 1.0 eq), CH₂Cl₂ (2 mL) and DIEA (27 mg, 0.2 mmol, 2.5 eq) at 25° C. After that, acryloyl chloride (6 mg, 0.07 mmol, 0.8 eq) was added dropwise at 0° C. The resulting mixture was stirred for 1 hour at 0° C. The reaction was quenched by the addition of water (4 mL) and extracted with CH₂Cl₂ (3×5 mL). The combined organic layers were washed with brine (5 mL), dried over anhydrous Na₂SO₄. After filtration, the filtrate was concentrated under vacuum. The residue was dissolved in tetrahydrofuran (1 mL) and water (1 mL). To the above mixture was added LiOH H₂O (14 mg, 0.3 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, water (0.1% FA) and CH₃CN (5% CH₃CN up to 100% in 10 min), Flow rate: 60 mL/min; Detector, 254/220 nm. Finally, 1-((1R,5S)-3-(7-(8-ethynyl-7-fluoro-3-hydroxynaphthalen-1-yl)-8-fluoro-2-(((2S,4R)-4-fluoro-1,2-dimethylpyrrolidin-2-yl)methoxy)pyrido[4,3-d]pyrimidin-4-yl)-3,8-diazabicyclo[3.2.1]octan-8-yl)prop-2-en-1-one was obtained as a yellow solid (5 mg, 9.1%). LC-MS (ESI, m/z) M+1: 643. ¹HNMR (300 MHz, Chloroform-d) δ 8.91 (s, 1H), 8.33 (s, 1H), 7.62 (dd, J=9.0, 5.7 Hz, 1H), 7.24-7.02 (m, 3H), 6.48 (s, 2H), 5.82 (s, 1H), 5.30 (d, J=6.3 Hz, 1H), 4.92 (s, 1H), 4.72-4.45 (m, 4H), 3.77 (s, 2H), 3.55 (t, J=11.7 Hz, 1H), 3.08 (dd, J=7.2, 5.1 Hz, 1H), 2.79 (d, J=8.4 Hz, 1H), 2.58 (d, J=10.2 Hz, 4H), 2.30-1.56 (m, 6H), 1.39 (d, J=9.0 Hz, 3H).

### Example 14: Preparation of 1-((1R,5S)-3-(7-(8-ethynyl-7-fluoro-3-hydroxynaphthalen-1-yl)-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octan-8-yl)prop-2-en-1-one

Synthesis of tert-butyl (1R,5S)-3-(8-fluoro-2-(((2R,4R)-4-fluoro-1,2-dimethylpyrrolidin-2-yl)methoxy)-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate: Into a 40 mL vial, were placed tert-butyl (1R,5S)-3-(7-chloro-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate (300 mg, 0.6 mmol, 1 eq), {2-[2-fluoro-6-(methoxymethoxy)-8-(4,4,5,5-tetramethyl-1,3,2-dioxaborolan-2-yl)naphthalen-1-yl]ethynyl}triisopropylsilane (342 mg, 0.7 mmol, 1.2 eq), dimethoxyethane (10 mL), water (1 mL), K₃PO₄ (236 mg, 1.1 mmol, 2.0 eq) and CataCXium A Pd G₃ (41 mg, 0.06 mmol, 0.1 eq) at 25° C. The resulting mixture was stirred for 16 hours at 80° C. under nitrogen atmosphere. The resulting mixture was diluted with water (10 mL) and extracted with CH₂Cl₂ (4×10 mL). The combined organic layers were washed with brine (10 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(8-fluoro-2-(((2R,4R)-4-fluoro-1,2-dimethylpyrrolidin-2-yl)methoxy)-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate as a brown solid (400 mg, 80.8%). LC-MS (ESI, m/z) M+1: 889/891.

Synthesis of tert-butyl (1R,5S)-3-(7-(8-ethynyl-7-fluoro-3-(methoxymethoxy)naphthalen-1-yl)-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate: Into a 40 mL vial, were placed tert-butyl (1R,5S)-3-(8-fluoro-2-(((2R,4R)-4-fluoro-1,2-dimethylpyrrolidin-2-yl)methoxy)-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate (300 mg, 0.3 mmol, 1.0 eq), CsF (513 mg, 3.3 mmol, 10.0 eq) and DMF (6 mL). The resulting mixture was stirred for 2 hours at 25° C. The resulting mixture was then quenched by the addition of water (6 mL) and extracted with ethyl acetate (4×6 mL). The combined organic layers were washed with brine (6 mL), dried over anhydrous Na₂SO₄. After filtration, the filtrate was concentrated under vacuum. The crude residue was applied onto a silica gel column and eluted with dichloromethane/methanol=10:1 to give tert-butyl (1R,5S)-3-(7-(8-ethynyl-7-fluoro-3-(methoxymethoxy)naphthalen-1-yl)-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate as a brown solid (180 mg, 72.8%). LC-MS (ESI, m/z) M+1: 733/735.

Synthesis of 4-(4-((1R,5S)-3,8-diazabicyclo[3.2.1]octan-3-yl)-8-fluoro-2-(((2R,4R)-4-fluoro-1,2-dimethylpyrrolidin-2-yl)methoxy)pyrido[4,3-d]pyrimidin-7-yl)-5-ethynyl-6-fluoronaphthalen-2-ol: Into an 8 mL vial, were placed tert-butyl (1R,5S)-3-(7-(8-ethynyl-7-fluoro-3-(methoxymethoxy)naphthalen-1-yl)-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octane-8-carboxylate (170 mg, 0.2 mmol, 1 eq) and CH₂Cl₂ (2 mL). After that, HCl (gas) in 1,4-dioxane (0.5 mL) was added dropwise at 0° C. The resulting mixture was stirred for 1 hour at 0° C. The mixture was neutralized to pH=8 with NH₃/Methanol (2 M). The resulting mixture was diluted water (5 mL) and extracted with CH₂Cl₂ (4×5 mL). The combined organic layers were washed with brine (5 mL), dried over anhydrous Na₂SO₄. After filtration, the filtrate was concentrated under vacuum at 0° C. to give 4-(4-((1R,5S)-3,8-diazabicyclo[3.2.1]octan-3-yl)-8-fluoro-2-(((2R,4R)-4-fluoro-1,2-dimethylpyrrolidin-2-yl)methoxy)pyrido[4,3-d]pyrimidin-7-yl)-5-ethynyl-6-fluoronaphthalen-2-ol as a yellow solid (150 mg, crude). LC-MS (ESI, m/z) M+1: 589.

Synthesis of 1-((1R,5S)-3-(7-(8-ethynyl-7-fluoro-3-hydroxynaphthalen-1-yl)-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octan-8-yl)prop-2-en-1-one: Into an 8 mL vial, were placed 4-(4-((1R,5S)-3,8-diazabicyclo[3.2.1]octan-3-yl)-8-fluoro-2-(((2R,4R)-4-fluoro-1,2-dimethylpyrrolidin-2-yl)methoxy)pyrido[4,3-d]pyrimidin-7-yl)-5-ethynyl-6-fluoronaphthalen-2-ol (140 mg, 0.2 mmol, 1.0 eq), CH₂Cl₂ (2 mL) and DIEA (77 mg, 0.6 mmol, 2.5 eq) at 25° C. After that, acryloyl chloride (17 mg, 0.2 mmol, 0.8 eq) was added dropwise at 0° C. The resulting mixture was stirred for 1 hour at 0° C. The reaction was quenched by the addition of water (4 mL) and extracted with CH₂Cl₂ (3×5 mL). The combined organic layers were washed with brine (5 mL), dried over anhydrous Na₂SO₄. After filtration, the filtrate was concentrated under vacuum. The residue was dissolved in tetrahydrofuran (1 mL) and water (1 mL). To the above mixture was added LiOH·H₂O (40 mg, 1.0 mmol, 4.0 eq) at 25° C. The resulting mixture was stirred for additional 1 hour at 25° C. The resulting mixture was concentrated under vacuum at 0° C. The crude product was purified by Prep-HPLC using the following conditions: Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min), Flow rate: 60 mL/min; Detector, 254/220 nm. Finally, 1-((1R,5S)-3-(7-(8-ethynyl-7-fluoro-3-hydroxynaphthalen-1-yl)-8-fluoro-2-(((2R,4R)-4-fluoro-1,2-dimethylpyrrolidin-2-yl)methoxy)pyrido[4,3-d]pyrimidin-4-yl)-3,8-diazabicyclo[3.2.1]octan-8-yl)prop-2-en-1-one was obtained as a yellow solid (35 mg, 19.9%). LC-MS (ESI, m/z) M+1: 643. ¹HNMR (300 MHz, Chloroform-d) δ 8.92 (d, J=9.3 Hz, 1H), 7.72-7.47 (m, 1H), 7.24-7.02 (m, 3H), 6.60-6.35 (m, 2H), 5.91-5.61 (m, 1H), 5.15 (d, J=4.5 Hz, 1H), 4.92 (s, 1H), 4.80-4.16 (m, 5H), 3.77 (t, J=5.4 Hz, 1H), 3.53 (s, 1H), 3.27 (dd, J=11.7, 7.5 Hz, 1H), 2.95 (dd, J=12.0, 10.2 Hz, 1H), 2.78 (s, 1H), 2.60-2.27 (m, 5H), 2.24-1.52 (m, 5H), 1.19 (s, 3H).

### Biological Example 1: KRas G12C Binding Assay

HIS-KRAS (G12C, aa 2-185, Sino biological) was diluted to 5 uM in EDTA buffer (20 mM HEPES, pH 7.4, 50 mM NaCl, 10 mM EDTA, 0.01% (v/v) Tween-20) and incubated for 30 min at 25° C. The EDTA pretreated HIS-KRAS (G12C) was diluted to 12 nM in assay buffer (25 mM HEPES, pH 7.4, 120 mM NaCl, 5 mM MgCl 2, 1 mM DTT, 0.01% (v/v) Tween 20, 0.1% (w/v) BSA) containing 120 nM GDP (Sigma) and MAb Anti 6HIS—Tb cryptate Gold (Cisbio) and incubated for 1 hour at 25° C. to prepare GDP-loaded HIS-KRAS (G12C). The GDP-loaded HIS-KRAS (G12C) was pre-incubation with diluted compounds in a 384-well plate (Greiner) for 1 hour, then purified SOS1 ExD (Flag tag, aa 564-1049) and BODIPY™ FL GTP (Invitrogen) were added to the assay wells (Final concentration: 3 nM HIS-KRAS (G12C), 2 uM SOS1 ExD, 80 nM BODIPY™ FL GTP, 21 ng/mL MAb Anti 6HIS—Tb cryptate Gold) and incubated for 4 hours at 25° C. TR-FRET signals were then read on Tecan Spark multimode microplate reader. The parameters were F486: Excitation 340 (35) nm, Emission 486 (10) nm, Lag time 100 μs, Integration time 200 μs; F515: Excitation 340 (35) nm, Emission 515 (10) nm, Lag time 100 μs, Integration time 200 μs. TR-FRET ratios for each individual wells were calculated by equation: TR-FRET ratio=(Signal F515/Signal F486)*10000. Then the data were analyzed using a 4-parameter logistic model to calculate IC₅₀ values. The following table lists the IC₅₀ values of certain compounds of the invention.

| Example | Kras G12C(nM) |
|---|---|
| 1 | 10.56 |
| 2 | 0.41 |
| 3 | 3.19 |
| 4 | 3.34 |
| 5 | 0.37 |
| 6 | 0.36 |
| 7 | 0.41 |
| 8 | 7.11 |
| 9 | 6.40 |
| 10 | 2.7 |
| 11 | 1.6 |
| 12 | 1.5 |
| 13 | 0.96 |
| 14 | 0.99 |

### Biological Example 2: KRas G12C In Vitro Covalent MS-Based Assay

GDP-loaded, histidine-tagged, truncated (1-169) KRAS proteins (G12C, WT, G13D, as indicated) at 2 uM final concentration were incubated with the test compounds at the doses and time points indicated in a buffer containing 20 mmol/L HEPES pH 7.5, 150 mmol/L NaCl, 1 mmol/L MgCl 2, and 1 mmol/L DTT. Reactions were quenched by adding formic acid to 0.2%. Samples were buffer exchanged and analyzed by denaturing SEC-MS (Orbitrap Eclipse), Mobile phase: 30% ACN, 0.1% formic acid, 0.02% TFA. Raw data were analyzed by charge state deconvolution with BioPharma Finder 4.1 Intact Mass, ReSpect and Sliding Window algorithms. The masses were calculated via software for KRAS_G12C theoretical sequence and experimental compounds. The follow table shows the result.

| Example | Binding Model |
|---|---|
| 2 | Covalent binding |
| 5 | Covalent binding |
| 8 | Covalent binding |
| 9 | Covalent binding |

### Biological Example 3: KRas G12V Binding Assay

HIS-KRAS (G12V) was diluted to 5 uM in EDTA buffer (20 mM HEPES, pH 7.4, 50 mM NaCl, 10 mM EDTA, 0.01% (v/v) Tween-20) and incubated for 30 min at 25° C. The EDTA pretreated HIS-KRAS (G12V) was diluted to 12 nM in assay buffer (25 mM HEPES, pH 7.4, 120 mM NaCl, 5 mM MgCl 2, 1 mM DTT, 0.01% (v/v) Tween 20, 0.1% (w/v) BSA) containing 120 nM GDP (Sigma) and MAb Anti 6HIS—Tb cryptate Gold (Cisbio) and incubated for 1 hour at 25° C. to prepare GDP-loaded HIS-KRAS (G12V). The GDP-loaded HIS-KRAS (G12V) was pre-incubation with diluted compounds in a 384-well plate (Greiner) for 1 hour, then purified SOS1 ExD (Flag tag, aa 564-1049) and BODIPY™ FL GTP (Invitrogen) were added to the assay wells (Final concentration: 3 nM HIS-KRAS (G12V), 2 uM SOS1 ExD, 80 nM BODIPY™ FL GTP, 21 ng/mL MAb Anti 6HIS—Tb cryptate Gold) and incubated for 4 hours at 25° C. TR-FRET signals were then read on Tecan Spark multimode microplate reader. The parameters were F486: Excitation 340 (35) nm, Emission 486 (10) nm, Lag time 100 μs, Integration time 200 μs; F515: Excitation 340 (35) nm, Emission 515 (10) nm, Lag time 100 μs, Integration time 200 μs. TR-FRET ratios for each individual wells were calculated by equation: TR-FRET ratio=(Signal F515/Signal F486)*10000. Then the data were analyzed using a 4-parameter logistic model to calculate IC₅₀ values. The following table lists the IC₅₀ values of certain compounds of the invention.

| Example | Kras G12V(nM) |
|---|---|
| 1 | 4.37 |
| 2 | 0.54 |
| 3 | 39.06 |
| 4 | 95.81 |
| 5 | 0.19 |
| 6 | 0.38 |
| 7 | 5.87 |
| 8 | 22.95 |
| 9 | 3.15 |
| 10 | 5.1 |
| 11 | 3.5 |
| 12 | 49.7 |
| 13 | 13.7 |

### Biological Example 4: In Vitro Anti-Proliferation Assay in Cancer Cell Line with Kras Mutation

Cell antiproliferation was assayed by PerkinElmer ATPlite™ Luminescence Assay System. Briefly, the various test cancer cell lines were plated at a density of about 1×10⁴ cells per well in Costar 96-well plates, and were incubated with different concentrations of compounds for about 72 hours in medium supplemented with 5% FBS or 10% normal human serum(NHS). One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at −700 rpm. This procedure was used to lyse the cells and to stabilize the ATP. Next, 50 μL substrate solution was added to the wells and microplate was shaken for five minutes in an orbital shaker at −700 rpm. Finally, the luminescence was measured by a PerkinElmer TopCount® Microplate Scintillation Counter. Such assays, carried out with a range of doses of test compounds, allowed the determination of the cellular anti-antiproliferative IC₅₀ of the compounds of the present invention.

### Biological Example 5: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example 6: In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach −100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

What is claimed is:

1. A compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof: 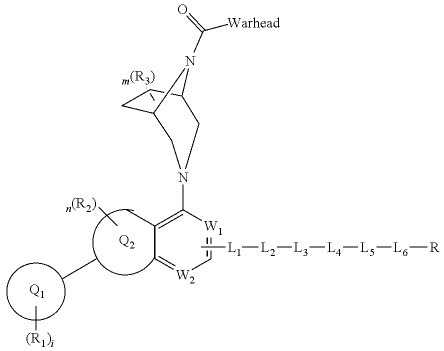
wherein
Warhead is chemical group that can form a covalent bond with a Cysteine of the target protein;
each of Q₁, and Q₂, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
R is R₀ or a small molecule (e.g., with a molecular weight of no more than 1000 Da, 800 Da, 500 Da, 300 Da, or 200 Da) E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}, with the priviso that at least one R₃ is not H;
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkenyl, bivalent alkynyl, bivalent cycloalkyl, bivalent cycloalkenyl, bivalent spirocycloalkyl, bivalent fused-carbocyclic, bivalent bridged-carbocyclic, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (1-A): 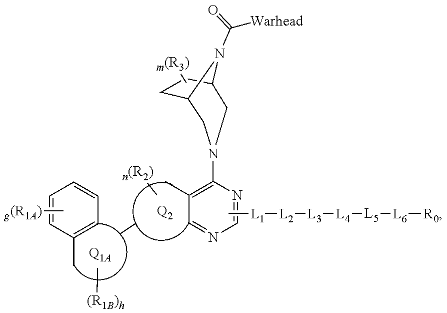
wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is 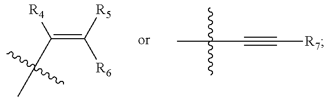
and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-B):

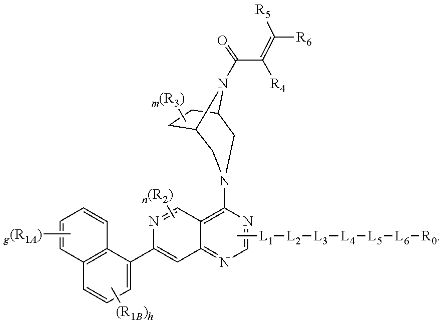

4. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-C):

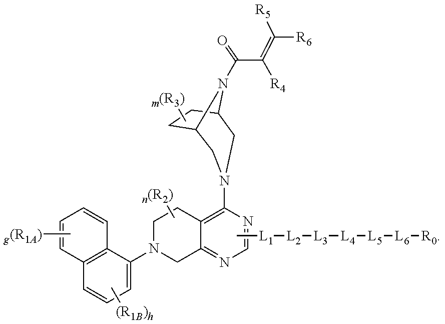

5. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-D):

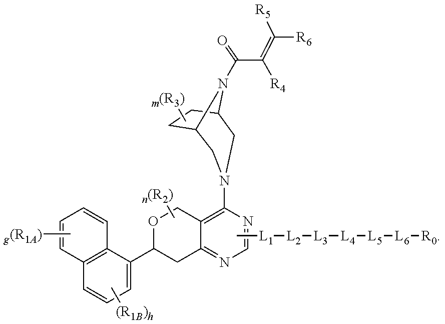

6. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-E):

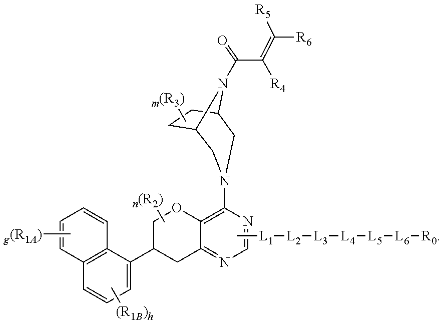

7. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-F):

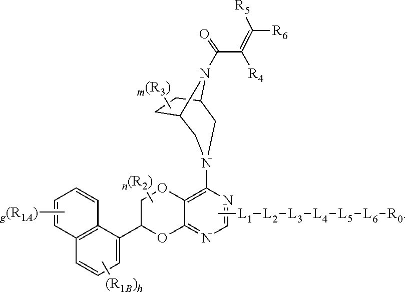

8. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-G):

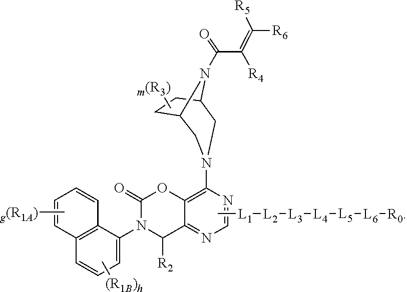

9. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-H):

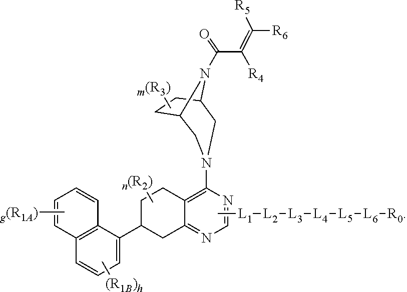

10. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-I):

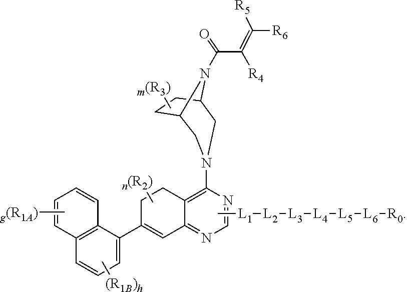

11. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-J): 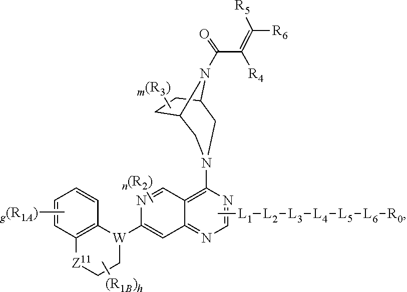
wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

12. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-K): 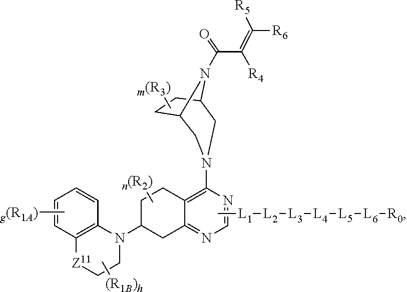
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

13. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-L): 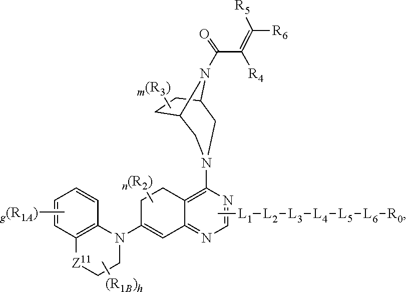
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

14. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-M): 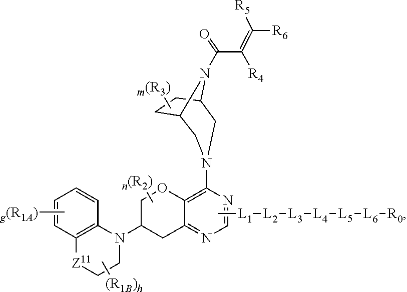
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

15. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (1-N): 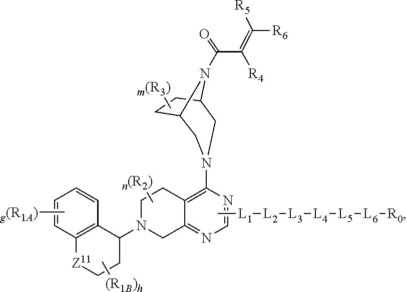
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

16. A pharmaceutical composition comprising a compound of Formula (1) or an N-oxide thereof as defined in any one of claims 1-15, or a pharmaceutically acceptable salt, solvate, polymorph, tautomer, stereoisomer, an isotopic form, or a prodrug of said compound of Formula (I) or an N-oxide thereof, and a pharmaceutically acceptable diluent or carrier.

17. A method of treating a neoplastic disease, autoimmune disease, and inflammatory disorder, comprising administering to a subject in need thereof an effective amount of a compound of Formula (1) or an N-oxide thereof as defined in any one of claims 1-15, or a pharmaceutically acceptable salt, solvate, polymorph, tautomer, stereoisomer, an isotopic form, or a prodrug of said compound of Formula (1) or an N-oxide thereof.

18. A compound of Formula (2), or an N-oxide thereof, or a pharmaceutically acceptable salt, solvate, polymorph, tautomer, stereoisomer, an isotopic form, or a prodrug of said compound of Formula (2) or N-oxide thereof: 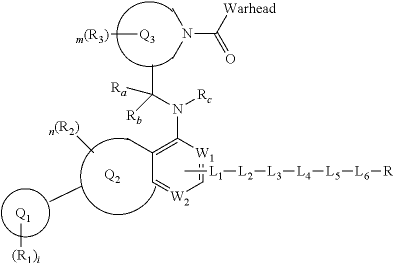
wherein
Warhead is chemical group that can form a covalent bond with a Cysteine of the target protein;
each of Q₁, and Q₂, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
Q₃ is heterocycloalkyl, heterocycloalkenyl, spiro-heterocyclic, fused-heterocyclic, or bridged-heterocyclic, each of the aforementioned is independently optionally substituted with one or more R_{d};
R is R₀ or a small molecule E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

19. The compound according to claim 18 or an N-oxide thereof, or a pharmaceutically acceptable salt, solvate, polymorph, tautomer, stereoisomer, an isotopic form, or a prodrug thereof, wherein the compound is represented by Formula (2-A): 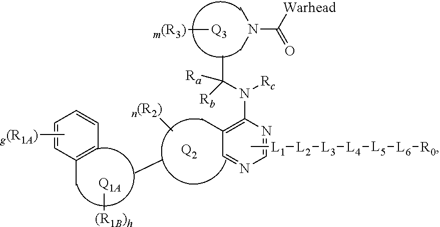
wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is 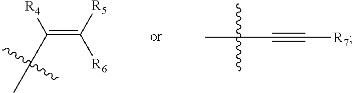
and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.

20. The compound according to claim 19 or an N-oxide thereof, or a pharmaceutically acceptable salt, solvate, polymorph, tautomer, stereoisomer, an isotopic form, or a prodrug thereof, wherein the compound is represented by Formula (2-B):

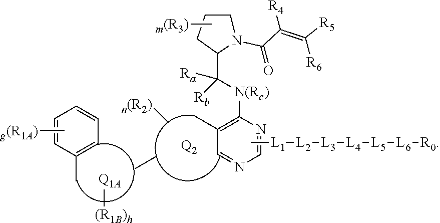

21. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-C):

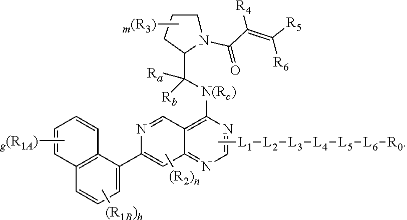

22. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-D):

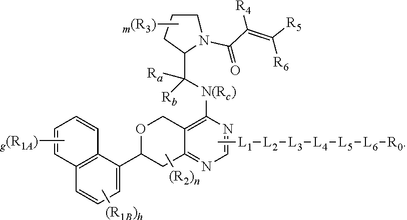

23. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-E):

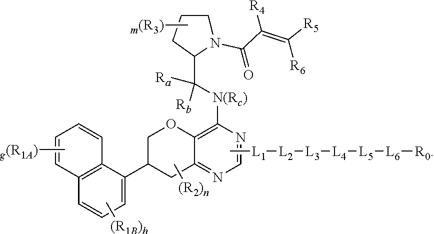

24. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-F):

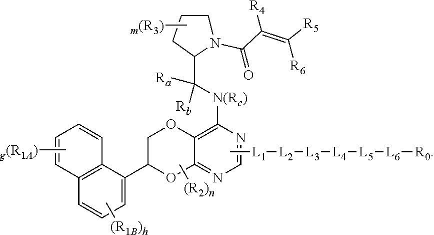

25. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-G):

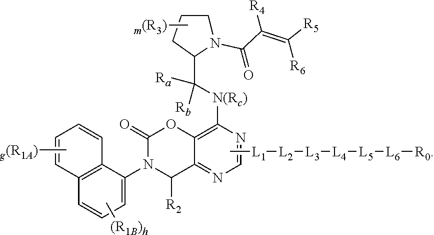

26. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-H):

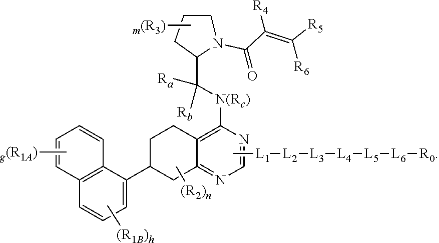

27. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-I):

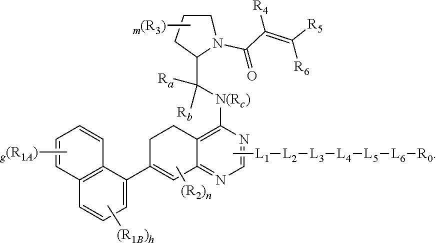

28. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-J): 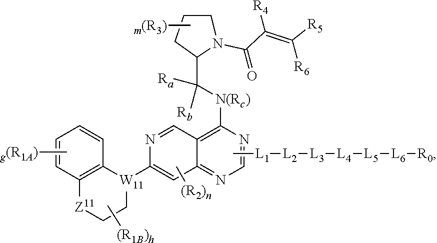
wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

29. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-K): 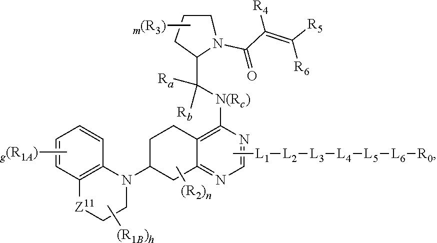
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

30. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-L): 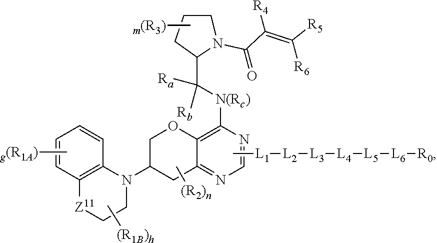
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

31. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-M): 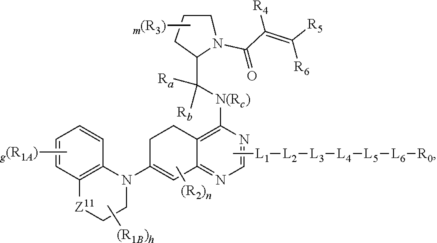
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

32. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-N): 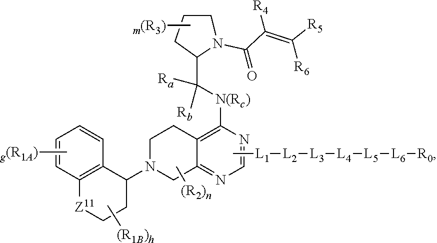
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

33. The compound according to claim 20 or an N-oxide thereof, or a pharmaceutically acceptable salt, solvate, polymorph, tautomer, stereoisomer, an isotopic form, or a prodrug thereof, wherein the compound is represented by Formula (2-O):

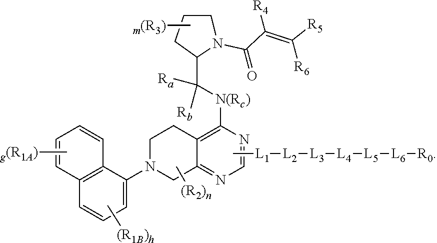

34. A pharmaceutical composition comprising a compound of Formula (2) or an N-oxide thereof as defined in any one of claims 18-33, or a pharmaceutically acceptable salt, solvate, polymorph, tautomer, stereoisomer, an isotopic form, or a prodrug of said compound of Formula (2) or an N-oxide thereof, and a pharmaceutically acceptable diluent or carrier.

35. A method of treating a neoplastic disease, autoimmune disease, and inflammatory disorder, comprising administering to a subject in need thereof an effective amount of a compound of Formula (2) or an N-oxide thereof as defined in any one of claims 18-33, or a pharmaceutically acceptable salt, solvate, polymorph, tautomer, stereoisomer, an isotopic form, or a prodrug of said compound of Formula (2) or an N-oxide thereof.

36. A compound of Formula (3), or an N-oxide thereof, or a pharmaceutically acceptable salt, solvate, polymorph, tautomer, stereoisomer, an isotopic form, or a prodrug of said compound of Formula (3) or N-oxide thereof: 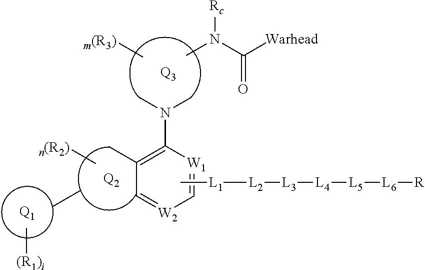
wherein
Warhead is chemical group that can form a covalent bond with a Cysteine of the target protein;
each of Q₁ and Q₂, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
Q₃ is heterocycloalkyl, heterocycloalkenyl, spiro-heterocyclic, fused-heterocyclic, bridged-heterocyclic, or heteroaryl, each of the aforementioned is independently optionally substituted with one or more R_{d};
R is R₀ or a small molecule E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

37. The compound according to claim 36 or an N-oxide thereof, or a pharmaceutically acceptable salt, solvate, polymorph, tautomer, stereoisomer, an isotopic form, or a prodrug thereof, wherein the compound is represented by Formula (3-A): 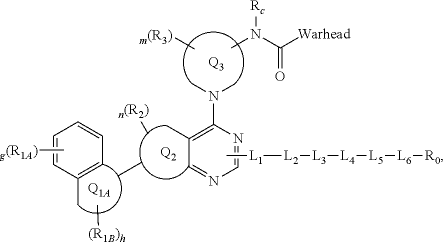
wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is 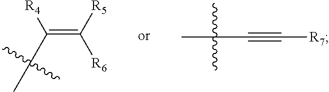
and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.

38. The compound according to claim 37 or an N-oxide thereof, or a pharmaceutically acceptable salt, solvate, polymorph, tautomer, stereoisomer, an isotopic form, or a prodrug thereof, wherein the compound is represented by Formula (3-B): 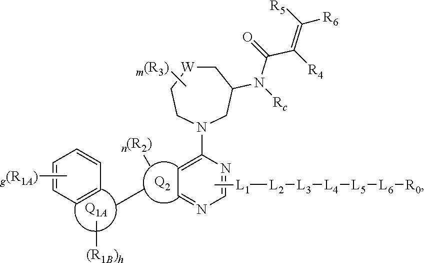
wherein
W is C(RₐR_{b}), N(R_{c}), O, S, or S(O₂).

39. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-C):

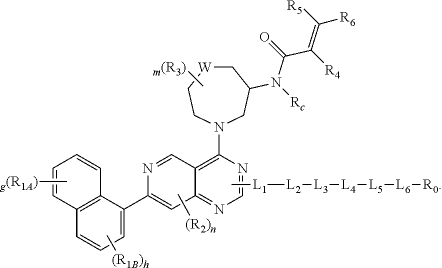

40. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-D):

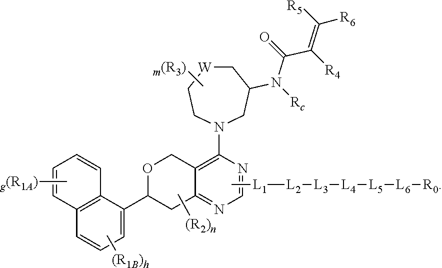

41. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-E):

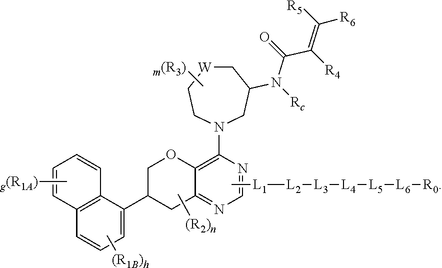

42. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-F):

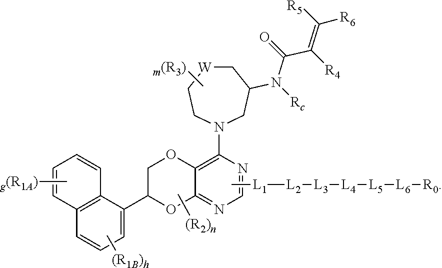

43. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-G):

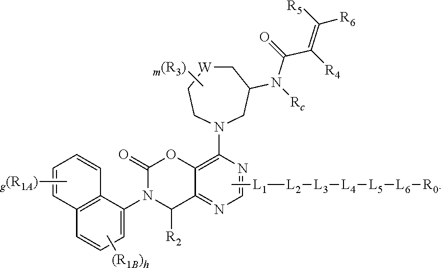

44. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-H):

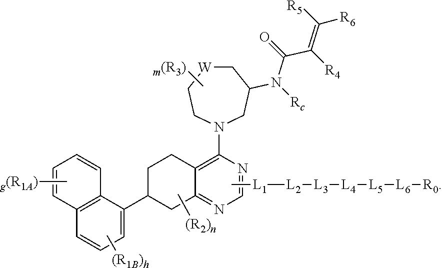

45. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-1):

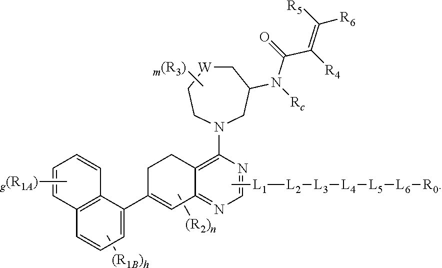

46. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-J): 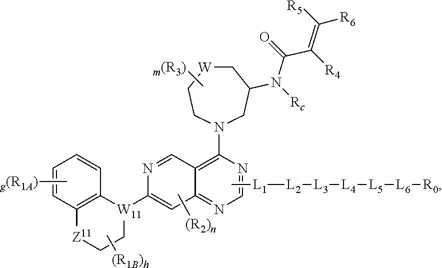
wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

47. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-K): 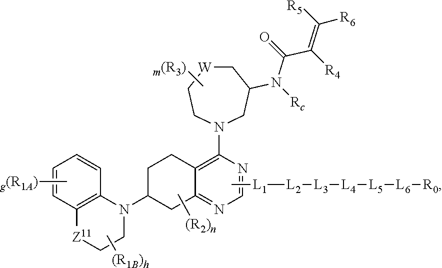
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

48. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-L): 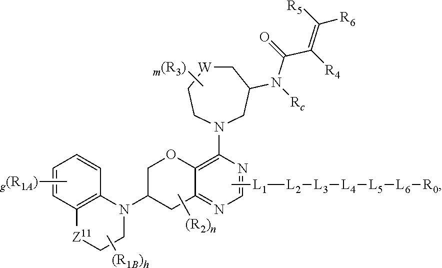
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

49. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-M): 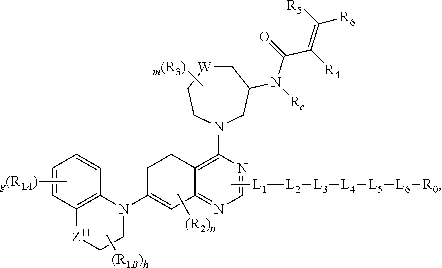
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

50. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-N): 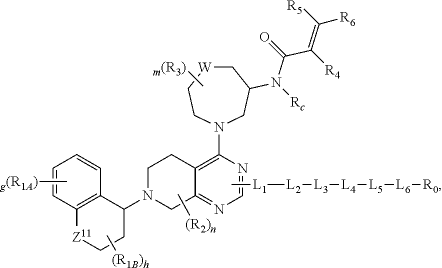
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

51. The compound according to claim 38 or an N-oxide thereof, or a pharmaceutically acceptable salt, solvate, polymorph, tautomer, stereoisomer, an isotopic form, or a prodrug thereof, wherein the compound is represented by Formula (3-O):

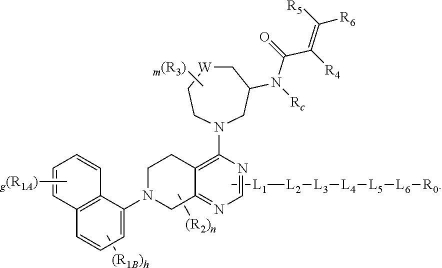

52. A pharmaceutical composition comprising a compound of Formula (3) or an N-oxide thereof as defined in any one of claim 36-51, or a pharmaceutically acceptable salt, solvate, polymorph, tautomer, stereoisomer, an isotopic form, or a prodrug of said compound of Formula (3) or an N-oxide thereof, and a pharmaceutically acceptable diluent or carrier.

53. A method of treating a neoplastic disease, autoimmune disease, and inflammatory disorder, comprising administering to a subject in need thereof an effective amount of a compound of Formula (3) or an N-oxide thereof as defined in any one of claims 36-51, or a pharmaceutically acceptable salt, solvate, polymorph, tautomer, stereoisomer, an isotopic form, or a prodrug of said compound of Formula (3) or an N-oxide thereof.

54. A compound of Formula (4), or an N-oxide thereof, or a pharmaceutically acceptable salt, solvate, polymorph, tautomer, stereoisomer, an isotopic form, or a prodrug of said compound of Formula (4) or N-oxide thereof: 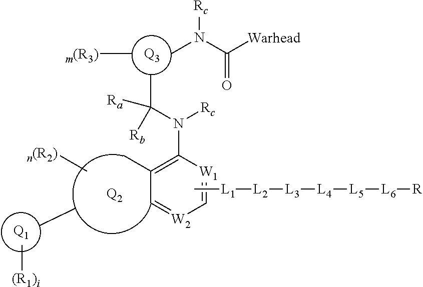
wherein
Warhead is chemical group that can form a covalent bond with a Cysteine of the target protein;
each of Q₁, Q₂ and Q₃, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
R is R₀ or a small molecule E3 ubiquitin ligase binding moiety that binds an E3 ubiquitin ligase;
each of R₀, R₁, R₂, and R₃, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of W₁, and W₂, independently, is N or C(Rₐ);
each of L₁, L₂, L₃, L₄, L₅, and L₆, independently, is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c)}C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkyl, bivalent alkenyl, bivalent alkynyl, bivalent cycloalkyl, bivalent cycloalkenyl, bivalent fused-carbocyclic, bivalent bridged-carbocyclic, bivalent spirocycloalkyl, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl;
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
Rₐ and R_{b}, groups, taken together with the atom to which they are attached, may optionally form a cycloalkyl or heterocycloalkyl optionally substituted with one or more Rₑ;
R_{b} and R_{c}, groups, taken together with the atom to which they are attached, may optionally form a cycloalkyl or heterocycloalkyl optionally substituted with one or more Rₑ;
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f}; and
each of m, n, and i, independently, is 0, 1, 2, 3, 4, 5, 6, 7, 8, 9, 10, 11, or 12.

55. The compound according to claim 54 or an N-oxide thereof, or a pharmaceutically acceptable salt, solvate, polymorph, tautomer, stereoisomer, an isotopic form, or a prodrug thereof, wherein the compound is represented by Formula (4-A): 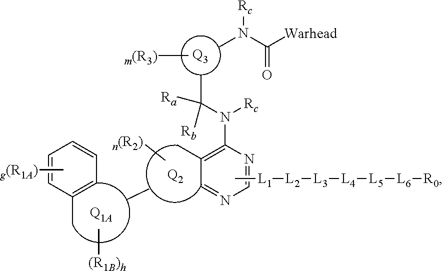
wherein
Q_{1A} is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
g is 0, 1, 2, 3, or 4; h is 0, 1, 2, or 3; and g+h=i;
R_{1A} and R_{1B}, independently, is R₁,
Warhead is 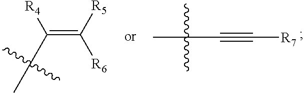
and
each of R₄, R₅, R₆, and R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, fused-carbocyclic, bridged-carbocyclic, spirocycloalkyl, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.

56. The compound according to claim 55 or an N-oxide thereof, or a pharmaceutically acceptable salt, solvate, polymorph, tautomer, stereoisomer, an isotopic form, or a prodrug thereof, wherein the compound is represented by Formula (4-B): 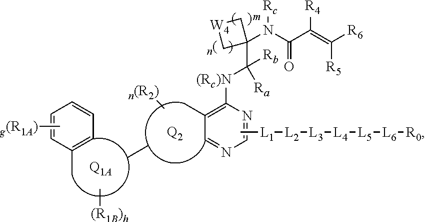
wherein
W₄ is C(RₐR_{b}) or N(Rₐ);
each of m, and n, independently, is 0, 1, 2, or 3.

57. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-C):

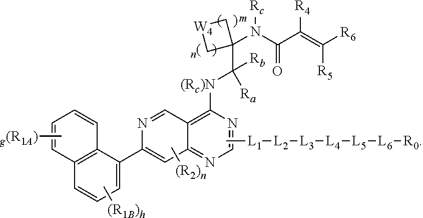

58. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-D):

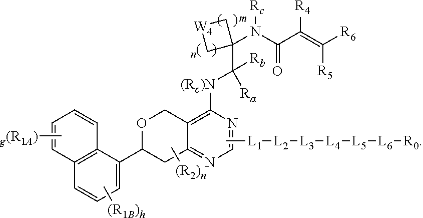

59. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-E):

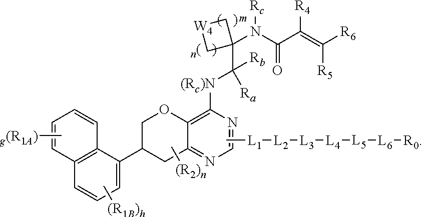

60. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-F):

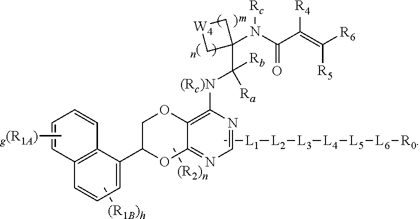

61. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-G):

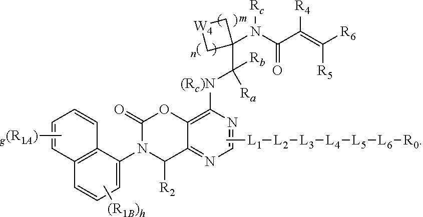

62. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-H):

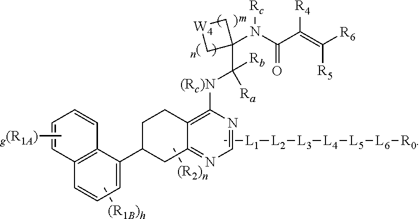

63. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-I):

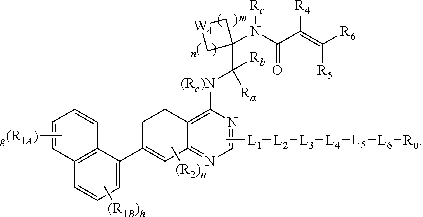

64. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-J): 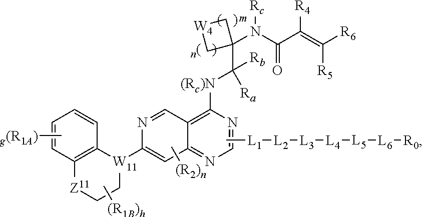
wherein
W₁₁ is N, or C(Rₐ); and
Z¹ is absent, a bond, (CRₐR_{b})ₚ, N(R_{c}), O, S, C(O), S(O₂), —O(CRₐR_{b})ₚ—, —N(R_{c})(CRₐR_{b})ₚ, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

65. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-K): 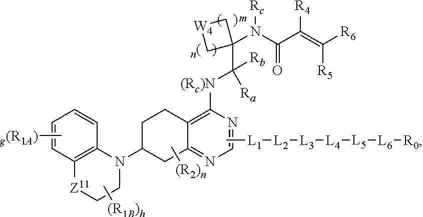
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

66. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-L): 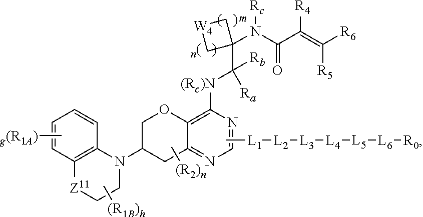
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

67. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-M): 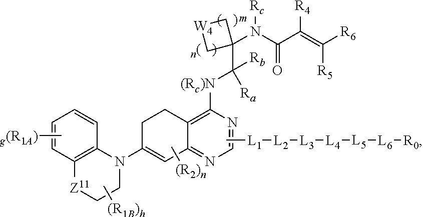
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

68. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-N): 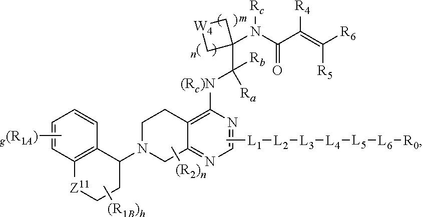
wherein Z¹¹ is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, or (CRₐR_{b})ₚC(O)N(R_{c})(CRₐR_{b})_{q}.

69. The compound according to claim 56 or an N-oxide thereof, or a pharmaceutically acceptable salt, solvate, polymorph, tautomer, stereoisomer, an isotopic form, or a prodrug thereof, wherein the compound is represented by Formula (4-O):

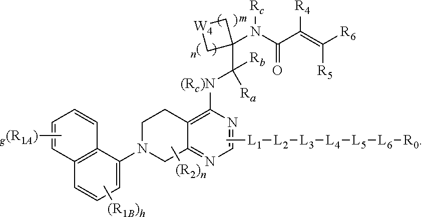

70. A pharmaceutical composition comprising a compound of Formula (4) or an N-oxide thereof as defined in any one of claims 54-69, or a pharmaceutically acceptable salt, solvate, polymorph, tautomer, stereoisomer, an isotopic form, or a prodrug of said compound of Formula (4) or an N-oxide thereof, and a pharmaceutically acceptable diluent or carrier.

71. A method of treating a neoplastic disease, autoimmune disease, and inflammatory disorder, comprising administering to a subject in need thereof an effective amount of a compound of Formula (4) or an N-oxide thereof as defined in any one of claims 54-69, or a pharmaceutically acceptable salt, solvate, polymorph, tautomer, stereoisomer, an isotopic form, or a prodrug of said compound of Formula (4) or an N-oxide thereof.
